(19) United States
(12) Patent Application Publication
(10) Pub. No.: US 2006/0183128 A1
(43) Pub. Date: Aug. 17, 2006

(54) Methods and compositions for differentiating tissues for cell types using epigenetic markers

(75) Inventors: Kurt Berlin, Stahnsdorf (DE); Alexander Olek, Berlin (DE); Stephan Beck, Cambridge (GB); Thomas Hildmann, Berlin (DE); Joern Lewin, Berlin (DE); Karen Novik, North Vancouver (CA)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Epigenomics AG, Berlin (DE)

(21) Appl. No.: 10/641,321

(22) Filed: Aug. 12, 2003

(51) Int. Cl. C12Q 1/68; C12P 19/34

(52) U.S. Cl. 435/6; 435/91.2

(57) ABSTRACT

The present invention provides, inter alia, a method for generating a genome-wide epigenomic map, comprising a correlation between methylation variable CpG positions (MVP) and genomic DNA sample types. MVP are those CpG positions that show a variable quantitative level of methylation between sample types. Particular genomic regions of interest (ROI) provide preferred marker sequences that comprise multiple, and preferably proximate MVP, and that have novel utility for distinguishing sample types. The epigenic maps have broad utility, for example, in identifying sample types, or for distinguishing between and among sample types. In a preferred embodiment the epigenomic map is based on methylation variable regions (MVP) within the major histocompatibility complex (MHC), and has utility, for example, in identifying the cell or tissue source of a genomic DNA sample, or for distinguishing one or more particular cell or tissue types among other cell or tissue types. Analysis of epigenetic characteristics of one, or of a set of nucleic acid sequences, in the context of an inventive epigenomic map, allows for the determination of an origin of the nucleic acids.

### FIELD OF THE INVENTION

The invention relates to the field of molecular diagnostic markers, and novel method for generating a genome-wide epigenomic map, comprising a correlation between methylation variable CpG positions (MVP) and genomic DNA sample types. The inventive epigenic maps have broad utility, for example, in identifying sample types, or for distinguishing between and among sample types. In particular preferred embodiments, the invention describes novel epigenetic characteristics of nucleic acid sequences derived from the major histocompatibility complex (MHC) and use of such markers to identify and/or differentiate tissues or cell types.

### SEQUENCE LISTING

A Sequence Listing, pursuant to 37 C.F.R. § 1.52(e)(5), has been provided on compact disc (1 of 1) as a 6.105 MB file, entitled 47675-49.txt, and which is incorporated by reference herein in its entirety.

### BACKGROUND

Genomic methylation. The genome contains approximately 40 million methylated cytosine (5-methylcytosine) bases, otherwise referred to herein as “fifth” bases, which are followed immediately by a guanine residue in the DNA sequence, with CpG dinucleotides comprising about 1.4% of the entire genome. An unusually high proportion of these bases is located in the regulatory and coding regions of genes. Methylation of cytosine residues in DNA is currently thought to play a direct role in controlling normal cellular development. Various studies have demonstrated that a close correlation exists between methylation and transcriptional inactivation. Regions of DNA that are actively engaged in transcription, however, lack 5-methylcytosine residues.

Methylation patterns, comprising multiple CpG dinucleotides, also correlate with gene expression, as well as with the phenotype of many of the most important common and complex human diseases. Methylation positions have, for example, not only been identified that correlate with cancer, as has been corroborated by many publications, but also with diabetes type II, arteriosclerosis, rheumatoid arthritis, and disease of the CNS. Likewise, methylation at other positions correlates with age, gender, nutrition, drug use, and probably a whole range of other environmental influences. Methylation is the only flexible (reversible) genomic parameter under exogenous influence that can change genome function, and hence constitutes the main (and so far missing) link between the genetics of disease and the environmental components that are widely acknowledged to play a decisive role in the etiology of virtually all human pathologies that are the focus of current biomedical research.

Methylation plays an important role in disease analysis because methylation positions vary as a function of a variety of different fundamental cellular processes. Additionally, however, many positions are methylated in a stochastic way, that does not contribute any relevant information.

Methylation content, levels, profiles and patterns. Genomic methylation can be characterized in distinguishable terms of methylation content, methylation level and methylation patterns. “Methylation content,” or “5-methylcytosine content,” as used herein refers to the total amount of 5-methylcytosine present in a DNA sample (i.e., a measure of base composition), and provides no information as to distribution of the fifth bases. Methylation content of the genome has been shown to differ, depending on the tissue source of the analyzed DNA (Ehrlich M, et al., Nucleic Acids Res. 10:2709, 1982). However, while Ehrlich et al showed tissue- and cell specific differences in methylation content among seven different normal human tissues and eight different types of homogeneous human cell populations, their analysis was neither specific with respect to particular genome regions, nor with respect to particular CpG positions. No genes or CpG positions were selected for the analysis, or identified by the analysis that could serve as markers for tissue or cell identification. Rather, only the level of the overall degree of genomic methylation (methylation content) was determined.

“Methylation level” or “methylation degree,” by contrast, refers to the average amount of methylation present at an individual CpG dinucleotide. Measurement of methylation levels at a plurality of different CpG dinucleotide postions creates either a methylation profile or a methylation pattern.

A methylation profile is created when average methylation levels of multiple CpGs (scattered throughout the genome) are collected. Each single CpG position is analyzed independently of the other CpGs in the genome, but is analyzed collectively across all homologous DNA molecules in a pool of differentially methylated DNA molecules (Huang et al., in The Epigenome, S. Beck and A. Olek, eds., Wiley-VCH Weinheim, p 58, 2003).

A methylation pattern, by contrast, is composed of the individual methylation levels of a number of CpG positions in proximity to each other. For example, a full methylation of 5-10 closely linked CpG positions may comprise a methylation pattern that, while rare, may be specific for a specific DNA source.

Prior art correlations involving DNA methylation. A correlation of individual gene methylation patterns with specific tissues has been suggested in the art (Grunau et al., Hum. Mol. Gen. 9:2651-2663, 2000). However, in this study, methylation patterns of only four specific genes were analyzed in tissues from only two different individuals, and the aim of the study was to analyze the correlation between known gene expression levels and their respective methylation patterns.

Adorjan et al published data indicating that tissues such as prostate and kidney could be distinguished by means of methylation markers (Adorjan et al., Nuc. Acids Res. 30: e 21, 2002). This study identified tumor markers, based on analysis of a large number of individuals (relatively large number of samples). Several CpG positions were identified that could be utilized as markers in an appropriate methylation assay to differentiate between kidney and prostate tissue, regardless of the tissue status as being diseased or healthy.

However both the Grunau et al., and Adorjan et al studies offer only a very limited selection of markers to detect a very small proportion of the many known different cell types.

Likewise, patent application WO 03/025215 to Carroll et al., for example, provides a method for creating a map of the methylome (referred to as “a genomic methylation signature”), based on methylation profile analyses, and employing methylation-sensitive restriction enzyme digests and digest-dependant amplification steps. The method description alleges to combine methylation profiling with mapping. This attempt is, however, severely limited for at least three reasons. First, the prior art method provides only a ‘yes or no’ qualitative assessment of the methylation status (methylated or unmethylated) of a cytosine at a genomic CpG position in the genome of interest.

Second, the method of Carroll et al is labor intensive, not being adaptable for high throughput, because it requires a second labor intensive step; namely, after completing the process of restriction enzyme-based methylation analysis to identify a particular amplificate as a potential methylation marker, each of these amplified digestion dependent markers (amplficates) needs to be cloned and sequenced for mapping to the genome.

Third, there are no means described by Carroll et al for utilizing the generated information as tissue markers. Specifically, while Carroll et al disclose that specific different tissues of mice have different ‘methylomes’ (WO 03/025215, FIG. 6), and that two different human tissues, sperm cells and blood cells, could be correlated with differing amplification profiles (Id, FIGS. 4 and 10, where CpG positions were identified that were unmethylated in one scenario and methylated in the other), there is no means or enablement to support use of this information as a specific tissue marker.

Prior art methods for determining tissue type that are based on protein or mRNA expression are limited by intrinsic disadvantages.

Protein expression-based prior art approaches. Immuno-histochemical assays are utilized as standard methods to determine a cell type or a tissue type of cellular origin in the context of an intact organism. Such methods are based on the detection of specific proteins. For example, the German Center for collection of microorganisms and cell cultures (DSMZ) routinely tests the expression of tissue markers on all arriving human cell lines with a panel of well-characterized monoclonal antibodies (mAbs) (Quentmeier H, et al., J Histochem. Cytochem. 49:1369-1378, 2001). Generally, the expression pattern of histological markers reflects that of the originating cell type. However, expression of the proteins, carbohydrate or lipid structures that are detected by individual mAbs, is not always stable over a long period of time.

Likewise, immunophenotyping, which can be performed both to confirm the histological origin of a cell line, and to provide customers with useful information for scientific applications, is based on testing the stability and intensity of cell surface marker expression. Immunophenotyping typically includes a two-step staining procedure, wherein antigen-specific murine mAbs are added to the cells in the first step, followed by assessment of binding of the mAbs by an immunofluorescence technique using FITC-conjugated anti-mouse Ig secondary antisera. Distribution of antigens is analyzed by flow cytometry and/or light microscopy.

A number of proteins appear to be expressed in a tissue- or organ-specific manner. However, not only is their use as markers restricted to the rather labor-intensive procedure of immunostaining, but these methods also limited by a requirement for intact cells and a sufficient amount of tissue material or cells, in a non-degraded/non-denatured state. With respect to serum, proteins, as well as RNAs, that are “exogenous” to the blood stream will be degraded fast and therefore are not adequately available in many instances for determination of the respective tissues of origin.

Additionally, one assay per protein is required to monitor the expression of the proteins. Therefore the process of determining a cell type or tissue type using these expression-based methods is not trivial, but rather complex. The more marker proteins are known the more precisely a cell's status of origin can be determined. Without the use of molecular biology techniques, such as RNA-based cDNA/oligo-microarrays or a complex proteomics experiment, which enable the simultaneous view of a higher number of changes, the identification of a specific cell type would require a sequence of tedious and time-consuming assays to detect a rather complex protein expression pattern. Finally, proteomic approaches have not overcome basic difficulties, such as reaching sufficient sensitivity.

RNA expression-based prior art approaches. RNA-based techniques to analyze expression patterns are well-known and widely used. In particular, microarray-based expression analysis studies to differentiate cell types and organs have been described, and used to show that precise patterns of differentially expressed genes are specific for a particular cell type.

A system of cluster analysis for genome-wide expression data from DNA microarray hybridization is described by Eisen et al. Proc. Natl. Acad. Sci. USA. 95:14863-8, 1998. Eisen et al teach clustering of gene expression data groups together, especially data for genes of known similar function, and interpretation of the patterns found as an indicator of the status of cellular processes. However, the teachings of Eisen are in the context of yeast and, therefore, cannot be extended to identify tissue or organ markers useful in human beings or other more developmentally complex organisms and animals. Likewise such teachings cannot be extended into the area of human disease prognostics and diagnostics.

Similarly, Ben-Dor et al describe an expression-based approach for tissue classification in humans. However, as in nearly all related publications, the scope is limited to markers for the identification of tumors (Ben-Dor et al. J Comput Biol. 7: 559-83, 2000).

Likewise, Enard et al. recently published a comparative analysis of expression patterns within specific tissue samples across different species, teaching different mRNA and protein expression patterns between different individuals of one species (intra-specific variation), as well as between different species (inter-specific variation). Enard et al did not however, teach or enable use of such expression levels for distinguishing between or among different tissues.

Both cDNA arrays and oligonucleotide-based-chips (e.g., Affymetrix™ chips) allow a complex and sensitive analysis of changes in the expression pattern of cells. However, the substantial drawback of these technologies is their dependency on RNA. Despite extensive research with RNA, the general problem of its instability is still not solved, and each single experiment with RNA must account for RNA degradation during the experimental procedure. This problem is aggravated by the fact that RNA expression levels change gradually, so that for the majority of genes, the actual expression changes are overlapping and blurred, because of random degradation.

Lack of acceptance of prior art methods by regulatory agencies. Significantly, regulatory agencies are currently not willing to accept a technology platform relying on an expression microarray due to the above-described shortcomings.

U.S. Pat. No. 6,581,011 to TissueInformatics Inc., teaches a tissue information database for profiling and classifying a broad range of normal tissues, and illustrates the need in the art for tools allowing classification of a tissue.

Prior art ‘tumor marker’ gene approaches. More and more nucleic acid-based assays are developed today for detecting the presence or absence of known tumor indicating proteins in blood or other bodily fluids, or of mRNAs of known tumor related genes; so-called tumor marker genes. Such assays are distinguished from those based on screening DNA for mutations indicative of hereditary diseases, wherein not only mRNA but also genomic DNA can be analyzed, but wherein no information can be gathered on the actual condition of the patient.

For detection of acute disease status using marker gene approaches, the analyzed DNA must be derived from a diseased cell, such as a tumor cell. The detection of cancer specific alterations of genes involved in carcinogenesis (e.g., oncogene mutations or deletions, tumor suppressor gene mutations or deletions, or microsatellite alterations) facilitates determining the probability that a patient carries a tumor or not (e.g., WO 95/16792 or U.S. Pat. No. 5,952,170 to Stroun et al.). Kits, in some instances, have been developed that allow for efficient and accurate screening of multiple samples. Such kits are not only of interest for improved preventive medicine and early cancer detection, but also utility in monitor a tumors progression/regression after therapy.

Marker gene hypermethylation. Hypermethylation of certain ‘tumor marker’ genes, especially of certain promoter regions thereof, is recognized as an important indicator of the presence or absence of a tumor. Significantly, however, such prior art methylation analyses are limited to those based on determination of the methylation status of known marker genes, and do not extent to genomic regions that have not been previously implicated based on function; ‘tumor marker’ genes are those genes known to play a role in the regulation of carcinogenesis, or are believed to determine the switching on and off of tumorigenesis.

Knowledge of the correlation of methylation of tumor marker genes and cancer is most advanced in the case of prostate cancer. For example, a method using DNA from a bodily fluid, and comprising the methylation analysis of the tumor marker gene GSTP1 as an predictive indicator of prostate cancer has been patented (U.S. Pat. No. 5,552,277).

Significantly, prior art tumor marker screening approaches are limited to certain types of diseases (e.g., cancer types). This is because they are limited to analysis of marker genes, or gene products which are highly specific for a kind of disease, mostly being cancer, when found in a specific kind of bodily fluid. For example, Usadel et al. teach detection of a tumor specific methylation in the promoter region of the adenomatous polyopsis coli (APC) gene in serum samples of lung cancer patients, but that no methylated APC promoter DNA is detected in serum samples of healthy donors (Usadel et al. Cancer Research 6:371-375, 2002). This marker thus qualifies as a reasonable indicator for lung cancer, and has utility for the screening of people diagnosed with lung cancer, or for monitoring of patients after surgical removal of a tumor for developing metastases in their lung.

Moreover the teachings of Usadel et al are also limited by the fact that the epigenetic APC gene alterations are not specific for lung cancer, but are common in other cancer, for example, in gastrointestinal tumor development. Therefore, a blood screen with only APC as a tumor marker has limited diagnostic utility to indicate that the patient is developing a tumor, but not where that tumor would be located or derived from. Consequently, a physician would not be informed with respect to a more detailed diagnosis of an specific organ, or even with respect to treatment options of the respective medical condition; most of the available diagnostic or therapeutic measures will be organ- or tumor source-specific. This is particularly true where the lesion is small in size, and it will be extremely difficult to target further diagnostics and therapies.

Given the nature of marker genes as previously implicated genes, prior art use of marker genes for early diagnosis has occurred where a specific medical condition is already in mind. For example, a physician suspicious of having a patient with a developed a colon cancer, can have the patient stool sample tested for the status of a cancer marker gene like K-ras. A patient suspected as having developed a prostate cancer, may have his ejaculate sample tested for a prostate cancer marker like GSTPi.

Significantly, however, there is no prior art method described for efficient and effective generally screening of patients, or bodily fluids thereof, where the patient has no specific prior indication or suspicion as to which organ or tissue might have developed a cell proliferative disease (e.g., an individual previsously exposed to a high level of radiation). In particular cases, the use of appropriate tissue specific markers however, may allow this kind of diagnosis (e.g., application PCT/EP03/02245 by Berlin and Sledziewski; teaches a method comprising performing methylation on nucleic acid samples isolated from bodily fluids, and wherein an increased level of circulating nucleic acids is detectable.

The major histocompatibility complex. The major histocompatibility complex (MHC) is essential to our immune system, and thus is associated with more diseases than any other region of the human genome. For example, factors affecting psoriasis, a common hereditary skin disease, are linked to the MHC. The primary immunological function of MHC molecules is to bind and ‘present’ antigenic peptides on the surfaces of cells for recognition (binding) by the antigen-specific T cell receptors (TCRs) of lymphocytes. Differential structural properties of MHC class I and class II molecules account for their respective roles in activating different populations of T lymphocytes; cytotoxic TC lymphocytes bind antigenic peptides presented by MHC class I molecules, whereas, helper TH lymphocytes bind antigenic peptides presented by MHC class II molecules.

The MHC is a region of a defined range, and as such is one of the best characterized regions in the human genome. Highly reliable sequence information is available throughout this range. It is not yet clear, however, which MHC regions might have utility for identification and/or distinguishing between or among disease states or conditions.

Inadequate genome-wide screening approaches. Unfortunately, prior art approaches to genome-wide assessment of CpG dinucleotides all employ the digestion of genomic DNA with methylation-sensitive enzymes, thereby limiting analysis to sites for which methylation-sensitive enzymes are available. Most of these techniques are highly labor intensive and cannot be automated.

There is, therefore, a substantial need in the art for a high-throughput approach for efficiently screening the entire genome to assess the methylation status and level of the CpG positions within many genes in parallel.

There is a substantial need for methods that are based on the relatively stable DNA molecule, rather than on easily degradable RNA molecules, and that are more sensitive and reliable than those based on RNA-dependent technologies.

There is a need for diagnostic platforms that are likely to be accepted by regulatory authorities.

There is a substantial need in the art to know which positions in the genome contain disease- or condition-relevant information.

There is a substantial need in the art for a functional map of the ‘epigenome’, displaying the flexible level of higher chromatin organization, and the methylation patterns of genomic segments in relation to external (e.g., environmental) and internal (e.g., cell-type-specific) influences over the course of a human life.

There is a substantial need in the art, including from the clinical perspective, to identify cell or tissue type and/or cell or tissue source. For example, there is a need in the art for efficient and effective typing of disseminated tumor cells, for determining the tissue of origin (i.e., the type of tissue or organ the tumor was derived from).

No such tools or methods, apart from a few disclosed isolated markers, are available in the prior art. Likewise, no generally applicable prior art methods are available for determining the cell- or tissue-type from which a genomic DNA sample was derived.

There is a need in the art for epigonomic methods comprising quantified methylation levels.

### SUMMARY OF THE INVENTION

Particular embodiments of the present invention disclose a method for constructing a functional map of the ‘epigenome.’ Analysis of gene expression (e.g., of RNA, cDNA or protein) is not a requirement for creating the epigenome map, as described and taught herein.

Analysis of genomic DNA bears the advantage of being a reliable method based on a rather robust material, that is much less sensitive to temperature changes and other environmental influences. For example, it is possible to detect genomic DNA derived from a certain organ in the blood stream or other bodily fluids of an individual, wherein they might indicate a disease at the tissue of origin. Accordingly, embodiments of the present invention are based on the relatively stable DNA molecule, rather than on easily degradable RNA molecules, and depends on a digital (0/1) signal (reflecting a binary base status being either methylated or not). Therefore, the present methods are more sensitive and reliable than those based on RNA-dependent technologies. Platform based on the present technology are likely to be accepted by regulatory authorities.

The present invention provides novel methods not only for determining qualitative information for generating methylaion profiles, but also for determining quantitative methylation patterns. The inventive methods provide quantitative information on methylation levels of cytosines at CpG positions within the genome of interest. Such quantitative methods are lacking in the prior art.

In particular embodiments, the invention provides a method for generating quantitative (absolute) methylation level values within a matrix, the matrix comprising along one axis a complete listing of all CpG positions within the human genome, and along another axis a complete listing of all cellular variables or indicia, including but not limited to, cell type, external influences (e.g., environmental influences), age, tissue source type, etc. along the other axis. The field encompassed by these axes is the methylation map of the epigenome (i.e., functional epigenomic map). In preferred exemplary embodiments, a method for generating methylation level values within a sub-matrix comprising all MHC CpG positions, or comprising the CpG positions of particular MHC subregions is provided, said sub-matrices having utility, inter alia, for identifying cell or tissue type, and/or for distinguishing among different cell or tissue types of the respective genomic DNA sources.

According to the present invention, methylation analysis at specific CpG positions allows the determination of the cell- or tissue-type of DNA origin, allowing initiation of further examination for determination of the right treatment in an accurate and efficient manner; particularly crucial where the disease is cancer.

The present invention provides, in particular embodiments, a method to identify a large number of markers, covering the entire genome. The basic method comprises, in particular embodiments, establishing ‘absolute’ values of methylation levels that can be compared across different DNA amplificates and different samples, allowing for a comparison of DNA methylation data corresponding to a diversity of genomic DNA sources and conditions (e.g., corresponding to different isolation methods, different efficiencies of bisulfite pretreatment of the DNA, different amplification/PCR conditions (e.g., different tubes, etc.)).

The present invention provides not only a method for the comprehensive identification of those regions in the genome that after pretreatment become useful markers, but also provides the tools (e.g., the marker nucleic acids and their tissue specific methylation patterns), to identify the organ, tissue or cell type source of the analyzed genomic DNA.

A particularly preferred exemplary embodiment provides a functional map of the major histocompatibility complex (MHC) epigenome, based on a correlation of genomic DNA methylation state or methylation level of particular marker regions with the tissue source of the DNA (i.e., tissue or cell specificity of DNA methylation; differential methylation), rather than on a correlation with environmental influences, like the difference between smoking and non-smoking cell donors. Internal influence in this aspect of the invention relates to the triggers and circumstances that determine a cell's development or differentiation towards a specific cell or tissue type. The method itself however is not limited in utility to tissue differentiation, but is useful to identify marker sequences for all kinds of cell classifications, internal and external.

In a preferred exemplary embodiment described herein, the inventive methods are applied to the human major histocompatibility complex (MHC) region of the genome in screening for tissue-specific markers; that is, for nucleic acid sequences that serve as markers for a specific cell type when used in an appropriate assay according to the present invention. According to the present invention, particular regions of the MHC have been identified that have substantial utility as markers, including as tissue-specific markers.

Specifically, The present invention provides a method for generating a genome-wide methylation map, comprising: obtaining, for each of at least two biological sample types, a plurality or group of biological samples having genomic DNA; pretreating the genomic DNA of the samples by contacting the samples, or isolated DNA from the samples, with an agent, or series of agents that modifies unmethylated cytosine but leaves methylated cytosine essentially unmodified; amplifying segments of the pretreated DNA, said amplified segments representing the entire genome, or a portion thereof, and comprising in each case at least one dinucleotide sequence position corresponding to a CpG dinucleotide position in the corresponding untreated genomic DNA, and wherein said amplification is by means of primer molecules that do not comprise a dinucleotide sequence position corresponding to a CpG dinucleotide position in the corresponding untreated genomic DNA; sequencing the amplified pretreated nucleic acids; analyzing the sequences to quantify a level of methylation at specific CpG positions; comparing said quantified levels of methylation at specific CpG positions between the different sample groups corresponding to the at least two biological sample types; and identifying methylation variable positions, wherein a methylation variable position is a genomic CpG position, for which there is a detectable difference in the quantified level of methylation between different biological sample types, and whereby an epigenomic map over the entire genome, or a portion thereof is, at least in part, afforded.

Preferably, the biological sample type is of a tissue, organ or cell. Preferably, the dinucleotide sequence position corresponding to a CpG dinucleotide position in the corresponding untreated genomic DNA is a CpG or a TpG dinucleotide sequence position. Preferably, sequencing comprises generating a sequence trace, or electropherogram for use in quantifying the level of methylation. Preferably, analyzing the sequences in comprises creating a profile of the quantified level of methylation over the entire genome, or a portion thereof. Preferably, quantifying the level of methylation involves the use of a software program suitable therefore. Preferably, the suitable software program is ESME, which considers or accounts for an unequal distribution of bases in bisulfite converted DNA and normalizes sequence traces (electropherograms) to allow for quantitation of methylation signals within the sequence traces. Preferably, the agent, or series of agents comprises a bisulfite reagent. Preferably, the agent, or series of agents of b) comprises an enzyme. Preferably, pretreating comprises modification of cytosine to uracil. Preferably, amplifying segments comprises amplification of at least one segment located in, or comprising a regulatory region of a gene. Preferably,amplifying in c) comprises use of a polymerase chain reaction (PCR).

Additional embodiments provide a nucleic acid or an oligomer comprising at least one contiguous base sequence having a length of at least 16 nucleotides that is complementary to, or hybridizes under moderately stringent or stringent conditions to a pretreated genomic DNA sequence selected from the group consisting of SEQ ID NOS:1-136, and sequences complementary thereto, wherein said contiguous sequence comprises at least one methylation variable position, or at least one CpG, tpG, or Cpa dinucleotide sequence, and wherein pretreatment comprises treating the genomic DNA with an agent, or series of agents, that modifies unmethylated, but leaves methylated, cytosine essentially unmodified.

Further embodiments provide a set of oligomers, said set comprising a first oligomer and a second oligomer, wherein the first oligomer, and the second oligomer each comprises at least one contiguous base sequence of at least 16 nucleotides in length that is complementary to, or hybridizes under moderately stringent or stringent conditions to a pretreated genomic DNA sequence selected from, in the case of the first oligomer, a first sequence group consisting of SEQ ID NOS:1-136, and selected from, in the case of the second oligomer, a second sequence group consisting of sequences complementary to the sequences of the first sequence group, and wherein pretreatment comprises treating the genomic DNA with an agent, or series of agents, that modifies unmethylated, but leaves methylated, cytosine essentially unmodified. Preferably, the set is suitable for use in generating nucleic acid amplificates.

Yet further embodiments provide a nucleic acid or oligomer, comprising a sequence selected from the group consisting of SEQ ID NOS:137 through 204 and SEQ ID NOS:206 through 221.

Additional embodiments provide a nucleic acid or an oligomer comprising at least one contiguous base sequence having a length of at least 16 nucleotides that is complementary to, or hybridizes under moderately stringent or stringent conditions to a pretreated genomic DNA sequence selected from a group consisting of SEQ ID NOS:1, 2, 69, 70; SEQ ID NOS:3, 4, 71, 72; SEQ ID NOS:5, 6, 73, 74; SEQ ID NOS:7, 8, 75, 76; SEQ ID NOS:9, 10, 77, 78; SEQ ID NOS:11, 12, 79, 80; SEQ ID NOS:13, 14, 81, 82; SEQ ID NOS:15, 16, 83, 84; SEQ ID NOS:17, 18, 85, 86; SEQ ID NOS:19, 20, 87, 88; SEQ ID NOS:21, 22, 89, 90; SEQ ID NOS:23, 24, 91, 92; SEQ ID NOS:25, 26, 93, 94; SEQ ID NOS:27, 28, 95, 96; SEQ ID NOS:29, 30, 97, 98; SEQ ID NOS:31, 32, 99, 100; SEQ ID NOS:33, 34, 101, 102; SEQ ID NOS:35, 36, 103, 104; SEQ ID NOS:37, 38, 105, 106; SEQ ID NOS:39, 40, 107, 108; SEQ ID NOS:41, 42, 109, 110; SEQ ID NOS:43, 44, 111, 112; SEQ ID NOS:45, 46, 113, 114; SEQ ID NOS:47, 48, 115, 116; SEQ ID NOS:49, 50, 117, 118; SEQ ID NOS:51, 52, 119, 120; SEQ ID NOS:53, 54, 121, 122; SEQ ID NOS:55, 56, 123, 124; SEQ ID NOS:57, 58, 125, 126; SEQ ID NOS:59, 60, 127, 128; SEQ ID NOS: 61, 62, 129, 130; SEQ ID NOS:63, 64, 131, 132; SEQ ID NOS:65, 66, 133, 134 and SEQ ID NOS:67, 68, 135, 136, and sequences complementary thereto, wherein said contiguous sequence comprises at least one methylation variable position, or at least one CpG, tpG, or Cpa dinucleotide sequence, and wherein pretreatment comprises treating the genomic DNA with an agent, or series of agents, that modifies unmethylated, but leaves methylated, cytosine essentially unmodified.

Additional embodiments provide a set of oligomers, said set comprising a first oligomer and a second oligomer, wherein the first oligomer, and the second oligomer each comprises at least one contiguous base sequence of at least 16 nucleotides in length that is complementary to, or hybridizes under moderately stringent or stringent conditions to a pretreated genomic DNA sequence selected from, in the case of the first oligomer, a sequence subgroup selected from a first group of 4-sequence subgroups consisting of SEQ ID NOS:1, 2, 69, 70; SEQ ID NOS:3, 4, 71, 72; SEQ ID NOS:5, 6, 73, 74; SEQ ID NOS:7, 8, 75, 76; SEQ ID NOS:9, 10, 77, 78; SEQ ID NOS:11, 12, 79, 80; SEQ ID NOS:13, 14, 81, 82; SEQ ID NOS:15, 16, 83, 84; SEQ ID NOS:17, 18, 85, 86; SEQ ID NOS:19, 20, 87, 88; SEQ ID NOS:21, 22, 89, 90; SEQ ID NOS:23, 24, 91, 92; SEQ ID NOS:25, 26, 93, 94; SEQ ID NOS:27, 28, 95, 96; SEQ ID NOS:29, 30, 97, 98; SEQ ID NOS:31, 32, 99, 100; SEQ ID NOS:33, 34, 101, 102; SEQ ID NOS:35, 36, 103, 104; SEQ ID NOS:37, 38, 105, 106; SEQ ID NOS:39, 40, 107, 108; SEQ ID NOS:41, 42, 109, 110; SEQ ID NOS:43, 44, 111, 112; SEQ ID NOS:45, 46, 113, 114; SEQ ID NOS:47, 48, 115, 116; SEQ ID NOS:49, 50, 117, 118; SEQ ID NOS:51, 52, 119, 120; SEQ ID NOS:53, 54, 121, 122; SEQ ID NOS:55, 56, 123, 124; SEQ ID NOS:57, 58, 125, 126; SEQ ID NOS:59, 60, 127, 128; SEQ ID NOS:61, 62, 129, 130; SEQ ID NOS:63, 64, 131, 132; SEQ ID NOS:65, 66, 133, 134 and SEQ ID NOS:67, 68, 135, 136, and selected from, in the case of the second oligomer, a corresponding complementary sequence subgroup selected from a second group of 4-sequence subgroups consisting of sequences complementary to the respective subgroup sequences of the first sequence group, and wherein pretreatment comprises treating the genomic DNA with an agent, or series of agents, that modifies unmethylated, but leaves methylated, cytosine essentially unmodified. Preferably, the set is suitable for use in generating nucleic acid amplificates.

Yet additional embodiment provide a method for at least one of identifying liver cells, organ or tissue, distinguishing liver cells, organ or tissue from one or more other cell or tissue types, or identifying liver cells, organ or tissue as the source of a DNA sample, comprising: obtaining at least one cell, tissue, bodily fluid or other sample, wherein the sample comprises genomic DNA; determining, for the at least one sample and using a suitable assay, a methylation state or a level of methylation for at least one methylation variable position within a genomic DNA sequence selected from the group consisting of SEQ ID NO:205, a fragment thereof at least 16 contiguous nucleotides in length, and sequences that are complementary to, or hybridize under moderately stringent or stringent conditions to SEQ ID NO:205 or to a fragment thereof at least 16 contiguous nucleotides in length; and comparing said at least one methylation state or level of methylation with a suitable standard or control, or comparing said at least one methylation state or level of methylation between or among corresponding methylation variable positions of the samples, whereby at least one of identifying liver cells, organ or tissue, distinguishing liver cells, organ or tissue from one or more other cell, organ or tissue types, or identifying liver cells, organ or tissue as the source of a DNA sample is, at least in part afforded. Preferably, determining in b), comprises at least one of: use of one or more nucleic acid or oligomers comprising, in each case, at least one contiguous base sequence having a length of at least 16 nucleotides that is complementary to, or hybridizes under moderately stringent or stringent conditions to a pretreated genomic DNA sequence selected from a group consisting of SEQ ID NOS:1, 2, 69, 70; SEQ ID NOS:7, 8, 75, 76; SEQ ID NOS:9, 10, 77, 78; SEQ ID NOS:11, 12, 79, 80; SEQ ID NOS:13, 14, 81, 82; SEQ ID NOS:25, 26, 93, 94; SEQ ID NOS:27, 28, 95, 96; SEQ ID NOS:35, 36, 103, 104; SEQ ID NOS:37, 38, 105, 106; SEQ ID NOS:51, 52, 119, 120; SEQ ID NOS:53, 54, 121, 122; SEQ ID NOS:59, 60, 127, 128; and sequences complementary thereto; or use of a methylation-sensitive restriction enzyme on a genomic DNA sequence selected from the group consisting of SEQ ID NO:205 or a fragment thereof at least 16 contiguous nucleotides in length, and sequences that are complementary to, or hybridize under moderately stringent or stringent conditions to SEQ ID NO: 205 or a fragment thereof at least 16 contiguous nucleotides in length.

Additional embodiments provide a method for at least one of identifying brain cells, organ or tissue, distinguishing brain cells, organ or tissue from one or more other cell or tissue types, or identifying brain cells, organ or tissue as the source of a DNA sample, comprising: obtaining at least one cell, tissue, bodily fluid or other sample, wherein the sample comprises genomic DNA; determining, for the at least one sample and using a suitable assay, a methylation state or a level of methylation for at least one methylation variable position within a genomic DNA sequence selected from the group consisting of SEQ ID NO:205, a fragment thereof at least 16 contiguous nucleotides in length, and sequences that are complementary to, or hybridize under moderately stringent or stringent conditions to SEQ ID NO:205 or to a fragment thereof at least 16 contiguous nucleotides in length; and comparing said at least one methylation state or level of methylation with a suitable standard or control, or comparing said at least one methylation state or level of methylation between or among corresponding methylation variable positions of the samples, whereby at least one of identifying brain cells, organ or tissue, distinguishing brain cells, organ or tissue from one or more other cell, organ or tissue types, or identifying brain cells, organ or tissue as the source of a DNA sample is, at least in part afforded. Preferably, determining), comprises at least one of: use of one or more nucleic acid or oligomers comprising, in each case, at least one contiguous base sequence having a length of at least 16 nucleotides that is complementary to, or hybridizes under moderately stringent or stringent conditions to a pretreated genomic DNA sequence selected from a group consisting of SEQ ID NOS:3, 4, 71, 72; SEQ ID NOS:17, 18, 85, 86; SEQ ID NOS:19, 20, 87, 88; SEQ ID NOS:29, 30, 97, 98; SEQ ID NOS: 49, 50, 117, 118; SEQ ID NOS:57, 58, 125, 126; SEQ ID NOS:61, 62, 129, 130; SEQ ID NOS:67, 68, 135, 136; and sequences complementary thereto; or use of a methylation-sensitive restriction enzyme on a genomic DNA sequence selected from the group consisting of SEQ ID NO:205 or a fragment thereof at least 16 contiguous nucleotides in length, and sequences that are complementary to, or hybridize under moderately stringent or stringent conditions to SEQ ID NO:205 or a fragment thereof at least 16 contiguous nucleotides in length.

Still Additional embodiments provide a method for at least one of identifying breast cells, organ or tissue, distinguishing breast cells, organ or tissue from one or more other cell or tissue types, or identifying breast cells, organ or tissue as the source of a DNA sample, comprising: obtaining at least one cell, tissue, bodily fluid or other sample, wherein the sample comprises genomic DNA; determining, for the at least one sample and using a suitable assay, a methylation state or a level of methylation for at least one methylation variable position within a genomic DNA sequence selected from the group consisting of SEQ ID NO:205, a fragment thereof at least 16 contiguous nucleotides in length, and sequences that are complementary to, or hybridize under moderately stringent or stringent conditions to SEQ ID NO:205 or to a fragment thereof at least 16 contiguous nucleotides in length; and comparing said at least one methylation state or level of methylation with a suitable standard or control, or comparing said at least one methylation state or level of methylation between or among corresponding methylation variable positions of the samples, whereby at least one of identifying breast cells, organ or tissue, distinguishing breast cells, organ or tissue from one or more other cell, organ or tissue types, or identifying breast cells, organ or tissue as the source of a DNA sample is, at least in part afforded. Preferably, determining comprises at least one of: use of one or more nucleic acid or oligomers comprising, in each case, at least one contiguous base sequence having a length of at least 16 nucleotides that is complementary to, or hybridizes under moderately stringent or stringent conditions to a pretreated genomic DNA sequence selected from a group consisting of SEQ ID NOS:3, 4, 71, 72; SEQ ID NOS:5, 6, 73, 74; SEQ ID NOS;15, 16, 83, 84; SEQ ID NOS:19, 20, 87, 88; SEQ ID NOS:21, 22, 89, 90; SEQ ID NOS:23, 24, 91, 92; SEQ ID NOS:29, 30, 97, 98; SEQ ID NOS:39, 40, 107, 108; SEQ ID NOS;41, 42, 109, 110; SEQ ID NOS;45, 46, 113, 114; SEQ ID NOS;63, 64, 131, 132; SEQ ID NOS:65, 66, 133, 134; SEQ ID NOS:67, 68, 135, 136; and sequences complementary thereto; or use of a methylation-sensitive restriction enzyme on a genomic DNA sequence selected from the group consisting of SEQ ID NO:205 or a fragment thereof at least 16 contiguous nucleotides in length, and sequences that are complementary to, or hybridize under moderately stringent or stringent conditions to SEQ ID NO:205 or a fragment thereof at least 16 contiguous nucleotides in length.

Additional embodiments provide a method for at least one of identifying muscle cells, organ or tissue, distinguishing muscle cells, organ or tissue from one or more other cell or tissue types, or identifying muscle cells, organ or tissue as the source of a DNA sample, comprising: obtaining at least one cell, tissue, bodily fluid or other sample, wherein the sample comprises genomic DNA; determining, for the at least one sample and using a suitable assay, a methylation state or a level of methylation for at least one methylation variable position within a genomic DNA sequence selected from the group consisting of SEQ ID NO:205, a fragment thereof at least 16 contiguous nucleotides in length, and sequences that are complementary to, or hybridize under moderately stringent or stringent conditions to SEQ ID NO:205 or to a fragment thereof at least 16 contiguous nucleotides in length; and comparing said at least one methylation state or level of methylation with a suitable standard or control, or comparing said at least one methylation state or level of methylation between or among corresponding methylation variable positions of the samples, whereby at least one of identifying muscle cells, organ or tissue, distinguishing muscle cells, organ or tissue from one or more other cell, organ or tissue types, or identifying muscle cells, organ or tissue as the source of a DNA sample is, at least in part afforded. Preferably, determining comprises at least one of: use of one or more nucleic acid or oligomers comprising, in each case, at least one contiguous base sequence having a length of at least 16 nucleotides that is complementary to, or hybridizes under moderately stringent or stringent conditions to a pretreated genomic DNA sequence selected from a group consisting of SEQ ID NOS:15, 16, 83, 84; SEQ ID NOS:19, 20, 87, 88; SEQ ID NOS:21, 22, 89, 90; SEQ ID NOS:27, 28, 95, 96; SEQ ID NOS:29, 30, 97, 98; SEQ ID NOS:43, 44, 111, 112; SEQ ID NOS:45, 46, 113, 114; SEQ ID NOS:47, 48, 115, 116; SEQ ID NOS:55, 56, 123, 124; SEQ ID NOS:57, 58, 125, 126; SEQ ID NOS:63, 64, 131, 132; and sequences complementary thereto; or use of a methylation-sensitive restriction enzyme on a genomic DNA sequence selected from the group consisting of SEQ ID NO:205 or a fragment thereof at least 16 contiguous nucleotides in length, and sequences that are complementary to, or hybridize under moderately stringent or stringent conditions to SEQ ID NO:205 or a fragment thereof at least 16 contiguous nucleotides in length.

Also provided is a method for at least one of identifying lung cells, organ or tissue, distinguishing lung cells, organ or tissue from one or more other cell, organ or tissue types, or identifying lung cells, organ or tissue as the source of a DNA sample, comprising: obtaining at least one cell, tissue, bodily fluid or other sample, wherein the sample comprises genomic DNA; determining, for the at least one sample and using a suitable assay, a methylation state or a level of methylation for at least one methylation variable position within a genomic DNA sequence selected from the group consisting of SEQ ID NO:205, a fragment thereof at least 16 contiguous nucleotides in length, and sequences that are complementary to, or hybridize under moderately stringent or stringent conditions to SEQ ID NO:205 or to a fragment thereof at least 16 contiguous nucleotides in length; and comparing said at least one methylation state or level of methylation with a suitable standard or control, or comparing said at least one methylation state or level of methylation between or among corresponding methylation variable positions of the samples, whereby at least one of identifying lung cells, organ or tissue, distinguishing lung cells, organ or tissue from one or more other cell, organ or tissue types, or identifying lung cells, organ or tissue as the source of a DNA sample is, at least in part afforded. Preferably, determining comprises at least one of: use of one or more nucleic acid or oligomers comprising, in each case, at least one contiguous base sequence having a length of at least 16 nucleotides that is complementary to, or hybridizes under moderately stringent or stringent conditions to a pretreated genomic DNA sequence selected from a group consisting of SEQ ID NOS:21, 22, 89, 99; SEQ ID NOS:29, 30, 97, 98; SEQ ID NOS:31, 32, 99, 100; SEQ ID NOS:33, 34, 101, 102; SEQ ID NOS:55, 56, 123, 124, and sequences complementary thereto; or use of a methylation-sensitive restriction enzyme on a genomic DNA sequence selected from the group consisting of SEQ ID NO:205 or a fragment thereof at least 16 contiguous nucleotides in length, and sequences that are complementary to, or hybridize under moderately stringent or stringent conditions to SEQ ID NO:205 or a fragment thereof at least 16 contiguous nucleotides in length.

Yet further embodiments comprise use of a nucleic acid or oligomer, in a method for the identification or distinguishing of liver cells, organ or tissue or a nucleic acid derived there from, or for the identification of liver cells, organ or tissue as the source of said nucleic acid, wherein said nucleic acid or oligomer comprises at least one contiguous base sequence having a length of at least 16 nucleotides that is complementary to, or hybridizes under moderately stringent or stringent conditions to a pretreated genomic DNA sequence selected from the group consisting of SEQ ID NOS:1, 2, 69, 70; SEQ ID NOS:7, 8, 75, 76; SEQ ID NOS:9, 10, 77, 78; SEQ ID NOS:11, 12, 79, 80; SEQ ID NOS:13, 14, 81, 82; SEQ ID NOS:25, 26, 93, 94; SEQ ID NOS:27, 28, 95, 96; SEQ ID NOS:35, 36, 103, 104; SEQ ID NOS:37, 38, 105, 106; SEQ ID NOS:51, 52, 119, 120; SEQ ID NOS:53, 54, 121, 122; SEQ ID NOS:59, 60, 127, 128; and sequences complementary thereto, said method comprising determining the level of methylation of at least one methylation variable positions (MVPs) within one or more sequences of the sequence group.

Additionally provided is use of a nucleic acid or oligomer, in a method for the identification or distinguishing of liver cells, organ or tissue, or a nucleic acid derived there from, or for the identification of liver cells, organ or tissue as the source of said nucleic acid, wherein said nucleic acid or oligomer comprises at least one contiguous base sequence at least 16 nucleotides in length selected from the group consisting of SEQ ID NOS:137, 138; 143, 144: 145, 146; 147, 148; 149, 150; 161, 162; 163, 164; 171, 172; 173, 174; 187, 188; 189, 190; 19, and SEQ ID NO:196.

Further embodiment comprise use of a nucleic acid or oligomer, in a method for the identification or distinguishing of brain cells, organ or tissue or a nucleic acid derived there from, or for the identification of brain cells, organ or tissue as the source of said nucleic acid, wherein said nucleic acid or oligomer comprises at least one contiguous base sequence having a length of at least 16 nucleotides that is complementary to, or hybridizes under moderately stringent or stringent conditions to a pretreated genomic DNA sequence selected from the group consisting of SEQ ID NOS:3, 4, 71, 72; SEQ ID NOS:17, 18, 85, 86; SEQ ID NOS:19, 20, 87, 88; SEQ ID NOS:29, 30, 97, 98; SEQ ID NOS:49, 50, 117, 118; SEQ ID NOS:57, 58, 125, 126; SEQ ID NOS:61, 62, 129, 130; SEQ ID NOS:67, 68, 135, 136; and sequences complementary thereto, said method comprising determining the level of methylation of at least one methylation variable positions (MVPs) within one or more sequences of the sequence group.

Additional embodiments comprise use of a nucleic acid or oligomer, in a method for the identification or distinguishing of brain cells, organ or tissue, or a nucleic acid derived there from, or for the identification of brain cells, organ or tissue as the source of said nucleic acid, wherein said nucleic acid or oligomer comprises at least one contiguous base sequence at least 16 nucleotides in length selected from the group consisting of SEQ ID NOS:139, 140; 153, 154; 155, 156; 157, 158; 165, 166; 185, 186; 193, 194; 197, 198; 203 and SEQ ID NO:204.

Further embodiment comprise use of a nucleic acid or oligomer, in a method for the identification or distinguishing of breast cells, organ or tissue or a nucleic acid derived there from, or for the identification of breast cells, organ or tissue as the source of said nucleic acid, wherein said nucleic acid or oligomer comprises at least one contiguous base sequence having a length of at least 16 nucleotides that is complementary to, or hybridizes under moderately stringent or stringent conditions to a pretreated genomic DNA sequence selected from the group consisting of SEQ ID NOS:3, 4, 71, 72; SEQ ID NOS:5, 6, 73, 74; SEQ ID NOS:15, 16, 83, 84; SEQ ID NOS:19, 20, 87, 88; SEQ ID NOS:21, 22, 89, 90; SEQ ID NOS:23, 24, 91, 92; SEQ ID NOS:29, 30, 97, 98; SEQ ID NOS:39, 40, 107, 108; SEQ ID NOS:41, 42, 109, 110; SEQ ID NOS:45, 46, 113, 114; SEQ ID NOS:63, 64, 131, 132; SEQ ID NOS:65, 66, 133, 134; SEQ ID NOS:67, 68, 135, 136; and sequences complementary thereto, said method comprising determining the level of methylation of at least one methylation variable positions (MVPs) within one or more sequences of the sequence group.

Even further embodiments comprise use of a nucleic acid or oligomer, in a method for the identification or distinguishing of breast cells, organ or tissue, or a nucleic acid derived there from, or for the identification of breast cells, organ or tissue as the source of said nucleic acid, wherein said nucleic acid or oligomer comprises at least one contiguous base sequence at least 16 nucleotides in length selected from the group consisting of SEQ ID NOS:139, 140; 141, 142; 151, 152; 155, 156, 157, 158; 159, 160; 165, 166, 175, 176; 177, 178; 181, 182; 199, 200; 201, 202; 203 and SEQ ID NO:204.

Additional embodiments comprise use of a nucleic acid or oligomer, in a method for the identification or distinguishing of muscle cells, organ or tissue or a nucleic acid derived there from, or for the identification of muscle cells, organ or tissue as the source of said nucleic acid, wherein said nucleic acid or oligomer comprises at least one contiguous base sequence having a length of at least 16 nucleotides that is complementary to, or hybridizes under moderately stringent or stringent conditions to a pretreated genomic DNA sequence selected from the group consisting of SEQ ID NOS:15, 16, 83, 84; SEQ ID NOS:19, 20, 87, 88; SEQ ID NOS:21, 22, 89, 90; SEQ ID NOS:27, 28, 95, 96; SEQ ID NOS:29, 30, 97, 98; SEQ ID NOS:43, 44, 111, 112; SEQ ID NOS:45, 46, 113, 114; SEQ ID NOS:47, 48, 115, 116; SEQ ID NOS:55, 56, 123, 124; SEQ ID NOS:57, 58, 125, 126; SEQ ID NOS:63, 64, 131, 132; and sequences complementary thereto, said method comprising determining the level of methylation of at least one methylation variable positions (MVPs) within one or more sequences of the sequence group.

Still further embodiments comprise use of a nucleic acid or oligomer, in a method for the identification or distinguishing of muscle cells, organ or tissue, or a nucleic acid derived there from, or for the identification of muscle cells, organ or tissue as the source of said nucleic acid, wherein said nucleic acid or oligomer comprises at least one contiguous base sequence at least 16 nucleotides in length selected from the group consisting of SEQ ID NOS:152, 152; 155, 156; 157, 158; 163, 164; 165, 166; 179, 180; 181, 182; 183, 184; 191, 192; 193, 194; 199 and SEQ ID NO:200.

Additional embodiments comprise se of a nucleic acid or oligomer, in a method for the identification or distinguishing of lung cells, organ or tissue or a nucleic acid derived there from, or for the identification of lung cells, organ or tissue as the source of said nucleic acid, wherein said nucleic acid or oligomer comprises at least one contiguous base sequence having a length of at least 16 nucleotides that is complementary to, or hybridizes under moderately stringent or stringent conditions to a pretreated genomic DNA sequence selected from the group consisting of SEQ ID NOS:19, 20, 87, 88; SEQ ID NOS:21, 22, 89, 99; SEQ ID NOS:29, 30, 97, 98; SEQ ID NOS:31, 32, 99, 100; SEQ ID NOS:33, 34, 101, 102; SEQ ID NOS:55, 56, 123, 124; and sequences complementary thereto, said method comprising determining the level of methylation of at least one methylation variable positions (MVPs) within one or more sequences of the sequence group.

Particular embodiments comprise use of a nucleic acid or oligomer, in a method for the identification or distinguishing of lung cells, organ or tissue, or a nucleic acid derived there from, or for the identification of lung cells, organ or tissue as the source of said nucleic acid, wherein said nucleic acid or oligomer comprises at least one contiguous base sequence at least 16 nucleotides in length selected from the group consisting of SEQ ID NOS:155, 156; 157, 158; 165, 166; 167, 168; 169, 170; 191 and SEQ ID NO:192.

In further aspects the invention comprises se of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:19, 20, 87, 88 and sequences complementary thereto, or use in said method of a nucleic acid or oligomer comprising at least one contiguous base sequence having a length of at least 16 nucleotides selected from a second group of SEQ ID NOS:155 and 156, said method comprising determining the methylation state or level of methylation of at least one methylation variable positions (MVPs) within one or more sequences of the first sequence group; wherein the first group of tissues or cells comprises breast, brain and muscle cells or tissues, and the second group of tissues or cells comprises liver; lung and prostate cells or tissues.

Also provided is use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:21, 22, 89, 90 and sequences complementary thereto, or use in said method of a nucleic acid or oligomer comprising at least one contiguous base sequence having a length of at least 16 nucleotides selected from a second group of SEQ ID NOS:157 and 158, said method comprising determining the methylation state or level of methylation of at least one methylation variable position (MVPs) within one or more sequences of the first sequence group; wherein the first group of tissues or cells comprises breast, liver and muscle cells or tissues, and the second group of tissues or cells comprises lung and brain cells or tissues.

Yet further embodiments comprise use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:27, 28, 95, 96 and sequences complementary thereto, or use in said method of a nucleic acid or oligomer comprising at least one contiguous base sequence having a length of at least 16 nucleotides selected from a second group of SEQ ID NOS:163 and 164, said method comprising determining the methylation state or level of methylation of at least one methylation variable position (MVPs) within one or more sequences of the first sequence group; wherein the first group of tissues or cells comprises liver and muscle cells or tissues, and the second group of tissues or cells comprises breast and brain cells or tissues.

In particular aspects, the present invention comprises use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:29, 30, 97, 98 and sequences complementary thereto, or use in said method of a nucleic acid or oligomer comprising at least one contiguous base sequence having a length of at least 16 nucleotides selected from a second group of SEQ ID NOS:165 and 166, said method comprising determining the methylation state or level of methylation of at least one methylation variable position (MVPs) within one or more sequences of the first sequence group; wherein the first group of tissues or cells comprises breast, brain and muscle cells or tissues, and the second group of tissues or cells comprises lung and prostate cells or tissues.

In further particular aspects, the present invention comprises use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:39, 40, 107, 108 and sequences complementary thereto, or use in said method of a nucleic acid or oligomer comprising at least one contiguous base sequence having a length of at least 16 nucleotides selected from a second group of SEQ ID NOS:175 and 176, said method comprising determining the methylation state or level of methylation of at least one methylation variable position (MVPs) within one or more sequences of the first sequence group; wherein the first group of tissues or cells comprises breast, and prostate cells or tissues, and the second group of tissues or cells comprises brain, lung and liver cells or tissues.

In yet further particular aspects, the present invention comprises use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:45, 46, 113, 114; 63, 64, 131, 132 and sequences complementary thereto, or use in said method of a nucleic acid or oligomer comprising at least one contiguous base sequence having a length of at least 16 nucleotides selected from a second group of SEQ ID NOS:181, 182, 199 and 200, said method comprising determining the methylation state or level of methylation of at least one methylation variable position (MVPs) within one or more sequences of the first sequence group; wherein the first group of tissues or cells comprises breast and muscle cells or tissues, and the second group of tissues or cells comprises lung, brain, liver and prostate cells or tissues.

In additional aspects, the present invention comprises use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:67, 68, 135, 136 and sequences complementary thereto, or use in said method of a nucleic acid or oligomer comprising at least one contiguous base sequence having a length of at least 16 nucleotides selected from a second group of SEQ ID NOS:203 and 204, said method comprising determining the methylation state or level of methylation of at least one methylation variable position (MVPs) within one or more sequences of the first sequence group; wherein the first group of tissues or cells comprises breast and brain cells or tissues, and the second group of tissues or cells comprises lung, muscle, liver and prostate cells or tissues.

In additional aspects, the present invention further comprises use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:57, 58, 125, 126 and sequences complementary thereto, or use in said method of a nucleic acid or oligomer comprising at least one contiguous base sequence having a length of at least 16 nucleotides selected from a second group of SEQ ID NOS:193 and 194, said method comprising determining the methylation state or level of methylation of at least one methylation variable position (MVPs) within one or more sequences of the first sequence group; wherein the first group of tissues or cells comprises brain and muscle cells or tissues, and the second group of tissues or cells comprises lung, breast, liver and prostate cells or tissues.

Additional embodiments comprise use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:17, 18, 85, 86 and sequences complementary thereto, or use in said method of a nucleic acid or oligomer comprising at least one contiguous base sequence having a length of at least 16 nucleotides selected from a second group of SEQ ID NOS:153 and 154, said method comprising determining the methylation state or level of methylation of at least one methylation variable position (MVPs) within one or more sequences of the first sequence group; wherein the first group of tissues or cells comprises breast and lung cells or tissues, and the second group of tissues or cells comprises brain, muscle, liver and prostate cells or tissues.

The present invention provides a method for diagnosing a condition or disease characterized by specific methylation levels or methylation states of one or more methylation variable genomic DNA positions in a disease-associated cell or tissue or in a sample derived from a bodily fluid, comprising: obtaining a test cell, tissue sample or bodily fluid sample comprising genomic DNA having one or more methylation variable positions in one or more regions thereof; determining the methylation state or quantified methylation level at the one or more methylation variable positions; and comparing said methylation state or level to that of a genome wide methylation map according to claim 1, said map comprising methylation level values for at least one of corresponding normal, or diseased cells or tissue, whereby a diagnosis of a condition or disease is, at least in part afforded.

Yet further embodiments provide a method for detecting the absence or presence of a medical condition in an organ, cell type or tissue, comprising: retrieving a bodily fluid sample; determining at least one of the amount or presence, of free-floating DNA that exhibits a tissue-, organ- or cell type-specific DNA methylation pattern by use of a nucleic acid or oligomer comprising at least one contiguous base sequence having a length of at least 16 nucleotides that is complementary to, or hybridizes under moderately stringent or stringent conditions to a pretreated genomic DNA sequence selected from the group consisting of SEQ ID NOS:1 through SEQ ID NO:204 and SEQ ID NOS:206 through SEQ ID NO:221, and sequences complementary thereto; and determining whether there is an abnormal level of free floating DNA that originates from said tissue, cell type or organ, thereby concluding, whether a medical condition associated with said tissue, cell type or organ is absent or present.

Also provided is a method for diagnosing a condition or disease of an individual characterized by the presence of organ- or tissue-specific free-floating DNA in said individual's bodily fluid, comprising: retrieving a bodily fluid sample; determining at least one of the amount or presence, of free floating DNA that exhibits a tissue-, organ- or cell type-characteristic DNA methylation pattern with the use of at least one nucleic acid or oligomer comprising at least one contiguous base sequence having a length of at least 16 nucleotides that is complementary to, or hybridizes under moderately stringent or stringent conditions to a pretreated genomic DNA sequence selected from the group consisting of SEQ ID NOS:1 through SEQ ID NO:204 and SEQ ID NOS:206 through SEQ ID NO:221, and sequences complementary thereto; and further determining, whether there is an abnormal level of free-floating DNA that originates from said tissue, cell type or organ, and, at least in part thereby, concluding whether a medical condition associated with said tissue, cell type or organ is absent or present.

In particular embodiments the invention provides a method for diagnosing a condition or disease of an individual characterized by the presence of organ- or tissue-specific free-floating DNA in said individual's bodily fluid, comprising: retrieving a bodily fluid sample; determining the methylation states or methylation levels of MVPs within at least one nucleic acid or oligomer comprising at least one contiguous base sequence having a length of at least 16 nucleotides that is complementary to, or hybridizes under moderately stringent or stringent conditions to a pretreated genomic DNA sequence selected from the group consisting of SEQ ID NOS:1 through SEQ ID NO:204 and SEQ ID NOS:206 through SEQ ID NO:221 and sequences complementary thereto; comparing said methylation states or levels to that of a genome-wide methylation map according to claim 1, said map comprising methylation level values of the corresponding nucleic acids for a plurality of normal organs, cells or tissues; and determining whether the methylation states or levels of b) match with known values and whether a specific organ or tissue is dominant, whereby a diagnosis of a condition or disease is, at least in part, afforded. Preferably, said free-floating DNA is derived from a tissue or organ selected from the group consisting of lung, liver, muscle, breast, brain or prostate.

Additional embodiments provide a method for at least one of choosing or monitoring a course of treatment, comprising, obtaining a diagnosis according to claims 49 to 52, whereby at least one of choosing or monitoring a course of treatment is, at least in part, afforded.

Also provided is use of a method according to any one of claims 49-53 for diagnosing a disease of an individual, diagnosing a condition of an individual, prognosing a disease of an individual, monitoring disease progression, monitoring treatment response, monitoring the occurrence of treatment side affects, or for classification, differentiation, grading, staging, or diagnosing of a cell proliferative disease or for a combination thereof.

Further embodiments provide a method for at least one of, identifying one organ, cell or tissue type, or distinguishing one organ, cell or tissue type from another as the source of a nucleic acid sample, comprising: obtaining a nucleic acid sample having genomic DNA; pretreating the genomic DNA, or a fragment thereof, with one or more agents to convert 5-position unmethylated cytosine bases to uracil or to another base that is detectably dissimilar to cytosine in terms of hybridization properties; contacting the pretreated genomic DNA, or the pretreated fragment thereof, with an amplification enzyme and at least one primer set, each said set comprising first and second primer each having a contiguous sequence at least 16 nucleotides in length that is complementary to, or hybridizes under moderately stringent or stringent conditions to a sequence selected from, in the case of the first primer, a first group consisting of SEQ ID NOS:1-136, and selected from, in the case of the second primer, a second group consisting of sequences complementary to the sequences of the first group, wherein the pretreated DNA, or the fragment thereof is either amplified to produce one or more amplificates, or is not amplified; and determining, based on the presence or absence of, or on a property of said amplificate, the methylation state or level of methylation of at least one MVP within the pretreated version of SEQ ID NO:205 or within a contiguous region thereof, or an average, or a value reflecting an average methylation state of a plurality of MVPs within the pretreated version of SEQ ID NO:205 or within a contiguous region thereof, whereby at least one of identifying one organ, cell or tissue type, or distinguishing one organ, cell or tissue type from another as the source of the nucleic acid sample is, at least in part afforded. Preferably, treating the genomic DNA, or the fragment thereof, comprises use of a solution selected from the group consisting of bisulfite, hydrogen sulfite, disulfite, and combinations thereof. Preferably, at least one of contacting, or determining comprises use of a method selected from the group consisting of MSP, MethyLight™, HeavyMethyl™, MS-SNuPE™, and combination thereof. Preferably, at least one of said primers comprises a sequence selected from the group consisting of SEQ ID NO:137 through SEQ ID NO:204. Preferably, the contiguous sequence of one or more of said primers comprises at least one 5′-CG-3′, 5′-tG-3′ or 5′-Ca-3′ dinucleotide. Preferably the methods comprise use of at least one oligomer comprising a contiguous sequence at least 16 nucleotides in length having one or more 5′-CG-3′, 5′-tG-3′ or 5′-Ca-3′ dinucleotides that were CG dinucleotides prior to pretreating in b) of claim 54, and wherein the contiguous sequence of said oligomer is complementary or identical to a sequence selected from the group consisting of SEQ ID NOS:1-136, and complements thereof, and wherein said oligomer suppresses amplification of the nucleic acid to which it is hybridized. Preferably, determining the methylation state, or level of methylation or the average methylation state or average level of methylation comprises use of at least one reporter or probe oligomer that hybridizes to one or more 5′-CG-3′, 5′-TG-3′ or 5′-CA-3′ dinucleotides, at positions which were 5′-CG-3′ dinucleotides prior to pretreating, whereby amplification of one or more target sequences is, at least in part, afforded.

Particular embodiments comprise use of the inventive methods for the analysis, characterization, classification, differentiation, grading, staging, diagnosis, or prognosis of cell proliferative disorders, or the predisposition to cell proliferative disorders, or combination thereof.

Particular embodiments comprise use of the inventive methods for the analysis, characterisation, classification, differentiation, grading, staging, or diagnosis or a combination thereof of prostate cancer, breast cancer, lung cancer, liver cancer or brain cancer, or the predisposition to said types of cancer.

Additional embodiments provide for a kit useful for identifying one tissue, organ or cell type as the source of a nucleic acid, or for distinguishing one tissue, organ or cell type from another among a group of tissue organ or cell types, as the source of a nucleic acid comprising: a bisulfite reagent or a methylation-sensitive deamination enzyme; and at least one oligomer comprising, in each case a contiguous sequence of at least 9 nucleotides in length that is complementary to, or hybridizes under moderately stringent or stringent conditions to a sequence selected from the group consisting of SEQ ID NOS:1-136, and complements thereof. Preferably, the tissue type group comprises at least two tissue types selected from the group consisting of prostate, breast, lung, liver, muscle and brain. Also provided is a kit useful for detecting, diagnosing, prognosing or differentiating cell proliferative disorders of the prostate, breast, lung, liver, muscle or brain, or for distinguishing between cell proliferative disorders of the prostate, breast, lung, liver, muscle or brain, comprising: a bisulfite reagent or a methylation sensitive deamination enzyme; and at least one nucleic acid molecule or peptide nucleic acid molecule comprising, in each case a contiguous sequence at least 9 nucleotides in length that is complementary to, or hybridizes under moderately stringent or stringent conditions to a sequence selected from the group consisting of SEQ ID NOS:1-136, and complements thereof.

Preferably, the kit comprises standard reagents for performing a methylation assay selected from the group consisting of MS-SNuPE™, MSP, MethylLight™, HeavyMethyl™, COBRA™, nucleic acid sequencing, and combinations thereof.

Yet further embodiments provide a method of providing diagnostic information relating to cancer, comprising: determining the relative amount of free-floating DNA derived from a specific organ or tissue within the total amount of free-floating DNA in a bodily fluid sample of a patient suspected of suffering from a cell proliferative disorder, wherein said determining comprises determination of the level of methylation of at least three MVPs or CpGs selected from the group identified in Tables 37-70 in said bodily fluid sample, and wherein a methylation pattern is provided; comparing said methylation pattern with methylation patterns found in a plurality of samples that have been identified to be characteristic for specific organs or tissues out of a group of other organs or tissues; determining, in relation to samples from healthy donors, whether the methylation pattern determined in a) indicates an increased relative amount of free-floating DNA derived from a specific organ or tissue within the total amount of free-floating DNA in said bodily fluid, whereby a conclusion as to whether said patient has an increased risk of developing cancer is, at least in part, afforded. Preferably, the methylation pattern comprises the levels of methylation of at least 5 CpG positions. Preferably, at least three MVPs or CpG positions of which the level of methylation is determined, are located within a 500 bp genomic region.

### BRIEF DESCRIPTION OF THE DRAWINGS

FIGS. 1-34 represent the levels of methylation at particular CpG positions that are unambiguously identifiable by the numbers at the left of the gray-scaled pattern. The numbers indicate the position, in nucleotides from the 5′-end of amplificate, of each CpG (more specifically, the position of the base, which was a cytosine, prior to pretreatment with a bisulfite reagent) within the amplified section when using the primers as presented in TABLE 1. The terms at the top of the Figure (brain, breast, liver, lung, muscle and prostate) indicate the tissue types from which the analyzed samples were derived. The methylation ‘pattern’ (see definitions below) is represented in the field within the gray shaded boxes. The shade of gray directly correlates with the level of methylation, as is disclosed in detail in FIG. 35. A black box represenets a methylation percentage of 100%, indicating that every single DNA molecule within the sample analyzed was methylated at the corresponding position. A very light gray box, however, indicates that all DNA molecules were unmethylated at the corresponding position. A white box indicates that no value was obtained.

FIG. 35 shows the correlation between the different shades of gray and the corresponding levels of methylation, expressed as percentages.

FIG. 36 displays the sequence traces of two bisulfite sequencing runs corresponding to an exemplary methylation variable position (MVP) identified in a ‘major histocompatibility complex’ (MHC) embodiment according to the present invention. Bisulfite-treated DNA of two different healthy tissues was analyzed by sequencing using the same primer. The left sequence shows the analysis of bisulfite-treated DNA, isolated from healthy lung tissue (indicated by the letter “L”), wherein the cytosine of interest was methylated in the untreated DNA. The right trace shows the analysis of bisulfite-treated DNA, isolated from healthy brain tissue (indicated by the letter “B”), wherein the corresponding cytosine position was unmethylated in the untreated DNA. Bisulfite sequencing is based on the conversion of all non-methylated cytosines to uracil, by treatment of genomic DNA with bisulfite. In the sequence trace, non-methylated cytosine appears therefore as T (effectively replaces U during amplification of the DNA with dNTPs prior to sequencing), while methylated C appears as C (effectively replaces 5-mCyt during amplification of the DNA with dNTPs prior to sequencing). The question as to whether a thymine signal herein represents a base that was a thymine prior to bisulfite treatment, or a converted cytosine requires a comparison of the sequence of pretreated DNA with that of the corresponding untreated genomic DNA. The different dotted lines represent the differentially colored lines in the original trace output file, as indicated in the figure.

### DETAILED DESCRIPTION OF THE INVENTION

Definitions:

For purposes of the present invention, “classes of DNA sources” refers to any distinct sets of samples containing DNA. Preferably said classes are of biological matter, and in such cases, they are referred to herein as ‘classes of biological samples’.

The term “tissue” in this context is meant to describe a group or layer of cells that are alike and that work together to perform a specific function.

The phrase “phenotypically distinct” shall be used to describe organisms, tissues, cells or components thereof, which can be distinguished by one or more characteristics, observable and/or detectable by current technologies. Each of such characteristics may also be defined as a parameter contributing to the definition of the phenotype. Wherein a phenotype is defined by one or more parameters an organism that does not conform to one or more of said parameters shall be defined to be distinct or distinguishable from organisms of said phenotype. Excluded from those characteristics are differences in the organisms' (or the components') cytosine methylation patterns and differences in their DNA sequences.

The term “abnormal” when used in the context of organisms, tissues, cells or components thereof, shall refer to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “oligomer” encompasses oligonucleotides, PNA-oligomers and LNA-oligomers, and is used whenever a term is needed to describe the alternative use of an oligonucleotide or a PNA-oligomer or LNA-oligomer, which cannot be described as oligonucleotide. Said oligomer can be modified as it is commonly known and described in the art. The term “oligomer” also encompasses oligomers carrying at least one detectable label, and preferably fluorescence labels are understood to be encompassed. It is however also understood that the label can be of any kind that is known and described in the art.

The term “Observed/Expected Ratio” (“O/E Ratio”) refers to the frequency of CpG dinucleotides within a particular DNA sequence, and corresponds to the [number of CpG sites/(number of C bases x number of G bases)]×band length for each fragment.

The term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5. CpG islands are typically, but not always, between about 0.2 to about 1 kb in length, and may be as large as about 3 kb in length.

The term “methylation state” or “methylation status” refers to the presence or absence of 5-methylcytosine (“5-mCyt”) at one or a plurality of CpG dinucleotides within a DNA sequence. Methylation states at one or more CpG methylation sites within a single allele's DNA sequence include “unmethylated,” “fully-methylated” and “hemi-methylated.”

The term “hemi-methylation” or “hemimethylation” refers to the methylation state of a CpG methylation site, where only one strand's cytosine of the CpG dinucleotide sequence is methylated (e.g., 5′-TTCMGTA-3′ (top strand): 3′-AAGCAT-5′ (bottom strand)).

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

“Methylation level” or “methylation degree” refers to the average amount of methylation present at an individual CpG dinucleotide. Methylation levels may be expressed as a percentage. Measurement of methylation levels at a plurality of different CpG dinucleotide positions creates either a methylation profile or a methylation pattern.

The term “methylation profile” refers to a profile that is created when average methylation levels of multiple CpGs (scattered throughout the genome) are collected. Each single CpG position is analyzed independently of the other CpGs in the genome, but is analyzed collectively across all homologous DNA molecules in a pool of differentially methylated DNA molecules.

The term “methylation pattern” refers to the description of methylation states of a number of CpG positions in proximity to each other. For example a full methylation of 5-10 closely linked CpG positions, may comprise a methylation pattern that is quite rare and might well be specific for a specific DNA molecule. The term “methylation pattern” can also refer to the description of methylation levels of such a number of proximate CpG positions when measured on a plurality of DNA molecules in a pool of differentially methylated DNA molecules. In that case a methylation level of 100% of 5-10 closely linked CpG positions may be a methylation pattern that is quite rare and will be specific for a specific DNA source, such as a type of tissue or cell.

The term “microarray” refers broadly to both “DNA microarrays” and “DNA chip(s),” and encompasses all art-recognized solid supports, and all art-recognized methods for affixing nucleic acid molecules thereto or for synthesis of nucleic acids thereon.

“Genetic parameters” as used herein are mutations and polymorphisms of genes and sequences further required for gene regulation. Exemplary mutations are, in particular, insertions, deletions, point mutations, inversions and polymorphisms and, particularly preferred, SNPs (single nucleotide polymorphisms).

“Epigenetic parameters” are, in particular, cytosine methylations. Further epigenetic parameters include, for example, the acetylation of histones which, however, cannot be directly analyzed using the described method but which, in turn, correlate with the DNA methylation.

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state or methylation level of one or more CpG dinucleotide sequences within a sequence of DNA.

The term “MS AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

The term “MethyLight™” refers to the art-recognized fluorescence-based real-time PCR technique described by Eads et al., Cancer Res. 59:2302-2306, 1999.

The term “HeavyMethyl™” assay, in the embodiment thereof implemented herein, refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones, Nucleic Acids Res. 25:2529-2531, 1997.

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146.

The term “COBRA” (Combined Bisulfite Restriction Analysis) refers to the art-recognized methylation assay described by Xiong & Laird, Nucleic Acids Res. 25:2532-2534, 1997.

The term “MCA” (Methylated CpG Island Amplification) refers to the methylation assay described by Toyota et al., Cancer Res. 59:2307-12, 1999, and in WO 00/26401A1.

The term “hybridization” is to be understood as the binder of a bond of an oligonucleotide to a complementary sequence along the lines of the Watson-Crick base pairings, including the pairing of a uracil with an adenine, in the sample DNA, forming a duplex structure.

“Stringent hybridization conditions”, as defined herein, involve hybridizing at 68° C. in 5×SSC/5× Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

The term “MVP” refers to a methylation variable position (MVP), which is a CpG position that is differentially methylated in different phenotypically distinct types of samples, such as, but not limited to different tissues, hence a CpG position that shows variable methylation between different tissues.

The phrase “sequence context” in the context of selected CpG dinucleotide sequences refers to a genomic region of from 2 nucleotide bases to about 3 Kb surrounding or including a differentially methylated CpG dinucleotide (MVP) identified by the genome-wide discovery method described herein. Said context region comprises, according to the present invention, at least one secondary differentially methylated CpG dinucleotide sequence, or comprises a pattern having a plurality of differentially methylated CpG dinucleotide sequences including the primary and at least one secondary differentially methylated CpG dinucleotide sequences. Preferably, the primary and secondary differentially methylated CpG dinucleotide sequences within such context region are comethylated in that they share the same methylation status in the genomic DNA of a given tissue sample. Preferably the primary and secondary CpG dinucleotide sequences are comethylated as part of a larger comethylated pattern of differentially methylated CpG dinucleotide sequences in the genomic DNA context. The size of such context regions varies, but will generally reflect the size of CpG islands as defined above, or the size of a gene promoter region, including the first one or two exons.

The term “MVP database” refers to a database containing the methylation levels and locations of differentially methylated CpG positions, in relation to the detailed description of samples including, for example, all, or a portion of all available phenotypical characteristics, and clinical parameters. The database is searchable, for example, for CpG positions that are differentially methylated between or among two or more phenotypically distinct types of tissues/samples.

With respect to the dinucleotde designations within the phrase “CpG, tpG and Cpa,” a small “t” is used to indicate a thymine at a cytosine position, whenever the cytosine was transformed to uracil by pretreatment, whereas, a capital “T” is used to indicate a thymine position that was a thymine prior to pretreatment). Likewise, a small “a” is used to indicate the adenine corresponding to such a small “t” located at a cytosine position, whereas a capital “A” is used to indicate an adenine that was adenine prior to pretreatment.

The term “tumor marker” refers to a distinguishing or characteristic substance that may be found in blood or other bodily fluids, or in tissues that is reflective of a particular tumor. The substance may, for example, be a protein, an enzyme, a RNA molecule or a DNA molecule. The term may alternately refer to a specific characteristic of said substance, such as but not limited to a specific methylation pattern, making the substance distinguishable from otherwise identical substances. A high level of a tumor marker may indicate that a certain type of cancer is developing in the body. Typically, this substance is derived from the tumor itself. Examples of tumor markers include, but are not limited to CA 125 (ovarian cancer), CA 15-3 (breast cancer), CEA (ovarian, lung, breast, pancreas, and gastrointestinal tract cancers), and PSA (prostate cancer).

The term “tissue marker” refers to a distinguishing or characteristic substance that may be found in blood or other bodily fluids, but mainly in cells of specific tissues. The substance may for example be a protein, an enzyme, a RNA molecule or a DNA molecule. The term may alternately refer to a specific characteristic of said substance, such as but not limited to a specific methylation pattern, making the substance distinguishable from otherwise identical substances. A high level of a tissue marker found in a cell may mean said cell is a cell of that respective tissue. A high level of a tissue marker found in a bodily fluid may mean that a respective type of tissue is either spreading cells that contain said marker into the bodily fluid, or is spreading the marker itself into the blood or other bodily fluids.

The term “ESME” refers to a novel and particularly preferred software program that considers or accounts for the unequal distribution of bases in bisulfite converted DNA and normalizes the sequence traces (electropherograms) to allow for quantitation of methylation signals within the sequence traces. Additionally, it calculates a bisulfite conversion rate, by comparing signal intensities of thymines at specific positions, based on the information about the corresponding untreated DNA sequence.

Unless defined otherwise, all technical and scientific terms used herein have the same meaning as commonly understood by one of ordinary skill in the art to which the invention pertains. Although any methods and materials similar or equivalent to those described herein can be used for testing of the present invention, the preferred materials and methods are described herein. All documents cited herein are thereby incorporated by reference.

Overview

The invention comprises, inter alia, a method for identifying, cataloguing and interpreting genome-wide DNA methylation patterns of all human genes in all major tissues. More precisely, the method is concerned with the identification of cytosines in the context of 5′-CG-3′ dinucleotides (i.e., CpG positions), that are differentially methylated in different sample types, for example, in different tissues, organs or cell types. Such differentially methylated cytosine bases are referred to herein as ‘Methylation Variable Positions’ (MVPs). Sample type-specific methylation patterns can be identified by comparing the levels of methylation at one, or preferably several MVPs within a selected genomic region, of DNA obtained from several different sample types. A distinct region of the genome, such as a region of interest (ROI), which comprises one or preferably several of these MVPs can be utilized as a marker (e.g., as a tissue type marker). It is particularly preferred that these MVPs are positioned close to each other. An isolated MVP may suffice as a marker, but it is highly preferred that several CpG positions closely linked to each other are analyzed simultaneously in a suitable methylation analysis assay, such as MethyLight™, HeavyMethyl™ or MSP™.

Particular embodiments of the present invention provide one or more markers selected by performing the inventive method as disclosed in EXAMPLE 1 herein below.

Additional embodiments provide exemplary novel uses of these tissue markers, as illustrated in EXAMPLES 2-6 herein below.

The robust discovery method described herein enables and otherwise provides for the discovery of MVPs and hence the discovery of distinguishing marker regions of genomic DNA.

Additional embodiments provide for comparative data evaluation across different experiments, and between and among different sample types and different genomic regions. The present methods differ from other well known and described methylation discovery methods, in that the present methods provide, inter alia, quantitative information (i.e. levels of methylation at specific sites; and not only a ‘yes or no’ information) on the methylation status of a CpG. As the inventive methods are based on DNA sequencing, they bear three additional advantages. Firstly, the identified MVPs can be instantly mapped to the genome, without a requirement for further experiments; that is, there is no subsequent cloning, and therefore no danger of losing or mixing up results in the process of cloning or sequencing of the amplificates.

Secondly, the inventive methods for identifying suitable markers, which are based on bisulfite amplification product sequencing, are suitable for high throughput processing, as has been demonstrated on an expansive practical scale by the large sequencing facilities involved in elucidating the sequence information of the human genome. The high throughput aspect is necessary, because obtaining accurate and useful results requires analyzing a sufficiently high number of samples derived from different representative well defined nucleic acid sources, such as defined human tissues, organs or cell lines.

A third advantage over prior art discovery methods is that the present methods allow simultaneous comparative analysis of methylation levels of a number of CpG positions that are located next to each other (i.e., analysis of ‘proximate’ CpG positions). Proximate CpG positions are typically co-methylated, but, significantly, are not necessarily so. The present sequencing discovery methods allow for identification of regions (comprising a plurality of CpG or MVP positions) as markers, instead of identification of only single CpG or MVP positions.

Significantly, in the prior art, only single CpGs have been identified to be differentially methylated, and alleged ‘markers’ comprising multiple CpGs have only been tentatively identified by relying on the assumption that proximate CpG positions are co-methylated.

The inventive method described herein, however, removes the necessity to rely on said assumption, and therefore provides markers having confirmed utilility as useful tools to distinguish sample types. Significantly, according to the present invention, the differentiating utility of the prior art single CpG analysis is substantially limited in comparison to that comprising quantitative analysis of several proximate CpG positions.

Additionally, and preferably, analysis of CpG positions within marker regions comprises quantitative analysis of corresponding individual positions in multiple samples of each sample type, improving the quality and hence utility of an identified marker region or of one or more proximate individual MVPs.

Particular embodiments provide a method for analysis of as many as several thousand loci, comprising, for example, all, or a portion of all genes of several chromosomes, or of all the human chromosomes for a number of different nucleic acid sources, and in a manner that allows an informative comparison between all of these levels of methylation.

According to the present invention, bisulfite sequencing provides sufficient robustness for high throughput applications, and quantification and standardization of the data is provided by one or more algorithms or a software program that allows for determination of quantitative methylation levels (as defined herein above). In particularly preferred embodiments describe herein, the algorithm or software program is ESME.

According to the present invention, correlations between specific methylation patterns and phenotypes such as age, gender or disease can be determined, as well as correlations between specific methylation patterns and different cell, tissue or organ types. The afforded knowledge of genome-wide methylation patterns also provides a novel resource for the understanding of fundamental biological processes such as gene regulation, imprinting of genes, development, genome stability, disease susceptibility and the interplay of genetics and environment. Moreover, such knowledge can be used to assess if and how methylation patterns respond to environmental influences, such as nutrition, or smoking, etc.

Moreover, the present invention enables correlations of DNA-methylation patterns with parameters such as tumorigenesis, progression and metastasis, stem cells and differentiation, proliferation and cell cycle, diseases and disorders, and metabolism to be generated.

In a preferred embodiment, the inventive methods are used to identify methylation positions and markers all over the genome, the level of methylation of which varies between different cell types. For this embodiment, sufficiently large sets of samples are analyzed, and a map of methylation variable positions (MVPs) containing information on said levels of methylation is produced. According to the present invention, non-variable CpG positions, the methylation of which is conserved between all the representative sample types tested, are unlikely to carry disease or tissue specific information.

The methylation data afforded and produced according to the present invention not only serves as a resource to the research community, but is also directly utilized to identify useful tools, such as tissue specific markers (e.g., the inventive MHC markers disclosed herein below in EXAMPLE 1).

According to the present invention, particular variable CpG positions (MVPs) identified in healthy tissues are altered in diseased tissue. This is tested and established by inventive methylation analysis of the MVPs in comparison to other positions for diseased tissues. Accordingly, a specific subset of MVPs that are of major importance in cell differentiation, and the alteration of which is correlated with disease is thereby establishable.

Methylation patterns of specific cell types that reflect the pattern of active genes within these cells, and therefore describe the tasks a certain cell performs at a given time are establishable with the novel methods described herein. Knowledge of these patterns enables new ways to discover diagnostic and therapeutic targets, to monitor cell differentiation (e.g., in tissue engineering) and to differentiate or distinguish generally between or among different cell types, healthy and diseased, by providing a set of differentially methylated genes. The latter provides the tools, for example, for enhanced development of diagnostic products, target identification, patient stratification in clinical trials and future personalized medicines and treatments.

Unlike prior art efforts in the methylation field, the present methods are not based on, or limited to a ‘candidate’ gene approach, but provide for the discovery and use of differential methylation patterns on a genome-wide basis. The methylation blueprint (map) produced not only contributes to an understanding of factors affecting the methylation of non-coding genomic regions, but also serves as a resource for virtually all methylation research on human samples by providing the quantitative methylation level of the 5′-CG-3′ positions that are actually variable in the genome.

Collecting Samples and Sample Information

Preferably, for the inventive methods, sufficient starting material (e.g., sufficient number of samples, or nucleic acids derived from a sufficient number of samples) is acquired. Preferably, all relevant and available information (indica) on the sample types used is collected and documented, to allow for pooling of samples whenever necessary. Sufficient background information allows for a sensible decision as to which samples or sample types can be pooled in order to gain as much information as possible from as little material as is available.

Preferably, as one step of the method, a sample matrix is designed, that relates or correlates specific properties of the pooled or un-pooled sample types with a number of different analytical ‘questions’ that can be addressed with the methylation analysis described herein below.

Loci Selection

As a first step of the inventive genome-wide methylation analysis method, the loci that are investigated during the subsequent steps are selected. A locus of interest (LOI) comprises a genomic region that contains a number of CpG positions. Preferably, loci are chosen that reside in non-coding genomic regions predicted to be implicated in the regulation of neighboring genes. Preferably, the loci are selected randomly, with the only selection criterion being that a representative coverage of the genome, or of a portion thereof is achieved.

Resulting Matrix and Sample Type Selection

A subsequenct step comprises listing all different sample types that have been selected for analysis, as sample type units. Preferably, said listing is of every phenotypically distinct and identifiable cell type as independent single units in one dimension, and listing all CpG positions within the selected loci, preferably all CpG positions within the entire genome in another dimension, resulting in a large two-dimensional matrix.

According to the present invention, a functional epigenomic map is generated by filling of the matrix with the relevant quantitative methylation level information. Generation of such a map is not trivial, because the high number of methylation analyses necessary can not be performed in one experiment. Rather, a large number of experiments must be standardized in a manner allowing for an informative comparison of methylation data across different experiments; that is, a broad analysis must be performed. A major requirement of a suitable broad analysis, like the inventive one described and enabled herein, is to provide a system that generates robust data, and that comprises a data evaluation tool that normalizes said broad analysis data to enable comparison of the results across different experiments.

For utility in gaining a defined value in the two-dimensional matrix of the inventive epigenome map, the methylation data needs to be comparable in two dimensions or aspects. First, methylation levels of different CpGs within the same tissue need to be comparable to each other.

Second, methylation levels of identical CpG positions, but measured in different sample types need to be comparable to each other. An informative and useful comparison is enabled only when these requirements are fulfilled and a relativization (normalization) of the data set can be achieved. According to the present invention, these requirements are met by using the bisulfite sequencing approach in combination with the novel data evaluation tool, such as with ESME in preferred embodiments. ESME is described herein below, and in detail in the patent application EP 02 090 203 (filed at the 5^{th} of June 2002), which is incorporated herein by reference.

DNA Isolation

The different biological samples utilized in the present invention comprise nucleic acids, preferably genomic DNA. Typically, the samples comprise a mixture of methylated and unmethylated cytosine bases per CpG position. Preferably, genomic DNA used for MVP screening is isolated prior to subsequenct pre-treatment (described below), and most preferably also purified prior to said pre-treatment. Alternatively, the nucleic acids of interest are pre-treated within the environment of the biological sample. The pretreatment itself, or an equivalent thereof, is a required step in the inventive “quantitative sequencing method” (although not for the the presently disclosed methods of use of such established markers and MVP).

DNA isolation may be performed by any art-recognized method. Such protocols are well known in the art and, for example, can be found in Sambrook, Fritsch and Maniatis, Molecular Cloning: A Laboratory Manual, CSH Press, 2nd edition, 1989: Isolation of genomic DNA from mammalian cells, Protocol I, p. 9.16-9.19. A useful tool for the isolation of nucleic acids from biological samples is the QIAamp DNA mini kit (Qiagen, Hilden, Germany), which provides the necessary agents and a protocol. DNA from plasma and serum samples is preferably extracted using a QIAamp Blood Kit (Qiagen, Hilden, Germany) and the ‘blood and body fluid’ protocol as recommended by the manufacturer. DNA Purification may be done, for example, on Qiagen columns supplied in the Qiamp Blood Kit.

Bisulfite Treatment

Preferably the genomic sequences of said regions of interest (ROI; that is, the sequences at the selected loci) are known and publicly available. In EXAMPLE 1 described herein below, the genomic sequence on which the inventive analysis is applied is the Major Histocompatibility Complex MHC (SEQ ID NO:205). It is impossible to distinguish between methylated and unmethylated cytosine bases within said sequences, given only the genomic sequencing data. Such differentiation, however, becomes possible by pretreatment of the nucleic acids with an agent, or series of agents, which differentiates between methylated and unmethylated cytosine bases. According to the present invention, such an agent could be, an enzyme that interacts specifically with the one form but not with the other, for example, a methylation-sensitive restriction enzyme or a methylation-sensitive deglycosylase or deaminase (e.g., the cytidine deaminase described in Bransteitter et al., Proc Natl Acad Sci USA. 100: 4102-7, 2003), or a chemical agent. In a preferred embodiment, the nucleic acids are pretreated in such a manner that cytosine bases which are unmethylated at the 5′-position are converted to uracil, thymine, or another base which is detectably dissimilar to cytosine in terms of hybridization behavior. It is preferred that the pretreatment of nucleic acids is carried out with a bisulfite reagent (sulfite, disulfite) and that a subsequent alkaline hydrolysis takes place, which results in a conversion of non-methylated cytosine nucleobases to uracil or to another base which is detectably dissimilar to cytosine in terms of base pairing behavior.

The bisulfite-mediated conversion of the genomic sequences into ‘bisulfite sequences’ may take place in any standard, art-recognized format. This includes, but is not limited to modification within agarose gel or in denaturing solvents. The nucleic acid may be, but is not required to be, concentrated and/or otherwise conditioned before the said nucleic acid sample is pretreated with said agent. The pretreatment with bisulfite can be performed within the sample or after the nucleic acids are isolated. Preferably, pretreatment with bisulfite is performed after DNA isolation, or after isolation and purification of the nucleic acids.

The double-stranded DNA is preferentially denatured prior to pretreatment with bisulfite. The bisulfite conversion thus consists of two important steps, the sulfonation of the cytosine, and the subsequent deamination thereof. The equilibra of the reaction are on the correct side at two different temperatures for each stage of the reaction. The temperatures and length at which each stage is carried out may be varied according to the specific requirements of the situation.

Preferably, sodium bisulfite is used as described in WO 02/072880. Particularly preferred, is the so called agarose-bead method, wherein the DNA is enclosed in a matrix of agarose, thereby preventing the diffusion and renaturation of the DNA (bisulfite only reacts with single-stranded DNA), and replacing all precipitation and purification steps with fast dialysis (Olek et al., Nucleic Acids Res. 24: 5064-5066, 1996). It is further preferred that the bisulfite pretreatment is carried out in the presence of a radical scavenger or DNA denaturing agent, such as oligoethylenglycoldialkylether or preferably Dioxan. The DNA may then be amplified without need for further purification steps.

Said chemical conversion, however, may also take place in any format standard in the art. This includes, but is not limited to modification within agarose gel, in denaturing solvents or within capillaries.

Generally, the bisulfite pretreatment transforms unmethylated cytosine bases, whereas methylated cytosine bases remain unchanged. In a 100% successful bisulfite pretreatment, a complete conversion of all unmethylated cytosine bases into uracil bases takes place. During subsequent hybridization steps, uracil bases behave as thymine bases, in that they form Watson-Crick base pairs with adenine bases. Only cytosine bases that are located in a CpG position (i.e., in a 5′-CG-3′ dinucleotide), are known to be possibly methylated (known to be normally methylatable in vivo). Therefore all other cytosines, not located in a CpG position, are unmethylated and are thus transformed into uracils that will pair with adenine during amplification cycles, and as such will appear as thymine bases in an amplified product (e.g., in a PCR product). Whenever a bisulfite-treated nucleic acid is amplified and/or sequence analyzed, the positions that appear as thymines in the sequence can either indicate a true thymine position or a (transformed or converted) cytosine position. These can only be distinguished by comparing the bisulfite sequence data with the untreated genomic sequence data that is already known.

However, cytosines in CpG positions must be regarded as potentially methylated, more precisely as potentially differentially methylated. Significantly, a 100% cytosine or 100% thymine signal at a CpG position will be rare, because biological samples always contain some kind of background DNA. Therefore, according to the inventive methods, the ratio of thymine to cytosine appearing at a specific CpG position is determined as accurately as possible. This is enabled, for example, by using the sequencing evaluation software tool ESME, which takes into account the falsification or bias of this ratio caused by incomplete conversion (see herein below, and see application EP 02 090 203, incorporated herein by reference.

Primer Design

Preferably, the bisulfite-pretreated DNA is not directly sequenced, but amplified first. Primer molecules are designed that will be utilized to amplify regions of interest (ROI). It is particularly preferred that the regions of interest are amplified by means of a polymerase chain reaction. This ensures that sufficient material for a qualitative automated sequencing process can be provided. Primer molecules for the amplification must be carefully designed, because priming at a genomic CpG position (i.e., a 5′-tG-3′, or 5′-CG-3′, or 5′-Ca-3′ dinucleotide in the bisulfite sequence) must be avoided (a capital T is used to indicate a thymine position that was a thymine prior to pretreatment, whereas a “t” is used to indicate a thymine at a cytosine position, whenever the cytosine was transformed to uracil by pretreatment and “a” is used to indicate the adenine corresponding to such a thymine located at a cytosine position). Primer molecules that cover a genomic CpG position when binding to the bisulfite-pretreated nucleic acids will introduce a bias towards amplifying one methylation status only, because they distinguish between ‘prior-to-pretreatment’ methylated and unmethylated nucleic acids as templates. Preferably, therefore, inventive unbiased primer molecules that are used to amplify nucleic acids pretreated with bisulfite consist of three different nucleotides only (i.e., A, T and C), and preferably only comprise a 5′-CA-3′ sequence if that corresponding complementary 5′-TG-3′ sequence was known to be a 5′-TG-3′ sequence prior to pretreatment, as, for example, the bisulfite pretreatment.

Preferably, therefore, the inventive primer molecules are designed not to cover any CpG position, to avoid a bias in amplification.

More details about the preferred primer design, especially if multiplex PCR experiments are performed on bisulfite treated nucleic acids, are found in German Patent Application DE 102 36 406, filed 02 Aug. 2002, and filed as a PCT application in English both of which are incorporated herein by reference.

Generally, the sense strand or the minus strand of the genomic DNA can be utilized to analyze the methylation levels of CpG positions within a genomic sequence. After bisulfite treatment, these strands differ from each other to such an extent that they are not corresponding (complementary) anymore, and they do not hybridize efficiently to each other. These are referred to herein as BISU 1 and BISU 2. Both can be used for methylation analysis, and that is why both strands are encompassed withing the teachings of the present invention. As the bisulfite sequences also differ depending on their prior corresponding genomic methylation status, both BISU sequences are disclosed once as up-methylated (every 5′-CG-3′ is methylated) and once as down-methylated (every 5′-CG-3′ is unmethylated). Accordingly, four bisulfite sequences are disclosed per genomic ROI.

In the sequence protocol herein, the two strands of the up-methylated versions of all 34 ROIs from EXAMPLE 1 are given first (SEQ ID NOS:1-68), where the odd numbers indicate the BISU 1, and the even numbers name the BISU 2 sequences. These are followed by the sequences of the corresponding down methylated versions of said ROIs (SEQ ID NOS:69-136). Again, the odd numbers indicate BISU 1 and even numbers indicate BISU 2 sequences. Nucleic acids and oligomers comprising a contiguous sequence of a length of at least 16 nucleotides or more (or at least 18, 20, 22, 23, 25, 30, or 35) nucleotides that hybridize under moderately stringent or stringent conditions to any of these four sequences can be used to analyze the methylation levels of specific CpGs or methylation patterns of short stretches of the nucleic acid within these regions of interest (ROI).

Designing primer molecules for only one of the strands, provides for a selection towards one strand. Amplification of the BISU1 version of the ROI is afforded by using a set of primer molecules designed for the bisulfite-treated sense strand BISU 1. These amplificates are typically just as useful for the determination of methylation levels at a genomic CpG position as amplificates of BISU 2. Therefore, it is understood that the scope of this application is not limited by describing the primer molecules that have been used for the analysis of only one strand.

The amplificates obtained are analyzed by sequencing as described in the next step. The double-stranded DNA amplificates (e.g., obtained by PCR) contain a thymine instead of an unmethylated cytosine in one strand and, correspondingly, an adenine in the inversely complementary strand. Consequently, by determining the thymine signal intensities at original cytosine positions in CpG position, the fraction of unmethylated cytosines can be determined at this CpG position in the present mixture. Each amplificate is bisulfite sequenced once from both ends, and in particularly preferred embodiments two sequence traces are generated thereby.

Sequencing primers may be designed specifically for that purpose, although it is preferred that if a PCR is employed to amplify the regions of interest, the original PCR amplification primers are used as the sequencing primers.

Preferably, both of these two sequence traces are analyzed with one or more algorithms or a software program that considers or accounts for any unequal distribution of bases in bisulfite-converted DNA and that normalizes the sequence traces (electropherograms) to allow for quantitation of methylation signals within the sequence traces. Preverably, the program is ESME as is described in detail in the following part, or is a functional equivalent thereof. Preferably, an average value from both of these traces for the methylation level at one CpG is calculated for every CpG position in the analyzed region.

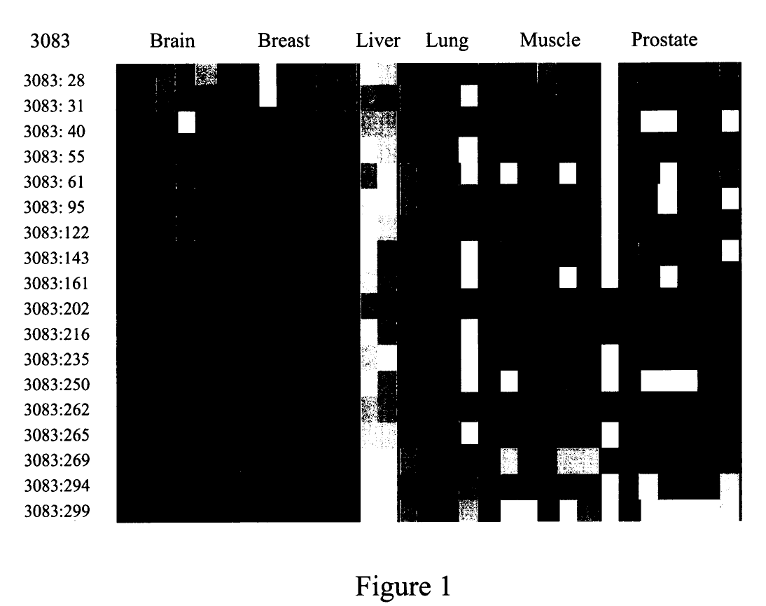

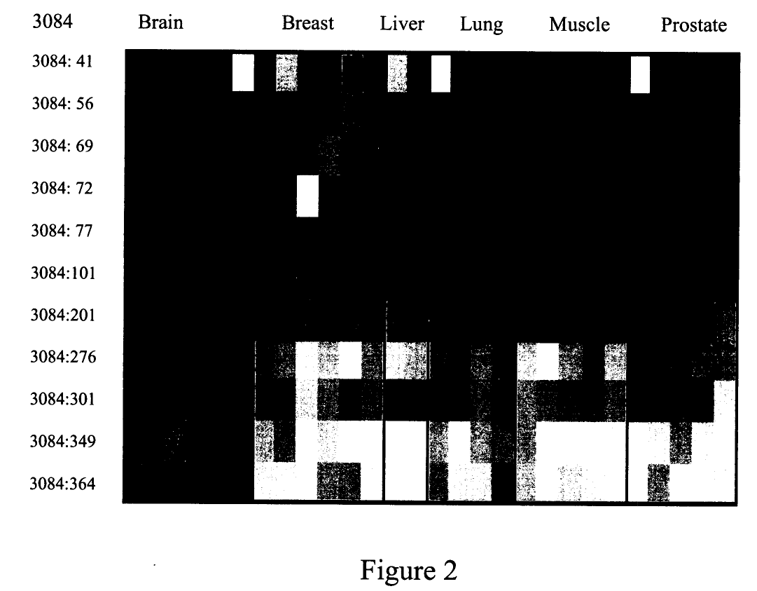

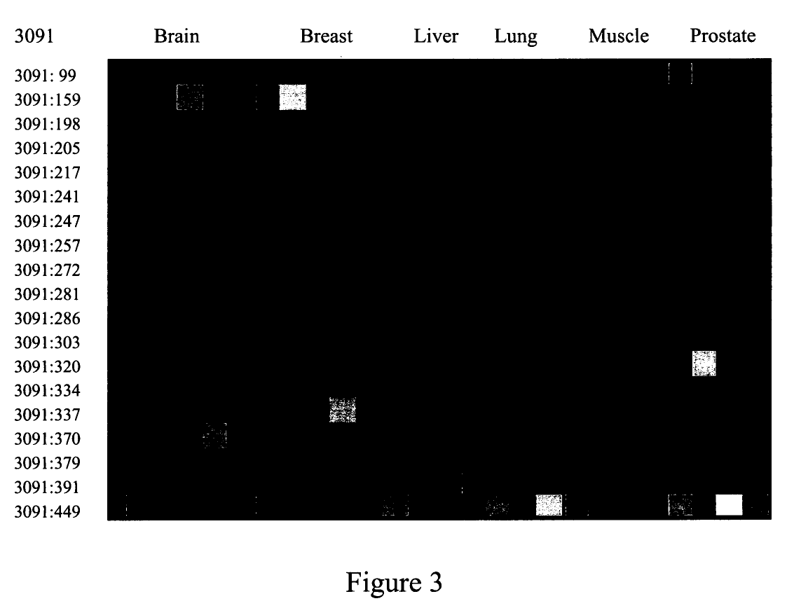

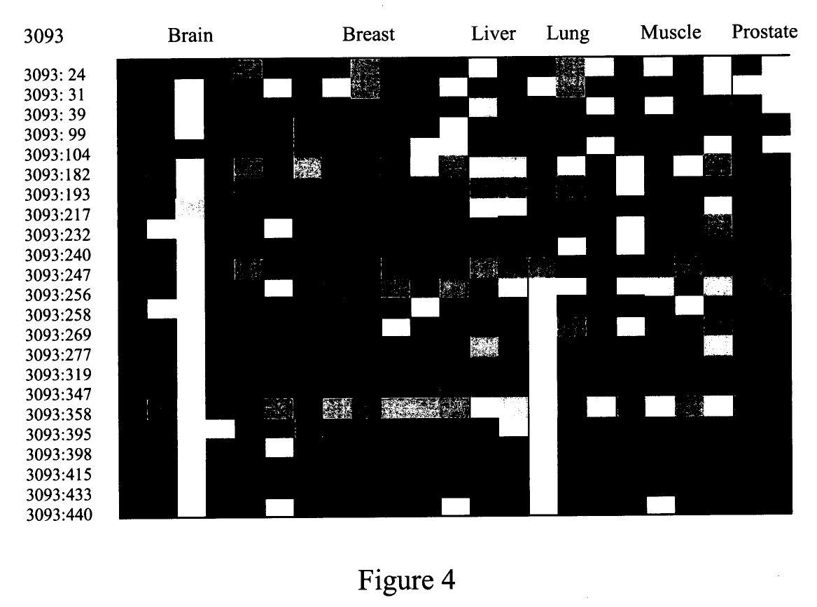

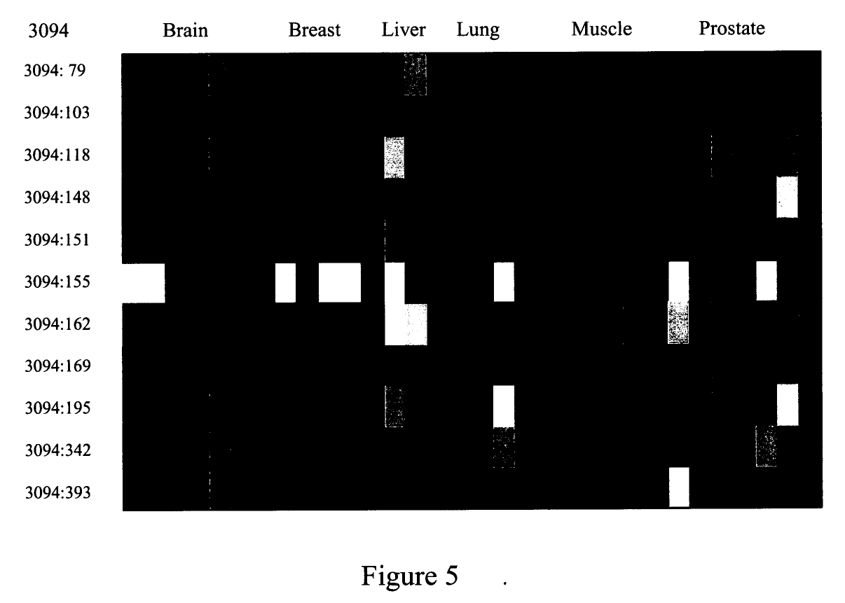

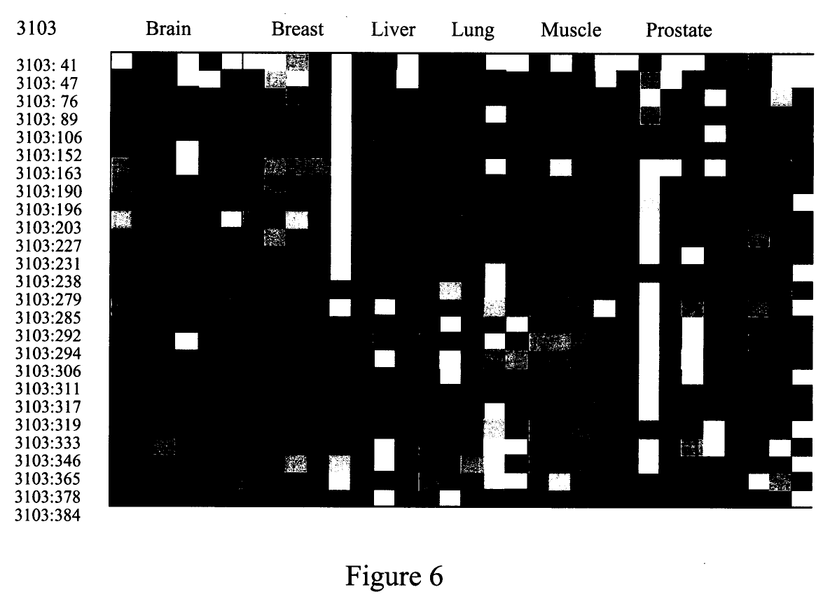

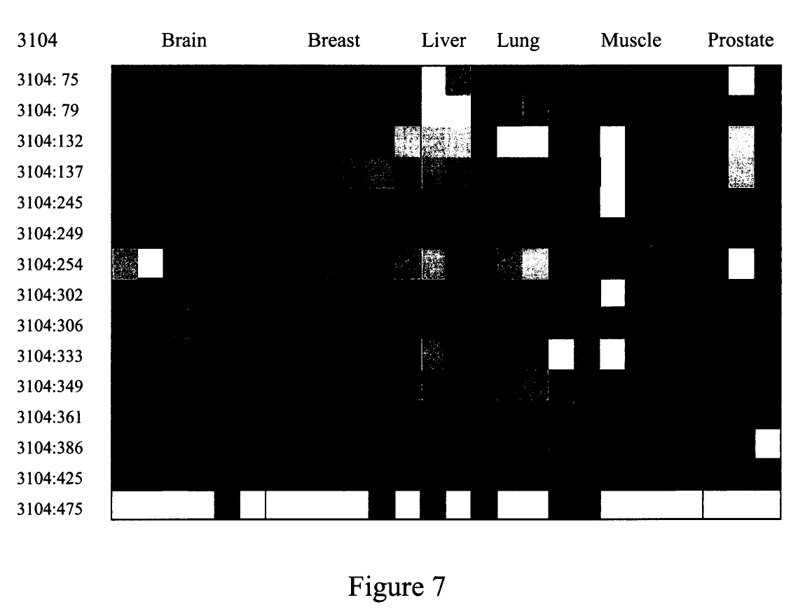

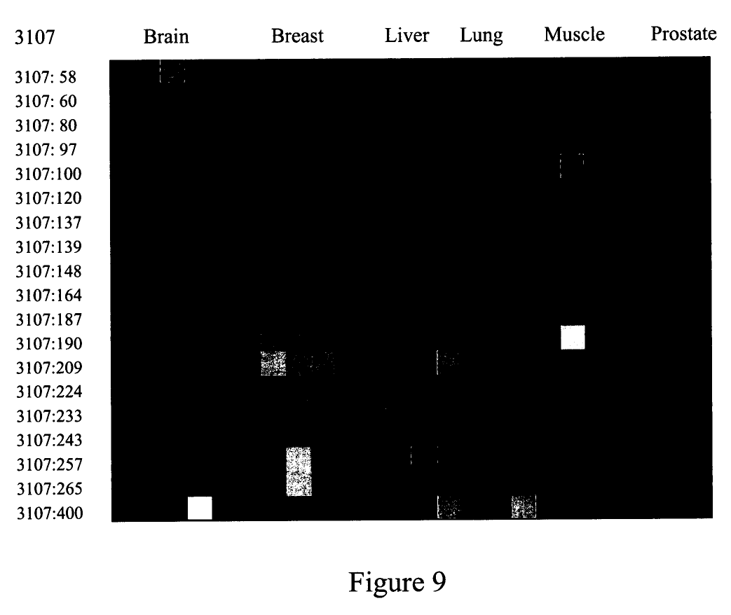

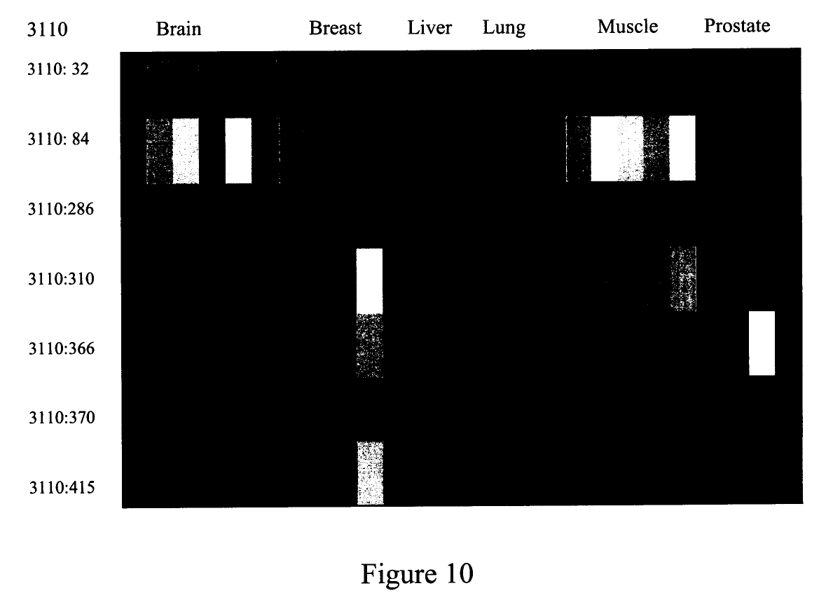

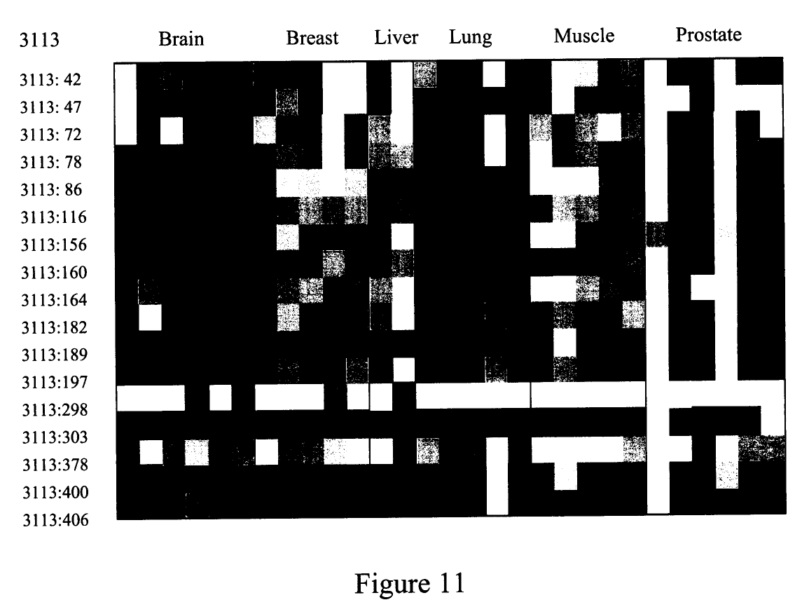

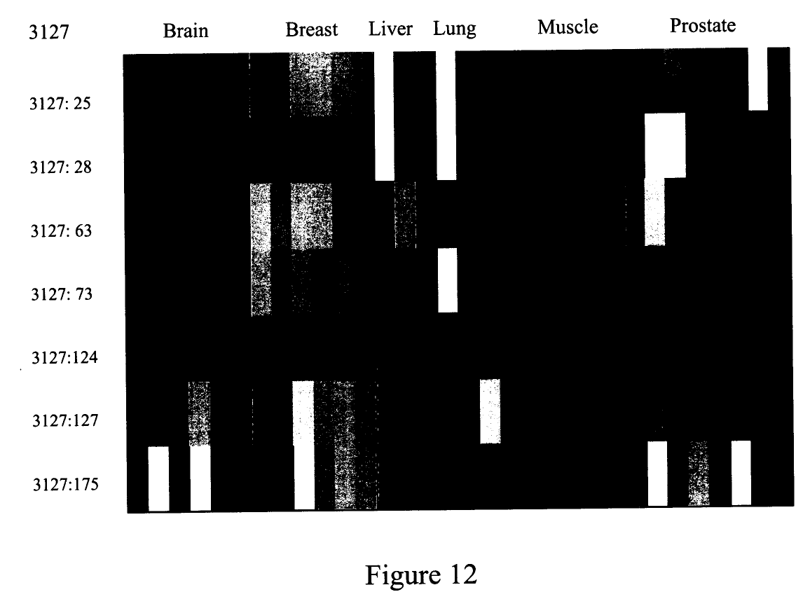

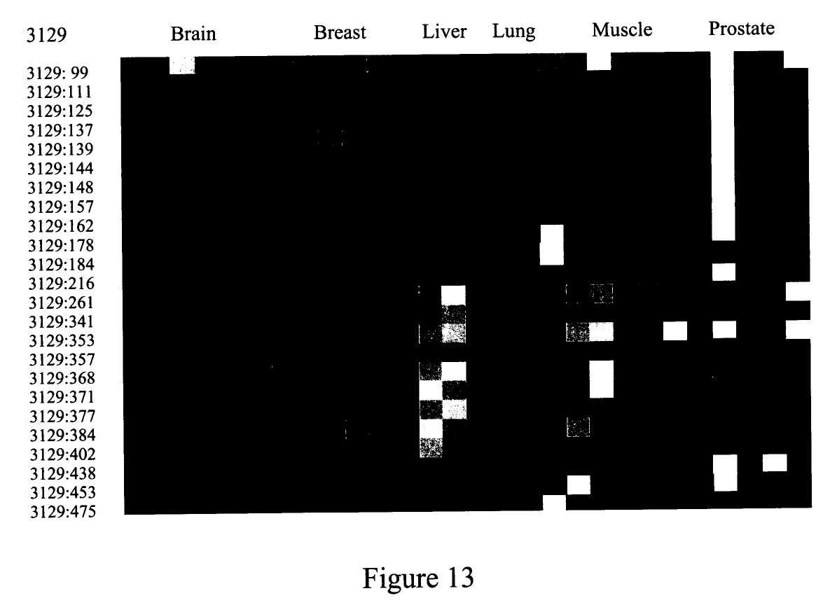

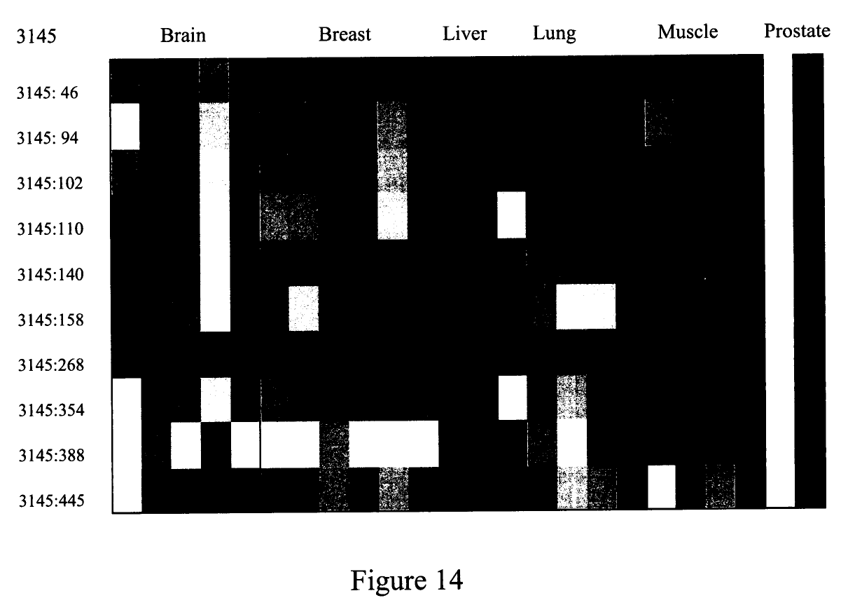

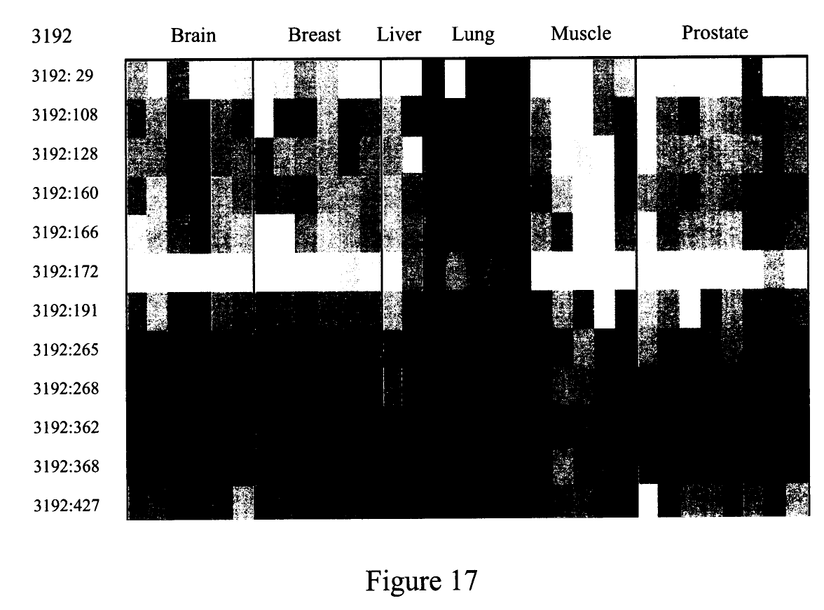

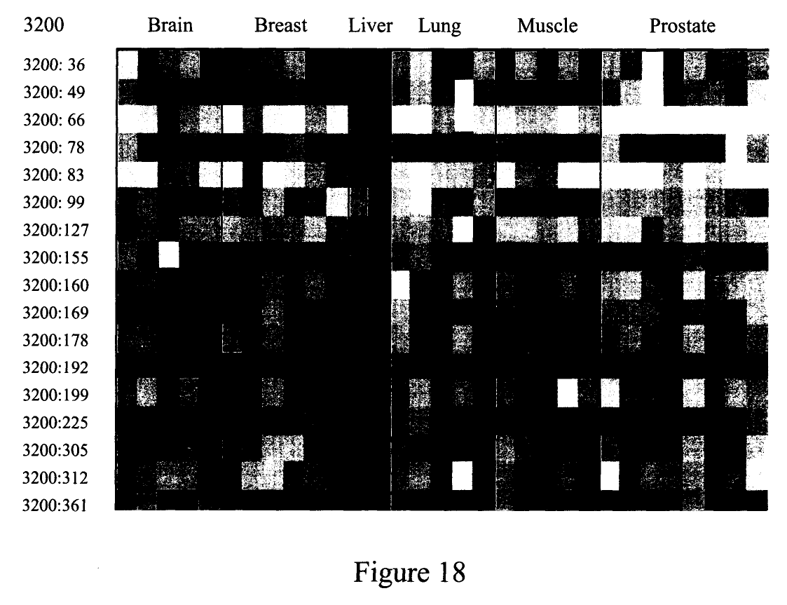

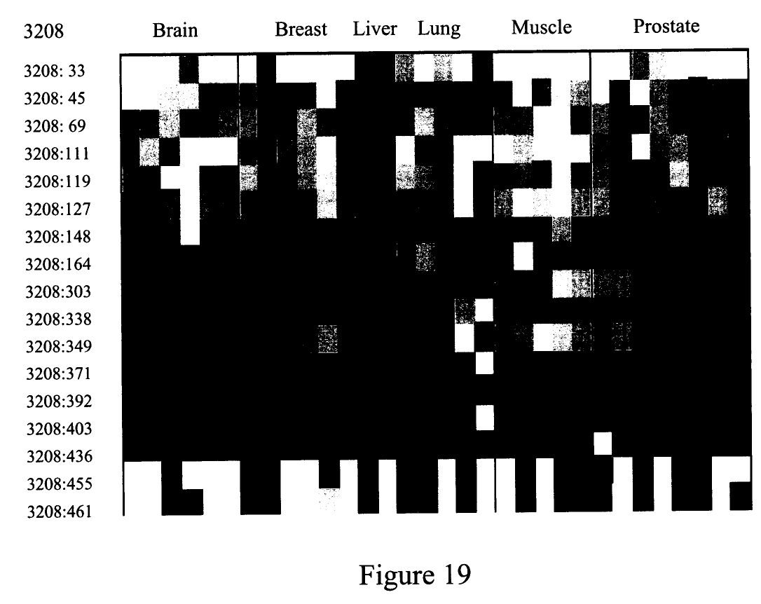

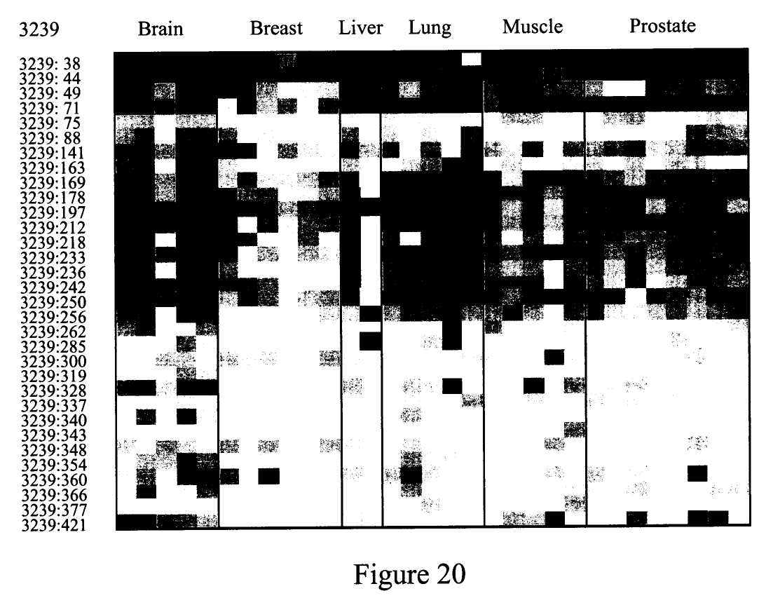

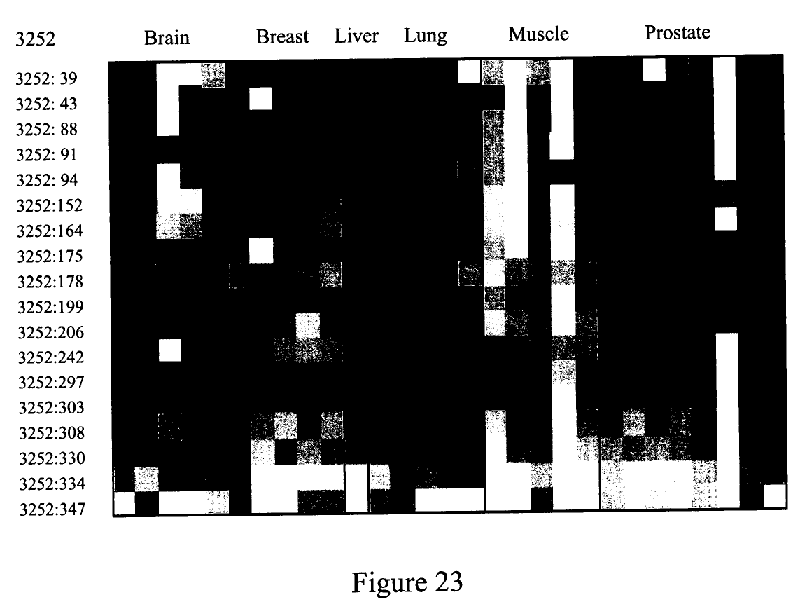

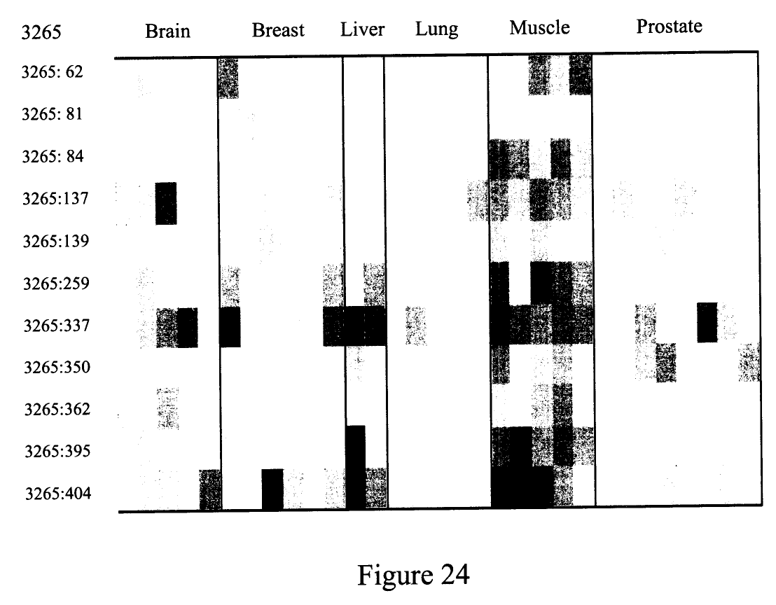

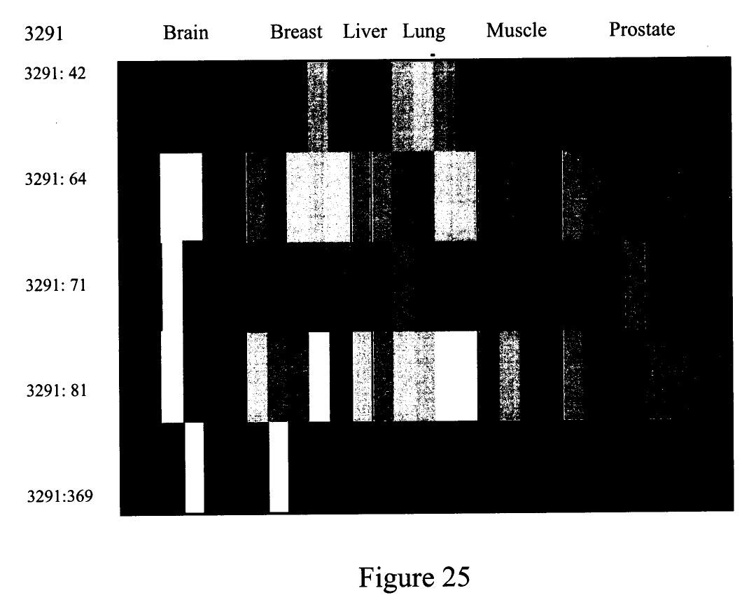

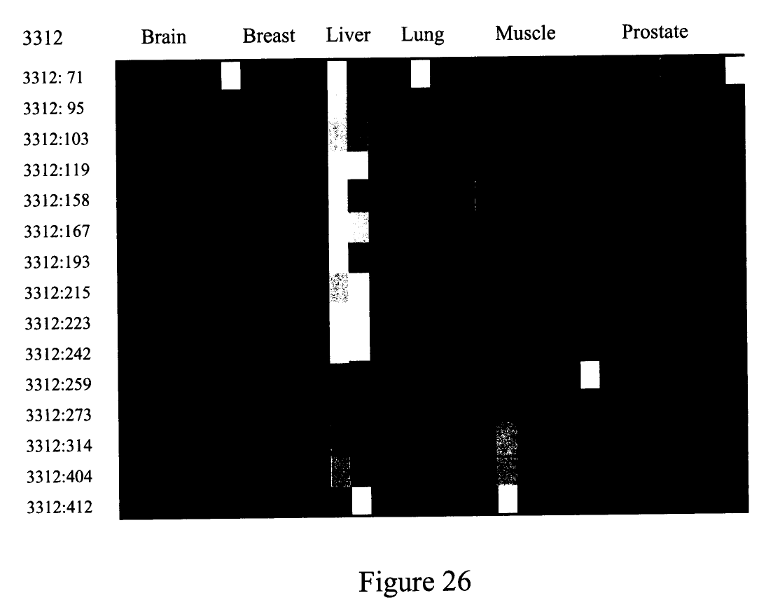

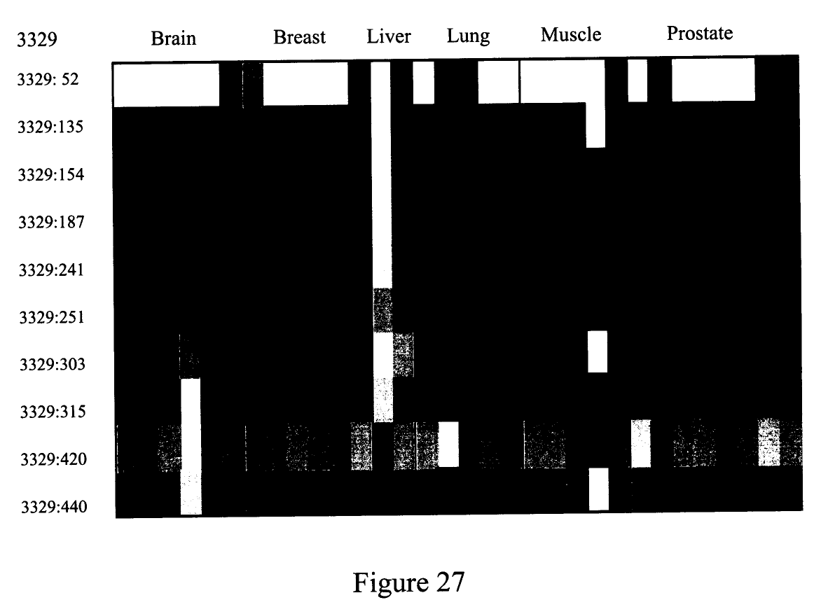

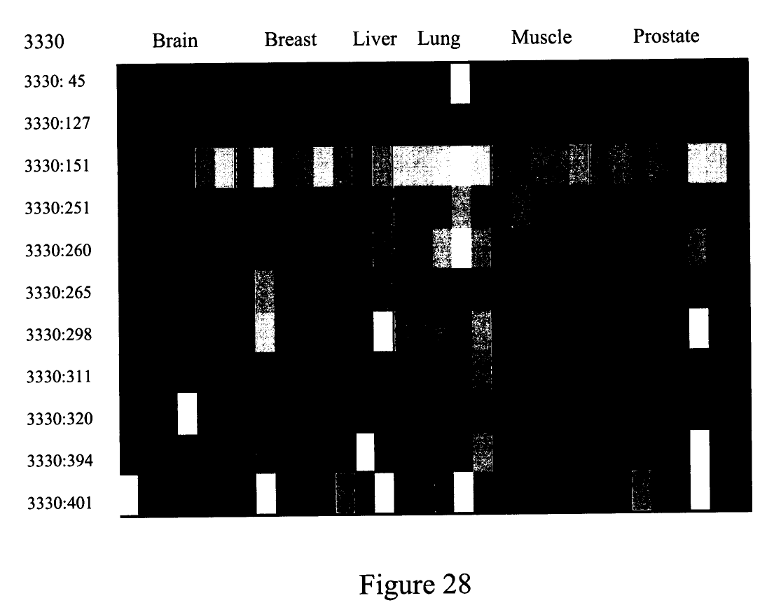

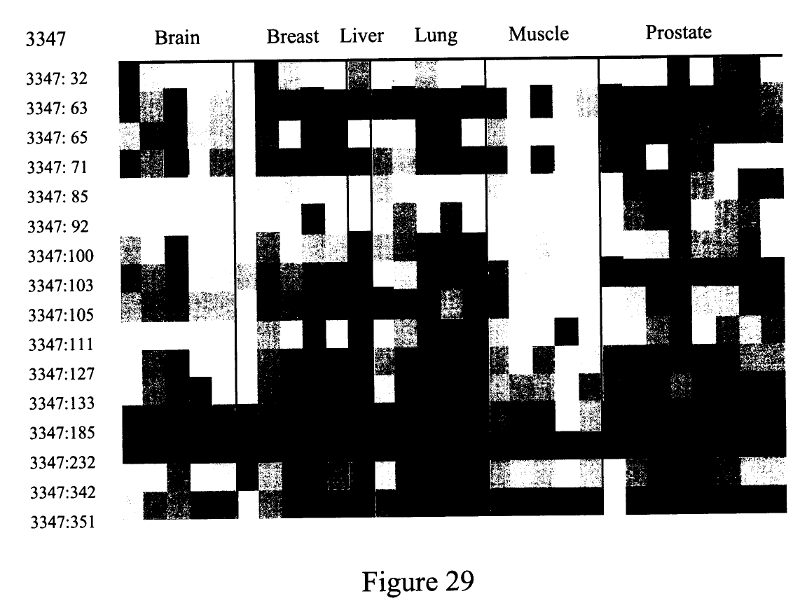

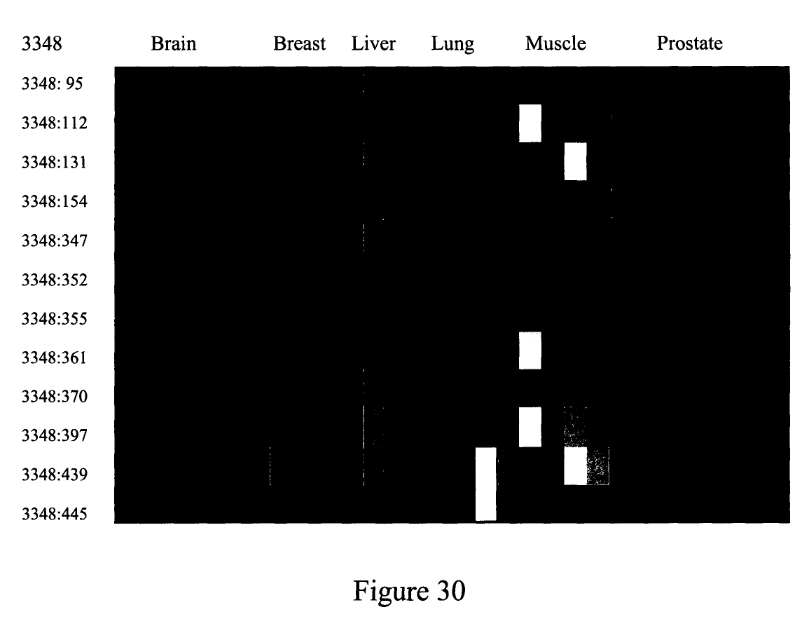

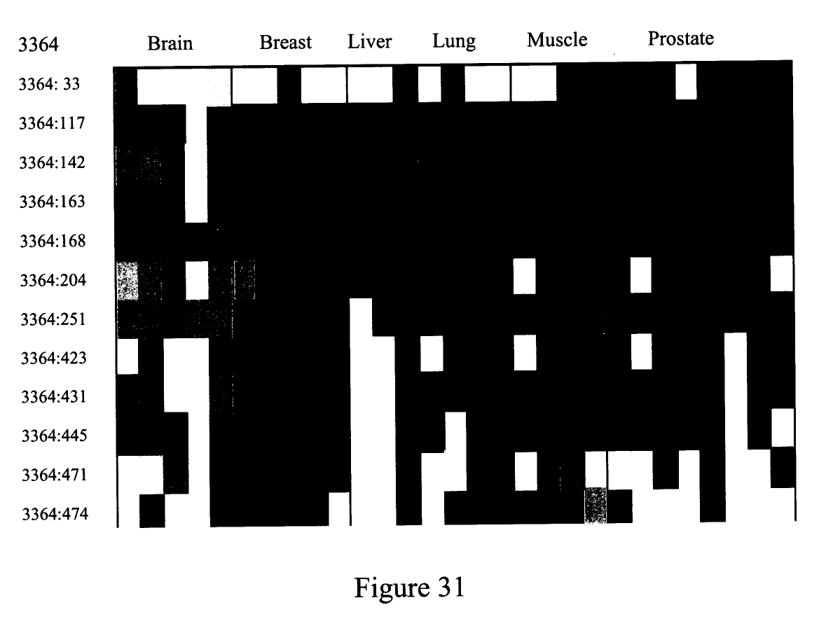

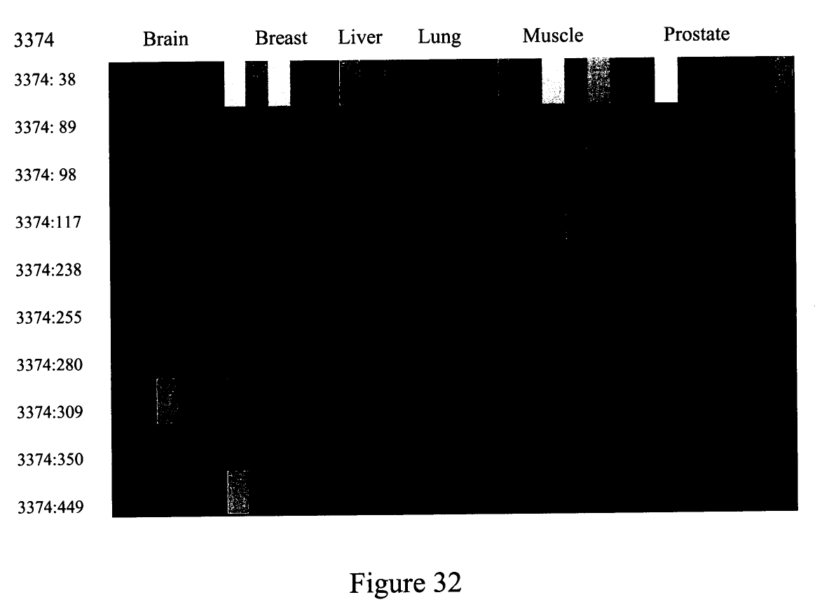

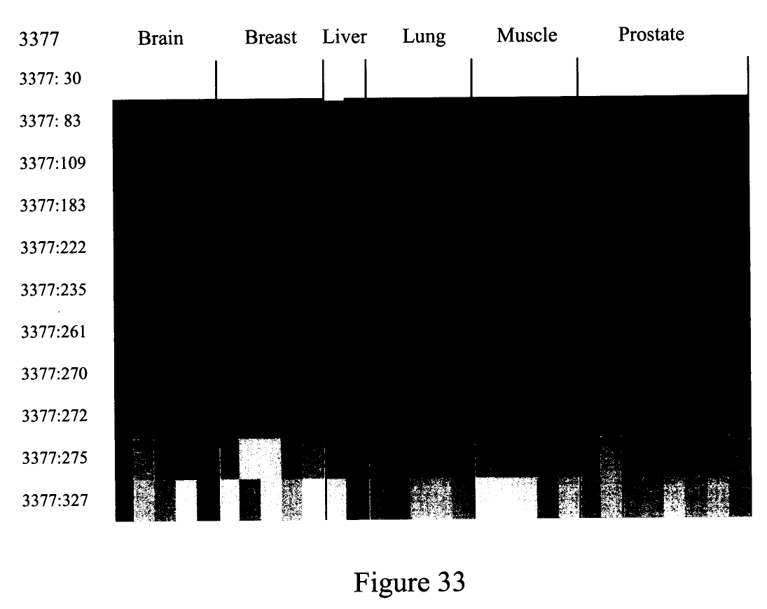

Averaged values for a number (between 5 and 32) of analyzed CpG positions in each of 34 ROIs are shown in EXAMPLE 1, herein below (see FIGS. 1-34, and Tables 3-36).

DNA Sequencing

According to the present invention, generating a genome-wide methylation map requires several thousand PCR amplificates and about twice as many sequence reads are produced and analyzed for differential methylation. Preferably, the amplificates of the pretreated nucleic acids are first sequenced according to the chain-termination method as described by Sanger et al. (Sanger F, et al., Proc Natl Acad Sci USA 74: 5463-5467, 1977), slightly adapted for bisulfite sequencing (Feil R, et al., Nucleic Acids Res. 22: 695-6, 1994)

The labeled reaction products are subsequently analyzed according to their size either in spatially separated lanes, or by different color labels distinguishable within one lane. For example, four different fluorescently-labeled ddNTPs may be used, but it is also possible to limit the analysis to the determination of fewer than four base sequences.

The sequence analysis results in an electropherogram which can only be used for a qualitative determination of the base sequence. With the use of the preferred sequence data evaluation tool ESME however, or a functional equivalent thereof, quantitative information with respect to the level of methylation of a cytosine can also be obtained from this electropherogram, and from the comparison of these data with the original sequence; that is, with the sequence of the corresponding DNA region not treated with bisulfite.

ESME

ESME calculates methylation levels at particular CpG positions by comparing signal intensities, and correcting for incomplete bisulphite conversion. ESME scores all cytosines (=methylated C) and C→T transitions (=non-methylated C) in bisulphite sequence traces, and furthermore calculates the % of methylation for all CpG sites. It allows the analysis of DNA mixtures both in individual cells as well as of DNA mixtures from a plurality of cells. The method can be applied to any bisulfite-pretreated nucleic acid for which the genomic nucleotide sequence of the corresponding DNA region not treated with bisulfite is known, and for which a sequence electropherogram (trace) can also be generated.

ESME utilizes the electropherograms for standardizing the average signal intensity of at least one base type (C, T, A or G) against the average signal intensity which is obtained for one or more of the remaining base types. Preferably, the cytosine signal intensities are standardized relative to the thymine signal intensities, and the ratio of the average signal intensity of cytosine to that of thymine is determined.

The average of a signal intensity is calculated by taking into account the signal intensities of several bases, which are present in a randomly defined region of the amplificate. The average of a plurality of positions of this base type is determined within an arbitrarily defined region of the amplificate. This region can comprise the entire amplificate, or a portion thereof.

Significantly, such averaging leads to mathematically reasonable and/or statistically reliable values.

Additionally, a basic feature of ESME comprises calculation of a ‘conversion rate’ (fcoN) of the conversion of cytosine to uracil (as a consequence of bisulfite treatment), based upon the standardized signal intensities. This is characterized as the ratio of at least one signal intensity standardized at positions which modify their hybridization behavior due to the pretreatment, to at least one other signal intensity. Preferably, it is the ratio of unmethylated cytosine bases, whose hybridization behavior was modified (into the hybridization behavior of thymine) by bisulfite treatment, to all unmethylated cytosine bases, independent of whether their hybridization behavior was modified or not, within a defined sequence region. The region to be considered can comprise the length of the total amplificate, or only a part of it, and both the sense sequence or its inversely-complementary sequence can be utilized therefore.

The calculation of standardizing factors, for standardizing signal intensities, as well as the calculation of a conversion rate are based on accurate knowledge of signal intensities. Preferably, such knowledge is as accurate as possible.

An electropherogram represents a curve that reflects the number of detected signals per unit of time, which in turn reflects the spatial distance between two bases (as an inherent characteristic of the sequencing method). Therefore, the signal intensity and thus the number of molecules that bear that signal can be calculated by the area under the peak (i.e., under the local maximum of this curve). The considered area is best described by integrating this curve. Such area measurements are determined by the integration limits X₁ and X₂; X₁, lying to the left of the local maximum, and by X₂, lying to the right of the local maximum.

Another basic feature of ESME is that it affords the determination of the actual methylation number f_{MET}, (“actual” as in significantly closer to reality than assuming the conversion rate is, e.g., 95%). Both, the standardized signal intensities as well as the conversion rates f_{CON} (obtained by considering said standardized signal intensities) are used for calculation of the actual degree (level) of methylation of a cytosine position in question.

According to a preferred embodiment, the % methylation levels are calculated by ESME, or an equivalent thereof, for all CpG positions representing the genome, and the information is linked to corresponding positions in the latest assembly of the human genome sequence, and be sorted according to tissue and disease state. In preferred embodiments, this information is made available for further research. In a particularly preferred embodiment, the information is utilized directly to provide specific markers for DNA derived from specific cell types (e.g., see EXAMPLE 1 herein below).

The methylation data, including the quantitative aspects thereof, is easily presented in a user friendly two-dimensional display, allowing for immediate identification of differentiating patterns. For example, the location of a CpG position within the genome is displayed along one axis, whereas the sample type is displayed along the other axis. When grouping the phenotypically distinct sample types side-by-side, methylation differences can be displayed in the field created by the two axes. Based on this visualized display, methylation variable positions (MVPs) can be identified (e.g., by eye) and it becomes easy to select the ROIs that can be utilized as effective markers. The exact location of the methylation variable positions i.e., the CpG positions that are differentially methylated between or among different groups of phenotypically distinct cell types could also be disclosed and analyzed using such a display.

Utility

Embodiments of the present invention have specific and substantial utility for any researcher involved with DNA analysis, including but not limited to technical developers, medicinal researchers, criminal investigators, and forensics scientists. The inventive methods and tools disclosed herein are extremely useful, for example in identifying the source of DNA found in a bodily fluid or DNA found at a crime scene, or more specifically, from which organ or tissue type the DNA originates from.

In additional embodiments the inventive markers are arranged as an appropriate set on a chip surface, and used to simultaneously detect specific methylation degrees (levels) of a large number of MVPs. The term ‘appropriate’ in this context is defined by the specificity of the markers used and their correlation towards the question raised. Such embodiments are particularly useful where the origin of DNA must be identified without any prior knowledge as to where it may have originated from. For these cases, sets of markers that are analyzed for their methylation degrees can create fingerprints or patterns that lead to a accurate identification of the DNA's origin.

However, according to the present invention, the use of a single marker ROI is often sufficient if the problem at hand involves distinguishing between two specific tissues in question. Likewise, if analysis of only a few different marker ROI will give sufficient information towards an unambiguous decision, any kind of methylation analysis assay, that allows for determination of the methylation levels at specific locations is sufficient. Such assays could be based on methylation-sensitive restriction enzyme assays, given that the informative MVPs were located in an appropriate recognition motif sequence. Alternatively, the assay could be based on bisulfite-pretreated DNA, or on DNA subjected to other pretreatments distinguishing between methylated and unmethylated cytosines. The pretreated DNA can then be analyzed by means of sequencing the pretreated DNA or by means of assays based on bisulfite sequencing (for example pyrosequencing or MS-SNuPE™). The pretreated DNA can also be analyzed by means of methylation-specific ligation assays, amplification with methylation specific primers (MSP), amplification using methylation-specific blockers (HM; HeavyMethyl™) or by methylation-specific detection of PCR products (MethyLight™), or by any combinations thereof.

The so-called HeavyMethyl™ (HM) assay comprises the use of at least one blocking oligomer; that is, a nucleic acid molecule or peptide nucleic acid molecule, comprising in each case a contiguous sequence at least 9 nucleotides in length that is complementary to, or hybridizes under moderately stringent or stringent conditions to a sequence comprising a CG, TG or CA dinucleotide, that was a CG dinucleotide prior to pretreatment, wherein hybridization of said nucleic acid to a target sequence hinders the amplification of the target sequence.

Preferably, this blocking oligomer is in each case modified at the 5′-end thereof to preclude degradation by an enzyme having 5′-3′ exonuclease activity. Preferably, said blocking oligomer is in each case lacking a 3′ hydroxyl group.

All of these methylation assay techniques are known and sufficiently described in the prior art.

The present invention is based, at least in part, on the discovery that quantitative measurements of the methylation levels of several genomic regions can be performed in a fast and high-throughput style on different sample types resulting in easily identifiable biomarkers.

In one embodiment, the present invention therefore provides a method for generating a genome-wide methylation map (epigenomic map) by identifying a significant number of methylation variable positions (MVPs) within the human genome, comprising several steps:

First, is collecting a number of phenotypically distinct biological samples, wherein such samples can be derived from different types of tissue, organs, bodily fluids or cells, or from patients suffering from different diseases, or from patients suffering from one disease, but to different degrees, and wherein such samples are characterized in containing genomic DNA.

Secondly, said genomic DNA is pretreated, before or after isolation and/or purifying, by contacting them with an agent, or series of agents, that modifies unmethylated cytosine, but does not modify methylated cytosines at all, or at least in the same manner.

Thirdly, segments of genomic regions, representing the whole or a chosen part of the genome, and each comprising at least one CpG position are amplified;wherein a CpG position is the position of a CG or TG dinucleotide, which was a CG dinucleotide prior to performing pretreatment in step two, and wherein said amplification is carried out using the pretreated nucleic acid as the template by means of primer molecules that do not distinguish between initially methylated and initially unmethylated DNA. This step is performed separately for every type of phenotypically distinct biological sample in question.

In a fourth step, said amplified pretreated nucleic acids are sequence analyzed.

In a fifth step, the sequence traces (e.g., electropherograms) derived for every type of biological sample are analyzed, to determine the quantitative level of methylation at several specific CpG positions, creating a pattern of the levels of methylation over said whole or said chosen part of the genome.

Next, said levels of methylation at several specific CpG positions are compared between different groups of at least two types of biological samples, and methylation variable positions (MVP) are identified, wherein a MVP comprises a CpG position, for which a difference in methylation levels can be detected between different types of biological samples.

Preferably, determining the quantitative level of methylation at several specific CpG positions, comprises the algorithms and principle ideas underlying the software program ESME™, or a functional equivanent thereof, as used for analysis of the sequence traces.

Preferably, pretreatment in step 2 comprises conversion of unmethylated cytosine to uracil, whereas methylated cytosine is not converted by said pretreatment.

It is also preferred that the agent, or series of agents of step 2 comprises a bisulfite reagent.

It is alternately preferred that the agent, or series of agents in step 2 comprises an enzyme, such as a cytidine deaminase.

Preferably, the genomice DNA segments selected in step 3 are located in or near the 5′-regulatory region of a gene.

It is particularly preferred that the amplifying step is by polymerase chain reaction (PCR).

Addtionaly embodiments of this invention comprise a nucleic acid or an oligomer, comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA selected from a group consisting of SEQ ID NOS:1-136, and sequences complementary thereto, wherein said nucleic acid or oligomer sequence comprises at least one methylation variable position.

These nucleotides and oligomers are extremely useful to analyze the methylation levels of said MVPs, for example, in sequencing analysis or in other quantifying assays, which detect the ratio of methylated versus non-methylated nucleotides (e.g., a MSP assay, employing methylation-sensitive primer molecules comprising at least one MVP, or a HeavyMethyl™ assay, employing methylation sensitive blocking oligonucleotides (as described in detail in WO 02/072880) or a MethyLight™ assay employing methylation sensitive detection oligonucleotides).

Another embodiment of this invention comprises a set of two oligomers that allows the generation of nucleic acid amplificates, wherein a first oligomer comprises at least one contiguous base sequence of at least 16 nucleotides in length (or at least 18, 20, 22, 23, 25, 30 or 35 nucleotides), which is complementary to, or hybridizes under moderately stringent or stringent conditions to a pretreated genomic DNA sequence selected from the group consisting of SEQ ID NOS:1-136, and the second oligomer comprises in each case at least one contiguous base sequence of at least 16 nucleotides in length (or at least 18, 20, 22, 23, 25, 30 or 35 nucleotides), which is essentially identical to said pretreated genomic DNA sequence selected from the group consisting of SEQ ID NOS:1-136, respectively.

Examples of inventive oligonucleotides of length X (in nucleotides), as indicated by polynucleotide positions with reference to, e.g., SEQ ID NO:1, include those corresponding to sets (e.g., sense and antisense) of consecutively overlapping oligonucleotides of length X, where the oligonucleotides within each consecutively overlapping set (corresponding to a given X value) are defined as the finite set of Z oligonucleotides from nucleotide positions:

n to (n+(X−1));

where n=1, 2, 3, . . . (Y−(X−1));

where Y equals the length (nucleotides or base pairs) of SEQ ID NO:1 (2,500);

where X equals the common length (in nucleotides) of each oligonucleotide in the set (e.g., X=20 for a set of consecutively overlapping 20-mers); and

where the number (Z) of consecutively overlapping oligomers of length X for a given SEQ ID NO of length Y is equal to Y−(X−1). For example Z=2,500−19=2,481 for either sense or antisense sets of SEQ ID NO:1, where X=20.

In particular embodiments, preferred sets are those limited to those oligomers that comprise at least one CpG, tpG or Cpa dinucleotide.

Examples of inventive 20-mer oligonucleotides include the following set of 2,481 oligomers (and the complementary antisense set), indicated by polynucleotide positions with reference to SEQ ID NO:1:

1-20, 2-21, 3-22, 4-23, 5-24 . . . 2,480-2,498, 2,481-2,499 and 2,481-2,500.

In particular embodiments, preferred sets are those limited to those oligomers that comprise at least one CpG, tpG or Cpa dinucleotide.

The present invention encompasses, for each of SEQ ID NO:1 to SEQ ID NO:136 (sense and antisense), multiple consecutively overlapping sets of oligonucleotides or modified oligonucleotides of at least length X, where, e.g., X=9, 10, 17, 18, 20, 22, 23, 25, 27, 30 or 35 nucleotides.

The oligonucleotides of the invention can also be modified by chemically linking the oligonucleotide to one or more moieties or conjugates to enhance the activity, stability or detection of the oligonucleotide. Such moieties or conjugates include chromophores, fluorophors, lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. The probes may also exist in the form of a PNA (peptide nucleic acid) which has particularly preferred pairing properties. Thus, the oligonucleotide may include other appended groups such as peptides, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a chromophore, fluorophor, peptide, hybridization-triggered cross-linking agent, transport agent, hybridization-triggered cleavage agent, etc.

The oligonucleotide may also comprise at least one art-recognized modified sugar and/or base moiety, or may comprise a modified backbone or non-natural internucleoside linkage.

In preferred embodiments, at least one, and more preferably all members of a set of oligonucleotides is bound to a solid phase.

In particular embodiments, it is preferred that an arrangement of different oligonucleotides and/or PNA-oligomers (a so-called “array”), made according to the present invention, is present in a manner that it is likewise bound to a solid phase. Such an array of different oligonucleotide- and/or PNA-oligomer sequences can be characterized, for example, in that it is arranged on the solid phase in the form of a rectangular or hexagonal lattice. The solid-phase surface is preferably composed of silicon, glass, polystyrene, aluminum, steel, iron, copper, nickel, silver, or gold. However, nitrocellulose as well as plastics such as nylon, which can exist in the form of pellets or also as resin matrices, may also be used.

Therefore, in further embodiments, the present invention provides a method for manufacturing an array fixed to a carrier material for analysis in connection with, for example, identification of cell or tissue types, or distinguishing one cell or tissue type among others, in which method at least one oligomer according to the present invention is coupled to a solid phase. Methods for manufacturing such arrays are known and described in, for example, U.S. Pat. No. 5,744,305 by means of solid-phase chemistry and photo labile protecting groups.

The present invention further provides a DNA chip for the analysis of, for example, identification of cell or tissue types, or for distinguishing one cell or tissue type among others. DNA chips are known and described in, for example, U.S. Pat. No. 5,837,832.

Especially preferred, is a nucleic acid or oligomer, consisting essentially of one of the sequences selected from the group consisting of SEQ ID NO:137 to SEQ ID NO:204. These preferred nucleic acid molecules were used as primer molecules in EXAMPLE 1, herein below, to generate amplificates that comprise at least two MVPs, and which can be used to differentiate tissues by for example sequencing said amplificates.

Another embodiment of this invention comprises a method for identifying a specific type of cells out of a group of other chosen cell types as the source of a nucleic acid analyzed, comprising determination of methylation state or the level of methylation of one or more MVPs within any sequence of the MHC selected from the group consisting of SEQ ID NO:205, a fragment thereof at least 16 (or at least 18, 20, 22, 23, 25, 30 or 35 nucleotides) contiguous nucleotides in length, and sequences that are complementary to, or hybridize under moderately stringent or stringent conditions to SEQ ID NO:205 or to a fragment thereof at least 16 (or at least 18, 20, 22, 23, 25, 30 or 35 nucleotides) contiguous nucleotides in length.

Preferably, said state or level of methylation is analyzed and determined by utilizing a nucleic acid or an oligomer comprising at least one base contiguous sequence having a length of at least 16 nucleotides (or at least 18, 20, 22, 23, 25, 30 or 35 nucleotides), which is complementary to, or hybridizes under moderately stringent or stringent conditions to a pretreated genomic DNA sequence selected from the group consisting of SEQ ID NOS:1-136, or sequences complementary thereto.

It is particularly preferred that said state or level of methylation is analyzed by utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:1-136, and sequences complementary thereto, wherein said nucleic acid or oligomer sequence comprises at least one methylation variable position.

It is also preferred that said state or level of methylation is analyzed by a method comprising utilizing a methylation-sensitive restriction enzyme analysis assay, and utilizing one or several of the 34 genomic nucleic acid sequences, or fragments thereof, corresponding to SEQ ID NOS:1-136, wherein said genomic sequences comprise at least one CpG position.

Another embodiment of this invention comprises a method for identifying liver DNA, cells or tissue, or for distinguishing liver cells among a group of other chosen cell or tissue types as the source of an analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:1, 2, 69, 70; 7, 8, 75, 76; 9, 10, 77, 78; 11, 12, 79, 80; 13, 14, 81, 82; 25, 26, 93, 94; 35, 36, 103, 104; 37, 38, 105, 106; 51, 52, 119, 120; 53, 54, 121, 122; 59, 60, 127 and 128, and sequences complementary thereto.

It is particularly preferred that said nucleic acid or oligomer sequence comprises at least one methylation variable position (MVP).

Another embodiment of this invention comprises a method for identifying brain DNA, cells or tissue, or for distinguishing brain cells among a group of other chosen cell or tissue types as the source of an analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:3, 4, 71, 72; 17, 18, 85, 86; 49, 50, 117, 118; 61, 62, 129 and 130, and sequences complementary thereto.

It is particularly preferred that said nucleic acid or oligomer sequence comprises at least one methylation variable position (MVP).

Another embodiment of this invention comprises a method for identifying breast DNA, cells or tissue, or for distinguishing breast cells among a group of other chosen cell or tissue types as the source of an analyzed nucleic acid, comprising an analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:3, 4, 71, 72; 5, 6, 73, 74; 15, 16, 83, 84; 23, 24, 91, 92; 41, 42, 109, 110; 65, 66, 133 and 134, and sequences complementary thereto.

It is particularly preferred that said nucleic acid or oligomer sequence comprises at least one methylation variable position (MVP).

Another embodiment of this invention comprises a method for identifying muscle DNA, cells or tissue, or for distinguishing muscle cells among a group of other chosen cell or tissue types as the source of an analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:15, 16, 83, 84; 43, 44, 111, 112; 47, 48, 115 and 116, and sequences complementary thereto.

It is particulary preferred that said nucleic acid or oligomer sequence comprises at least one methylation variable position (MVP).

Another embodiment of this invention comprises a method for identifying lung DNA, cells or tissue, or for distinguishing lung cells or tissue among a group of other chosen cell or tissue types as the source of an analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:31, 32, 99, 100; 33, 34, 101 and 102, and sequences complementary thereto.

It is particularly preferred that said nucleic acid or oligomer sequence comprises at least one methylation variable position (MVP).

Another embodiment of this invention comprises a method for identifying the DNA, cells or tissues of breast or muscle, or for distinguishing breast or muscle cells or tissue out of a group of other chosen cell or tissue types as the source of an analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:45, 46, 113, 114; 63, 64, 131, and 132, and sequences complementary thereto.

It is particularly preferred that said nucleic acid or oligomer sequence comprises at least one methylation variable position (MVP).

Another embodiment of this invention comprises a method for identifying brain or muscle DNA, cells or tissue, or for distinguishing brain or muscle cells or tissue among a group of other chosen cell types as the source of an analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:57, 58, 125 and 126, and sequences complementary thereto.

It is particularly preferred that said nucleic acid or oligomer sequence comprises at least one methylation variable position (MVP).

Another embodiment of this invention comprises a method for identifying brain or breast DNA, cells or tissues, or for distinguishing brain or breast cells or tissue among a group of other chosen cell types as the source of an analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:67, 68, 135, 136, and sequences complementary thereto.

It is particularly preferred that said nucleic acid or oligomer sequence comprises at least one methylation variable position (MVP).

Another embodiment of this invention comprises a method for identifying breast or lung DNA, cells or tissues, or for distinguishing breast or lung cells or tissue among a group of other chosen cell types as the source of an analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:17, 18, 85, 86, and sequences complementary thereto.

It is particularly preferred that said nucleic acid or oligomer sequence comprises at least one methylation variable position (MVP).

Another embodiment of this invention comprises a method for distinguishing lung from muscle cells or tissue as the source of an analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:55, 56, 123 and 124, and sequences complementary thereto.

It is particularly preferred that said nucleic acid or oligomer sequence comprises at least one methylation variable position (MVP).

Another embodiment of this invention comprises a method for distinguishing brain, breast and muscle cells or tissue from liver, lung and prostate cells or tissue as the source of an analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:19, 20, 87 and 88, and sequences complementary thereto.

It is particularly preferred that said nucleic acid or oligomer sequence comprises at least one methylation variable position (MVP).

Another embodiment of this invention comprises a method for distinguishing brain, breast and muscle cells or tissue from lung and prostate cells or tissue as the source of analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:29, 30, 97 and 98, and sequences complementary thereto.

It is particularly preferred that said nucleic acid or oligomer sequence comprises at least one methylation variable position (MVP).

Another embodiment of this invention comprises a method for distinguishing liver, breast and muscle cells or tissue from brain and lung cells or tissue as the source of an analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:21, 22, 89 and 90, and sequences complementary thereto.

It is particularly preferred that said nucleic acid or oligomer sequence comprises at least one methylation variable position (MVP).

Another embodiment of this invention comprises a method for distinguishing liver and muscle cells or tissue from brain and breast cells or tissue as the source of an analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:27, 28, 95 and 96 and sequences complementary thereto.

It is particularly preferred that said nucleic acid or oligomer sequence comprises at least one methylation variable position (MVP).

Another embodiment of this invention comprises a method for distinguishing brain, liver and lung cells or tissues from prostate and breast cells or tissues as the source of an analyzed nucleic acid, comprising analysis of the state or level of methylation of one or more MVPs utilizing a nucleic acid or an oligomer comprising at least one contiguous base sequence having a length of at least 16 nucleotides (or at least 18, 20, 22, 23, 25, 30 or 35 nucleotides), which is complementary to, or hybridizes under moderately stringent or stringent conditions to a pretreated genomic DNA according to SEQ ID NOS:39, 40, 107 and 108, and sequences complementary thereto.

It is particulary preferred that said nucleic acid or oligomer sequence comprises at least one methylation variable position (MVP).

### EXAMPLE 1

### MVPs and Markers Comprising Multiple MVPs in the Major Histocompatability Complex (MHC) were Identified According to Methods of the Present Invention

The selected loci of this example are all located within the major histocompatability complex (MHC), as is disclosed in SEQ ID NO:205.

Cloned DNA cannot be used for sequencing for present purposes, because the methylation information is lost during cloning. Therefore, protocols for the design of primers and for the generation of amplificates of genes within the MHC were developed. Available sequence information from the MHC was used for this purpose, and specific primer-sets were designed to be used to amplify (gene-derived) fragments or regions comprising putative variable methylation information. The amplificates were obtained in multiplex PCR experiments, and the primer molecules designed therefore (see herein above) are listed in Table 1, and referring to the sequence protocol SEQ ID NO:137 through SEQ ID NO:204 (see Sequence Listing).

Table 1 lists the SEQ ID numbers of the primer pairs that were used to amplify specific regions of the pretreated DNA (third column), according to the ROI identifier number (listed in the first column). The ROI identifier number links the sequence information (as given in Table 2, below, as ROI SEQ ID numbers) with information (given in Tables 3-36 and FIGS. 1-34) about the methylation levels measured at the majority of CpG sites within these regions and specifically with information about the methylation levels at specific methylation variable CpG sites (MVP) within these regions.

The second column in Table 1 gives the name of the gene to which the genomic sequence analyzed is related, as the ROI may either lie within the gene, or close to its 5′-end. If no gene name is known, the name of the genomic clone is given instead. The regions amplified with primers, as disclosed herein, comprise one or more MVPs (i.e., differentially methylated CpG positions). The last two columns of Table 1 provide the SEQ ID numbers of those 2 versions of said ROI that can be used as template for the respective specific primer pair.

TABLE 1

| | | | | | amplificate | |
|---|---|---|---|---|---|---|
| | | | | | located within | |
| | | | | | ROI SEQ ID | |
| ROI | related gene | primer | FIG. no | | up and down | |
| identifier | name | SEQ ID | \overline{Table no} | strand | methylated | |
| 3083 | BF | 137 | 1 | BISU 2 | 2 | 70 |
| | | 138 | 3 | | | |
| 3084 | BF | 139 | 2 | BISU 2 | 4 | 72 |
| | | 140 | 4 | | | |
| 3091 | C2 | 141 | 3 | BISU 2 | 6 | 74 |
| | | 142 | 5 | | | |
| 3093 | C4B | 143 | 4 | BISU 1 | 7 | 75 |
| | | 144 | 6 | | | |
| 3094 | C4B | 145 | 5 | BISU 2 | 10 | 78 |
| | | 146 | 7 | | | |
| 3103 | CYP21A2 | 147 | 6 | BISU 2 | 12 | 80 |
| | | 148 | 8 | | | |
| 3104 | CYP21A2 | 149 | 7 | BISU 1 | 13 | 81 |
| | | 150 | 9 | | | |
| 3105 | DAXX | 151 | 8 | BISU 1 | 15 | 83 |
| | | 152 | 10 | | | |
| 3107 | DDAH2 | 153 | 9 | BISU 1 | 17 | 85 |
| | | 154 | 11 | | | |
| 3110 | DDR1 | 155 | 10 | BISU 2 | 20 | 88 |
| | | 156 | 12 | | | |
| 3113 | DOM3-Z | 157 | 11 | BISU 2 | 22 | 90 |
| | | 158 | 13 | | | |
| 3127 | G6d | 159 | 12 | BISU 2 | 24 | 92 |
| | | 160 | 14 | | | |
| 3129 | G7a | 161 | 13 | BISU 2 | 26 | 94 |
| | | 162 | 15 | | | |
| 3145 | HLA-A | 163 | 14 | BISU 2 | 28 | 96 |
| | | 164 | 16 | | | |
| 3152 | HLA-DMA | 165 | 15 | BISU 1 | 29 | 97 |
| | | 166 | 17 | | | |
| 3170 | HLA-DRB3 | 167 | 16 | BISU 2 | 32 | 100 |
| | | 168 | 18 | | | |
| 3192 | MICB | 169 | 17 | BISU 2 | 34 | 102 |
| | | 170 | 19 | | | |
| 3200 | NG22 | 171 | 18 | BISU 1 | 35 | 103 |
| | | 172 | 20 | | | |
| 3208 | PBX2 | 173 | 19 | BISU 1 | 37 | 105 |
| | | 174 | 21 | | | |
| 3239 | TAPBP | 175 | 20 | BISU 1 | 39 | 107 |
| | | 176 | 22 | | | |
| 3243 | TNF | 177 | 21 | BISU 2 | 42 | 110 |
| | | 178 | 23 | | | |
| 3244 | TNXB | 179 | 22 | BISU 2 | 44 | 112 |
| | | 180 | 24 | | | |
| 3252 | ZNF297 | 181 | 23 | BISU 2 | 46 | 114 |
| | | 182 | 25 | | | |
| 3265 | dJ570F3 | 183 | 24 | BISU 2 | 48 | 116 |
| | | 184 | 26 | | | |
| 3291 | BTNL2 | 185 | 25 | BISU 2 | 50 | 118 |
| | | 186 | 27 | | | |
| 3312 | SKIV2L | 187 | 26 | BISU 2 | 52 | 120 |
| | | 188 | 28 | | | |
| 3329 | C2 | 189 | 27 | BISU 1 | 53 | 121 |
| | | 190 | 29 | | | |
| 3330 | ABCB2 | 191 | 28 | BISU 1 | 55 | 123 |
| | | 192 | 30 | | | |
| 3347 | dJ570F3 | 193 | 29 | BISU 2 | 58 | 126 |
| | | 194 | 31 | | | |
| 3348 | DDX16 | 195 | 30 | BISU 2 | 60 | 128 |
| | | 196 | 32 | | | |
| 3364 | TNXB | 197 | 31 | BISU 2 | 62 | 130 |
| | | 198 | 33 | | | |
| 3374 | RAB2L | 199 | 32 | BISU 2 | 64 | 132 |
| | | 200 | 34 | | | |
| 3377 | BAT2 | 201 | 33 | BISU 2 | 66 | 134 |
| | | 202 | 35 | | | |
| 3382 | DDX16 | 203 | 34 | BISU 1 | 67 | 135 |
| | | 204 | 36 | | | |

The listing of the primer molecules of Table 1, however, is not to be understood as limiting the scope of the method to the use of only those primer molecules. Rather, the listing is meant to illustrate and enable the example given. It will be obvious to one skilled in the relevant art that primer molecules that will amplify, preferably by means of a PCR, the other bisulfite pretreated strand (for example BISU 2) also provide the means to analyze the methylation levels of exactly the same CpGs within these genomic regions. Therefore, it is understood, that the use of amplification of such other strands is also enabled, even though the explicit sequences are not listed in Table 1.

Further embodiments of the present invention comprise primers and primer sets used to amplify ROI regions, based upon disclosure of the genomic region of the MHC, specification of the regions of interest (ROI) by disclosing BISU 1 (or BISU 2 respectively) of those ROIs, and otherwise disclosing methods to optimally design those primers to achieve an unbiased amplification of the sections containing the listed MVPs.

An especially preferred selection of primer pairs is disclosed in Table 1.

The obtained PCR amplificates were subjected to high-throughput bisulfite DNA sequencing and methylation analysis, as described above.

In this example, 253 genomic regions were amplified and sequenced, both in forward and reverse direction, in 32 different samples resulting in a minimum of 16,192 sequencing reads. Analyzing the trace files of those reads with ESME (described herein above), the methylation levels at all 3,302 CpG positions in the 6 tissues (prostate, muscle, lung, liver, breast and brain) were determined, and candidate methylation variable positions (MVPs) were identified.

Each amplificate was bisulfite sequenced once from both ends using the original PCR primers, ABI Prism™ BigDye terminator chemistry and 3700/3730 capillary sequencers to ensure maximum accuracy. The individual reads were base-called using the PHRED algorithm which provides quality values for each base. Bisulfite sequences that passed the internal quality test were analyzed with the ESME software. Raw sequencing data were calibrated and normalized.

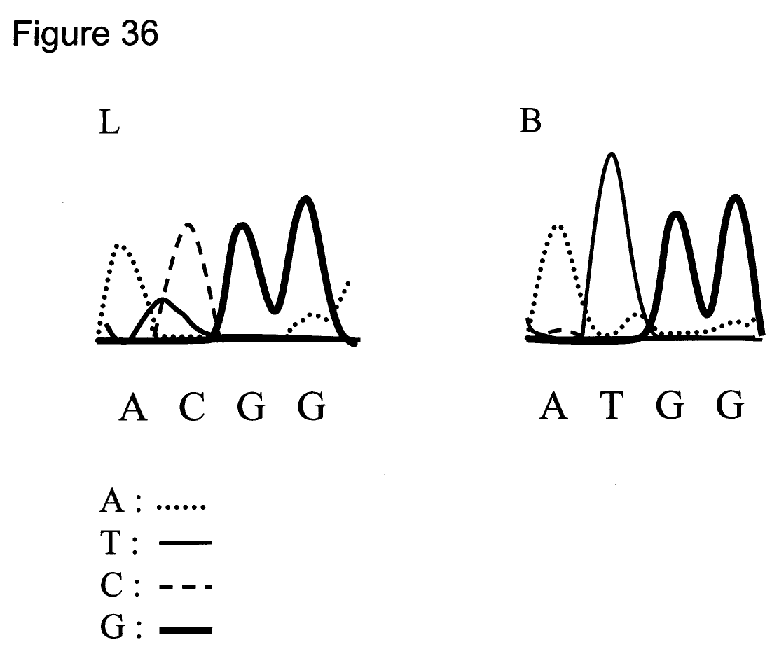

An example of an MVP identified in the present MHC study by bisulfite sequencing is shown in FIG. 36. Two different healthy tissues were analyzed. The left sequence trace shows the analysis of DNA isolated from healthy lung tissue, wherein the cytosine of interest is methylated. The right trace shows the analysis of DNA isolated from healthy brain tissue, wherein the corresponding cytosine position is unmethylated. Bisulfite sequencing is based on the conversion of all non-methylated cytosines to uracil, by treatment of genomic DNA with bisulfite. In the sequence trace, non-methylated cytosine appears therefore as T, while methylated C appears as C (see FIG. 36).

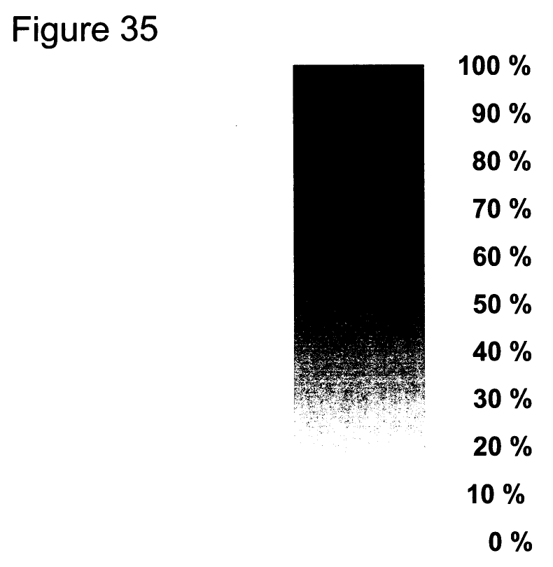

Levels of methylation identified at particular CpG sites are given as percentages in Tables 3-36. For an improved visualization, however, the data were also entered into a matrix display showing, on a gray scale, methylation levels for each analyzed position in the roughly 25 samples according to the 6 different sample types represented (see FIGS. 1-34). The shade of gray directly correlated to the level of methylation, as can be seen in FIG. 35. A black box represents a methylation percentage of 100%, indicating that, at this position, every single DNA molecule within the sample analyzed was methylated. A very light gray box, however, indicates that all DNA molecules were unmethylated at this position. A white box indicates that no value was obtained. In the Tables 3-36, these positions are labeled as “NA” (not applicable).

In Tables 3-36, the related CpG positions within the ROI sequence are given. As all four sequences of the bisulfite versions (i.e., all four bisulfite sequences, corresponding to the fully up-methylated and the fully down-methylated variants) of each respective ROI are disclosed in the sequence listing, all CpG locations, including the MVP locations, within the sequences can easily be identified. The question as to whether or not a particular ROI is a useful marker or not can be answered by examination of the methylation levels disclosed numerically in Tables 3-36, as represented by different shades of gray in the corresponding Figures. A low-level of methylation at a specific data point, determined by the tissue sample and the CpG position analyzed, is represented as a square in light gray color, whereas a high-level of methylation is indicated in dark gray. FIG. 35 shows how the different levels of methylation correlate with the scale of gray in FIGS. 1-34. The data points are represented as groups of the samples from the same tissue, thereby facilitating the decision as to which sections of the ROI, comprising which CpG positions, can be utilized as effective markers for distinguishing the specific tissue or group of tissues from others. If, in the FIGS. 1-34, the gray scaled pattern is evidently lighter or darker at an area for only one or even two kinds of tissues when compared to the remaining tissues, then this ROI is a methylation marker for said tissue, and in particular embodiments, can be used as a tissue marker in suitable assays, as described in EXAMPLES 2-6, herein below. Occasionally, only some specific CpG positions out of the about 10-15 positions analyzed show different methylation levels, depending on the tissue type the analyzed DNA was derived from.

P-values were calculated that are indicative of the differentiating power of each single CpG position, and are also given in the Tables 3-36. This value, while indicative of the ‘marking ability’ of each CpG position, however, is only meant to illustrate the statistical relevance of this data set. Preferably, the actual quality of a methylation marker is ultimately determined by the accumulation of a plurality of differentiating CpG positions within a section of about 200-500 bp. Especially preferred are those sections that comprise more than two differentially-methylated CpG positions, within a total of about 5 CpG positions located next to each other (within a total of about 5 proximate CpG positions).

Two different P-values are given for each CpG position in cases where a marker ROI is comprised of two different sections that could each, independently, be used to differentiate between different tissues or tissue groups, as for example ROI 3105.

A selection of the ROIs identified by visual examination of the methylation pattern analysis, and hence a first indication of their usefulness, is given in Table 2.

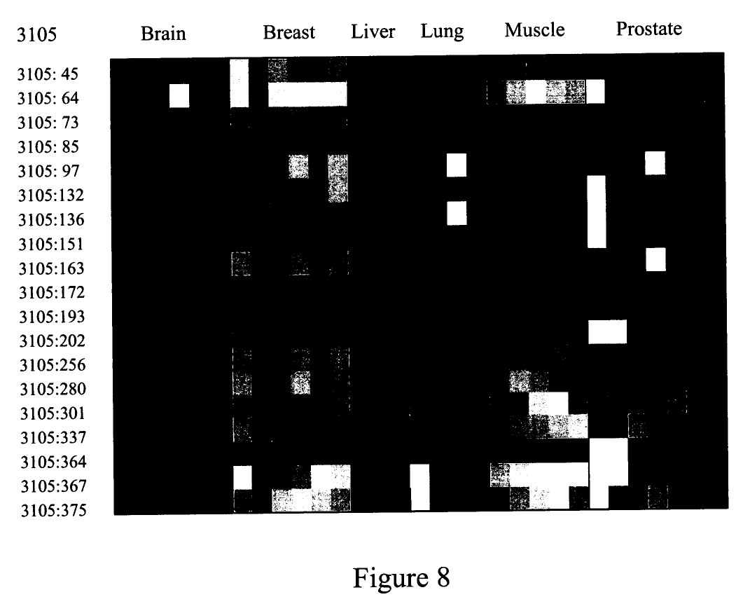

For example, FIG. 8 displays the levels of methylation of CpGs located in the amplificate 3105 of ROI 3105. The numbers at the left hand side indicate the position of the CpGs analyzed within said amplificate. 3105_{—}45, for example, states that the cytosine of said CpG is the 45th nucleotide from the 5′-end of amplificate 3105. The positions of said MVPs within the amplificate (for example, the MVP positions within the ROI 3105 amplificate as given in the CpG identifier column of Table 10) are disclosed in the CpG identifier in the Tables 3-36 and in FIGS. 1-34. The position of the amplificate 3105 within the ROI 3105 is determined by the binding position of its amplification primers. The primer pair given for ROI 3105 (primer SEQ ID NO:151 and primer SEQ ID NO:152) are priming either at ROI SEQ ID NO:15 or ROI SEQ ID NO:83 as given in Table 1. The primer that hybridizes to the first copy of the amplified strand, and that therefore is identical to the bisulfite sequence itself, usually is referred to as the forward primer, because it marks the beginning of the amplificate sequence within the ROI. The position of the first nucleotide of this primer is the start of the amplificate within the ROI, and is also given in Table 2. Therefore, the position of the MVP within the ROI (which is disclosed with a SEQ ID NO) can easily and accurately be identified by simply adding these two numbers.

Additionally, the explicit positions of each CpG and MVP within the ROI are given in Tables 3-36.

TABLE 2

| | ROI SEQ | | | | | | | |
|---|---|---|---|---|---|---|---|---|
| | IDs | | ROI SEQ IDs | | FIG. | Start of | | from other |
| ROI Identifier | up | | down | | Number | amplificate | identifies | types |
| 3083 | 1 | 2 | 69 | 70 | 1 | 414 | liver | all |
| 3084 | 3 | 4 | 71 | 72 | 2 | 976 | brain | all |
| 3084 | 3 | 4 | 71 | 72 | 2 | 976 | breast | all |
| 3091 | 5 | 6 | 73 | 74 | 3 | 1667 | breast | all |
| 3093 | 7 | 8 | 75 | 76 | 4 | 1098 | liver | all |
| 3094 | 9 | 10 | 77 | 78 | 5 | 470 | liver | all |
| 3103 | 11 | 12 | 79 | 80 | 6 | 1711 | liver | all |
| 3104 | 13 | 14 | 81 | 82 | 7 | 1743 | liver | all |
| 3105-1 | 15 | 16 | 83 | 84 | 8 | 255 | breast | all |
| 3105-2 | 15 | 16 | 83 | 84 | 8 | 255 | muscle | all, but |
| | | | | | | | | breast |
| 3107 | 17 | 18 | 85 | 86 | 9 | 278 | brain | breast, lung |
| 3107 | 17 | 18 | 85 | 86 | 9 | 278 | breast, lung | all |
| 3110 | 19 | 20 | 87 | 88 | 10 | 1901 | brain, breast, | liver, lung, |
| | | | | | | | muscle | prostate |
| 3113 | 21 | 22 | 89 | 90 | 11 | 19 | breast, liver, | brain, lung |
| | | | | | | | muscle | |
| 3127 | 23 | 24 | 91 | 92 | 12 | 1731 | breast | all |
| 3129 | 25 | 26 | 93 | 94 | 13 | 1900 | liver | all |
| 3145 | 27 | 28 | 95 | 96 | 14 | 618 | liver, muscle | breast, |
| | | | | | | | | brain |
| 3152 | 29 | 30 | 97 | 98 | 15 | 1795 | brain, breast, | lung, |
| | | | | | | | muscle | prostate |
| 3170 | 31 | 32 | 99 | 100 | 16 | 1688 | lung | all |
| 3192 | 33 | 34 | 101 | 102 | 17 | 346 | lung | all, but |
| | | | | | | | | brain |
| 3200 | 35 | 36 | 103 | 104 | 18 | 1861 | liver | all |
| 3208 | 37 | 38 | 105 | 106 | 19 | 696 | liver | all |
| 3239 | 39 | 40 | 107 | 108 | 20 | 585 | breast, | brain, lung, |
| | | | | | | | prostate | liver |
| 3243 | 41 | 42 | 109 | 110 | 21 | 1519 | breast | all |
| 3244 | 43 | 44 | 111 | 112 | 22 | 101 | muscle | all |
| 3252 | 45 | 46 | 113 | 114 | 23 | 701 | breast, | all |
| | | | | | | | muscle | |
| 3265 | 47 | 48 | 115 | 116 | 24 | 654 | muscle | all |
| 3291 | 49 | 50 | 117 | 118 | 25 | 205 | brain | all |
| 3312 | 51 | 52 | 119 | 120 | 26 | 1427 | liver | all |
| 3329 | 53 | 54 | 121 | 122 | 27 | 1099 | liver | all |
| 3330 | 55 | 56 | 123 | 124 | 28 | 1988 | lung | muscle |
| 3347 | 57 | 58 | 125 | 126 | 29 | 1875 | muscle, | all |
| | | | | | | | brain | |
| 3348 | 59 | 60 | 127 | 128 | 30 | 1556 | liver | all |
| 3364 | 61 | 62 | 129 | 130 | 31 | 1888 | brain | all |
| 3374 | 63 | 64 | 131 | 132 | 32 | 941 | breast, | all |
| | | | | | | | muscle | |
| 3377 | 65 | 66 | 133 | 134 | 33 | 2006 | breast | all |
| 3382 | 67 | 68 | 135 | 136 | 34 | 1191 | brain, breast | all |

The utilities of said MVPs (within the according ROIs) for distinguishing between or among which tissue types can be determined from examination of FIGS. 1-34, and from the Tables 3-36 (below).

The ROIs can now be scored, for example, according to the number of CpG positions that seem to discriminate between specific tissues. The more discriminating MVPs there are in one ROI the better. Another way to score the ROIs is to more highly score those markers comprising adjacent or proximate MVPs. A third way to identify those ROIs that would be most useful for the identification, differentiation or for distinguishing between cell types or tissue types is to use the data given in Tables 3-36 to calculate the P-values for those differing methylation levels.

Each particularly useful MVP and its particular utility is given in the Tables 37-70 (below). These MVPs, and nucleotide sequences comprising a contiguous sequence of at least 16 nucleotides in length (or at least 18, 20, 22, 23, 25, 30 or 35 nucleotides in length) comprising the three bases 5′ to the MVP and the three bases 3′ to the MVP are a preferred embodiment of the present invention. Especially preferred are those oligomers comprising a MVP which qualifies as a “good marker position” as indicated in Tables 37-70, (P-value smaller than 0.05). However, the P-values given here have mainly been calculated for differentiation of one tissue against the group of all other tissue samples, for example the P-values for ROI 3091 were calculated by comparing the methylation levels of the breast samples against those of all other samples, and the P-values might have been better for comparing these breast samples with liver samples only. That is why this selection is not understood as limiting the scope of the present invention to only those MVPs that have P-values as given that are smaller than 0.05.

Additionally, the use of those sequences comprising these MVPs to identify the tissue that shows a distinguished methylation pattern is a preferred embodiment of this invention. Particularly preferred are those nucleic acid and oligomer sequences comprising a contiguous sequence of at least 16 nucleotides in length (or at least 18, 20, 22, 23, 25, 30 or 35 nucleotides in length) comprising comprising said MVPs, and particularly comprising the three bases 5′ to the MVP and the three bases 3′ to the MVP.

Tables 3-36, and Tables 37-70 follow next:

TABLE 3
(3083):

| | MVP | | | | | | | | | | | | |
|---|---|---|---|---|---|---|---|---|---|---|---|---|---|
| CpG | Position | | | | | | | | | | | | |
| identifier | in ROI | Prostate | Prostate | Prostate | Prostate | Prostate | Prostate | Prostate | Muscle | Muscle | Muscle | Muscle | Muscle |
| 3083: 28 | 442 | 0.91 | 1 | 0.49 | 1 | 0.5 | 1 | NA | 1 | 0.9 | 1 | 0.41 | 1 |
| 3083: 31 | 445 | 1 | 1 | 1 | 1 | 0.55 | 1 | NA | 1 | 1 | 1 | 0.5 | 1 |
| 3083: 40 | 454 | 1 | 1 | NA | NA | 1 | NA | NA | 1 | 1 | 1 | 0.78 | 1 |
| 3083: 55 | 469 | 1 | 1 | 1 | 1 | 1 | 0.76 | NA | 1 | 0.81 | 0.58 | 0.83 | 0.71 |
| 3083: 61 | 475 | 1 | 1 | 0.45 | NA | 1 | 1 | NA | 1 | NA | 0.73 | 1 | NA |
| 3083: 95 | 509 | 0.65 | 1 | NA | 0 | 1 | 0.75 | NA | 0.61 | 1 | 0.88 | 1 | 1 |
| 3083: 122 | 536 | 1 | 1 | 1 | 1 | 0.87 | 1 | NA | 1 | 1 | 1 | 1 | 1 |
| 3083: 143 | 557 | 1 | 0.5 | NA | 0.5 | 1 | 0.5 | NA | 1 | 1 | 1 | 1 | 0.5 |
| 3083: 161 | 575 | 1 | 1 | 0.75 | NA | 0.96 | 1 | NA | 0.87 | NA | 0.85 | 0.93 | 1 |
| 3083: 202 | 616 | 1 | 1 | 1 | 1 | 1 | 1 | 1 | 1 | 1 | 1 | 1 | 1 |
| 3083: 216 | 630 | 1 | 0.83 | 0.87 | 0.83 | 1 | 0.8 | 1 | 0.91 | 0.84 | 0.94 | 1 | 0.87 |
| 3083: 235 | 649 | 0.92 | 1 | 0.51 | 1 | 0.88 | 1 | NA | 1 | 1 | 1 | 0.86 | 1 |
| 3083: 250 | 664 | 0.6 | NA | 0.47 | NA | 0.79 | NA | NA | 0.69 | 0.46 | 0.46 | 0.92 | NA |
| 3083: 262 | 676 | 0.92 | 0.62 | 0.79 | 0.57 | 1 | 0.74 | 0.74 | 0.85 | 0.63 | 0.69 | 0.97 | 0.65 |
| 3083: 265 | 679 | 1 | 0.8 | 0.63 | 0.82 | 1 | 0.82 | 0 | 0.95 | 0.91 | 0.95 | 0.97 | 0.91 |
| 3083: 269 | 683 | 0.8 | 0.61 | 0.61 | 0.6 | 0.69 | 0.55 | 1 | 0.21 | 0.21 | 0.75 | 0.63 | 0.19 |
| 3083: 294 | 708 | 0.86 | 0.72 | 0.21 | 0.59 | 0.93 | 0.17 | NA | 0.79 | 0.5 | 0.74 | 0.75 | 0.49 |
| 3083: 299 | 713 | NA | NA | 0.22 | NA | 1 | NA | NA | 0.44 | NA | NA | 0.9 | NA |

| CpG | MVP | | | | | | | | | | | | |
|---|---|---|---|---|---|---|---|---|---|---|---|---|---|
| identifier | Position in ROI | Lung | Lung | Lung | Lung | Lung | Liver | Liver | Breast | Breast | Breast | Breast | Breast |
| 3083: 28 | 442 | 1 | 0.5 | 1 | 1 | 1 | 0.29 | 0.15 | 0.5 | 0.5 | 0.82 | NA | 0.65 |
| 3083: 31 | 445 | 0.5 | 1 | NA | 1 | 0.8 | 0.55 | 0.42 | 0.5 | 0.5 | 1 | NA | 1 |
| 3083: 40 | 454 | 1 | 1 | 1 | 1 | 1 | 0.3 | 0.31 | 1 | 1 | 1 | 1 | 1 |
| 3083: 55 | 469 | 1 | 1 | 0 | 1 | 1 | 0.24 | 0.11 | 1 | 1 | 0.83 | 1 | 0.86 |
| 3083: 61 | 475 | 0.43 | 1 | NA | 1 | 1 | 0.14 | 0.41 | 1 | 1 | 1 | 1 | 1 |
| 3083: 95 | 509 | 0.4 | 0.78 | 0.42 | 0.8 | 0.86 | 0.094 | 0.16 | 1 | 1 | 1 | 0.8 | 0.69 |
| 3083: 122 | 536 | 1 | 1 | 1 | 0.93 | 1 | 0.23 | 0.11 | 0.88 | 0.96 | 1 | 0.93 | 0.75 |
| 3083: 143 | 557 | 1 | 1 | NA | 1 | 1 | 0.66 | 0.14 | 1 | 0.94 | 1 | 1 | 1 |
| 3083: 161 | 575 | 0.83 | 0.97 | NA | 1 | 0.95 | 0.44 | 0.19 | 0.93 | 0.92 | 0.89 | 1 | 1 |
| 3083: 202 | 616 | 1 | 1 | 1 | 1 | 1 | 0.68 | 0.47 | 1 | 1 | 1 | 1 | 1 |
| 3083: 216 | 630 | 0.91 | 1 | NA | 1 | 1 | 0.45 | 0.12 | 1 | 0.97 | 0.99 | 1 | 1 |
| 3083: 235 | 649 | 1 | 0.94 | NA | 0.91 | 0.9 | 0.11 | 0.25 | 0.83 | 0.91 | 0.84 | 0.96 | 0.8 |
| 3083: 250 | 664 | 0.54 | 0.9 | NA | 0.88 | 0.91 | 0.38 | 0.12 | 0.8 | 0.89 | 0.89 | 0.8 | 0.82 |
| 3083: 262 | 676 | 0.89 | 0.98 | 0.42 | 0.97 | 0.99 | 0.38 | 0.27 | 0.96 | 1 | 0.99 | 0.95 | 0.93 |
| 3083: 265 | 679 | 0.96 | 0.98 | NA | 0.98 | 0.98 | 0.21 | 0.21 | 0.96 | 1 | 0.99 | 0.89 | 0.97 |
| 3083: 269 | 683 | 0.38 | 0.82 | 0.64 | 0.87 | 0.76 | 0.079 | 0.052 | 0.76 | 0.65 | 0.81 | 0.71 | 0.58 |
| 3083: 294 | 708 | 0.66 | 0.94 | 0.4 | 0.87 | 0.91 | 0.16 | 0.065 | 0.94 | 0.91 | 0.89 | 0.84 | 0.73 |
| 3083: 299 | 713 | 0.42 | 1 | 0.28 | 0.58 | 0.99 | 0.14 | 0 | 1 | 1 | 0.93 | 0.99 | 0.84 |

| CpG | MVP | | | | | | | |
|---|---|---|---|---|---|---|---|---|
| identifier | Position in ROI | Breast | Brain | Brain | Brain | Brain | Brain | Brain |
| 3083: 28 | 442 | 1 | 1 | 0.39 | 1 | 0.5 | 0.88 | 1 |
| 3083: 31 | 445 | 0.86 | 1 | 0.6 | 1 | 0.5 | 1 | 1 |
| 3083: 40 | 454 | 0.65 | 1 | 0.84 | NA | 1 | 1 | 1 |
| 3083: 55 | 469 | 1 | 1 | 0.9 | 1 | 0.9 | 1 | 1 |
| 3083: 61 | 475 | 1 | 1 | 1 | 0.5 | 1 | 0.8 | 1 |
| 3083: 95 | 509 | 1 | 0.86 | 1 | 1 | 0.9 | 0.92 | 1 |
| 3083: 122 | 536 | 0.89 | 0.98 | 1 | 0.5 | 1 | 0.95 | 1 |
| 3083: 143 | 557 | 1 | 0.92 | 1 | 1 | 0.97 | 0.94 | 0.95 |
| 3083: 161 | 575 | 0.95 | 0.95 | 1 | 1 | 0.93 | 0.93 | 0.93 |
| 3083: 202 | 616 | 1 | 1 | 1 | 1 | 1 | 1 | 1 |
| 3083: 216 | 630 | 0.91 | 1 | 1 | 1 | 1 | 0.9 | 1 |
| 3083: 235 | 649 | 0.77 | 0.97 | 0.98 | 1 | 0.91 | 0.73 | 0.94 |
| 3083: 250 | 664 | 0.71 | 0.96 | 0.89 | 1 | 0.85 | 0.9 | 0.95 |
| 3083: 262 | 676 | 0.96 | 1 | 1 | 1 | 0.98 | 1 | 0.97 |
| 3083: 265 | 679 | 0.91 | 0.97 | 0.99 | 1 | 0.97 | 0.99 | 1 |
| 3083: 269 | 683 | 0.56 | 0.79 | 0.7 | 1 | 0.96 | 0.5 | 0.66 |
| 3083: 294 | 708 | 0.66 | 0.81 | 0.89 | 1 | 0.9 | 0.67 | 0.89 |
| 3083: 299 | 713 | 0.74 | 0.96 | 1 | 0.78 | 0.93 | 1 | 0.96 |

TABLE 4
(3084):

| | MVP | | | | | | | | | | | | | | |
|---|---|---|---|---|---|---|---|---|---|---|---|---|---|---|---|
| | Position | | | | | | | | | | | | | | |
| CpG identifier | in ROI | Prostate | Prostate | Prostate | Prostate | Prostate | Muscle | Muscle | Muscle | Muscle | Muscle | Lung | Lung | Lung | Lung |
| 3084: 41 | 1017 | 0.89 | 0.9 | 0.91 | 1 | 0.13 | 1 | 0.83 | 0.9 | 0.86 | 0.94 | 0 | 1 | 1 | 1 |
| 3084: 56 | 1032 | 0.92 | 0.72 | 0.95 | 1 | 0.94 | 0.89 | 1 | 0.53 | 0.73 | 0.82 | 0.87 | 1 | 0.83 | 0.71 |
| 3084: 69 | 1045 | 1 | 0.91 | 0.88 | 1 | 0.97 | 0.93 | 1 | 0.96 | 0.91 | 0.89 | 0.88 | 1 | 0.96 | 0.87 |
| 3084: 72 | 1048 | 0.95 | 0.83 | 0.95 | 0.92 | 0.84 | 0.93 | 0.97 | 0.89 | 0.89 | 0.81 | 0.93 | 1 | 0.95 | 0.83 |
| 3084: 77 | 1053 | 1 | 0.7 | 1 | 1 | 1 | 0.83 | 1 | 0.63 | 0.59 | 0.6 | 1 | 1 | 0.79 | 0.77 |
| 3084: 101 | 1077 | 1 | 0.88 | 0.92 | 1 | 0.94 | 0.97 | 0.89 | 0.78 | 0.86 | 0.91 | 0.91 | 0.98 | 0.91 | 0.91 |
| 3084: 201 | 1177 | 0.7 | 0.36 | 1 | 0.88 | 0.97 | 0.62 | 0.72 | 0.79 | 0.79 | 0.8 | 0.79 | 0.89 | 0.84 | 0.91 |
| 3084: 276 | 1252 | 0.36 | 0.38 | 0.43 | 0.53 | 1 | 0.23 | 0.42 | 0.038 | 0.28 | 0.22 | 0.4 | 0.42 | 0.32 | 0.45 |
| 3084: 301 | 1277 | 0.61 | 0.2 | 0.43 | 0.69 | 0.96 | 0.37 | 0.45 | 0.37 | 0.4 | 0.33 | 0.6 | 0.72 | 0.37 | 0.49 |
| 3084: 349 | 1325 | 0.19 | 0.14 | 0.36 | 0.2 | 0.13 | 0.17 | 0.12 | 0.0047 | 0.12 | 0.36 | 0.32 | 0.4 | 0.33 | 0.16 |
| 3084: 364 | 1340 | 0.15 | 0.19 | 0 | 0.38 | 0.085 | 0.18 | 0.21 | 0.13 | 0.26 | 0.32 | 0.41 | 0.64 | 0.24 | 0.23 |

| CpG | | | | | | | | | | | | | | | |
|---|---|---|---|---|---|---|---|---|---|---|---|---|---|---|---|
| identifier | MVP Position in ROI | Liver | Liver | Breast | Breast | Breast | Breast | Breast | Breast | Brain | Brain | Brain | Brain | Brain | Brain |
| 3084: 41 | 1017 | 0.7 | 0.25 | 0.68 | 1 | 0.51 | 1 | 0.29 | 0.87 | 0 | 1 | 0.85 | 1 | 0.83 | 1 |
| 3084: 56 | 1032 | 0.69 | 0.81 | 0.56 | 0.78 | 0.43 | 0.64 | 0.61 | 0.85 | 1 | 0.81 | 0.88 | 0.84 | 0.79 | 1 |
| 3084: 69 | 1045 | 0.84 | 1 | 0.52 | 0.91 | 0.63 | 0.41 | 0.75 | 0.88 | 1 | 0.87 | 0.95 | 0.86 | 0.96 | 0.75 |
| 3084: 72 | 1048 | 0.84 | 0.9 | 0 | 0.93 | 0.62 | 0.95 | 0.81 | 0.8 | 1 | 0.88 | 0.88 | 0.88 | 0.95 | 0.87 |
| 3084: 77 | 1053 | 1 | 1 | 0.67 | 1 | 0.62 | 0.76 | 0.69 | 1 | 1 | 0.78 | 0.87 | 1 | 0.8 | 1 |
| 3084: 101 | 1077 | 1 | 0.93 | 0.5 | 0.87 | 0.8 | 0.74 | 0.88 | 0.87 | 1 | 0.92 | 0.89 | 0.92 | 0.9 | 0.76 |
| 3084: 201 | 1177 | 0.49 | 0.45 | 0.45 | 0.75 | 0.48 | 0.72 | 0.75 | 0.72 | 1 | 0.86 | 0.64 | 0.83 | 0.88 | 0.58 |
| 3084: 276 | 1252 | 0.24 | 0.19 | 0.17 | 0.33 | 0 | 0.27 | 0.35 | 0.43 | 0.71 | 0.64 | 0.72 | 0.46 | 0.53 | 0.82 |
| 3084: 301 | 1277 | 0.81 | 0.57 | 0.22 | 0.4 | 0.66 | 0.38 | 0.55 | 0.47 | 0.95 | 0.8 | 0.85 | 0.79 | 0.83 | 0.78 |
| 3084: 349 | 1325 | 0.097 | 0.045 | 0.094 | 0.11 | 0.15 | 0.25 | 0.4 | 0.29 | 0.93 | 0.64 | 0.8 | 0.41 | 0.69 | 0.55 |
| 3084: 364 | 1340 | 0.09 | 0.17 | 0.19 | 0.19 | 0.42 | 0.38 | 0.21 | 0.22 | 0.9 | 0.71 | 1 | 0.82 | 0.83 | 0.54 |

TABLE 5
(3091):

| | MVP | | | | | | | | | | | | | |
|---|---|---|---|---|---|---|---|---|---|---|---|---|---|---|
| CpG | Position | | | | | | | | | | | | | |
| identifier | in ROI | Prostate | Prostate | Prostate | Prostate | Muscle | Muscle | Muscle | Muscle | Lung | Lung | Lung | Lung | Liver |
| 3091: 99 | 1766 | 1 | 1 | 0.45 | 1 | 0.84 | 0.88 | 0.78 | 0.81 | 0.85 | 1 | 0.97 | 1 | 1 |
| 3091: 159 | 1826 | 0.63 | 0.89 | 1 | 1 | 0.58 | 0.68 | 0.49 | 0.61 | 0.66 | 0.5 | 0.71 | 1 | 0.89 |
| 3091: 198 | 1865 | 1 | 1 | 1 | 1 | 1 | 1 | 1 | 1 | 0.86 | 1 | 1 | 1 | 1 |
| 3091: 205 | 1872 | 1 | 0.98 | 1 | 1 | 1 | 1 | 0.93 | 1 | 1 | 1 | 1 | 0.89 | 1 |
| 3091: 217 | 1884 | 1 | 1 | 1 | 0.95 | 1 | 1 | 1 | 1 | 1 | 1 | 1 | 1 | 1 |
| 3091: 241 | 1908 | 1 | 0.96 | 1 | 1 | 0.98 | 0.82 | 1 | 1 | 1 | 1 | 0.91 | 1 | 1 |
| 3091: 247 | 1914 | 1 | 0.92 | 1 | 1 | 1 | 0.83 | 1 | 1 | 0.78 | 1 | 1 | 1 | 1 |
| 3091: 257 | 1924 | 0.72 | 0.95 | 1 | 0.98 | 0.72 | 0.95 | 1 | 0.9 | 0.67 | 1 | 0.86 | 1 | 0.8 |
| 3091: 272 | 1939 | 1 | 1 | 1 | 1 | 0.97 | 1 | 1 | 1 | 0.95 | 1 | 1 | 1 | 1 |
| 3091: 281 | 1948 | 0.89 | 0.96 | 1 | 0.92 | 1 | 0.83 | 1 | 0.91 | 1 | 0.89 | 1 | 1 | 1 |
| 3091: 286 | 1953 | 1 | 0.94 | 1 | 1 | 1 | 0.85 | 1 | 0.97 | 0.67 | 1 | 1 | 1 | 1 |
| 3091: 303 | 1970 | 1 | 1 | 1 | 0.94 | 0.81 | 0.96 | 1 | 0.77 | 0.67 | 1 | 1 | 1 | 1 |
| 3091: 320 | 1987 | 1 | 1 | 1 | 0.18 | 1 | 0.72 | 1 | 0.88 | 0.98 | 1 | 1 | 0.87 | 0.97 |
| 3091: 334 | 2001 | 0.96 | 0.85 | 1 | 0.94 | 0.87 | 0.57 | 0.94 | 1 | 0.77 | 1 | 0.98 | 1 | 1 |
| 3091: 337 | 2004 | 1 | 0.82 | 0.92 | 1 | 0.68 | 0.81 | 0.56 | 0.56 | 0.67 | 0.7 | 0.74 | 0.91 | 1 |
| 3091: 370 | 2037 | 0.89 | 0.81 | 0.77 | 0.82 | 0.91 | 1 | 0.87 | 0.89 | 0.64 | 0.77 | 1 | 0.78 | 0.86 |
| 3091: 379 | 2046 | 0.95 | 0.82 | 1 | 1 | 0.97 | 0.72 | 1 | 0.88 | 0.73 | 1 | 0.93 | 1 | 0.93 |
| 3091: 391 | 2058 | 1 | 1 | 0.93 | 1 | 0.9 | 0.77 | 1 | 0.92 | 0.46 | 1 | 0.93 | 0.98 | 0.84 |
| 3091: 449 | 2116 | 0.45 | 0.0081 | 0.37 | 0.5 | 0.69 | 0.98 | 0.56 | 0.47 | 0.54 | 0.22 | 0.62 | 0.36 | 0.96 |

| CpG | MVP | | | | | | | | | | | | | |
|---|---|---|---|---|---|---|---|---|---|---|---|---|---|---|
| identifier | Position in ROI | Liver | Breast | Breast | Breast | Breast | Breast | Breast | Brain | Brain | Brain | Brain | Brain | Brain |
| 3091: 99 | 1766 | 1 | 0.88 | 0.66 | 0.88 | 0.92 | 0.98 | 0.93 | 0.93 | 1 | 1 | 1 | 0.87 | 0.94 |
| 3091: 159 | 1826 | 1 | 0.55 | 0.94 | 0.6 | 0.23 | 0.51 | 0.69 | 0.77 | 1 | 0.41 | 1 | 0.63 | 0.93 |
| 3091: 198 | 1865 | 1 | 1 | 1 | 1 | 0.75 | 1 | 1 | 1 | 1 | 1 | 1 | 0.97 | 1 |
| 3091: 205 | 1872 | 1 | 0.97 | 0.78 | 1 | 0.76 | 1 | 1 | 1 | 1 | 0.92 | 1 | 1 | 1 |
| 3091: 217 | 1884 | 1 | 1 | 1 | 1 | 0.74 | 1 | 1 | 1 | 1 | 1 | 1 | 1 | 1 |
| 3091: 241 | 1908 | 1 | 0.91 | 0.92 | 0.97 | 0.83 | 1 | 1 | 1 | 1 | 1 | 0.96 | 0.83 | 1 |
| 3091: 247 | 1914 | 1 | 0.97 | 0.95 | 0.98 | 1 | 1 | 1 | 1 | 1 | 1 | 0.98 | 1 | 1 |
| 3091: 257 | 1924 | 0.55 | 0.73 | 0.55 | 0.57 | 0.81 | 0.91 | 0.73 | 0.97 | 1 | 0.76 | 0.94 | 0.83 | 0.96 |
| 3091: 272 | 1939 | 1 | 1 | 0.84 | 1 | 0.65 | 1 | 0.79 | 1 | 0.93 | 0.87 | 0.87 | 0.83 | 1 |
| 3091: 281 | 1948 | 0.97 | 0.76 | 0.82 | 0.86 | 0.75 | 1 | 1 | 1 | 1 | 0.89 | 1 | 0.86 | 0.92 |
| 3091: 286 | 1953 | 1 | 1 | 0.82 | 0.98 | 1 | 1 | 1 | 1 | 1 | 1 | 1 | 1 | 1 |
| 3091: 303 | 1970 | 1 | 0.86 | 0.83 | 0.83 | 0.87 | 0.73 | 0.59 | 1 | 1 | 1 | 0.88 | 0.97 | 1 |
| 3091: 320 | 1987 | 1 | 0.94 | 0.85 | 0.71 | 0.68 | 0.65 | 0.66 | 1 | 1 | 1 | 1 | 1 | 1 |
| 3091: 334 | 2001 | 1 | 0.94 | 0.9 | 0.78 | 1 | 0.94 | 1 | 1 | 1 | 1 | 0.94 | 0.97 | 0.91 |
| 3091: 337 | 2004 | 1 | 0.57 | 0.67 | 0.3 | 0.79 | 0.6 | 0.7 | 0.92 | 1 | 1 | 1 | 0.9 | 0.93 |
| 3091: 370 | 2037 | 0.84 | 0.72 | 0.63 | 0.59 | 1 | 0.85 | 0.71 | 0.9 | 0.38 | 0.75 | 0.91 | 0.71 | 0.87 |
| 3091: 379 | 2046 | 1 | 0.85 | 0.65 | 0.61 | 0.95 | 0.91 | 0.82 | 1 | 1 | 0.88 | 1 | 0.73 | 1 |
| 3091: 391 | 2058 | 1 | 0.8 | 0.56 | 0.65 | 1 | 0.79 | 0.79 | 1 | 0.96 | 0.6 | 0.98 | 0.84 | 1 |
| 3091: 449 | 2116 | 0.87 | 0.42 | 0.64 | 0.64 | 0.76 | 0.55 | 0.56 | 0.8 | 0.79 | 1 | 0.52 | 0.52 | 0.91 |

TABLE 6
(3093):

| CpG | MVP | | | | | | | | | | | | |
|---|---|---|---|---|---|---|---|---|---|---|---|---|---|
| identifier | Position in ROI | Prostate | Prostate | Muscle | Muscle | Muscle | Muscle | Lung | Lung | Lung | Liver | Liver | Breast |
| 3093: 24 | 1122 | NA | 0.66 | NA | 0 | 0.66 | 1 | 0.67 | 0.14 | 0.37 | 0.53 | 0 | 1 |
| 3093: 31 | 1129 | NA | NA | NA | 0.6 | 1 | 1 | NA | 1 | 0.32 | 0.78 | 0.5 | NA |
| 3093: 39 | 1137 | NA | 0.59 | 0.5 | NA | 0.76 | 1 | 0.9 | NA | 1 | 1 | 0.18 | 1 |
| 3093: 99 | 1197 | 1 | 1 | 0.78 | 0.77 | 0.97 | 1 | 1 | 1 | 1 | 0.82 | 0.8 | NA |
| 3093: 104 | 1202 | NA | 1 | NA | 0.92 | 1 | 1 | 0.96 | NA | 1 | 1 | 1 | NA |
| 3093: 182 | 1280 | 1 | 1 | 0.35 | 0.63 | 0 | NA | 1 | 1 | NA | 0.17 | 0 | 0.41 |
| 3093: 193 | 1291 | 1 | 0.95 | 1 | 0.62 | 0.62 | NA | 0.93 | 1 | 0.41 | 0.4 | 0.44 | 0.85 |
| 3093: 217 | 1315 | 1 | 1 | NA | 1 | 1 | 1 | 0.92 | 1 | 1 | NA | 0 | 1 |
| 3093: 232 | 1330 | 0.89 | 0.9 | 0.34 | 0.93 | 0.64 | NA | 1 | 0.96 | 0.58 | 0.69 | 0.62 | 0.88 |
| 3093: 240 | 1338 | 1 | 0.65 | 0.61 | 0.93 | 1 | NA | 1 | 0.76 | NA | 0.84 | 0.63 | 0.87 |
| 3093: 247 | 1345 | 0.77 | 0.5 | 0.51 | 0.63 | 0.34 | 0.78 | 0.34 | 0.91 | 0.71 | 0.38 | 0.32 | 0.7 |
| 3093: 256 | 1354 | 0.39 | 0.6 | 0.19 | 0.15 | 0.8 | NA | 0 | 0.6 | NA | 0.15 | 0.64 | 0.33 |
| 3093: 258 | 1356 | 1 | 1 | 0.64 | 0.98 | NA | 1 | NA | 1 | 1 | 0.76 | 0.74 | 0.95 |
| 3093: 269 | 1367 | 1 | 0.75 | 0.41 | 0.74 | 1 | 0 | NA | 1 | 0.36 | 1 | 0.57 | 1 |
| 3093: 277 | 1375 | 0.84 | 0.91 | 0.17 | 0.93 | 0.83 | 0.75 | NA | 1 | 0.91 | 0.43 | 0.27 | 0.7 |
| 3093: 319 | 1417 | 1 | 1 | 0.56 | 0.98 | 1 | 1 | NA | 1 | 1 | 0.89 | 0.73 | 1 |
| 3093: 347 | 1445 | 0.95 | 0.96 | 0.62 | 0.88 | 1 | 1 | NA | 1 | 0.94 | 0.76 | 0.53 | 0.45 |
| 3093: 358 | 1456 | 0.76 | 0.45 | 0 | 0 | 0.31 | 0.43 | NA | 0.12 | 0.54 | 0.18 | 0 | 0.32 |
| 3093: 395 | 1493 | 1 | 1 | 0.6 | 0.81 | 1 | 1 | NA | 1 | 1 | 0 | 1 | 1 |
| 3093: 398 | 1496 | 1 | 1 | 0.65 | 0.94 | 1 | 1 | NA | 1 | 1 | 1 | 1 | 1 |
| 3093: 415 | 1513 | 1 | 1 | 0.73 | 1 | 1 | 1 | NA | 1 | 1 | 1 | 1 | 1 |
| 3093: 433 | 1531 | 1 | 1 | 1 | 1 | 1 | 1 | NA | 1 | 1 | 1 | 1 | 1 |
| 3093: 440 | 1538 | 1 | 0.86 | 1 | NA | 1 | 1 | NA | 1 | 1 | 1 | 0.89 | NA |

| | MVP | | | | | | | | | | | |
|---|---|---|---|---|---|---|---|---|---|---|---|---|
| CpG identifier | Position in ROI | Breast | Breast | Breast | Breast | Breast | Brain | Brain | Brain | Brain | Brain | Brain |
| 3093: 24 | 1122 | 1 | 0.61 | 1 | 0.67 | 0.39 | 1 | 0.44 | 0.54 | 0.94 | 1 | 1 |
| 3093: 31 | 1129 | 1 | 0.79 | NA | 0.77 | 0.35 | NA | 0.72 | 1 | 0 | 1 | 0.63 |
| 3093: 39 | 1137 | 0.72 | 0.56 | 1 | 0.81 | 0.97 | 1 | 0.75 | 1 | 0 | 1 | 1 |
| 3093: 99 | 1197 | 0.76 | 0.85 | 0.67 | 0.5 | 0.89 | 1 | 0.89 | 1 | 0 | 1 | 0.86 |
| 3093: 104 | 1202 | NA | 1 | 1 | 0.5 | 1 | 1 | 1 | 1 | 1 | 1 | 0.89 |
| 3093: 182 | 1280 | NA | 0.5 | 0.56 | 0.29 | 1 | 1 | 0.39 | 0.96 | NA | 0.5 | 0.89 |
| 3093: 193 | 1291 | 0.55 | 0.62 | 0.66 | 0.7 | 0.91 | 0.66 | 0.49 | 1 | NA | 0.85 | 0.94 |
| 3093: 217 | 1315 | 1 | 0.95 | 1 | 1 | 1 | 1 | 1 | 1 | 0.23 | 1 | 0.8 |
| 3093: 232 | 1330 | 0.85 | 0.66 | 0.77 | 0.87 | 0.95 | NA | 0.85 | 1 | NA | NA | 0.92 |
| 3093: 240 | 1338 | 1 | 0.57 | 1 | 0.88 | 0.97 | 1 | 0.85 | 0.86 | NA | 1 | 0.96 |
| 3093: 247 | 1345 | 0.75 | 0.44 | 0.73 | 0.77 | 0.94 | 0.64 | 0.39 | 1 | NA | 0.62 | 1 |
| 3093: 256 | 1354 | 1 | 0.39 | 0.46 | 0.66 | 0.84 | NA | 0.8 | 0.89 | NA | 0.65 | 0.76 |
| 3093: 258 | 1356 | NA | 1 | 1 | 1 | 1 | 1 | 0.94 | 1 | NA | NA | 1 |
| 3093: 269 | 1367 | 1 | NA | 1 | 1 | 0.92 | 1 | 0.87 | 0.78 | NA | 1 | 0.89 |
| 3093: 277 | 1375 | 0.77 | 0.71 | 0.85 | 0.86 | 1 | 1 | 0.9 | 1 | NA | 0.89 | 0.98 |
| 3093: 319 | 1417 | 1 | 1 | 1 | 1 | 0.92 | 1 | 0.97 | 0.99 | NA | 1 | 0.98 |
| 3093: 347 | 1445 | 0.96 | 0.94 | 1 | 1 | 0.82 | 0.95 | 1 | 1 | NA | 1 | 0.87 |
| 3093: 358 | 1456 | 0.24 | 0.24 | 0.26 | 0.82 | 0.42 | 0.35 | 0.57 | 1 | NA | 0.48 | 1 |
| 3093: 395 | 1493 | 0.94 | 1 | 0.98 | 0.5 | 0.9 | 1 | 0.93 | 0.11 | NA | 1 | 1 |
| 3093: 398 | 1496 | 1 | 1 | 1 | 1 | 1 | NA | 0.93 | 0.54 | NA | 1 | 1 |
| 3093: 415 | 1513 | 1 | 1 | 0.96 | 1 | 1 | 1 | 1 | 1 | NA | 1 | 0.97 |
| 3093: 433 | 1531 | 0.88 | 1 | 1 | 1 | 1 | 1 | 1 | 1 | NA | 1 | 1 |
| 3093: 440 | 1538 | 1 | 0.96 | 0.86 | 1 | 1 | NA | 1 | 1 | NA | 0.9 | 1 |

TABLE 7
(3094):

| | MVP | | | | | | | | | | | | |
|---|---|---|---|---|---|---|---|---|---|---|---|---|---|
| CpG | Position in | | | | | | | | | | | | |
| identifier | ROI | Prostate | Prostate | Prostate | Prostate | Prostate | Prostate | Prostate | Prostate | Muscle | Muscle | Muscle | Muscle |
| 3094: 79 | 549 | 0.85 | 1 | 0.91 | 1 | 1 | 1 | 1 | 1 | 0.98 | 1 | 1 | 0.9 |
| 3094: 103 | 573 | 0.93 | 1 | 1 | 1 | 0.62 | 1 | 0.75 | 1 | 1 | 1 | 1 | 1 |
| 3094: 118 | 588 | 0.4 | 0.79 | 0.85 | 0.97 | 0.65 | 1 | 0.44 | 1 | 0.95 | 0.91 | 0.82 | 1 |
| 3094: 148 | 618 | 0.18 | 1 | 0.99 | 1 | 1 | 0.99 | 1 | 1 | 1 | 0.66 | 1 | 1 |
| 3094: 151 | 621 | 0.63 | 1 | 1 | 1 | 1 | 1 | 1 | 1 | 1 | 0.91 | 1 | 1 |
| 3094: 155 | 625 | 0.48 | NA | 0.62 | 0.57 | NA | 0.48 | 0.76 | 0.9 | 0.61 | 0.66 | 0.72 | 0.83 |
| 3094: 162 | 632 | 1 | 0.63 | 0.66 | 0.9 | 0.23 | 0.89 | 1 | 0.88 | 0.7 | 0.41 | 0.65 | 0.58 |
| 3094: 169 | 639 | 0.72 | 1 | 1 | 1 | 1 | 1 | 0.54 | 1 | 1 | 0.94 | 1 | 1 |
| 3094: 195 | 665 | 0.15 | 0.87 | 0.89 | 0.95 | 0.66 | 0.98 | 0.52 | 0.79 | 0.83 | 0.93 | 0.71 | 0.92 |
| 3094: 342 | 812 | 0.51 | 0.33 | 0.7 | 1 | 0.96 | 0.95 | 1 | 1 | 0.86 | 1 | 0.82 | 0.43 |
| 3094: 393 | 863 | 1 | 1 | 1 | 0.82 | NA | 0.94 | 1 | 1 | 0.82 | 0.78 | 0.72 | 0.72 |

| | MVP | | | | | | | | | | | | | | |
|---|---|---|---|---|---|---|---|---|---|---|---|---|---|---|---|
| CpG | Position | | | | | | | | | | | | | | |
| identifier | in ROI | Muscle | Lung | Lung | Lung | Lung | Lung | Liver | Liver | Breast | Breast | Breast | Breast | Breast | Breast |
| 3094: 79 | 549 | 0.93 | 0.96 | 1 | 1 | 0.9 | 1 | 0.4 | 0.78 | 0.93 | 0.92 | 1 | 1 | 1 | 1 |
| 3094: 103 | 573 | 1 | 1 | 1 | 1 | 1 | 0.88 | 1 | 0.59 | 1 | 1 | 1 | 1 | 1 | 1 |
| 3094: 118 | 588 | 0.85 | 0.89 | 0.94 | 0.81 | 0.91 | 0.94 | 1 | 0.23 | 0.94 | 0.87 | 1 | 0.7 | 0.83 | 0.84 |
| 3094: 148 | 618 | 0.97 | 0.98 | 1 | 1 | 1 | 0.98 | 0.79 | 0.56 | 1 | 1 | 1 | 1 | 0.96 | 1 |
| 3094: 151 | 621 | 1 | 1 | 1 | 1 | 1 | 1 | 0.67 | 0.51 | 1 | 1 | 1 | 1 | 1 | 1 |
| 3094: 155 | 625 | 0.66 | 0.6 | 1 | NA | 0.61 | 1 | 0.88 | NA | 0.77 | NA | NA | NA | 0.66 | 0.54 |
| 3094: 162 | 632 | 0.57 | 0.77 | 0.87 | 0.67 | 0.83 | 0.89 | 0.19 | 0.12 | 0.6 | 0.65 | 0.82 | 0.62 | 0.63 | 0.64 |
| 3094: 169 | 639 | 0.96 | 1 | 1 | 1 | 1 | 1 | 0.6 | 0.75 | 1 | 1 | 1 | 1 | 1 | 1 |
| 3094: 195 | 665 | 0.8 | 0.79 | 1 | 0.091 | 0.92 | 1 | 0.54 | 0.36 | 0.94 | 0.89 | 0.96 | 0.9 | 0.87 | 0.75 |
| 3094: 342 | 812 | 0.84 | 0.97 | 1 | 0.37 | 0.85 | 0.96 | 0.86 | 1 | 0.82 | 0.98 | 0.97 | 1 | 0.63 | 0.97 |
| 3094: 393 | 863 | 1 | 0.91 | 0.93 | 1 | 0.96 | 0.85 | 0.94 | 0.88 | 0.92 | 0.94 | 0.89 | 0.92 | 0.87 | 1 |

| | MVP Position | | | | | | |
|---|---|---|---|---|---|---|---|
| | in ROI | Brain | Brain | Brain | Brain | Brain | Brain |
| 3094: 79 | 549 | 0.9 | 0.5 | 1 | 0.92 | 1 | 1 |
| 3094: 103 | 573 | 1 | 1 | 1 | 1 | 1 | 1 |
| 3094: 118 | 588 | 1 | 0.5 | 1 | 0.89 | 0.87 | 0.87 |
| 3094: 148 | 618 | 1 | 1 | 0.92 | 0.97 | 1 | 1 |
| 3094: 151 | 621 | 1 | 1 | 1 | 1 | 1 | 1 |
| 3094: 155 | 625 | 0.9 | 1 | 0.61 | 0.81 | NA | NA |
| 3094: 162 | 632 | 1 | 0.91 | 0.93 | 0.62 | 0.7 | 0.75 |
| 3094: 169 | 639 | 1 | 1 | 1 | 1 | 1 | 1 |
| 3094: 195 | 665 | 0.94 | 0.5 | 0.89 | 0.89 | 0.9 | 0.92 |
| 3094: 342 | 812 | 1 | 0.5 | 0.95 | 0.79 | 0.9 | 1 |
| 3094: 393 | 863 | 0.95 | 0.5 | 1 | 0.97 | 1 | 0.96 |

TABLE 8
(3103):

| | MVP | | | | | | | | | | | | |
|---|---|---|---|---|---|---|---|---|---|---|---|---|---|
| CpG | Position in | | | | | | | | | | | | |
| identifier | ROI | Prostate | Prostate | Prostate | Prostate | Prostate | Prostate | Prostate | Prostate | Muscle | Muscle | Muscle | Muscle |
| 3103: 41 | 1752 | NA | 1 | NA | 0.5 | NA | NA | 1 | 1 | NA | 0.12 | 0 | 0.62 |
| 3103: 47 | 1758 | NA | 0.76 | NA | 0.58 | 0 | 1 | 1 | 0.39 | 0.65 | NA | 0.58 | 0.5 |
| 3103: 76 | 1787 | 0.24 | 1 | 1 | 0.86 | 1 | 1 | 0 | 0.056 | 1 | 0.64 | 1 | 0.83 |
| 3103: 89 | 1800 | 1 | 1 | 0.83 | 0.99 | 1 | 1 | 0.98 | 0.34 | 1 | 1 | 1 | 1 |
| 3103: 106 | 1817 | 1 | 1 | 0.53 | 0.98 | 1 | 1 | 0 | 1 | 0.94 | 0.63 | 0.77 | 0.95 |
| 3103: 152 | 1863 | 1 | 0.67 | 1 | 0.98 | 1 | 1 | 0.98 | 0.75 | 0.94 | 1 | 1 | 0.83 |
| 3103: 163 | 1874 | 0.44 | 0.83 | 1 | 1 | 0 | 1 | 0 | 0.095 | 0.51 | 0.42 | 0.12 | 0.47 |
| 3103: 190 | 1901 | 1 | 0.58 | 0.84 | 0.78 | 1 | 1 | 0.92 | 0.0041 | 0.71 | 0.52 | 0.81 | 0.8 |
| 3103: 196 | 1907 | 1 | 1 | NA | 0.87 | 1 | 1 | 1 | 0.16 | 0.9 | 0.95 | 0.88 | 0.94 |
| 3103: 203 | 1914 | 1 | 0.54 | 0.83 | 1 | 1 | 1 | 0.84 | 0 | 0.74 | 0.57 | 0.44 | 0.55 |
| 3103: 227 | 1938 | 1 | 0.35 | 1 | 0.84 | 1 | 1 | 0.85 | 0.14 | 0.69 | 0.61 | 0.62 | 0.61 |
| 3103: 231 | 1942 | 1 | 1 | 0.74 | 1 | 1 | 0 | 0.9 | 0.1 | 0.83 | 0.93 | 0.58 | 0.69 |
| 3103: 238 | 1949 | 1 | 1 | NA | 0.94 | 0.95 | 1 | 0.96 | 0.94 | 0.73 | 1 | 0.84 | 0.91 |
| 3103: 279 | 1990 | 0.96 | 0.86 | 0.45 | 0.96 | 0.68 | 0.51 | 1 | 0.011 | 0.57 | 1 | 0.42 | 0.6 |
| 3103: 285 | 1996 | 0.47 | 0.33 | NA | 0.76 | 0.94 | 0.36 | 0.91 | 0.024 | 0.43 | 0 | 0.47 | 0.39 |
| 3103: 292 | 2003 | 1 | 1 | 0.48 | 1 | 0.93 | NA | 0.99 | 0 | 0.69 | 0.8 | 0.76 | 0.83 |
| 3103: 294 | 2005 | 0.51 | 0.42 | 0.68 | 1 | 0.78 | NA | 0.98 | 0 | 0.49 | 0.61 | 0.3 | 0.4 |
| 3103: 306 | 2017 | 0.95 | 0.9 | 0.84 | 0.92 | 0.6 | 0.099 | 1 | 0 | 0.84 | 0.93 | 0.4 | 0.78 |
| 3103: 311 | 2022 | 1 | 1 | NA | 1 | 1 | NA | 0.95 | 0.096 | 0.83 | 1 | 0.8 | 0.86 |
| 3103: 317 | 2028 | 0.83 | 1 | 0.65 | 1 | 1 | 1 | 1 | 0 | 0.93 | 0.5 | 0.91 | 0.95 |
| 3103: 319 | 2030 | 0.75 | 1 | 1 | 0.99 | 0.96 | 1 | 0.96 | 0.13 | 0.84 | 1 | 0.84 | 0.89 |
| 3103: 333 | 2044 | 1 | 0.69 | NA | 0.98 | 0.96 | 1 | 0 | 0.5 | 0.55 | 0.73 | 0.51 | 0.38 |
| 3103: 346 | 2057 | 0.035 | 0.77 | 0.61 | 1 | 1 | 0.3 | 0.012 | 0.023 | 0.73 | 0.76 | 0.42 | 0.68 |
| 3103: 365 | 2076 | 1 | 0.68 | NA | 1 | 1 | 1 | 1 | 0.013 | 0.49 | 0.56 | 0.46 | 0.4 |
| 3103: 378 | 2089 | 0.35 | NA | 0.83 | 1 | 0.96 | 1 | 1 | 0.67 | 0.65 | 0.53 | 0.18 | 0.58 |
| 3103: 384 | 2095 | 1 | 0.68 | NA | 1 | 1 | 1 | 1 | 0.77 | 0.71 | 0.88 | 0.7 | 0.8 |

| | MVP | | | | | | | | | | | | | | |
|---|---|---|---|---|---|---|---|---|---|---|---|---|---|---|---|
| CpG | Position | | | | | | | | | | | | | | |
| identifier | in ROI | Muscle | Lung | Lung | Lung | Lung | Lung | Liver | Liver | Breast | Breast | Breast | Breast | Breast | Breast |
| 3103: 41 | 1752 | 0.61 | 1 | 0 | NA | 1 | 1 | NA | 1 | 1 | NA | 1 | 0.14 | NA | 0.36 |
| 3103: 47 | 1758 | 0.88 | 0.82 | 1 | 1 | 1 | 1 | 0 | 1 | 1 | NA | 1 | 0.3 | 1 | NA |
| 3103: 76 | 1787 | 1 | 1 | 1 | 0.68 | 0.76 | 1 | 1 | 1 | 0.93 | NA | 0.77 | 1 | 1 | 0.5 |
| 3103: 89 | 1800 | 1 | 1 | 1 | NA | 1 | 1 | 1 | 1 | 1 | NA | 0.94 | 1 | 1 | 1 |
| 3103: 106 | 1817 | 0.64 | 1 | 1 | 1 | 1 | 1 | 1 | 1 | 0.55 | NA | 0.75 | 0.96 | 0.94 | 0.75 |
| 3103: 152 | 1863 | 0.84 | 1 | 1 | 0.73 | 1 | 1 | 1 | 1 | 0.79 | NA | 0.89 | 0.83 | 1 | 0.9 |
| 3103: 163 | 1874 | 0.55 | 0.88 | 1 | NA | 0.87 | 1 | 1 | 1 | 0.68 | NA | 0.36 | 0.31 | 0.63 | 0.37 |
| 3103: 190 | 1901 | 1 | 0.97 | 1 | 0.65 | 0.98 | 1 | 1 | 1 | 0.9 | NA | 0.84 | 0.43 | 0.85 | 0.89 |
| 3103: 196 | 1907 | 0.91 | 1 | 1 | 0.52 | 0.98 | 1 | 1 | 1 | 0.91 | NA | 0.83 | 0.93 | 0.77 | 0.83 |
| 3103: 203 | 1914 | 0.58 | 0.66 | 1 | 1 | 0.48 | 1 | 1 | 1 | 0.71 | NA | 0.59 | 0.46 | 0.48 | 0.2 |
| 3103: 227 | 1938 | 0.62 | 0.56 | 1 | 0.89 | 0.58 | 0.8 | 1 | 1 | 0.83 | NA | 0.69 | 0.31 | 0.69 | 0.53 |
| 3103: 231 | 1942 | 0.87 | 0.9 | 1 | 0.49 | 0.9 | 0.89 | 1 | 1 | 0.88 | NA | 0.89 | 0.82 | 0.79 | 0.78 |
| 3103: 238 | 1949 | 0.95 | 1 | 1 | NA | 1 | 1 | 1 | 1 | 0.66 | NA | 0.82 | 0.94 | 0.9 | 1 |
| 3103: 279 | 1990 | 0.57 | 0.75 | 0.62 | NA | 0.74 | 0.2 | 1 | 1 | 0.78 | 0.74 | 0.75 | 0.62 | 0.57 | 0.77 |
| 3103: 285 | 1996 | 0.47 | 0.63 | 0.54 | 0.24 | 0.64 | 0.56 | 1 | NA | 0.78 | 0.056 | 0.73 | 0.65 | 0.76 | 0.61 |
| 3103: 292 | 2003 | 0.87 | 0.9 | NA | 0.53 | 0.88 | NA | 1 | 1 | 1 | 0.75 | 0.93 | 0.97 | 0.88 | 0.97 |
| 3103: 294 | 2005 | 0.32 | 0.54 | 1 | NA | 0.56 | 1 | 0.86 | 1 | 0.5 | 0.62 | 0.52 | 0.52 | 0.59 | 0.48 |
| 3103: 306 | 2017 | 0.83 | 0.86 | 0.27 | 0.4 | 0.83 | 0.003 | 1 | NA | 0.87 | 0.56 | 0.87 | 1 | 0.85 | 0.63 |
| 3103: 311 | 2022 | 0.9 | 0.83 | 1 | 0.67 | 0.87 | NA | 1 | 1 | 0.92 | 0.57 | 0.86 | 0.76 | 0.91 | 0.88 |
| 3103: 317 | 2028 | 1 | 1 | 1 | 1 | 1 | 1 | 1 | 1 | 0.98 | 1 | 0.97 | 1 | 0.97 | 0.96 |
| 3103: 319 | 2030 | 0.93 | 0.97 | 1 | NA | 1 | 1 | 1 | 1 | 0.9 | 1 | 0.94 | 0.82 | 0.96 | 0.96 |
| 3103: 333 | 2044 | 0.4 | 0.6 | 1 | 0.22 | 0.55 | 1 | 1 | 1 | 0.55 | 0.53 | 0.58 | 0.56 | 0.53 | 0.59 |
| 3103: 346 | 2057 | 0.43 | 0.5 | NA | 0 | 0.78 | 1 | 1 | NA | 0.77 | 0.64 | 0.82 | 0.56 | 0.76 | 0.76 |
| 3103: 365 | 2076 | 0.45 | 0.56 | 1 | NA | 0.34 | 1 | 1 | NA | 0.75 | 0.23 | 0.6 | 0.52 | 0.67 | 0.29 |
| 3103: 378 | 2089 | 0.55 | 0.45 | NA | NA | 0.52 | 1 | 1 | 1 | 0.79 | 0 | 0.69 | 1 | 0.54 | 1 |
| 3103: 384 | 2095 | 0.56 | 0.56 | 1 | 0.5 | 0.62 | NA | 1 | NA | 0.85 | 0.59 | 0.77 | 0.81 | 0.74 | 0.55 |

| CpG | MVP Position in | | | | | | |
|---|---|---|---|---|---|---|---|
| identifier | ROI | Brain | Brain | Brain | Brain | Brain | Brain |
| 3103: 41 | 1752 | NA | 1 | NA | 1 | 0.95 | NA |
| 3103: 47 | 1758 | 1 | 0 | NA | 1 | 0.82 | 1 |
| 3103: 76 | 1787 | 1 | 1 | 1 | 1 | 1 | 1 |
| 3103: 89 | 1800 | 1 | 1 | 1 | 1 | 1 | 1 |
| 3103: 106 | 1817 | 1 | 1 | 1 | 1 | 1 | 1 |
| 3103: 152 | 1863 | 0.87 | 0.86 | 0.13 | 1 | 0.79 | 1 |
| 3103: 163 | 1874 | 0.76 | 0.97 | 0.12 | 0.95 | 0.76 | 0.39 |
| 3103: 190 | 1901 | 0.96 | 1 | 1 | 1 | 0.75 | 0.43 |
| 3103: 196 | 1907 | 0.93 | 1 | 1 | 1 | 0.9 | 0.84 |
| 3103: 203 | 1914 | NA | 1 | 0.91 | 0.76 | 0.71 | 0.28 |
| 3103: 227 | 1938 | 0.58 | 0.78 | 0.64 | 0.78 | 0.69 | 0.65 |
| 3103: 231 | 1942 | 0.93 | 0.93 | 1 | 0.95 | 0.78 | 0.96 |
| 3103: 238 | 1949 | 0.94 | 0.84 | 1 | 0.91 | 0.86 | 0.96 |
| 3103: 279 | 1990 | 0.82 | 0.88 | 0.98 | 1 | 0.72 | 0.74 |
| 3103: 285 | 1996 | 0.5 | 0.77 | 0.66 | 0.68 | 0.7 | 0.47 |
| 3103: 292 | 2003 | 0.89 | 0.87 | 1 | 1 | 0.88 | 0.88 |
| 3103: 294 | 2005 | 0.55 | 0.5 | 0.022 | 0.73 | 0.52 | 0.75 |
| 3103: 306 | 2017 | 1 | 0.87 | 0.97 | 0.9 | 0.82 | 0.77 |
| 3103: 311 | 2022 | 0.94 | 0.9 | 1 | 1 | 0.9 | 0.96 |
| 3103: 317 | 2028 | 0.96 | 0.97 | 1 | 1 | 0.95 | 1 |
| 3103: 319 | 2030 | 0.95 | 0.97 | 0.96 | 1 | 0.88 | 0.98 |
| 3103: 333 | 2044 | 0.61 | 0.7 | 0.93 | 0.65 | 0.65 | 0.81 |
| 3103: 346 | 2057 | 0.76 | 0.86 | 0.92 | 0.39 | 0.61 | 0.91 |
| 3103: 365 | 2076 | 0.77 | 1 | 1 | 1 | 0.6 | 0.65 |
| 3103: 378 | 2089 | 0.54 | 0.5 | 0.62 | 0.53 | 0.67 | 1 |
| 3103: 384 | 2095 | 0.83 | 0.85 | 1 | 0.93 | 0.73 | 1 |

TABLE 9
(3104):

| CpG | MVP Position in | | | | | | | | | | | | |
|---|---|---|---|---|---|---|---|---|---|---|---|---|---|
| identifier | ROI | Prostate | Prostate | Prostate | Muscle | Muscle | Muscle | Muscle | Muscle | Lung | Lung | Lung | Lung |
| 3104: 75 | 1818 | 0.54 | 0 | 0.96 | 0.89 | 0.85 | 0.96 | 1 | 0.93 | 0.82 | 1 | 0.69 | 1 |
| 3104: 79 | 1822 | 0.75 | 0.93 | 1 | 0.86 | 1 | 0.89 | 0.95 | 0.97 | 0.95 | 1 | 0.45 | 1 |
| 3104: 132 | 1875 | 0.93 | 0.21 | 0.86 | 0.68 | 0.83 | 0.019 | 0.69 | 0.81 | 0.85 | 1 | 0 | 0 |
| 3104: 137 | 1880 | 1 | 0.25 | 0.72 | 0.75 | 0.84 | NA | 0.77 | 0.91 | 0.52 | 1 | 0.74 | 0.74 |
| 3104: 245 | 1988 | 1 | 1 | 1 | 0.96 | 0.79 | 0 | 0.78 | 0.9 | 1 | 1 | 0.92 | 1 |
| 3104: 249 | 1992 | 1 | 1 | 1 | 0.96 | 0.47 | 1 | 1 | 1 | 1 | 1 | 0.71 | 0.82 |
| 3104: 254 | 1997 | 0.92 | 0 | 0.66 | 0.59 | 1 | 1 | 0.48 | 0.64 | 0.61 | 1 | 0.19 | 0.33 |
| 3104: 302 | 2045 | 0.87 | 1 | 1 | 1 | 1 | NA | 1 | 1 | 0.96 | 1 | 1 | 1 |
| 3104: 306 | 2049 | 1 | 1 | 1 | 1 | 1 | 0.47 | 0.87 | 0.74 | 1 | 1 | 0.91 | 0.69 |
| 3104: 333 | 2076 | 1 | 0.97 | 1 | 0.72 | 1 | 0 | 0.84 | 0.47 | 0.81 | 0.13 | 1 | 1 |
| 3104: 349 | 2092 | 1 | 0.67 | 0.93 | 0.75 | 1 | 1 | 0.63 | 0.55 | 0.83 | 1 | 0.34 | 0.36 |
| 3104: 361 | 2104 | 1 | 1 | 1 | 0.78 | 0.9 | 0.65 | 0.92 | 1 | 1 | 0.5 | 0.91 | 1 |
| 3104: 386 | 2129 | NA | 1 | 1 | 0.87 | 1 | 0.86 | 0.87 | 0.67 | 0.92 | 0.5 | 1 | 1 |
| 3104: 425 | 2168 | 1 | 0.96 | 1 | 0.68 | 1 | 1 | 1 | 0.69 | 0.7 | 0.63 | 1 | 1 |
| 3104: 475 | 2218 | NA | NA | NA | NA | NA | NA | NA | 1 | 1 | 0.92 | NA | NA |

| | MVP | | | | | | | | | | | | | | |
|---|---|---|---|---|---|---|---|---|---|---|---|---|---|---|---|
| CpG | Position | | | | | | | | | | | | | | |
| identifier | in ROI | Liver | Liver | Breast | Breast | Breast | Breast | Breast | Breast | Brain | Brain | Brain | Brain | Brain | Brain |
| 3104: 75 | 1818 | 0.44 | 0.15 | 0.88 | 0.58 | 0.82 | 1 | 0.86 | 0.6 | 0.98 | 0.98 | 1 | 1 | 1 | 1 |
| 3104: 79 | 1822 | 0 | 0.13 | 0.92 | 0.77 | 0.87 | 0.97 | 0.99 | 0.81 | 1 | 1 | 1 | 1 | 1 | 1 |
| 3104: 132 | 1875 | 0.27 | 0.32 | 0.28 | 0.54 | 0.55 | 0.72 | 0.68 | 0.61 | 0.73 | 0.95 | 1 | 0.9 | 0.57 | 0.59 |
| 3104: 137 | 1880 | 0.42 | 0.41 | 0.6 | 0.41 | 0.43 | 0.73 | 0.74 | 0.6 | 0.82 | 0.74 | 1 | 0.75 | 0.8 | 0.69 |
| 3104: 245 | 1988 | 0.75 | 0.6 | 0.96 | 1 | 1 | 0.97 | 0.94 | 0.92 | 1 | 1 | 1 | 0.89 | 0.62 | 0.95 |
| 3104: 249 | 1992 | 0.55 | 0.61 | 1 | 1 | 0.91 | 1 | 1 | 1 | 1 | 1 | 1 | 1 | 1 | 1 |
| 3104: 254 | 1997 | 0.55 | 0.31 | 0.39 | 0.67 | 0.49 | 0.58 | 1 | 0.5 | 0.6 | 0.94 | 1 | 0.78 | NA | 0.38 |
| 3104: 302 | 2045 | 0.93 | 1 | 1 | 1 | 0.78 | 0.95 | 1 | 1 | 1 | 1 | 1 | 1 | 1 | 1 |
| 3104: 306 | 2049 | 1 | 0.76 | 0.94 | 0.96 | 1 | 1 | 1 | 1 | 1 | 1 | 1 | 0.5 | 0.76 | 1 |
| 3104: 333 | 2076 | 0.64 | 0.38 | 0.9 | 0.85 | 0.8 | 1 | 0.73 | 0.85 | 1 | 1 | 1 | 1 | 1 | 1 |
| 3104: 349 | 2092 | 0.7 | 0.49 | 0.48 | 0.9 | 0.66 | 0.88 | 0.83 | 0.56 | 0.82 | 1 | 1 | 1 | 0.63 | 0.63 |
| 3104: 361 | 2104 | 1 | 1 | 1 | 1 | 1 | 1 | 1 | 1 | 1 | 1 | 1 | 1 | 1 | 1 |
| 3104: 386 | 2129 | 1 | 1 | 1 | 1 | 1 | 1 | 0.72 | 1 | 1 | 1 | 1 | 1 | 1 | 1 |
| 3104: 425 | 2168 | 0.85 | 1 | 0.85 | 1 | 1 | 1 | 0.79 | 0.83 | 0.88 | 1 | 1 | 0.9 | 1 | 1 |
| 3104: 475 | 2218 | NA | 1 | NA | 0.87 | NA | NA | NA | NA | NA | 0.9 | 0 | NA | NA | NA |

TABLE 10
(3105):

| | MVP | | | | | | | | | | | | |
|---|---|---|---|---|---|---|---|---|---|---|---|---|---|
| CpG | Position in | | | | | | | | | | | | |
| identifier | ROI | Prostate | Prostate | Prostate | Prostate | Prostate | Prostate | Prostate | Muscle | Muscle | Muscle | Muscle | Muscle |
| 3105: 45 | 300 | 1 | 1 | 1 | 0.74 | 1 | 1 | 0.75 | 0.95 | 1 | 1 | 0.5 | 1 |
| 3105: 64 | 319 | 0.76 | 0.51 | 0.59 | 1 | 0.66 | 0.55 | 0 | 0.29 | 0.23 | 0.25 | 0.074 | 0.42 |
| 3105: 73 | 328 | 1 | 1 | 1 | 0.61 | 1 | 1 | 0.88 | 1 | 1 | 1 | 0.97 | 1 |
| 3105: 85 | 340 | 1 | 0.95 | 1 | 1 | 1 | 1 | 0.86 | 0.94 | 0.92 | 1 | 1 | 1 |
| 3105: 97 | 352 | 0.87 | 1 | 0.42 | 0.67 | 0.74 | 0 | 0.88 | 0.9 | 0.67 | 0.79 | 0.68 | 0.69 |
| 3105: 132 | 387 | 0.97 | 1 | 1 | 1 | 0.98 | 1 | 0.026 | 0.86 | 0.78 | 1 | 1 | 0.94 |
| 3105: 136 | 391 | 1 | 0.95 | 1 | 0.61 | 0.94 | 1 | 0.075 | 0.78 | 1 | 0.9 | 0.8 | 0.96 |
| 3105: 151 | 406 | 1 | 1 | 1 | 0.73 | 1 | 1 | 0.081 | 1 | 1 | 1 | 1 | 1 |
| 3105: 163 | 418 | 1 | 0.69 | 0.74 | 0.65 | 0.77 | 0.08 | 0.84 | 0.71 | 0.76 | 0.96 | 0.71 | 0.92 |
| 3105: 172 | 427 | 1 | 1 | 1 | 1 | 1 | 1 | 0.86 | 1 | 1 | 1 | 1 | 1 |
| 3105: 193 | 448 | 1 | 1 | 1 | 0.56 | 1 | 1 | 0.73 | 0.96 | 1 | 1 | 0.91 | 1 |
| 3105: 202 | 457 | 1 | 1 | 1 | 0.13 | 0.98 | 0.84 | 0 | 1 | 0.9 | 1 | 0.84 | 1 |
| 3105: 256 | 511 | 0.96 | 0.94 | 0.98 | 0.76 | 1 | 0.79 | 0.91 | 0.68 | 0.36 | 0.7 | 0.67 | 0.95 |
| 3105: 280 | 535 | 1 | 0.83 | 0.82 | 0.77 | 0.88 | 0.67 | 0.95 | 0.53 | 0.86 | 0.26 | 0.33 | 0.61 |
| 3105: 301 | 556 | 0.97 | 1 | 0.38 | 0.5 | 0.94 | 0.5 | 0.95 | 0.44 | 0 | 0.51 | 0.19 | 0.45 |
| 3105: 337 | 592 | 1 | 0.93 | 1 | 1 | 0.36 | 0.81 | 0.85 | 0.19 | 0.25 | 0.38 | 0.33 | 0.48 |
| 3105: 364 | 619 | 1 | 1 | 1 | 0.06 | 1 | 1 | 0 | 0.9 | 1 | 1 | 0.9 | 1 |
| 3105: 367 | 622 | 0.92 | 0.57 | 1 | 0 | 0.79 | 1 | 0 | 0.14 | 0 | 0.18 | 0.026 | 0.31 |
| 3105: 375 | 630 | 1 | 1 | 1 | 0.67 | 0.93 | 0.4 | 0 | 0.43 | 0 | 0.39 | 0.24 | 0.92 |

| | MVP | | | | | | | | | | | | | | |
|---|---|---|---|---|---|---|---|---|---|---|---|---|---|---|---|
| CpG | Position in | | | | | | | | | | | | | | |
| identifier | ROI | Lung | Lung | Lung | Lung | Lung | Liver | Liver | Breast | Breast | Breast | Breast | Breast | Breast | Brain |
| 3105: 45 | 300 | 1 | 1 | 1 | 1 | 1 | 1 | 1 | 0.5 | 0.62 | 0.58 | 0.74 | 0.23 | 0.41 | 1 |
| 3105: 64 | 319 | 0.77 | 0.79 | 1 | 0.91 | 0.9 | 1 | 0.88 | 0.2 | 0.098 | 0.1 | 0.79 | 0.18 | 0.19 | 0.9 |
| 3105: 73 | 328 | 1 | 0.94 | 1 | 1 | 1 | 1 | 1 | 0.53 | 0.61 | 0.5 | 0.96 | 0.53 | 0.79 | 1 |
| 3105: 85 | 340 | 1 | 1 | 1 | 1 | 0.97 | 1 | 1 | 0.66 | 0.87 | 0.69 | 0.71 | 0.78 | 0.58 | 0.72 |
| 3105: 97 | 352 | 0.98 | 0.5 | 0 | 1 | 1 | 1 | 1 | 0.29 | 0.83 | 0.26 | 0.92 | 0.78 | 0.75 | 0.99 |
| 3105: 132 | 387 | 1 | 1 | 1 | 0.99 | 1 | 1 | 1 | 0.32 | 0.64 | 0.59 | 0.92 | 0.68 | 0.72 | 0.98 |
| 3105: 136 | 391 | 1 | 1 | 0 | 1 | 1 | 1 | 1 | 0.53 | 0.56 | 0.76 | 0.83 | 0.57 | 0.49 | 0.96 |
| 3105: 151 | 406 | 1 | 1 | 1 | 1 | 1 | 1 | 1 | 0.71 | 0.93 | 0.77 | 1 | 0.96 | 0.93 | 1 |
| 3105: 163 | 418 | 1 | 0.9 | 1 | 0.82 | 1 | 1 | 1 | 0.44 | 0.6 | 0.39 | 0.65 | 0.38 | 0.48 | 0.97 |
| 3105: 172 | 427 | 1 | 1 | 1 | 1 | 1 | 1 | 1 | 0.88 | 0.94 | 1 | 1 | 0.93 | 1 | 1 |
| 3105: 193 | 448 | 1 | 0.97 | 1 | 1 | 1 | 1 | 1 | 0.67 | 0.9 | 0.81 | 0.88 | 0.69 | 0.72 | 1 |
| 3105: 202 | 457 | 1 | 0.98 | 1 | 1 | 1 | 1 | 1 | 0.63 | 0.82 | 0.67 | 0.94 | 0.66 | 0.74 | 1 |
| 3105: 256 | 511 | 0.87 | 0.9 | 1 | 0.97 | 1 | 0.89 | 0.9 | 0.41 | 0.56 | 0.4 | 0.87 | 0.45 | 0.6 | 0.95 |
| 3105: 280 | 535 | 1 | 0.6 | 1 | 1 | 0.96 | 1 | 1 | 0.45 | 0.68 | 0.33 | 0.84 | 0.37 | 0.5 | 1 |
| 3105: 301 | 556 | 1 | 0.77 | 1 | 1 | 0.5 | 1 | 1 | 0.47 | 0.49 | 0.62 | 0.96 | 0.51 | 0.74 | 1 |
| 3105: 337 | 592 | 1 | 1 | 1 | 1 | 1 | 0.95 | 1 | 0.48 | 0.62 | 0.88 | 0.59 | 0.38 | 0.47 | 1 |
| 3105: 364 | 619 | 1 | 1 | 1 | 1 | 1 | 1 | 1 | 0.81 | 0.85 | 0.84 | 1 | 0.81 | 0.83 | 1 |
| 3105: 367 | 622 | 0.85 | 1 | 1 | 1 | 0 | 1 | 1 | 0.24 | 0 | 0.42 | 1 | 0 | 0.74 | 0.93 |
| 3105: 375 | 630 | 0.92 | 0.83 | 1 | 0.97 | NA | 0.94 | 0.96 | 0.4 | 0.29 | 0.22 | 1 | 0.46 | 0.29 | 1 |

| CpG | MVP Position in | | | | | |
|---|---|---|---|---|---|---|
| identifier | ROI | Brain | Brain | Brain | Brain | Brain |
| 3105: 45 | 300 | 1 | 1 | 1 | 1 | 0.92 |
| 3105: 64 | 319 | 0.87 | 0.027 | 0.68 | 0.81 | 0.8 |
| 3105: 73 | 328 | 1 | 1 | 1 | 1 | 0.97 |
| 3105: 85 | 340 | 0.97 | 1 | 1 | 1 | 1 |
| 3105: 97 | 352 | 0.91 | 1 | 1 | 0.99 | 0.93 |
| 3105: 132 | 387 | 1 | 1 | 0.8 | 1 | 0.9 |
| 3105: 136 | 391 | 1 | 1 | 1 | 0.97 | 1 |
| 3105: 151 | 406 | 1 | 1 | 1 | 1 | 1 |
| 3105: 163 | 418 | 1 | 1 | 1 | 1 | 1 |
| 3105: 172 | 427 | 1 | 1 | 1 | 1 | 1 |
| 3105: 193 | 448 | 1 | 1 | 1 | 1 | 1 |
| 3105: 202 | 457 | 1 | 1 | 0.98 | 1 | 0.98 |
| 3105: 256 | 511 | 1 | 0.95 | 1 | 1 | 0.97 |
| 3105: 280 | 535 | 1 | 1 | 1 | 1 | 1 |
| 3105: 301 | 556 | 0.9 | 1 | 1 | 1 | 1 |
| 3105: 337 | 592 | 1 | 1 | 1 | 0.89 | 0.85 |
| 3105: 364 | 619 | 1 | 1 | 0.9 | 1 | 1 |
| 3105: 367 | 622 | 1 | 1 | 1 | 0.93 | 0.87 |
| 3105: 375 | 630 | 1 | 1 | 1 | 1 | 1 |

TABLE 11
(3107):

| | MVP | | | | | | | | | | | | |
|---|---|---|---|---|---|---|---|---|---|---|---|---|---|
| CpG | Position in | | | | | | | | | | | | |
| identifier | ROI | Prostate | Prostate | Muscle | Muscle | Muscle | Muscle | Muscle | Lung | Lung | Lung | Lung | Liver |
| 3107: 58 | 336 | 1 | 1 | 1 | 0.83 | 1 | 1 | 0.81 | 1 | 1 | 1 | 1 | 0.72 |
| 3107: 60 | 338 | 0.97 | 1 | 1 | 1 | 1 | 1 | 1 | 1 | 1 | 1 | 1 | 1 |
| 3107: 80 | 358 | 1 | 1 | 1 | 0.94 | 1 | 1 | 1 | 1 | 0.59 | 1 | 1 | 1 |
| 3107: 97 | 375 | 0.99 | 1 | 1 | 1 | 1 | 1 | 0.96 | 1 | 0.66 | 1 | 0.97 | 1 |
| 3107: 100 | 378 | 1 | 1 | 1 | 0.96 | 0.38 | 1 | 0.97 | 1 | 0.9 | 1 | 0.98 | 1 |
| 3107: 120 | 398 | 1 | 0.82 | 0.77 | 0.97 | 0.57 | 1 | 0.91 | 0.95 | 0.94 | 0.85 | 0.88 | 0.97 |
| 3107: 137 | 415 | 0.98 | 0.95 | 0.94 | 1 | 1 | 1 | 0.82 | 1 | 1 | 0.95 | 0.96 | 0.99 |
| 3107: 139 | 417 | 0.98 | 1 | 1 | 1 | 1 | 1 | 1 | 0.86 | 1 | 1 | 0.96 | 0.98 |
| 3107: 148 | 426 | 1 | 0.98 | 0.81 | 1 | 0.99 | 0.98 | 0.95 | 1 | 1 | 0.92 | 0.97 | 1 |
| 3107: 164 | 442 | 1 | 0.95 | 1 | 1 | 1 | 0.95 | 0.75 | 0.98 | 1 | 1 | 0.72 | 0.98 |
| 3107: 187 | 465 | 0.82 | 0.98 | 0.92 | 1 | 0.57 | 0.83 | 0.81 | 0.91 | 0.99 | 1 | 0.92 | 0.79 |
| 3107: 190 | 468 | 0.71 | 0.94 | 1 | 0.81 | 0.15 | 0.75 | 0.77 | 0.85 | 1 | 0.66 | 0.89 | 0.75 |
| 3107: 209 | 487 | 0.95 | 0.87 | 0.65 | 0.69 | 0.91 | 0.68 | 0.53 | 0.33 | 1 | 0.63 | 0.64 | 0.59 |
| 3107: 224 | 502 | 0.84 | 0.93 | 1 | 1 | 0.97 | 0.97 | 0.84 | 0.79 | 0.97 | 0.88 | 0.93 | 0.97 |
| 3107: 233 | 511 | 0.76 | 0.83 | 0.55 | 0.84 | 0.69 | 0.77 | 0.68 | 0.58 | 0.65 | 0.83 | 0.68 | 0.49 |
| 3107: 243 | 521 | 1 | 0.96 | 0.88 | 0.97 | 0.98 | 0.93 | 0.95 | 0.83 | 0.82 | 0.73 | 0.89 | 0.68 |
| 3107: 257 | 535 | 0.82 | 1 | 0.78 | 0.72 | 1 | 0.72 | 0.79 | 0.44 | 0.56 | 0.58 | 0.74 | 0.43 |
| 3107: 265 | 543 | 0.95 | 0.94 | 1 | 0.98 | 0.96 | 0.87 | 1 | 0.69 | 0.64 | 0.65 | 0.79 | 0.54 |
| 3107: 400 | 678 | 0.65 | 0.94 | 0.81 | 1 | 0.98 | 1 | 0.99 | 0.37 | 0.34 | 0.53 | 0.76 | 0.84 |

| | MVP | | | | | | | | | | | | |
|---|---|---|---|---|---|---|---|---|---|---|---|---|---|
| CpG | Position in | | | | | | | | | | | | |
| identifier | ROI | Liver | Breast | Breast | Breast | Breast | Breast | Breast | Brain | Brain | Brain | Brain | Brain |
| 3107: 58 | 336 | 1 | 1 | 1 | 1 | 0.91 | 1 | 1 | 0.88 | 1 | 0.5 | 1 | 1 |
| 3107: 60 | 338 | 1 | 1 | 0.96 | 1 | 1 | 1 | 1 | 1 | 1 | 1 | 1 | 1 |
| 3107: 80 | 358 | 1 | 1 | 0.94 | 1 | 1 | 1 | 1 | 1 | 1 | 1 | 1 | 1 |
| 3107: 97 | 375 | 1 | 1 | 1 | 1 | 1 | 1 | 1 | 1 | 1 | 1 | 1 | 1 |
| 3107: 100 | 378 | 1 | 0.95 | 0.96 | 0.99 | 0.85 | 0.93 | 1 | 0.93 | 1 | 1 | 0.94 | 1 |
| 3107: 120 | 398 | 1 | 0.84 | 0.78 | 0.87 | 0.84 | 0.88 | 0.72 | 0.89 | 1 | 0.91 | 0.88 | 0.95 |
| 3107: 137 | 415 | 1 | 0.9 | 0.85 | 0.98 | 0.96 | 0.89 | 0.93 | 0.92 | 0.94 | 1 | 0.95 | 0.98 |
| 3107: 139 | 417 | 1 | 0.93 | 0.88 | 0.99 | 0.97 | 0.98 | 0.98 | 0.95 | 0.98 | 0.93 | 0.95 | 0.98 |
| 3107: 148 | 426 | 1 | 0.88 | 0.88 | 0.94 | 0.86 | 0.94 | 0.91 | 0.92 | 1 | 1 | 0.85 | 0.92 |
| 3107: 164 | 442 | 1 | 0.88 | 0.96 | 0.87 | 0.67 | 0.93 | 0.54 | 0.96 | 1 | 1 | 0.85 | 0.89 |
| 3107: 187 | 465 | 0.94 | 0.8 | 0.79 | 0.72 | 0.64 | 0.75 | 0.72 | 0.91 | 0.96 | 0.78 | 0.89 | 0.93 |
| 3107: 190 | 468 | 0.93 | 0.58 | 0.63 | 0.56 | 0.45 | 0.8 | 0.46 | 0.79 | 0.93 | 0.68 | 0.66 | 0.61 |
| 3107: 209 | 487 | 0.88 | 0.61 | 0.7 | 0.41 | 0.3 | 0.66 | 0.42 | 0.73 | 1 | 0.77 | 0.74 | 0.78 |
| 3107: 224 | 502 | 0.93 | 0.86 | 0.78 | 0.73 | 0.9 | 0.94 | 0.83 | 0.95 | 0.95 | 0.96 | 0.93 | 1 |
| 3107: 233 | 511 | 0.81 | 0.49 | 0.76 | 0.52 | 0.52 | 0.7 | 0.5 | 0.77 | 0.83 | 0.84 | 0.8 | 0.85 |
| 3107: 243 | 521 | 0.87 | 0.7 | 0.78 | 0.75 | 0.56 | 0.8 | 0.71 | 0.81 | 0.96 | 0.88 | 0.91 | 0.84 |
| 3107: 257 | 535 | 0.94 | 0.62 | 0.91 | 0.61 | 0.53 | 0.64 | 0.28 | 0.7 | 0.97 | 0.79 | 0.83 | 0.86 |
| 3107: 265 | 543 | 0.92 | 0.66 | 0.69 | 0.8 | 0.53 | 0.89 | 0.3 | 0.97 | 0.82 | 0.85 | 0.87 | 0.88 |
| 3107: 400 | 678 | 1 | 0.88 | 0.93 | 1 | 0.77 | 1 | 1 | 0.93 | NA | 1 | 1 | 1 |

TABLE 12
(3110):

| | MVP | | | | | | | | | | | | |
|---|---|---|---|---|---|---|---|---|---|---|---|---|---|
| CpG | Position in | | | | | | | | | | | | |
| identifier | ROI | Prostate | Prostate | Prostate | Prostate | Prostate | Muscle | Muscle | Muscle | Muscle | Muscle | Lung | Lung |
| 3110: 32 | 1933 | 0.82 | NA | 0.86 | 0.72 | 0.9 | 0.66 | 0.73 | 0.94 | 0.73 | 0.9 | 0.83 | 1 |
| 3110: 84 | 1985 | 0.83 | NA | 1 | 0.7 | 1 | 0.12 | 0.34 | 0 | 0.2 | 0.38 | 0.86 | 1 |
| 3110: 286 | 2187 | 1 | NA | 1 | 1 | 1 | 0.95 | 0.71 | 0.9 | 0.97 | 0.79 | 1 | 1 |
| 3110: 310 | 2211 | 1 | NA | 1 | 0.87 | 1 | 0.28 | 0.43 | 0.43 | 0.59 | 0.6 | 0.9 | 0.97 |
| 3110: 366 | 2267 | 1 | 1 | 0 | 0.84 | 1 | 0.74 | 0.68 | 0.91 | 0.86 | 0.97 | 1 | 1 |
| 3110: 370 | 2271 | 1 | 0.68 | 1 | 0.92 | 1 | 0.67 | 0.69 | 0.93 | 0.88 | 1 | 1 | 1 |
| 3110: 415 | 2316 | 1 | 0.53 | 1 | 0.79 | 1 | 0.61 | 0.55 | 1 | 0.79 | 1 | 1 | 1 |

| | MVP | | | | | | | | | | | | | | |
|---|---|---|---|---|---|---|---|---|---|---|---|---|---|---|---|
| CpG | Position in | | | | | | | | | | | | | | |
| identifier | ROI | Lung | Lung | Liver | Liver | Breast | Breast | Breast | Breast | Breast | Brain | Brain | Brain | Brain | Brain |
| 3110: 32 | 1933 | 0.84 | 0.86 | 1 | 0.88 | 0.87 | 1 | 0.87 | 0.83 | 0.89 | 0.55 | 0.63 | 1 | 0.52 | 0.51 |
| 3110: 84 | 1985 | 1 | 1 | 1 | 0.86 | 1 | 0.8 | 0.75 | 0.86 | 0.5 | 0.51 | 0.17 | 1 | 0.23 | 0.4 |
| 3110: 286 | 2187 | 1 | 1 | 1 | 1 | 0.98 | 0.81 | 1 | 1 | 1 | 0.78 | 0.84 | 1 | 0.7 | 0.88 |
| 3110: 310 | 2211 | 0.91 | 0.95 | 1 | 0.94 | 0.61 | 0 | 0.63 | 0.69 | 0.76 | 0.54 | 0.7 | 1 | 0.54 | 0.7 |
| 3110: 366 | 2267 | 0.93 | 1 | 1 | 1 | 0.78 | 0.35 | 0.84 | 0.98 | 0.84 | 0.87 | 0.84 | 1 | 0.71 | 0.86 |
| 3110: 370 | 2271 | 0.92 | 1 | 1 | 1 | 0.79 | 0.61 | 0.91 | 1 | 0.95 | 0.89 | 0.85 | 1 | 0.71 | 0.93 |
| 3110: 415 | 2316 | 0.93 | 0.89 | 0.68 | 1 | 0.6 | 0.27 | 0.65 | 1 | 0.66 | 0.88 | 0.65 | 1 | 0.67 | 0.79 |

| | MVP | |
|---|---|---|
| CpG | Position in | |
| identifier | ROI | Brain |
| 3110: 32 | 1933 | 0.69 |
| 3110: 84 | 1985 | 0.83 |
| 3110: 286 | 2187 | 0.91 |
| 3110: 310 | 2211 | 0.8 |
| 3110: 366 | 2267 | 0.87 |
| 3110: 370 | 2271 | 1 |
| 3110: 415 | 2316 | 1 |

TABLE 13
(3113):

| | MVP | | | | | | | | | | | | |
|---|---|---|---|---|---|---|---|---|---|---|---|---|---|
| CpG | Position in | | | | | | | | | | | | |
| identifier | ROI | Prostate | Prostate | Prostate | Prostate | Prostate | Prostate | Muscle | Muscle | Muscle | Muscle | Muscle | Lung |
| 3113: 42 | 61 | 0.7 | 1 | NA | 0.82 | 1 | NA | 0.42 | 0.55 | 0 | 0.23 | 0.79 | 0.35 |
| 3113: 47 | 66 | NA | NA | NA | NA | 1 | NA | 1 | 0.5 | 0 | 0.8 | 0.65 | 1 |
| 3113: 72 | 91 | 0.89 | NA | 0 | 0.78 | 1 | NA | 0.37 | 0.076 | 0.59 | 0.3 | 0.26 | 0.85 |
| 3113: 78 | 97 | 0.47 | 1 | 0 | 0.75 | 1 | NA | 0.5 | 0.51 | 0.6 | 0.36 | 0.033 | 0.77 |
| 3113: 86 | 105 | 0.66 | 1 | 0 | 0.83 | 1 | NA | 0.5 | 0.4 | 0 | 0 | 0 | 0.79 |
| 3113: 116 | 135 | 0.63 | 1 | 0 | 0.6 | 1 | 0.081 | 0.4 | 0.69 | 0.24 | 0.31 | 0.48 | 0.59 |
| 3113: 156 | 175 | 0.96 | 0.96 | 0.18 | 0.73 | 1 | 0.36 | 0.5 | 0.56 | 0.067 | 0.46 | 0.12 | 1 |
| 3113: 160 | 179 | 0.65 | 0.58 | 0 | 1 | 1 | NA | 0.41 | 0.61 | 0.64 | 0.59 | 1 | 0.95 |
| 3113: 164 | 183 | 0.79 | 0.78 | 0 | 0.5 | 0 | 0 | 0.49 | 0.38 | 0.082 | 0.27 | 0.12 | 1 |
| 3113: 182 | 201 | 0.76 | 1 | 0 | 0.68 | 1 | NA | 0.24 | 0.56 | 0.34 | 0.47 | 0.54 | 0.91 |
| 3113: 189 | 208 | 1 | 1 | 0.086 | 0.92 | 1 | NA | 0.8 | 0.82 | 0 | 0.85 | 0.64 | 1 |
| 3113: 197 | 216 | 1 | 1 | 0 | 0.88 | 1 | NA | 0.84 | 0.8 | 0.32 | 0.74 | 0.83 | 1 |
| 3113: 298 | 317 | NA | NA | NA | 0 | NA | NA | NA | NA | NA | NA | NA | NA |
| 3113: 303 | 322 | 0.57 | 0.037 | 0.78 | 0.82 | 1 | NA | 0.76 | 0.68 | 0.73 | 0.85 | 0.88 | 0.95 |
| 3113: 378 | 397 | 0.35 | 0.37 | 0 | 0 | 1 | NA | 0.28 | 0.1 | 0.14 | 0 | 0 | 0.28 |
| 3113: 400 | 419 | 1 | 0.81 | 0.25 | 1 | 1 | NA | 0.73 | 0.84 | 0.18 | 0.95 | 0.62 | 0.75 |
| 3113: 406 | 425 | 0.92 | 1 | 1 | 0.94 | 1 | NA | 0.95 | 0.68 | 1 | 0.79 | 0.93 | 0.99 |

| | MVP | | | | | | | | | | | | | |
|---|---|---|---|---|---|---|---|---|---|---|---|---|---|---|
| CpG | Position in | | | | | | | | | | | | | |
| identifier | ROI | Lung | Lung | Lung | Lung | Liver | Liver | Breast | Breast | Breast | Breast | Breast | Brain | Brain |
| 3113: 42 | 61 | 1 | 0 | 1 | 1 | NA | 0.83 | 0 | NA | 0.77 | 0.54 | 0.89 | 0.92 | 1 |
| 3113: 47 | 66 | 1 | 1 | 1 | 0.82 | NA | 1 | NA | NA | 1 | 1 | 0.37 | 0.8 | 0.78 |
| 3113: 72 | 91 | 1 | NA | 1 | 1 | 0 | 0.33 | 0.55 | 0 | 0.58 | 0.21 | 0.58 | 0.67 | 0.62 |
| 3113: 78 | 97 | 1 | NA | 1 | 1 | 0.31 | 0.37 | 0.55 | 0 | 0.5 | 0.6 | 0.4 | 0.92 | 1 |
| 3113: 86 | 105 | 0.82 | 0.85 | 1 | 0.93 | 0.56 | 0.75 | 0.23 | 0 | 0.21 | 0.7 | 0.17 | 0.98 | 1 |
| 3113: 116 | 135 | 0.91 | 1 | 0.79 | 1 | 0.45 | 0.59 | 0.31 | 0.41 | 0.31 | 0.53 | 0.43 | 0.8 | 1 |
| 3113: 156 | 175 | 1 | 0.52 | 1 | 1 | 0 | 0.63 | 0.56 | 1 | 0.82 | 0.74 | 0.22 | 1 | 1 |
| 3113: 160 | 179 | 1 | 0.66 | 1 | 0.78 | 0.37 | 0.75 | 0.62 | 0.29 | 0.65 | 0.63 | 0.6 | 0.9 | 0.87 |
| 3113: 164 | 183 | 0.91 | 1 | 1 | 0.84 | NA | 0.31 | 0.43 | 0.75 | 0.29 | 0.72 | 0.39 | 1 | 0.75 |
| 3113: 182 | 201 | 1 | 0.44 | 1 | 1 | 0 | 0.43 | 0.47 | 0.78 | 0.53 | 0.63 | 0.26 | 1 | 0.9 |
| 3113: 189 | 208 | 1 | 1 | 1 | 1 | 1 | 0.87 | 0.57 | 1 | 0.76 | 0.8 | 0.52 | 1 | 1 |
| 3113: 197 | 216 | 1 | 0.31 | 1 | 1 | NA | 0.88 | 0.37 | 1 | 0.61 | 0.7 | 0.41 | 1 | 1 |
| 3113: 298 | 317 | NA | NA | NA | NA | 1 | 0 | NA | 0.46 | NA | NA | NA | 0.57 | NA |
| 3113: 303 | 322 | 0.97 | 0.84 | 0.91 | 1 | 1 | 0.93 | 0.56 | 0.56 | 0.75 | 0.59 | 0.62 | 0.92 | 0.95 |
| 3113: 378 | 397 | 0.48 | NA | 0.49 | 0.46 | 0.52 | 0 | 0.15 | 0.21 | 0.39 | 0.065 | 0.45 | 0.4 | 0.56 |
| 3113: 400 | 419 | 1 | NA | 1 | 1 | 1 | 1 | 0.6 | 0.87 | 0.71 | 0.92 | 0.64 | 0.94 | 0.96 |
| 3113: 406 | 425 | 1 | NA | 1 | 1 | 1 | 1 | 0.57 | 0.91 | 0.66 | 0.73 | 0.56 | 0.97 | 1 |

| | MVP | | | | |
|---|---|---|---|---|---|
| CpG | Position in | | | | |
| identifier | ROI | Brain | Brain | Brain | Brain |
| 3113: 42 | 61 | 1 | 0.5 | 0.85 | NA |
| 3113: 47 | 66 | 0.74 | 1 | 0.66 | NA |
| 3113: 72 | 91 | 0.87 | 0 | 0.75 | 0 |
| 3113: 78 | 97 | 0.86 | 1 | 0.85 | 1 |
| 3113: 86 | 105 | 0.93 | 1 | 1 | 1 |
| 3113: 116 | 135 | 0.74 | 0.87 | 0.6 | 1 |
| 3113: 156 | 175 | 0.95 | 1 | 1 | 1 |
| 3113: 160 | 179 | 1 | 0.83 | 1 | 1 |
| 3113: 164 | 183 | 0.92 | 0.89 | 0.43 | 1 |
| 3113: 182 | 201 | 0.87 | 1 | NA | 0.71 |
| 3113: 189 | 208 | 1 | 1 | 1 | 1 |
| 3113: 197 | 216 | 1 | 1 | 1 | 0.9 |
| 3113: 298 | 317 | 0.92 | NA | NA | NA |
| 3113: 303 | 322 | 0.89 | 0.91 | 0.76 | 0.94 |
| 3113: 378 | 397 | 0.22 | 0.43 | NA | 0.6 |
| 3113: 400 | 419 | 1 | 1 | 1 | 1 |
| 3113: 406 | 425 | 0.47 | 0.96 | 1 | 0.96 |

TABLE 14
(3127):

| | MVP | | | | | | | | | | | | |
|---|---|---|---|---|---|---|---|---|---|---|---|---|---|
| CpG | Position in | | | | | | | | | | | | |
| identifier | ROI | Prostate | Prostate | Prostate | Prostate | Prostate | Prostate | Prostate | Prostate | Muscle | Muscle | Muscle | Muscle |
| 3127: 25 | 1756 | NA | 0.93 | 0.86 | 1 | 0.61 | 0.44 | 0.57 | 1 | 0.88 | 0.78 | 0.82 | 0.93 |
| 3127: 28 | 1759 | 1 | 0.92 | 0.87 | 1 | NA | NA | 0.73 | 0.84 | 0.89 | 0.85 | 0.95 | 0.95 |
| 3127: 63 | 1794 | 0.8 | 0.62 | 0.77 | 0.7 | 0.17 | 0.57 | 0.47 | 0.46 | 0.79 | 0.53 | 0.54 | 0.71 |
| 3127: 73 | 1804 | 0.96 | 0.84 | 0.86 | 1 | 0.72 | 0.72 | 0.62 | 0.98 | 0.87 | 0.78 | 0.85 | 0.95 |
| 3127: 124 | 1855 | 0.94 | 1 | 0.86 | 0.79 | 0.79 | 0.63 | 0.6 | 0.88 | 0.74 | 0.83 | 0.77 | 0.86 |
| 3127: 127 | 1858 | 0.8 | 0.77 | 0.6 | 0.88 | 0.41 | 0.58 | 0.44 | 0.71 | 0.66 | 0.64 | 0.75 | 0.69 |
| 3127: 175 | 1906 | 0.65 | NA | 0.67 | 0.68 | NA | 0.5 | 0.34 | 0.85 | 0.81 | 0.69 | 0.72 | 0.83 |

| | MVP | | | | | | | | | | | | | | |
|---|---|---|---|---|---|---|---|---|---|---|---|---|---|---|---|
| CpG | Position in | Mus- | | | | | | | | | | | | | |
| identifier | ROI | cle | Lung | Lung | Lung | Lung | Lung | Liver | Liver | Breast | Breast | Breast | Breast | Breast | Breast |
| 3127: 25 | 1756 | 0.86 | 0.73 | 0.89 | 1 | 0.87 | NA | 0.76 | NA | 0.51 | 0.46 | 0.38 | 0.79 | 0.52 | 0.38 |
| 3127: 28 | 1759 | 0.82 | 0.86 | 0.91 | 1 | 0.87 | NA | 0.76 | NA | 0.71 | 0.7 | 0.61 | 1 | 0.65 | 0.63 |
| 3127: 63 | 1794 | 0.58 | 0.73 | 0.83 | 0.61 | 0.79 | 1 | 0.38 | 0.85 | 0.61 | 0.52 | 0.34 | 0.45 | 0.28 | 0.32 |
| 3127: 73 | 1804 | 0.86 | 0.89 | 0.96 | 0.56 | 0.94 | NA | 0.74 | 1 | 0.72 | 0.48 | 0.68 | 0.76 | 0.34 | 0.42 |
| 3127: 124 | 1855 | 0.8 | 0.8 | 0.99 | 0.67 | 0.68 | 0.68 | 0.71 | 0.59 | 0.57 | 0.61 | 0.63 | 0.71 | 0.6 | 0.67 |
| 3127: 127 | 1858 | 0.54 | 0.7 | 0.82 | 0.18 | 0.68 | 0.72 | 0.51 | 0.54 | 0.44 | 0.36 | 0.41 | 0.5 | 0.5 | 0.2 |
| 3127: 175 | 1906 | 0.81 | 0.69 | 0.94 | 0.54 | 0.65 | 0.66 | 0.59 | 0.53 | 0.42 | 0.38 | 0.49 | 0.78 | 0.58 | NA |

| | MVP | | | | | | |
|---|---|---|---|---|---|---|---|
| CpG | Position in | | | | | | |
| identifier | ROI | Brain | Brain | Brain | Brain | Brain | Brain |
| 3127: 25 | 1756 | 0.97 | 0.97 | 1 | 0.92 | 0.96 | 1 |
| 3127: 28 | 1759 | 0.95 | 0.92 | 1 | 0.89 | 0.91 | 0.99 |
| 3127: 63 | 1794 | 0.75 | 0.85 | 0.69 | 0.75 | 0.62 | 0.86 |
| 3127: 73 | 1804 | 1 | 0.99 | 1 | 0.92 | 0.83 | 0.87 |
| 3127: 124 | 1855 | 0.9 | 0.87 | 0.9 | 0.87 | 0.97 | 0.99 |
| 3127: 127 | 1858 | 0.82 | 0.82 | 0.33 | 0.81 | 0.65 | 0.9 |
| 3127: 175 | 1906 | 0.78 | 0.78 | NA | 0.8 | NA | 0.97 |

TABLE 15
(3129):

| | MVP | | | | | | | | | | | | |
|---|---|---|---|---|---|---|---|---|---|---|---|---|---|
| CpG | Position in | | | | | | | | | | | | |
| identifier | ROI | Prostate | Prostate | Prostate | Prostate | Prostate | Muscle | Muscle | Muscle | Muscle | Muscle | Lung | Lung |
| 3129: 99 | 1999 | 1 | 0.14 | 0.75 | 0.76 | NA | 0.77 | 0.7 | 0.16 | 0.71 | 0.55 | 1 | 0.44 |
| 3129: 111 | 2011 | 1 | 1 | 0.97 | 1 | NA | 1 | 1 | 0.97 | 1 | 1 | 1 | 1 |
| 3129: 125 | 2025 | 1 | 0.95 | 1 | 1 | NA | 1 | 1 | 1 | 1 | 1 | 1 | 1 |
| 3129: 137 | 2037 | 1 | 0.97 | 1 | 1 | NA | 1 | 1 | 1 | 0.95 | 0.95 | 1 | 1 |
| 3129: 139 | 2039 | 0.89 | 0.78 | 0.73 | 1 | NA | 1 | 1 | 1 | 1 | 0.64 | 0.76 | 1 |
| 3129: 144 | 2044 | 1 | 0.98 | 1 | 1 | NA | 0.79 | 0.92 | 1 | 0.92 | 0.57 | 1 | 1 |
| 3129: 148 | 2048 | 1 | 1 | 1 | 1 | NA | 1 | 1 | 1 | 1 | 0.85 | 1 | 1 |
| 3129: 157 | 2057 | 0.75 | 1 | 0.77 | 0.92 | NA | 0.69 | 1 | 0.77 | 1 | 1 | 0.8 | 1 |
| 3129: 162 | 2062 | 1 | 0.93 | 0.85 | 0.52 | NA | 1 | 1 | 0.95 | 0.56 | 0.88 | 1 | 0.84 |
| 3129: 178 | 2078 | 0.92 | 0.84 | 0.85 | 0.91 | NA | 0.83 | 1 | 0.88 | 1 | 0.72 | 0.9 | NA |
| 3129: 184 | 2084 | 0.86 | 0.9 | 0.73 | 0.93 | 1 | 1 | 0.96 | 1 | 0.83 | 0.92 | 1 | 0 |
| 3129: 216 | 2116 | 0.95 | 0.98 | 0.91 | 0.92 | NA | 1 | 1 | 1 | 0.86 | 1 | 0.83 | 1 |
| 3129: 261 | 2161 | 1 | 0.13 | 0.86 | 0.82 | 0.71 | 0.66 | 0.49 | 0.32 | 0.97 | 0.42 | 1 | 0.7 |
| 3129: 341 | 2241 | 0.94 | 1 | 1 | 1 | 1 | 1 | 0.79 | 0.93 | 1 | 1 | 1 | 0.93 |
| 3129: 353 | 2253 | 0.46 | 0.05 | 0.69 | 0.79 | NA | 0.034 | 0.92 | 0.16 | 0.77 | 0.29 | 1 | 0.97 |
| 3129: 357 | 2257 | 1 | 1 | 1 | 0.98 | 1 | 1 | 1 | 1 | 1 | 1 | 1 | 1 |
| 3129: 368 | 2268 | 0.83 | 0.86 | 1 | 0.91 | 0.45 | 0.57 | 0.9 | 0.08 | 0.63 | 0.59 | 1 | 0.96 |
| 3129: 371 | 2271 | 1 | 0.86 | 0.79 | 0.86 | 0.86 | 0.77 | 0.59 | 0.055 | 1 | 1 | 1 | 1 |
| 3129: 377 | 2277 | 1 | 1 | 1 | 1 | 0.77 | 0.92 | 0.82 | 1 | 1 | 0.78 | 1 | 1 |
| 3129: 384 | 2284 | 1 | 0.93 | 0.98 | 0.88 | 0.76 | 0.84 | 0.81 | 1 | 0.55 | 0.31 | 1 | 0.94 |
| 3129: 402 | 2302 | 1 | 1 | 1 | 0.92 | 1 | 0.97 | 1 | 1 | 0.89 | 1 | 1 | 0.91 |
| 3129: 438 | 2338 | NA | 0.77 | 0.57 | 1 | 0 | 1 | 1 | 0.64 | 0.87 | 0.8 | 1 | 1 |
| 3129: 453 | 2353 | 1 | 1 | 0.94 | 1 | NA | 1 | 1 | 0.86 | 1 | 0 | 0.97 | 1 |
| 3129: 475 | 2375 | 0.99 | 1 | 1 | 1 | 1 | 1 | 1 | 0.91 | 1 | 0.85 | 1 | NA |

| | MVP | | | | | | | | | | | | | | |
|---|---|---|---|---|---|---|---|---|---|---|---|---|---|---|---|
| CpG | Position in | | | | | | | | | | | | | | |
| identifier | ROI | Lung | Lung | Liver | Liver | Breast | Breast | Breast | Breast | Breast | Breast | Brain | Brain | Brain | Brain |
| 3129: 99 | 1999 | 0.79 | 1 | 1 | 0.64 | 0.74 | 0.52 | 1 | 0.65 | 1 | 1 | 1 | 0.85 | 0.68 | 0.27 |
| 3129: 111 | 2011 | 1 | 1 | 1 | 1 | 1 | 1 | 1 | 1 | 1 | 1 | 1 | 1 | 1 | 1 |
| 3129: 125 | 2025 | 1 | 1 | 1 | 1 | 1 | 1 | 1 | 1 | 1 | 0.92 | 1 | 1 | 1 | 1 |
| 3129: 137 | 2037 | 1 | 1 | 1 | 1 | 1 | 0.97 | 1 | 0.91 | 1 | 0.95 | 1 | 1 | 1 | 1 |
| 3129: 139 | 2039 | 1 | 0.95 | 1 | 1 | 0.8 | 1 | 1 | 0.82 | 1 | 0.5 | 0.79 | 1 | 0.73 | 1 |
| 3129: 144 | 2044 | 1 | 1 | 1 | 1 | 0.98 | 1 | 1 | 0.93 | 0.92 | 0.83 | 0.97 | 1 | 1 | 1 |
| 3129: 148 | 2048 | 1 | 1 | 1 | 1 | 1 | 1 | 1 | 1 | 1 | 1 | 1 | 1 | 1 | 1 |
| 3129: 157 | 2057 | 0.72 | 1 | 1 | 1 | 0.8 | 0.79 | 1 | 0.75 | 0.73 | 1 | 1 | 1 | 0.82 | 1 |
| 3129: 162 | 2062 | 0.87 | 0.65 | 0.89 | 1 | 0.92 | 1 | 1 | 1 | 1 | 0.78 | 1 | 1 | 0.86 | 0.65 |
| 3129: 178 | 2078 | 0.85 | 1 | 1 | 1 | 0.91 | 0.81 | 1 | 0.87 | 1 | 0.82 | 0.85 | 0.88 | 0.87 | 1 |
| 3129: 184 | 2084 | 0.89 | 1 | 1 | 1 | 0.89 | 0.95 | 1 | 0.97 | 0.87 | 1 | 0.87 | 0.86 | 0.9 | 0.97 |
| 3129: 216 | 2116 | 1 | 1 | 0.92 | 1 | 1 | 1 | 1 | 0.98 | 1 | 1 | 1 | 1 | 1 | 1 |
| 3129: 261 | 2161 | 0.89 | 1 | 0.066 | 0.48 | 0.74 | 0.66 | 0.74 | 0.91 | 0.59 | 0.99 | 0.78 | 0.74 | 0.94 | 1 |
| 3129: 341 | 2241 | 1 | 1 | 0.4 | 0.64 | 0.93 | 0.94 | 0.96 | 1 | 1 | 1 | 0.9 | 1 | 0.83 | 0.99 |
| 3129: 353 | 2253 | 1 | 0.96 | 0.27 | 0.36 | 0.63 | 0.58 | 0.61 | 0.9 | 0.67 | 0.93 | 0.78 | 0.8 | 0.86 | 0.94 |
| 3129: 357 | 2257 | 1 | 1 | 0.56 | 0.78 | 1 | 1 | 1 | 1 | 1 | 1 | 1 | 1 | 1 | 1 |
| 3129: 368 | 2268 | 0.9 | 0.98 | 0.064 | 0.34 | 0.68 | 0.6 | 0.53 | 0.57 | 0.54 | 0.82 | 0.86 | 0.97 | 0.82 | 0.95 |
| 3129: 371 | 2271 | 1 | 0.91 | 0.42 | 0.12 | 0.95 | 0.96 | 0.75 | 0.9 | 0.97 | 0.9 | 1 | 0.83 | 0.88 | 0.87 |
| 3129: 377 | 2277 | 1 | 1 | 0.24 | 0.4 | 1 | 1 | 0.92 | 1 | 1 | 0.78 | 0.91 | 1 | 1 | 1 |
| 3129: 384 | 2284 | 0.95 | 0.96 | 0.44 | 0.17 | 0.83 | 0.98 | 0.49 | 0.98 | 0.77 | 1 | 0.96 | 0.85 | 1 | 0.91 |
| 3129: 402 | 2302 | 1 | 0.93 | 0.49 | 0.31 | 0.85 | 0.93 | 0.62 | 0.93 | 0.9 | 1 | 0.97 | 0.97 | 1 | 0.99 |
| 3129: 438 | 2338 | 1 | 0.5 | 0.78 | 0.94 | 1 | 1 | 1 | 0.87 | 1 | 0.95 | 1 | 0.8 | 1 | 1 |
| 3129: 453 | 2353 | 0.93 | 1 | 1 | 1 | 0.83 | 0.98 | 0.93 | 1 | 1 | 0.93 | 0.96 | 0.73 | 1 | 1 |
| 3129: 475 | 2375 | 1 | 1 | 1 | 1 | 1 | 1 | 1 | 0.75 | 1 | 0.91 | 1 | 1 | 1 | 1 |

| | MVP | | |
|---|---|---|---|
| CpG | Position in | | |
| identifier | ROI | Brain | Brain |
| 3129: 99 | 1999 | 1 | 0.7 |
| 3129: 111 | 2011 | 1 | 1 |
| 3129: 125 | 2025 | 1 | 0.98 |
| 3129: 137 | 2037 | 1 | 0.89 |
| 3129: 139 | 2039 | 1 | 1 |
| 3129: 144 | 2044 | 1 | 1 |
| 3129: 148 | 2048 | 1 | 1 |
| 3129: 157 | 2057 | 1 | 0.78 |
| 3129: 162 | 2062 | 1 | 0.98 |
| 3129: 178 | 2078 | 0.84 | 0.64 |
| 3129: 184 | 2084 | 0.87 | 0.98 |
| 3129: 216 | 2116 | 1 | 1 |
| 3129: 261 | 2161 | 0.73 | 0.92 |
| 3129: 341 | 2241 | 1 | 0.92 |
| 3129: 353 | 2253 | 0.86 | 0.91 |
| 3129: 357 | 2257 | 1 | 0.93 |
| 3129: 368 | 2268 | 0.97 | 1 |
| 3129: 371 | 2271 | 0.79 | 0.85 |
| 3129: 377 | 2277 | 0.88 | 0.89 |
| 3129: 384 | 2284 | 0.91 | 1 |
| 3129: 402 | 2302 | 0.96 | 0.92 |
| 3129: 438 | 2338 | 1 | 1 |
| 3129: 453 | 2353 | 0.82 | 1 |
| 3129: 475 | 2375 | 1 | 1 |

TABLE 16
(3145):

| | MVP | | | | | | | | | | | | |
|---|---|---|---|---|---|---|---|---|---|---|---|---|---|
| CpG | Position in | | | | | | | | | | | | |
| identifier | ROI | Prostate | Muscle | Muscle | Muscle | Muscle | Muscle | Lung | Lung | Lung | Lung | Liver | Liver |
| 3145: 46 | 664 | 1 | 1 | 1 | 0.87 | 1 | 1 | 1 | 0.93 | 0.73 | 0.67 | 1 | 1 |
| 3145: 94 | 712 | 0.9 | 0.93 | 1 | 0.38 | 0.84 | 1 | 1 | 0.45 | 0.51 | 0.64 | 1 | 0.91 |
| 3145: 102 | 720 | 0.67 | 1 | 0.82 | 1 | 0.92 | 1 | 1 | 0.57 | 0.45 | 0.57 | 1 | 1 |
| 3145: 110 | 728 | 1 | 0.91 | 1 | 0.95 | 0.95 | 1 | 0.13 | 0.67 | 0.48 | 0.8 | 1 | 1 |
| 3145: 140 | 758 | 0.82 | 0.95 | 0.7 | 1 | 0.86 | 0.95 | 1 | 0.62 | 0.46 | 0.44 | 1 | 0.93 |
| 3145: 158 | 776 | 0.85 | 0.92 | 0.9 | 1 | 0.73 | 0.63 | 0.83 | 0.15 | 0.14 | 0.41 | 1 | 0.77 |
| 3145: 268 | 886 | 1 | 0.9 | 1 | 0.76 | 0.95 | 1 | 0.94 | 0.85 | 0.45 | 0.68 | 1 | 0.89 |
| 3145: 354 | 972 | 0.73 | 0.82 | 0.89 | 0.83 | 0.63 | 0.78 | 0.019 | 0.54 | 0.25 | 0.55 | 0.91 | 0.82 |
| 3145: 388 | 1006 | 1 | 1 | 1 | 1 | 1 | 1 | 1 | 0.73 | 0 | 0.4 | 1 | 1 |
| 3145: 445 | 1063 | 0.84 | 1 | 0.37 | NA | 0.68 | 0.9 | 0.83 | 0.37 | 0.28 | 0.69 | 0.92 | 0.94 |

| | MVP | | | | | | | | | | | |
|---|---|---|---|---|---|---|---|---|---|---|---|---|
| CpG | Position in | | | | | | | | | | | |
| identifier | ROI | Breast | Breast | Breast | Breast | Breast | Breast | Brain | Brain | Brain | Brain | Brain |
| 3145: 46 | 664 | 0.92 | 0.86 | 1 | 1 | 0.65 | 0.91 | 0.97 | 0.5 | 1 | 1 | 0.57 |
| 3145: 94 | 712 | 0.59 | 0.37 | 0.89 | 0.5 | 0.52 | 0.81 | 0.88 | 0.29 | 0.77 | 0.88 | 0.07 |
| 3145: 102 | 720 | 0.48 | 0.3 | 0.71 | 0.84 | 0.5 | 0.82 | 0.79 | 0.2 | 0.58 | 0.78 | 0.46 |
| 3145: 110 | 728 | 0.64 | 0.21 | 0.79 | 0.39 | 0.36 | 0.78 | 0.85 | 0.084 | 0.58 | 0.92 | 0.58 |
| 3145: 140 | 758 | 0.76 | 0.76 | 0.89 | 0.56 | 0.54 | 0.63 | 0.74 | 0.13 | 0.61 | 0.7 | 1 |
| 3145: 158 | 776 | 0.68 | 0.62 | 0.73 | 0.2 | 0.67 | 0.63 | 0.59 | 0 | 0.5 | 0.7 | 0.83 |
| 3145: 268 | 886 | 0.69 | 0.7 | 0.78 | 0.59 | 0.56 | 0.84 | 0.88 | 0.91 | 0.69 | 0.92 | 0.97 |
| 3145: 354 | 972 | 0.51 | 0.73 | 0.56 | 0.59 | 0.45 | 0.74 | 0.7 | 0.18 | 0.51 | 0.69 | NA |
| 3145: 388 | 1006 | 0.00049 | 0.014 | 0.016 | 0 | 0 | 0.42 | 0.0043 | 1 | 0.15 | 0.5 | NA |
| 3145: 445 | 1063 | 0.67 | 0.37 | 0.8 | 0.82 | 0.58 | 0.42 | 0.96 | 1 | 0.55 | 0.87 | NA |

TABLE 17
(3152):

| | MVP | | | | | | | | | | | | |
|---|---|---|---|---|---|---|---|---|---|---|---|---|---|
| CpG | Position in | | | | | | | | | | | | |
| identifier | ROI | Prostate | Prostate | Prostate | Prostate | Prostate | Prostate | Prostate | Prostate | Muscle | Muscle | Muscle | Muscle |
| 3152: 23 | 1818 | 4.3e−05 | 0.099 | 0.18 | 0 | 0 | 0.33 | NA | 0.15 | 0 | 0 | 0.23 | 0.059 |
| 3152: 56 | 1851 | 0.00013 | 0.34 | 0.19 | 0 | 0.026 | 0.56 | NA | 0.23 | 0.17 | 0.24 | 0.46 | 0.16 |
| 3152: 138 | 1933 | 0 | 0.07 | 0.0042 | 0.084 | 0 | 0.041 | NA | 0.087 | 0.25 | 0.18 | 0.087 | 0.05 |
| 3152: 234 | 2029 | 0.072 | 0.58 | 0.4 | 0 | 0 | 0.61 | 0.59 | 0.063 | 0.85 | 0.79 | 0.79 | 0.8 |
| 3152: 283 | 2078 | 0.0092 | 0.65 | 0.44 | 0.11 | 0 | 0.64 | 0.74 | 0 | 0.73 | 1 | 0.83 | 1 |
| 3152: 361 | 2156 | 0.17 | 0.67 | 0.28 | 0.33 | 0 | 0.4 | 1 | 0.84 | 0.67 | 1 | 0.87 | 0.69 |

| | MVP | | | | | | | | |
|---|---|---|---|---|---|---|---|---|---|
| CpG | Position in | | | | | | | | |
| identifier | ROI | Lung | Lung | Breast | Breast | Breast | Brain | Brain | Brain |
| 3152: 23 | 1818 | 0.0087 | 0.32 | NA | 0.76 | 0.31 | 0.34 | 0 | NA |
| 3152: 56 | 1851 | 0.00062 | 0.08 | NA | 0.49 | 0.29 | 1 | 0.35 | NA |
| 3152: 138 | 1933 | 0 | 0.19 | 0.71 | 0.079 | 0.037 | 0.19 | 0.047 | NA |
| 3152: 234 | 2029 | 0.089 | 0.25 | 0.73 | 0.91 | 0.67 | 1 | 0.68 | NA |
| 3152: 283 | 2078 | 0.012 | 0.22 | 0.49 | 0.92 | 0.84 | 1 | 0.77 | NA |
| 3152: 361 | 2156 | 0.69 | 0.19 | 1 | 0.86 | 0.72 | 1 | 0.6 | 1 |

TABLE 18
(3170):

| | MVP | | | | | | | | | | | | |
|---|---|---|---|---|---|---|---|---|---|---|---|---|---|
| CpG | Position in | | | | | | | | | | | | |
| identifier | ROI | Prostate | Prostate | Prostate | Prostate | Prostate | Prostate | Prostate | Muscle | Muscle | Muscle | Muscle | Muscle |
| 3170: 170 | 1858 | 0 | 0.54 | 0.55 | 0.78 | 0.93 | 0 | 0 | 0.4 | 0.37 | 0 | 0.39 | 0.57 |
| 3170: 175 | 1863 | 0 | 0.072 | 0.13 | 0.65 | NA | 0 | 0 | 0 | 0.22 | 0 | 0.022 | 0.093 |
| 3170: 353 | 2041 | 0.87 | 0.64 | 1 | 0.9 | NA | 0.97 | NA | 0.87 | NA | 1 | 0.62 | 0.95 |
| 3170: 385 | 2073 | NA | 0.43 | 0.58 | 0.69 | NA | NA | 1 | 0.51 | NA | NA | 0.34 | 0.61 |
| 3170: 396 | 2084 | NA | 0.67 | 0.7 | 0.86 | NA | NA | NA | 1 | NA | NA | 0.97 | 0.93 |
| 3170: 409 | 2097 | 0.57 | 0.49 | 0.79 | 0.82 | NA | 0.67 | NA | 1 | NA | 1 | 0.91 | 1 |
| 3170: 412 | 2100 | 0.64 | 0.66 | 0.97 | 0.81 | NA | 0.83 | NA | 0.94 | NA | 1 | 0.74 | 0.95 |

| | MVP | | | | | | | | | | | | | | |
|---|---|---|---|---|---|---|---|---|---|---|---|---|---|---|---|
| CpG | Position in | | | | | | | | | | | | | | |
| identifier | ROI | Lung | Lung | Lung | Lung | Lung | Liver | Liver | Breast | Breast | Breast | Breast | Breast | Breast | Brain |
| 3170: 170 | 1858 | 0.84 | 0.42 | 0.51 | 0.22 | 0 | 0.62 | 0.86 | 0.53 | NA | 0 | 0.061 | 0.49 | 0.97 | 1 |
| 3170: 175 | 1863 | 0.15 | 0.052 | 0.23 | 0.013 | 0 | 0.33 | 0.49 | 0.21 | 0 | 0 | 0.12 | 0.29 | 0 | 0.6 |
| 3170: 353 | 2041 | 1 | NA | 0.28 | 0.21 | 0.55 | 0.89 | 0.88 | 0.71 | 1 | 0.87 | 0.87 | 0.78 | 1 | 0.87 |
| 3170: 385 | 2073 | NA | 0 | 0.35 | 0.2 | NA | 0.7 | 0.87 | 0.55 | NA | 1 | NA | 0.51 | NA | 0.69 |
| 3170: 396 | 2084 | NA | 0.023 | 0.37 | 0.36 | NA | 0.91 | 0.97 | 0.86 | NA | NA | NA | 0.74 | NA | 0.88 |
| 3170: 409 | 2097 | 0.88 | 0.32 | 0.36 | 0.16 | 0.41 | 0.67 | 0.88 | 0.81 | 1 | 0.52 | 0.35 | 0.72 | 1 | 0.88 |
| 3170: 412 | 2100 | 1 | 0.42 | 0.2 | 0.22 | 0.42 | 0.68 | 0.74 | 0.67 | 1 | 0.62 | 0.7 | 0.76 | 1 | 0.75 |

| | MVP | | | | | |
|---|---|---|---|---|---|---|
| CpG | Position in | | | | | |
| identifier | ROI | Brain | Brain | Brain | Brain | Brain |
| 3170: 170 | 1858 | 0 | 0 | 1 | 0.81 | 0.82 |
| 3170: 175 | 1863 | 0.013 | 0 | 0.63 | 0.91 | 0.48 |
| 3170: 353 | 2041 | NA | 1 | 1 | 1 | 0.94 |
| 3170: 385 | 2073 | NA | NA | 0.67 | 0.72 | 0.63 |
| 3170: 396 | 2084 | NA | NA | 0.67 | 1 | 1 |
| 3170: 409 | 2097 | NA | 0.54 | 0.95 | 0.83 | 0.93 |
| 3170: 412 | 2100 | NA | 1 | 0.81 | 0.98 | 0.74 |

TABLE 19
(3192):

| | MVP | | | | | | | | | | | | |
|---|---|---|---|---|---|---|---|---|---|---|---|---|---|
| CpG | Position in | | | | | | | | | | | | |
| identifier | ROI | Prostate | Prostate | Prostate | Prostate | Prostate | Prostate | Prostate | Prostate | Muscle | Muscle | Muscle | Muscle |
| 3192: 29 | 375 | 0.13 | 0.49 | 0.19 | 0.12 | 0.2 | 0 | 0.1 | 0 | 0.28 | 0.38 | 0 | 0 |
| 3192: 108 | 454 | 0.49 | 0.47 | 0.41 | 0.35 | 0.38 | 0.5 | 0.32 | 0.15 | 0.47 | 0.38 | 0 | 0.099 |
| 3192: 128 | 474 | 0.48 | 0.35 | 0.37 | 0.3 | 0.33 | 0.33 | 0.34 | 0.18 | 0.52 | 0.082 | 0 | 0.2 |
| 3192: 160 | 506 | 0.59 | 0.52 | 0.49 | 0.37 | 0.38 | 0.45 | 0.33 | 0.32 | 0.58 | 0.14 | 0.27 | 0.15 |
| 3192: 166 | 512 | 0.5 | 0.44 | 0.41 | 0.26 | 0.41 | 0.32 | 0.31 | 0.17 | 0.4 | 0.079 | 0.44 | 0.048 |
| 3192: 172 | 518 | 0.29 | 0.18 | 0.18 | 0.077 | 0.086 | 0.048 | 0.17 | 0.075 | 0.12 | 0.11 | 0.097 | 0 |
| 3192: 191 | 537 | 0.59 | 0.48 | 0.43 | 0.33 | 0.36 | 0.15 | 0.53 | 0.25 | 0.54 | 0.1 | 0.3 | 0.46 |
| 3192: 265 | 611 | 0.54 | 0.54 | 0.49 | 0.37 | 0.49 | 0.44 | 0.43 | 0.31 | 0.85 | 0.76 | 0.69 | 0.31 |
| 3192: 268 | 614 | 0.69 | 0.64 | 0.66 | 0.5 | 0.64 | 0.57 | 0.51 | 0.8 | 0.68 | 0.84 | 0.34 | 0.38 |
| 3192: 362 | 708 | 0.63 | 0.66 | 0.56 | 0.5 | 0.73 | 0.55 | 0.57 | 0.62 | 0.76 | 0.76 | 0.82 | 0.47 |
| 3192: 368 | 714 | 0.64 | 0.64 | 0.58 | 0.69 | 0.66 | 0.64 | 0.52 | 0.44 | 0.74 | 0.44 | 0.34 | 0.52 |
| 3192: 427 | 773 | 0.68 | 0.41 | 0.35 | 0.87 | 0.51 | 0.4 | 0.41 | 0.12 | 0.78 | 0.54 | 0.43 | 0.42 |

| | MVP | | | | | | | | | | | | | | |
|---|---|---|---|---|---|---|---|---|---|---|---|---|---|---|---|
| CpG | Position in | Mus- | | | | | | | | | | | | | |
| identifier | ROI | cle | Lung | Lung | Lung | Lung | Lung | Liver | Liver | Breast | Breast | Breast | Breast | Breast | Breast |
| 3192: 29 | 375 | 0.19 | 1 | 1 | 0.72 | 1 | 0 | NA | NA | 0 | 0.12 | 0.32 | 0.26 | 0 | 0.37 |
| 3192: 108 | 454 | 0.34 | 0.69 | 1 | 1 | 0.87 | 0.63 | 0.62 | 0.29 | 0.43 | 0.51 | 0.32 | 0.57 | 0.13 | 0.58 |
| 3192: 128 | 474 | 0.38 | 0.58 | 1 | 1 | 0.64 | 0.6 | NA | 0.33 | 0.37 | 0.62 | 0.36 | 0.32 | 0.47 | 0.37 |
| 3192: 160 | 506 | 0.47 | 0.64 | 0.81 | 0.54 | 0.73 | 0.69 | 0.41 | 0.26 | 0.38 | 0.34 | 0.34 | 0.43 | 0.49 | 0.55 |
| 3192: 166 | 512 | 0.32 | 0.58 | 0.91 | 0.59 | 0.54 | 0.69 | 0.38 | 0.26 | 0.41 | 0.33 | 0.29 | 0 | 0 | 0.39 |
| 3192: 172 | 518 | 0.064 | 0.53 | 0.68 | 0.44 | 0.45 | 0.35 | 0.38 | 0.1 | 0.17 | 0.22 | 0.11 | 0 | 0 | 0.034 |
| 3192: 191 | 537 | 0.52 | 0.64 | 0.84 | 0.67 | 0.7 | 0.67 | 0.68 | 0.28 | 0.44 | 0.46 | 0.45 | 0.44 | 0.52 | 0.56 |
| 3192: 265 | 611 | 0.67 | 0.77 | 1 | 0.92 | 1 | 0.88 | 1 | 0.5 | 0.54 | 0.76 | 0.6 | 0.72 | 0.67 | 0.59 |
| 3192: 268 | 614 | 0.75 | 0.76 | 0.95 | 0.87 | 0.91 | 0.8 | 1 | 0.42 | 0.64 | 0.84 | 0.7 | 0.78 | 0.59 | 0.81 |
| 3192: 362 | 708 | 0.62 | 0.88 | 0.97 | 1 | 0.94 | 0.8 | 0.83 | 0.78 | 0.67 | 0.63 | 0.72 | 0.84 | 0.54 | 0.76 |
| 3192: 368 | 714 | 0.69 | 0.76 | 0.91 | 0.87 | 0.93 | 0.76 | 0.93 | 0.63 | 0.61 | 0.55 | 0.61 | 0.77 | 0.56 | 0.61 |
| 3192: 427 | 773 | 0.55 | 0.7 | 0.71 | 0.51 | 0.85 | 0.76 | 1 | 0.73 | 0.64 | 0.51 | 0.55 | 0.47 | 0.83 | 0.77 |

| | MVP | | | | | | |
|---|---|---|---|---|---|---|---|
| CpG | Position in | | | | | | |
| identifier | ROI | Brain | Brain | Brain | Brain | Brain | Brain |
| 3192: 29 | 375 | 0.25 | 0 | 0.085 | 0.46 | NA | 0.38 |
| 3192: 108 | 454 | 0.46 | 0.39 | 1 | 1 | 0.36 | 0.5 |
| 3192: 128 | 474 | 0.38 | 0.41 | 0.93 | 0.61 | 0.38 | 0.36 |
| 3192: 160 | 506 | 0.39 | 0.33 | 0.97 | 0.65 | 0.27 | 0.46 |
| 3192: 166 | 512 | 0.3 | 0.35 | 1 | 0.43 | 0.3 | 0.23 |
| 3192: 172 | 518 | 0.13 | 0 | 0 | 0.14 | 0.12 | 0.051 |
| 3192: 191 | 537 | 0.46 | 0.4 | 0.96 | 0.68 | 0.31 | 0.5 |
| 3192: 265 | 611 | 0.56 | 0.56 | 0.96 | 0.94 | 0.63 | 0.57 |
| 3192: 268 | 614 | 0.68 | 0.66 | 1 | 0.86 | 0.57 | 0.62 |
| 3192: 362 | 708 | 0.75 | 0.82 | 0.87 | 1 | 0.74 | 0.62 |
| 3192: 368 | 714 | 0.65 | 0.7 | 0.83 | 1 | 0.61 | 0.75 |
| 3192: 427 | 773 | 0.33 | 0.51 | 1 | 0.67 | 0.47 | 0.51 |

TABLE 20
(3200):

| | MVP | | | | | | | | | | | | |
|---|---|---|---|---|---|---|---|---|---|---|---|---|---|
| CpG | Position in | | | | | | | | | | | | |
| identifier | ROI | Prostate | Prostate | Prostate | Prostate | Prostate | Prostate | Prostate | Prostate | Muscle | Muscle | Muscle | Muscle |
| 3200: 36 | 1897 | 0.46 | 0.48 | 0.39 | 0.35 | 0.47 | 0 | 1 | 0.32 | 0.54 | 0.33 | 0.31 | 0.71 |
| 3200: 49 | 1910 | 0.65 | 0.39 | 0.27 | 0.42 | 0.28 | 0 | 0.48 | 0.43 | 0.93 | 0.91 | 1 | 0.93 |
| 3200: 66 | 1927 | 0.11 | 0.15 | 0.084 | 0.083 | 0.16 | 0 | 0 | 0.078 | 0.28 | 0.14 | 0.25 | 0.23 |
| 3200: 78 | 1939 | 0.057 | 0.46 | 0.36 | 0.48 | 0.6 | 0.7 | 0.53 | 0.26 | 0.51 | 0.6 | 0.68 | 0.75 |
| 3200: 83 | 1944 | 0.11 | 0.25 | 0 | 0.068 | 0.092 | 0.11 | 0.28 | 0.15 | 0.13 | 0.1 | 0.34 | 0.37 |
| 3200: 99 | 1960 | 0.39 | 0.34 | 0.52 | 0.25 | 0.32 | 0.29 | 0.35 | 0.27 | 0.53 | 0.46 | 0.58 | 0.56 |
| 3200: 127 | 1988 | 0.29 | 0.3 | 0.24 | 0.2 | 0.19 | 0.41 | 0.31 | 0.19 | 0.37 | 0.2 | 0.21 | 0.28 |
| 3200: 155 | 2016 | 0.49 | 0.46 | 0.42 | 0.39 | 0.45 | 0.62 | 0.57 | 0.63 | 0.87 | 0.56 | 0.7 | 0.85 |
| 3200: 160 | 2021 | 0.3 | 0.4 | 0.26 | 0.22 | 0.23 | 0.39 | 0.47 | 0.27 | 0.54 | 0.34 | 0.64 | 0.53 |
| 3200: 169 | 2030 | 0.5 | 0.47 | 0.29 | 0.42 | 0.36 | 0.39 | 0.49 | 0.36 | 0.74 | 0.83 | 0.92 | 0.59 |
| 3200: 178 | 2039 | 0.54 | 0.61 | 0.39 | 0.29 | 0.32 | 0.44 | 0.55 | 0.4 | 0.54 | 0.54 | 0.41 | 0.64 |
| 3200: 192 | 2053 | 0.74 | 0.92 | 0.64 | 0.49 | 0.71 | 0.84 | 0.86 | 0.7 | 1 | 1 | 0.97 | 0.88 |
| 3200: 199 | 2060 | 0.3 | 0.44 | 0.37 | 0.23 | 0.42 | 0.42 | 0.5 | 0.18 | 0.36 | 0.13 | 0.61 | 0.51 |
| 3200: 225 | 2086 | 0.45 | 0.68 | 0.39 | 0.48 | 0.55 | 0.48 | 0.66 | 0.59 | 0.78 | 0.56 | 0.66 | 0.71 |
| 3200: 305 | 2166 | 0.53 | 0.5 | 0.3 | 0.3 | 0.45 | 0.63 | 0.74 | 0.39 | 0.47 | 0.51 | 0.41 | 0.52 |
| 3200: 312 | 2173 | 0.44 | 0.53 | 0.24 | 0.36 | 0.53 | 0.38 | 0.41 | 0.16 | 0.51 | 0.4 | 0.6 | 0.58 |
| 3200: 361 | 2222 | 0.6 | 0.96 | 0.79 | 0.41 | 0.52 | 0.64 | 0.83 | 0.73 | 0.92 | 0.94 | 0.67 | 0.52 |

| | MVP | | | | | | | | | | | | | | |
|---|---|---|---|---|---|---|---|---|---|---|---|---|---|---|---|
| CpG | Position in | Mus- | | | | | | | | | | | | | |
| identifier | ROI | cle | Lung | Lung | Lung | Lung | Lung | Liver | Liver | Breast | Breast | Breast | Breast | Breast | Breast |
| 3200: 36 | 1897 | 1 | 0.39 | 0.33 | 0.63 | 0.77 | 0.26 | 1 | 0.91 | 0.68 | 0.84 | 0.37 | 0.51 | 0.67 | 0.44 |
| 3200: 49 | 1910 | 0.76 | 0.45 | 0.46 | NA | 0.52 | 0.29 | 0.87 | 0.81 | 0.71 | 0.86 | 0.49 | 0.62 | 0.9 | 0.57 |
| 3200: 66 | 1927 | 0.2 | 0.17 | 0.26 | NA | 0.35 | 0.12 | 0.91 | 0.55 | 0.18 | 0.34 | 0.093 | 0.37 | 0.064 | 0.19 |
| 3200: 78 | 1939 | 0.45 | 0.52 | 0.46 | 0.46 | 0.54 | 0.49 | 0.96 | 0.83 | 0.79 | 0.5 | 0.41 | 0.77 | 0.62 | 0.51 |
| 3200: 83 | 1944 | 0.18 | 0.2 | 0.35 | 0.25 | 0.26 | 0.12 | 0.56 | 0.75 | 0.49 | 0.35 | 0.22 | 0.56 | 0.082 | 0.082 |
| 3200: 99 | 1960 | 0.52 | 0.24 | 0.3 | 0.44 | 0.45 | NA | 0.96 | 0.39 | 0.2 | 0.48 | 0.5 | 0.66 | 0.43 | 0.29 |
| 3200: 127 | 1988 | 0.22 | 0.32 | 0.69 | 0.12 | 0.39 | 0.29 | 0.93 | 0.67 | 0.44 | 0.28 | 0.37 | 0.34 | 0.31 | 0.41 |
| 3200: 155 | 2016 | 0.62 | 0.43 | 0.75 | 0.71 | 0.65 | 0.35 | 0.85 | 0.65 | 0.58 | 0.74 | 0.86 | 0.79 | 0.86 | 0.55 |
| 3200: 160 | 2021 | 0.35 | 0.15 | 0.62 | 0.28 | 0.5 | 0.51 | 1 | 0.86 | 0.53 | 0.36 | 0.47 | 0.79 | 0.89 | 0.39 |
| 3200: 169 | 2030 | 0.5 | 0.27 | 0.59 | 0.57 | 0.58 | 0.65 | 1 | 0.84 | 0.66 | 0.51 | 0.54 | 0.81 | 0.66 | 0.36 |
| 3200: 178 | 2039 | 0.53 | 0.3 | 0.61 | 0.28 | 0.46 | 0.49 | 0.9 | 0.91 | 0.66 | 0.57 | 0.65 | 0.71 | 0.41 | 0.38 |
| 3200: 192 | 2053 | 0.94 | 0.51 | 0.84 | 0.82 | 0.78 | 0.61 | 1 | 0.91 | 0.97 | 0.87 | 0.82 | 0.89 | 1 | 0.77 |
| 3200: 199 | 2060 | 0.36 | 0.45 | 0.44 | 0.34 | 0.47 | 0.28 | 0.88 | 0.83 | 0.65 | 0.86 | 0.71 | 0.77 | 0.76 | 0.33 |
| 3200: 225 | 2086 | 0.8 | 0.42 | 0.65 | 0.53 | 0.63 | 0.38 | 1 | 0.87 | 0.78 | 0.47 | 0.7 | 0.95 | 0.84 | 0.66 |
| 3200: 305 | 2166 | 0.31 | 0.45 | 0.8 | 0.63 | 0.5 | 0.63 | 1 | 1 | 0.7 | 0.55 | 0.29 | 0.43 | 0.46 | 0.3 |
| 3200: 312 | 2173 | 0.38 | 0.42 | 0.65 | 0.14 | 0.7 | 0.36 | 1 | 0.93 | 0.55 | 0.44 | 0.49 | 0.3 | 0.71 | 0.29 |
| 3200: 361 | 2222 | 0.4 | 0.5 | 0.61 | 0.64 | 0.59 | 0.69 | 1 | 0.91 | 1 | 0.73 | 0.63 | 0.9 | 0.79 | 0.85 |

| | MVP | | | | | |
|---|---|---|---|---|---|---|
| CpG | Position in | | | | | |
| identifier | ROI | Brain | Brain | Brain | Brain | Brain |
| 3200: 36 | 1897 | 1 | 0.36 | 0.45 | 0.63 | 0.23 |
| 3200: 49 | 1910 | 0.54 | 0.72 | 0.69 | 0.62 | 0.42 |
| 3200: 66 | 1927 | 0.28 | 0.37 | 0.58 | 0.23 | 0.18 |
| 3200: 78 | 1939 | 0.66 | 0.5 | 0.6 | 0.57 | 0.35 |
| 3200: 83 | 1944 | 0.2 | 0.42 | 0.45 | 0.088 | 0.2 |
| 3200: 99 | 1960 | 0.66 | 0.5 | 0.69 | 0.4 | 0.51 |
| 3200: 127 | 1988 | 0.35 | 0.36 | 0.48 | 0.41 | 0.53 |
| 3200: 155 | 2016 | 0.67 | 0.73 | NA | 0.56 | 0.41 |
| 3200: 160 | 2021 | 0.59 | 0.55 | 0.57 | 0.44 | 0.52 |
| 3200: 169 | 2030 | 0.81 | 0.64 | 0.63 | 0.68 | 0.57 |
| 3200: 178 | 2039 | 0.77 | 0.53 | 0.57 | 0.42 | 0.46 |
| 3200: 192 | 2053 | 0.91 | 1 | 0.95 | 0.85 | 0.89 |
| 3200: 199 | 2060 | 0.49 | 0.37 | 0.53 | 0.31 | 0.43 |
| 3200: 225 | 2086 | 0.86 | 0.73 | 0.82 | 0.66 | 0.66 |
| 3200: 305 | 2166 | 0.75 | 0.46 | 0.56 | 0.53 | 0.53 |
| 3200: 312 | 2173 | 0.73 | 0.4 | 0.39 | 0.45 | 0.53 |
| 3200: 361 | 2222 | 0.48 | 0.91 | 0.94 | 0.45 | 0.75 |

TABLE 21
(3208):

| | MVP Position in | | | | | | | | | | | | |
|---|---|---|---|---|---|---|---|---|---|---|---|---|---|
| CpG identifier | ROI | Prostate | Prostate | Prostate | Prostate | Prostate | Prostate | Prostate | Prostate | Muscle | Muscle | Muscle | Muscle |
| 3208: 33 | 729 | 0 | NA | 0 | 0 | 0.066 | 0.42 | 0.25 | 0 | 0 | 0.05 | 0 | NA |
| 3208: 45 | 741 | 0.5 | 0.81 | 0.5 | 0.67 | 0.58 | 0 | 0.39 | NA | 0.34 | 0.2 | 0 | 0.52 |
| 3208: 69 | 765 | 0.51 | 0.77 | 0.6 | 0.53 | 0.52 | 1 | 0.36 | 0.37 | 0.58 | 0.15 | 0.43 | NA |
| 3208: 111 | 807 | 0.51 | 0.7 | 0.42 | 0.33 | 0.64 | NA | 0.4 | 0.42 | 0.16 | 0.098 | 0.27 | NA |
| 3208: 119 | 815 | 0.54 | 0.8 | 0.65 | 0.23 | 0.56 | 0.54 | 0.61 | 0.37 | 0.55 | 0.16 | 0.4 | 0.6 |
| 3208: 127 | 823 | 0.29 | 0.81 | 0.54 | 0.44 | 0.45 | 0.71 | 0.46 | 0.3 | 0.3 | 0.15 | 0 | 0.19 |
| 3208: 148 | 844 | 0.69 | 0.86 | 0.66 | 0.59 | 0.64 | 0.76 | 0.64 | 0.59 | 1 | 0.28 | 0.52 | 0.87 |
| 3208: 164 | 860 | 0.57 | 0.86 | 0.52 | 0.7 | 0.62 | 0.55 | 0.63 | 0.5 | 0.58 | 0.71 | 0 | 0.5 |
| 3208: 303 | 999 | 0.69 | 0.93 | 0.8 | 0.83 | 0.35 | 0.5 | 0.81 | 0.34 | 0.26 | 0.18 | 0.71 | 0.56 |
| 3208: 338 | 1034 | 0.83 | 1 | 0.85 | 0.84 | 0.93 | 0.88 | 1 | 0.81 | 0.54 | 0.5 | 0.46 | 0.81 |
| 3208: 349 | 1045 | 0.75 | 0.93 | 0.8 | 0.84 | 0.34 | 0.75 | 0.71 | 0.48 | 0.36 | 0.22 | 0.37 | 0.12 |
| 3208: 371 | 1067 | 1 | 1 | 1 | 0.96 | 1 | 1 | 1 | 1 | 0.97 | 1 | 0.87 | 0.98 |
| 3208: 392 | 1088 | 1 | 1 | 1 | 1 | 1 | 1 | 1 | 1 | 0.84 | 0.78 | 0.72 | 0.54 |
| 3208: 403 | 1099 | 1 | 1 | 1 | 1 | 1 | 1 | 1 | 1 | 1 | 1 | 1 | 1 |
| 3208: 436 | 1132 | 1 | 0.88 | 1 | 0.97 | 0.5 | 1 | 0.7 | NA | 1 | 1 | 1 | 1 |
| 3208: 455 | 1151 | NA | 1 | NA | 1 | NA | 1 | NA | 1 | 1 | 1 | 1 | NA |
| 3208: 461 | 1157 | NA | 1 | 1 | 1 | 0 | 1 | NA | 1 | 0.73 | 1 | 1 | NA |

| | MVP | | | | | | | | | | | | | | |
|---|---|---|---|---|---|---|---|---|---|---|---|---|---|---|---|
| CpG identifier | Position in ROI | Muscle | Lung | Lung | Lung | Lung | Lung | Liver | Liver | Breast | Breast | Breast | Breast | Breast | Breast |
| 3208: 33 | 729 | 0 | 0.36 | 0.64 | 0.011 | 0.32 | 0 | 0.7 | 0.64 | NA | 0 | 0 | 0.91 | 0.079 | 0.15 |
| 3208: 45 | 741 | 0.56 | 0.62 | 0.86 | 0.53 | 0.76 | 0.55 | 1 | 1 | 0.59 | 0.15 | 0.63 | 0.96 | 0.5 | 0.67 |
| 3208: 69 | 765 | 0.4 | 0.66 | 0.95 | 0.49 | 0.49 | 0.27 | 1 | 1 | 0.6 | 0.47 | 0.34 | 1 | 0.36 | 0.63 |
| 3208: 111 | 807 | 0 | 0.16 | NA | 0.074 | 1 | 0.62 | 0.63 | 1 | 0.65 | 0.14 | 0.35 | 1 | 0.49 | 0.47 |
| 3208: 119 | 815 | 0.62 | 0.27 | 0.59 | 0 | 0.5 | 0.34 | 0.89 | 1 | 0.58 | 0.21 | 0.35 | 0.48 | 0.26 | 0.66 |
| 3208: 127 | 823 | 0.3 | 0.55 | 0.71 | 0.13 | 0.66 | 0.44 | 0.89 | 0.85 | 0.47 | 0.23 | 0.6 | 0.63 | 0.43 | 0.7 |
| 3208: 148 | 844 | 0.8 | 0.73 | 0.84 | 0.52 | 0.8 | 0.84 | 0.96 | 1 | 0.78 | 0.53 | 0.7 | 1 | 0.84 | 0.78 |
| 3208: 164 | 860 | 0.48 | 0.5 | 0.58 | 0.51 | 0.82 | 0.29 | 1 | 0.89 | 0.83 | 0.64 | 0.71 | 0.86 | 0.56 | 0.62 |
| 3208: 303 | 999 | 0.52 | 0.81 | 1 | 0.98 | 0.91 | 0.7 | 1 | 1 | 0.75 | 0.85 | 0.69 | 0.94 | 0.59 | 0.76 |
| 3208: 338 | 1034 | 0.5 | 0.84 | NA | 0.33 | 0.9 | 1 | 0.92 | 0.86 | 0.59 | 0.89 | 0.65 | 0.94 | 0.76 | 0.86 |
| 3208: 349 | 1045 | 0.45 | 0.81 | 1 | 0.069 | 0.72 | 0.81 | 1 | 0.84 | 0.62 | 0.33 | 0.45 | 0.95 | 0.71 | 0.77 |
| 3208: 371 | 1067 | 0.88 | 1 | NA | 0.56 | 0.95 | 1 | 1 | 1 | 1 | 1 | 0.94 | 1 | 0.9 | 0.96 |
| 3208: 392 | 1088 | 1 | 0.89 | 1 | 1 | 0.95 | 0.78 | 0.76 | 1 | 1 | 1 | 1 | 1 | 1 | 1 |
| 3208: 403 | 1099 | 1 | 1 | NA | 0.66 | 1 | 1 | 1 | 1 | 1 | 1 | 1 | 1 | 0.94 | 1 |
| 3208: 436 | 1132 | 0.86 | 1 | 1 | 1 | 1 | 0.91 | 1 | 1 | 1 | 1 | 0.92 | 0.87 | 0.88 | 1 |
| 3208: 455 | 1151 | NA | 1 | NA | 1 | NA | 1 | NA | 1 | NA | 1 | NA | 1 | 1 | NA |
| 3208: 461 | 1157 | NA | 1 | NA | 1 | NA | 0.55 | NA | 1 | NA | 0.24 | NA | 1 | 1 | NA |

| CpG identifier | MVP Position in ROI | Brain | Brain | Brain | Brain | Brain | Brain |
|---|---|---|---|---|---|---|---|
| 3208: 33 | 729 | 0.12 | 0.2 | 0.5 | 0.23 | 0.034 | 0 |
| 3208: 45 | 741 | 0.65 | 0.65 | 0.24 | 0.27 | 0 | 0.054 |
| 3208: 69 | 765 | 0.38 | 0.53 | 1 | 0.3 | 0.52 | 1 |
| 3208: 111 | 807 | 0 | 0 | 0.12 | 0.5 | 0.28 | 0.85 |
| 3208: 119 | 815 | 0.66 | 0.71 | 0 | NA | 0.63 | 0.48 |
| 3208: 127 | 823 | 0.49 | 0.43 | 0.16 | 0.48 | 0.62 | 0.67 |
| 3208: 148 | 844 | 0.9 | 0.91 | 0.14 | 0.82 | 0.88 | 0.85 |
| 3208: 164 | 860 | 0.67 | 0.58 | 1 | 0.84 | 0.73 | 0.69 |
| 3208: 303 | 999 | 0.94 | 0.99 | 1 | 0.91 | 0.83 | 0.77 |
| 3208: 338 | 1034 | 0.72 | 0.75 | 0.94 | 1 | 0.8 | 0.71 |
| 3208: 349 | 1045 | 1 | 0.85 | 0.76 | 0.65 | 0.91 | 0.87 |
| 3208: 371 | 1067 | 1 | 1 | 1 | 1 | 1 | 1 |
| 3208: 392 | 1088 | 1 | 1 | 0.94 | 1 | 1 | 1 |
| 3208: 403 | 1099 | 1 | 1 | 0.97 | 1 | 1 | 1 |
| 3208: 436 | 1132 | 1 | 1 | 1 | 1 | 0.7 | 1 |
| 3208: 455 | 1151 | NA | NA | 0 | 1 | NA | NA |
| 3208: 461 | 1157 | NA | NA | 1 | 1 | NA | NA |

TABLE 22
(3239):

| | MVP Position in | | | | | | | | | | | | |
|---|---|---|---|---|---|---|---|---|---|---|---|---|---|
| CpG identifier | ROI | Prostate | Prostate | Prostate | Prostate | Prostate | Prostate | Prostate | Prostate | Muscle | Muscle | Muscle | Muscle |
| 3239: 38 | 623 | 0.9 | 0.76 | 0.82 | 0.73 | 0.73 | 0.96 | 0.76 | 0.89 | 0.91 | 1 | 0.88 | 0.89 |
| 3239: 44 | 629 | 0.99 | 0.98 | 1 | 0.95 | 0.95 | 1 | 0.96 | 0.97 | 1 | 0.43 | 1 | 1 |
| 3239: 49 | 634 | 0.34 | 0.49 | 0.4 | 0.4 | 0.15 | 0.051 | 0.45 | 0.28 | 0.36 | 0.37 | 0.69 | 0.45 |
| 3239: 71 | 656 | 0.59 | 0.59 | 0.65 | 0.56 | 0.59 | 0.54 | 0.51 | 0.55 | 0.59 | 0.46 | 0.55 | 0.62 |
| 3239: 75 | 660 | 0.24 | 0.18 | 0.23 | 0.17 | 0.27 | 0.3 | 0.11 | 0.12 | 0.09 | 0 | 0.27 | 0.28 |
| 3239: 88 | 673 | 0.37 | 0.42 | 0.35 | 0.2 | 0.091 | 0.09 | 0.088 | 0.11 | 0.075 | 0.0093 | 0 | 0.063 |
| 3239: 141 | 726 | 0.43 | 0.49 | 0.41 | 0.29 | 0.42 | 0.63 | 0.24 | 0.33 | 0.39 | 0.099 | 0.23 | 0.47 |
| 3239: 163 | 748 | 0.12 | 0.25 | 0.13 | 0.28 | 0.16 | 0.25 | 0.032 | 0.22 | 0.18 | 0 | 0.23 | 0.14 |
| 3239: 169 | 754 | 0.42 | 0.57 | 0.55 | 0.5 | 0.36 | 0.26 | 0.45 | 0.49 | 0.58 | 0.48 | 0.18 | 0.73 |
| 3239: 178 | 763 | 0.58 | 0.54 | 0.64 | 0.5 | 0.49 | 0.78 | 0.43 | 0.58 | 0.76 | 0.31 | 0.63 | 0.8 |
| 3239: 197 | 782 | 0.63 | 0.61 | 0.3 | 0.4 | 0.44 | 0.85 | 0.26 | 0.52 | 0.67 | 0.2 | 0.24 | 0.73 |
| 3239: 212 | 797 | 0.59 | 0.63 | 0.58 | 0.52 | 0.5 | 0.5 | 0.5 | 0.6 | 0.75 | 0.24 | 0.27 | 0.74 |
| 3239: 218 | 803 | 0.43 | 0.52 | 0.52 | 0.41 | 0.38 | 0.33 | 0.37 | 0.46 | 0.41 | 0.16 | 0 | 0.37 |
| 3239: 233 | 818 | 0.41 | 0.69 | 0.59 | 0.48 | 0.3 | 0.42 | 0.33 | 0.54 | 0.56 | 0.71 | 0.37 | 0.73 |
| 3239: 236 | 821 | 0.46 | 0.42 | 0.39 | 0.39 | 0.22 | 0.44 | 0.24 | 0.44 | 0.36 | 0 | 0.25 | 0.31 |
| 3239: 242 | 827 | 0.41 | 0.41 | 0.35 | 0.27 | 0.2 | 0.41 | 0.12 | 0.36 | 0.49 | 0.08 | 0.43 | 0.57 |
| 3239: 250 | 835 | 0.57 | 0.31 | 0.52 | 0.4 | 0.47 | 0.16 | 0.46 | 0.54 | 0.59 | 0.45 | 0.33 | 0.78 |
| 3239: 256 | 841 | 0.37 | 0.27 | 0.42 | 0.39 | 0.21 | 0.18 | 0.27 | 0.44 | 0.4 | 0 | 0.9 | 0.29 |
| 3239: 262 | 847 | 0.17 | 0 | 0.27 | 0 | 0.075 | 0.015 | 0.064 | 0.11 | 0.19 | 0 | 0 | 0.1 |
| 3239: 285 | 870 | 0.13 | 0 | 0 | 0.27 | 0.052 | 0.0058 | 0.005 | 0.035 | 0.042 | 0 | 0 | 0.0028 |
| 3239: 300 | 885 | 0.1 | 0.25 | 0 | 0.056 | 0 | 0 | 0 | 0.1 | 0.18 | 0.5 | 0.17 | 0.064 |
| 3239: 319 | 904 | 0 | 0 | 0.03 | 0 | 0 | 0.0054 | 0 | 0 | 0 | 0 | 0 | 0 |
| 3239: 328 | 913 | 0.086 | 0.15 | 0.15 | 0 | 0.19 | 0.25 | 0.059 | 0.13 | 0.35 | 0 | 0.019 | 0.46 |
| 3239: 337 | 922 | 0.14 | 0 | 0.12 | 0.21 | 0.18 | 0.19 | 0.14 | 0.24 | 0 | 0 | 0.02 | 0.23 |
| 3239: 340 | 925 | 0 | 0 | 0 | 0 | 0 | 0.17 | 0.064 | 0 | 0 | 0 | 0 | 0 |
| 3239: 343 | 928 | 0 | 0.05 | 0 | 0.067 | 0 | 0.13 | 0.097 | 0 | 0.37 | 0 | 0.033 | 0 |
| 3239: 348 | 933 | 0 | 0.31 | 0.14 | 0.095 | 0 | 0.098 | 0.089 | 0 | 0 | 0.34 | 0.2 | 0 |
| 3239: 354 | 939 | 0.073 | 0 | 0.1 | 0 | 0 | 0 | 0 | 0.08 | 0 | 0 | 0 | 0 |
| 3239: 360 | 945 | 0.17 | 0.62 | 0.11 | 0 | 0.18 | 0.26 | 0.082 | 0.33 | 0.19 | 0.28 | 0.027 | 0.2 |
| 3239: 366 | 951 | 0.27 | 0 | 0.3 | 0.11 | 0.11 | 0.3 | 0.12 | 0.24 | 0.24 | 0 | 0.029 | 0.15 |
| 3239: 377 | 962 | 0 | 0 | 0 | 0 | 0.045 | 0.057 | 0 | 0.14 | 0.35 | 0 | 0 | 0.0039 |
| 3239: 421 | 1006 | 0.54 | 1 | 0.17 | 0 | 0 | 0.5 | 0.26 | 0.06 | 0.065 | 1 | 0.39 | 0.35 |

| | MVP | | | | | | | | | | | | | | |
|---|---|---|---|---|---|---|---|---|---|---|---|---|---|---|---|
| CpG | Position in | | | | | | | | | | | | | | |
| identifier | ROI | Muscle | Lung | Lung | Lung | Lung | Lung | Liver | Liver | Breast | Breast | Breast | Breast | Breast | Breast |
| 3239: 38 | 623 | 0.85 | 0.87 | NA | 1 | 0.86 | 0.89 | 1 | 0.99 | 0.68 | 0.62 | 0.43 | 0.97 | 0.87 | 0.69 |
| 3239: 44 | 629 | 0.97 | 0.96 | 0.87 | 0.5 | 0.98 | 1 | 1 | 1 | 0.69 | 0.7 | 0.75 | 0.95 | 0.54 | 0.72 |
| 3239: 49 | 634 | 0.37 | 0.54 | 0.74 | 0.78 | 0.42 | 0.25 | 0.75 | 0.73 | 0.24 | 0.2 | 0.18 | 1 | 0.66 | 0.31 |
| 3239: 71 | 656 | 0.38 | 0.63 | 0.66 | 0.76 | 0.64 | 0.85 | 1 | 0.76 | 0.45 | 0.17 | 0.4 | 0.78 | 0.17 | 0.28 |
| 3239: 75 | 660 | 0.078 | 0.21 | 0.046 | 0.0054 | 0.16 | 0.17 | 0 | 0.15 | 0.045 | 0.05 | 0.073 | 0 | 0 | 0 |
| 3239: 88 | 673 | 0.12 | 0.26 | 0.45 | 0 | 0.23 | 0.06 | 0 | 0.39 | 0.097 | 0.018 | 0.055 | 0 | 0.39 | 0.22 |
| 3239: 141 | 726 | 0.3 | 0.47 | 0.5 | 0.13 | 0.42 | 0.13 | 0.3 | 0.63 | 0.2 | 0.23 | 0.38 | 0.55 | 0.72 | 0.016 |
| 3239: 163 | 748 | 0.071 | 0.13 | 0.6 | 1 | 0.33 | 0.24 | NA | 0.28 | 0.084 | 0.099 | 0.064 | 0 | 0.24 | 0.13 |
| 3239: 169 | 754 | 0.51 | 0.64 | 0.78 | 1 | 0.62 | 0.78 | 0 | 0.87 | 0.45 | 0.28 | 0.23 | 0.5 | 0.23 | 0.22 |
| 3239: 178 | 763 | 0.65 | 0.65 | 0.86 | 1 | 0.7 | 0.88 | NA | 0.86 | 0.31 | 0.19 | 0.061 | 0.37 | 0.89 | 0.41 |
| 3239: 197 | 782 | 0.5 | 0.8 | 0.94 | 1 | 0.77 | 0.58 | 0.75 | 0.97 | 0.61 | 0.45 | 0.33 | 0.59 | 0.7 | 0.46 |
| 3239: 212 | 797 | 0.57 | 0.74 | 0.92 | 0.93 | 0.75 | 0.62 | NA | 0.96 | 0.42 | 0.39 | 0.19 | 0.17 | 0.73 | 0.46 |
| 3239: 218 | 803 | 0.39 | 0.43 | 0.75 | 0.8 | 0.53 | 0.12 | NA | 0.86 | 0.13 | 0.4 | 0.091 | 0.5 | 0.61 | 0.16 |
| 3239: 233 | 818 | 0.48 | 0.62 | 0.56 | 0.97 | 0.57 | 0.73 | NA | 0.77 | 0.21 | 0.3 | 0.16 | 0.13 | 0.73 | 0.31 |
| 3239: 236 | 821 | 0.34 | 0.46 | 0.6 | 0.89 | 0.46 | 0.75 | NA | 0.57 | 0.1 | 0.0083 | 0 | 0 | 0.35 | 0.075 |
| 3239: 242 | 827 | 0.42 | 0.46 | 0.73 | 0.84 | 0.43 | 0.54 | NA | 0.57 | 0.1 | 0.16 | 0.14 | 0.48 | 0.77 | 0.37 |
| 3239: 250 | 835 | 0.64 | 0.53 | 0.61 | 0.91 | 0.51 | 0.52 | 0 | 0.68 | 0.35 | 0.3 | 0 | 0.56 | 0.27 | 0.38 |
| 3239: 256 | 841 | 0.31 | 0.28 | 0.31 | 0.49 | 0.39 | 0.43 | 1 | 0.25 | 0.28 | 0 | 0 | 0 | 0.0058 | 0.084 |
| 3239: 262 | 847 | 0.37 | 0.12 | 0.00095 | 0.8 | 0.07 | 0.11 | NA | 0.04 | 0.046 | 0 | 0 | 0 | 0 | 0 |
| 3239: 285 | 870 | 0.038 | 0.053 | 0 | 0.7 | 0.25 | 0 | 1 | 0 | 0 | 0 | 0 | 0 | 0 | 0.11 |
| 3239: 300 | 885 | 0.18 | 0.12 | 0.21 | 0 | 0.035 | 0 | NA | 0.19 | 0.34 | 0.14 | 0.061 | 0.21 | 0.31 | 0.25 |
| 3239: 319 | 904 | 0 | 0.037 | 0 | 0 | 0.084 | 0.069 | NA | 0 | 0 | 0 | 0.077 | 0 | 0 | 0 |
| 3239: 328 | 913 | 0.056 | 0 | 0 | 0.84 | 0.23 | 0.26 | NA | 0.3 | 0 | 0 | 0 | 0 | 0 | 0.086 |
| 3239: 337 | 922 | 0 | 0 | 0.34 | 0 | 0.13 | 0.2 | NA | 0.1 | 0 | 0 | 0 | 0 | 0 | 0.1 |
| 3239: 340 | 925 | 0 | 0 | 0 | 0 | 0.12 | 0.29 | NA | 0 | 0 | 0 | 0 | 0 | 0 | 0.067 |
| 3239: 343 | 928 | 0 | 0 | 0.086 | 0 | 0.047 | 0.065 | NA | 0 | 0 | 0 | 0 | 0 | 0 | 0.099 |
| 3239: 348 | 933 | 0 | 0 | 0.17 | 0 | 0.2 | 0.26 | NA | 0.24 | 0.32 | 0.2 | 0.12 | 0.2 | 0.36 | 0.34 |
| 3239: 354 | 939 | 0 | 0 | 0 | 0.27 | 0.24 | 0.37 | NA | 0.0094 | 0 | 0 | 0 | 0 | 0 | 0.22 |
| 3239: 360 | 945 | 0 | 0.32 | 0.066 | 0 | 0.28 | 0.57 | NA | 0.29 | 0 | 0.11 | 0.018 | 0.037 | 0.47 | 0.75 |
| 3239: 366 | 951 | 0 | 0 | 0 | 0.016 | 0.17 | 0.38 | NA | 0 | 0 | 0 | 0 | 0.052 | 0 | 0 |
| 3239: 377 | 962 | 0 | 0 | 0 | 0 | 0.31 | 0 | NA | 0.018 | 0 | 0 | 0 | 0 | 0 | 0 |
| 3239: 421 | 1006 | 0 | 0 | 0 | NA | 0.076 | 0.14 | NA | 0.25 | 0 | 0 | NA | 0.13 | 0.036 | 0.19 |

| CpG identifier | MVP Position in ROI | Brain | Brain | Brain | Brain | Brain |
|---|---|---|---|---|---|---|
| 3239: 38 | 623 | 0.98 | 0.87 | 0.8 | 1 | 0.94 |
| 3239: 44 | 629 | 1 | 1 | 1 | 1 | 0.93 |
| 3239: 49 | 634 | 0.77 | 0.98 | 0.33 | 0.74 | 0.72 |
| 3239: 71 | 656 | 0.86 | 1 | 0.46 | 0.89 | 0.93 |
| 3239: 75 | 660 | 0.25 | 0.25 | 0.16 | 0.28 | 0.27 |
| 3239: 88 | 673 | 0.49 | 0.47 | 0 | 0.45 | 0.32 |
| 3239: 141 | 726 | 0.67 | 1 | 0.36 | 0.66 | 0.85 |
| 3239: 163 | 748 | 0.59 | 0.58 | 0 | 0.53 | 0.45 |
| 3239: 169 | 754 | 0.95 | 0.92 | 0.34 | 0.84 | 0.68 |
| 3239: 178 | 763 | 0.94 | 0.86 | 0.3 | 0.81 | 0.94 |
| 3239: 197 | 782 | 1 | 1 | 0.67 | 0.96 | 1 |
| 3239: 212 | 797 | 0.98 | 1 | 0.55 | 0.97 | 0.98 |
| 3239: 218 | 803 | 0.8 | 1 | 0.088 | 0.78 | 0.65 |
| 3239: 233 | 818 | 0.95 | 1 | 0.45 | 0.92 | 0.92 |
| 3239: 236 | 821 | 0.68 | 0.69 | 0.16 | 0.66 | 0.62 |
| 3239: 242 | 827 | 0.71 | 1 | 0.54 | 0.7 | 0.65 |
| 3239: 250 | 835 | 0.75 | 0.83 | 0.44 | 0.66 | 0.75 |
| 3239: 256 | 841 | 0.54 | 0.8 | 0 | 0.49 | 0.5 |
| 3239: 262 | 847 | 0.37 | 0.17 | 0.048 | 0.48 | 0.37 |
| 3239: 285 | 870 | 0.22 | 0.39 | 0.036 | 0.17 | 0.14 |
| 3239: 300 | 885 | 0.17 | 0.27 | 0.29 | 0.064 | 0 |
| 3239: 319 | 904 | 0.22 | 0.34 | 0 | 0.14 | 0.084 |
| 3239: 328 | 913 | 0.66 | 0.78 | 0.26 | 0.62 | 0.45 |
| 3239: 337 | 922 | 0.15 | 0.2 | 0.19 | 0.12 | 0 |
| 3239: 340 | 925 | 0.2 | 0.55 | 0 | 0.42 | 0.0049 |
| 3239: 343 | 928 | 0.12 | 0.11 | 0.11 | 0.011 | 0 |
| 3239: 348 | 933 | 0.17 | 0.27 | 0.36 | 0.076 | 0.28 |
| 3239: 354 | 939 | 0.4 | 0.56 | 0.32 | 0.33 | 0.082 |
| 3239: 360 | 945 | 0.52 | 0.6 | 0.11 | 0.41 | 0.25 |
| 3239: 366 | 951 | 0.44 | 0.21 | 0 | 0.51 | 0.2 |
| 3239: 377 | 962 | 0.057 | 0.24 | 0 | 0 | 0 |
| 3239: 421 | 1006 | 0.38 | 0.46 | 0.49 | 1 | 1 |

TABLE 23
(3243):

| | MVP Position in | | | | | | | | | | | | |
|---|---|---|---|---|---|---|---|---|---|---|---|---|---|
| CpG identifier | ROI | Prostate | Prostate | Prostate | Prostate | Prostate | Prostate | Prostate | Prostate | Muscle | Muscle | Muscle | Muscle |
| 3243: 57 | 1576 | NA | 1 | NA | NA | NA | 1 | NA | 1 | 1 | NA | NA | 1 |
| 3243: 63 | 1582 | NA | 1 | 1 | 1 | 1 | 1 | NA | NA | NA | 1 | 1 | 1 |
| 3243: 132 | 1651 | 0.72 | 0.47 | 0.81 | 0.73 | 0.75 | 0.84 | 0.75 | 0.65 | 0.64 | 1 | 0.84 | 0.69 |
| 3243: 138 | 1657 | 0.66 | 0.43 | 0.97 | 0.83 | 0.75 | 0.77 | 0.73 | 0.71 | 0.87 | 1 | 0.88 | 0.94 |
| 3243: 140 | 1659 | 0.78 | 0.68 | 1 | 0.71 | 1 | 0.64 | 0.5 | 1 | 0.86 | 1 | 0.92 | 1 |
| 3243: 155 | 1674 | 1 | 0.46 | 0.94 | 1 | 1 | 1 | 0.89 | 0.78 | 1 | 1 | 0.73 | 0.93 |
| 3243: 182 | 1701 | 0.62 | 0.75 | 0.9 | 0.82 | 0.87 | 0.87 | 0.82 | 0.81 | 0.74 | 1 | 1 | 0.76 |
| 3243: 229 | 1748 | 0.36 | 0.26 | 0.54 | 0.63 | NA | 0.9 | 0.3 | 0.55 | NA | 0.58 | 0.51 | 0.64 |
| 3243: 252 | 1771 | 0.39 | 0.25 | 0.3 | 0.29 | 0.47 | 0.82 | 0.45 | 0.19 | 0.18 | 0.16 | NA | 0.41 |
| 3243: 263 | 1782 | 0.56 | 0.29 | 0.41 | 0.24 | 0.54 | 0.71 | 0.7 | 0.27 | 0.21 | 1 | 0.33 | 0.58 |
| 3243: 311 | 1830 | 0.71 | 0.26 | 0.77 | 0.47 | 0.74 | 0.6 | 0.77 | 0.86 | 0.62 | 0 | 0.69 | 0.43 |
| 3243: 392 | 1911 | NA | NA | NA | 0.51 | NA | NA | NA | NA | NA | NA | 1 | 0.84 |

| | MVP | | | | | | | | | | | | | | |
|---|---|---|---|---|---|---|---|---|---|---|---|---|---|---|---|
| CpG identifier | Position in ROI | Muscle | Lung | Lung | Lung | Lung | Lung | Liver | Liver | Breast | Breast | Breast | Breast | Breast | Brain |
| 3243: 57 | 1576 | NA | 1 | 0 | 1 | NA | 1 | NA | 1 | NA | 0.9 | NA | NA | NA | 1 |
| 3243: 63 | 1582 | 1 | 1 | NA | 1 | NA | 1 | NA | NA | NA | 1 | NA | NA | NA | 1 |
| 3243: 132 | 1651 | 0.83 | 0.76 | 0.6 | 0.49 | 0.85 | 0.8 | 0.17 | 0.85 | 0.72 | 0.47 | 0.48 | 0.72 | 0.74 | 0.64 |
| 3243: 138 | 1657 | 1 | 0.89 | 0.5 | 0.39 | 0.66 | 0.69 | 0.24 | 0.7 | 0.54 | 0.69 | 0.48 | 0.52 | 0.44 | 1 |
| 3243: 140 | 1659 | 1 | 1 | 0.41 | 0.47 | 1 | 0.91 | 0 | 1 | 0.76 | 0.66 | 0.51 | 0.68 | 0.26 | 1 |
| 3243: 155 | 1674 | 0.93 | 1 | 0.89 | 0.55 | 0.92 | 0.86 | 0.037 | 0.91 | 0.31 | 0.35 | 0.3 | 0.73 | 0.24 | 1 |
| 3243: 182 | 1701 | 0.87 | 0.81 | 0.69 | 0.6 | 0.75 | 0.94 | 0 | 1 | 0.65 | 0.59 | 0.57 | 0.6 | 0.41 | 0.98 |
| 3243: 229 | 1748 | 0.4 | 0.74 | 0.98 | 0.39 | 0.73 | 0.75 | 0 | 0.68 | 0.27 | 0.3 | 0.24 | 0.25 | 0.38 | 0.88 |
| 3243: 252 | 1771 | 0.49 | 0.62 | 0.15 | 0.27 | 0.64 | 0.66 | 0 | 0.81 | 0.5 | 0.15 | 0.25 | 0 | 0.22 | 0.75 |
| 3243: 263 | 1782 | 0.94 | 0.75 | 0.78 | 0 | 0.53 | 0.8 | NA | 1 | 0.29 | 0.35 | 0.23 | 0 | 0.55 | 0.91 |
| 3243: 311 | 1830 | 0.36 | 0.58 | NA | 0 | 0.87 | 0.71 | 0.2 | 0.67 | 0.14 | 0.23 | 0.26 | 0 | 0.24 | 0.86 |
| 3243: 392 | 1911 | NA | NA | NA | NA | NA | NA | NA | NA | NA | NA | NA | NA | NA | NA |

| CpG identifier | MVP Position in ROI | Brain | Brain | Brain | Brain | Breast |
|---|---|---|---|---|---|---|
| 3243: 57 | 1576 | NA | NA | 1 | 1 | 1 |
| 3243: 63 | 1582 | 1 | 1 | 1 | 1 | 1 |
| 3243: 132 | 1651 | 0.62 | 0.58 | 0.93 | 0.72 | 0.57 |
| 3243: 138 | 1657 | 0.92 | 0.78 | 0.97 | 0.85 | 0.51 |
| 3243: 140 | 1659 | 0.78 | 0.78 | 1 | 1 | 0.63 |
| 3243: 155 | 1674 | 0.82 | 0.74 | 1 | 1 | 0.32 |
| 3243: 182 | 1701 | 1 | 0.77 | 0.92 | 0.92 | 0.65 |
| 3243: 229 | 1748 | 1 | 0.6 | 0.91 | 0.65 | 0.33 |
| 3243: 252 | 1771 | 0.72 | 0.44 | 0.76 | 0.69 | 0.34 |
| 3243: 263 | 1782 | 1 | 0.56 | 1 | 1 | 0.31 |
| 3243: 311 | 1830 | 0.81 | 0.5 | 1 | 0.67 | 0.6 |
| 3243: 392 | 1911 | NA | 0.65 | 1 | 1 | NA |

TABLE 24
(3244):

| | MVP Position in | | | | | | | | | | | | |
|---|---|---|---|---|---|---|---|---|---|---|---|---|---|
| CpG identifier | ROI | Prostate | Prostate | Prostate | Prostate | Prostate | Prostate | Prostate | Prostate | Muscle | Muscle | Muscle | Muscle |
| 3244: 40 | 141 | 1 | 1 | 0.75 | 0.97 | 0.71 | 0.92 | 0.69 | 0.91 | 0.54 | 0.5 | 0.88 | 0.5 |
| 3244: 79 | 180 | 0.93 | 0.86 | 0.91 | 1 | 0.95 | 0.9 | 0.92 | 0.91 | 0.87 | 0.5 | 0.64 | 1 |
| 3244: 173 | 274 | 0.61 | 0.59 | 0.63 | 0.63 | 0.65 | 0.53 | 0.87 | 0.67 | 0.45 | 0.13 | 0.41 | 0.21 |
| 3244: 208 | 309 | 0.62 | 0.59 | 0.58 | 0.59 | 0.5 | 0.89 | 0.6 | 0.64 | 0.27 | 0 | 0.19 | 0.11 |
| 3244: 217 | 318 | 0.63 | 0.7 | 0.6 | 0.64 | 0.53 | 0.65 | 0.77 | 0.58 | 0.36 | 0.14 | 0.33 | 0.21 |
| 3244: 223 | 324 | 0.56 | 0.57 | 0.55 | 0.56 | 0.54 | 0.5 | 0.82 | 0.59 | 0.31 | 0.12 | 0.13 | 0.16 |
| 3244: 228 | 329 | 0.62 | 0.66 | 0.29 | 0.68 | 0.75 | 0.71 | 0.81 | 0.69 | 0.59 | 0.28 | 0.4 | 0.25 |
| 3244: 240 | 341 | 0.87 | 0.95 | 0.74 | 0.86 | 0.83 | 0.87 | 0.86 | 0.88 | 0.4 | 0.76 | 0.62 | 0.15 |

| | MVP | | | | | | | | | | | | | | |
|---|---|---|---|---|---|---|---|---|---|---|---|---|---|---|---|
| CpG identifier | Position in ROI | Muscle | Lung | Lung | Lung | Lung | Lung | Liver | Liver | Breast | Breast | Breast | Breast | Breast | Breast |
| 3244: 40 | 141 | 0.5 | 1 | 0.93 | 0.82 | 1 | 0.5 | 1 | 0.7 | 1 | 0.84 | 0.66 | 0.81 | 1 | 1 |
| 3244: 79 | 180 | 1 | 0.8 | 0.89 | 0.74 | 1 | 0.84 | 0.73 | 0.9 | 1 | 0.86 | 0.92 | 0.79 | 0.9 | 0.85 |
| 3244: 173 | 274 | 0.19 | 0.51 | 0.54 | 0.78 | 0.56 | 0.78 | 0.43 | 0.65 | 0.78 | 0.61 | 0.64 | 0.38 | 0.77 | 0.6 |
| 3244: 208 | 309 | 0.14 | 0.46 | 0.69 | 0.71 | 0.56 | 0.78 | 1 | 0.49 | 0.64 | 0.59 | 0.58 | 0.22 | 0.5 | 0.48 |
| 3244: 217 | 318 | 0.16 | 0.46 | 0.67 | 0.62 | 0.53 | 0.71 | 0.71 | 0.52 | 0.74 | 0.5 | 0.65 | 0.66 | 0.65 | 0.64 |
| 3244: 223 | 324 | 0.15 | 0.51 | 0.58 | 0.35 | 0.47 | 0.41 | 0.77 | 0.43 | 0.78 | 0.48 | 0.52 | 0.77 | 0.36 | 0.37 |
| 3244: 228 | 329 | 0.35 | 0.74 | 0.57 | 0.64 | 0.26 | 0.67 | NA | 0.63 | 0.84 | 0.59 | 0.75 | 1 | 0.64 | 0.62 |
| 3244: 240 | 341 | 0.19 | 0.82 | 0.88 | 0.93 | 0.91 | 0.97 | 0.2 | 0.83 | 0.83 | 0.54 | 0.9 | 0.78 | 0.78 | 1 |

| CpG identifier | MVP Position in ROI | Brain | Brain | Brain | Brain | Brain | Brain |
|---|---|---|---|---|---|---|---|
| 3244: 40 | 141 | 1 | 0.4 | 1 | 0.9 | 0.5 | NA |
| 3244: 79 | 180 | 1 | 0.43 | 0.7 | 0.87 | 1 | 1 |
| 3244: 173 | 274 | 0.8 | 0.72 | 0.73 | 0.38 | 0.81 | 0.78 |
| 3244: 208 | 309 | 0.6 | 0.46 | 0.65 | 0.81 | 0.61 | 0.47 |
| 3244: 217 | 318 | 0.73 | 0.41 | 0.72 | 0.48 | 0.78 | 0.67 |
| 3244: 223 | 324 | 0.87 | 0.19 | 0.42 | 0.34 | 0.79 | 0.6 |
| 3244: 228 | 329 | 0.68 | 0.27 | 0.55 | 0.43 | 0.83 | 0.77 |
| 3244: 240 | 341 | 0.85 | 0.85 | 0 | 0.8 | 0.71 | 0.71 |

TABLE 25
(3252):

| | MVP Position in | | | | | | | | | | | | |
|---|---|---|---|---|---|---|---|---|---|---|---|---|---|
| CpG identifier | ROI | Prostate | Prostate | Prostate | Prostate | Prostate | Prostate | Prostate | Prostate | Muscle | Muscle | Muscle | Muscle |
| 3252: 39 | 740 | 1 | NA | 0.82 | 0.51 | 1 | NA | 1 | 1 | 0.68 | NA | NA | 0.35 |
| 3252: 43 | 744 | 1 | NA | 1 | 1 | 1 | 1 | 1 | 1 | 1 | NA | NA | 0.97 |
| 3252: 88 | 789 | 0.95 | NA | 1 | 0.97 | 1 | 1 | 0.94 | 0.91 | 0.69 | NA | NA | 0.86 |
| 3252: 91 | 792 | 1 | NA | 0.85 | 0.91 | 0.87 | 1 | 0.9 | 0.85 | 0.57 | 0 | NA | 0.55 |
| 3252: 94 | 795 | 1 | NA | 0.73 | 0.73 | 1 | 0.67 | 1 | 0.93 | 0.78 | 1 | NA | 0.64 |
| 3252: 152 | 853 | 0.72 | 0.41 | 0.48 | 0.6 | 0.63 | 0.73 | 0.8 | 0.68 | 0.44 | 0 | 0 | 0.53 |
| 3252: 164 | 865 | 0.86 | NA | 0.59 | 0.66 | 0.64 | 0.52 | 0.71 | 0.77 | 0.46 | 0.17 | NA | 0.6 |
| 3252: 175 | 876 | 0.87 | 0.94 | 0.82 | 0.81 | 0.7 | 0.78 | 0.83 | 0.8 | 0.54 | 0.087 | NA | 0.63 |
| 3252: 178 | 879 | 0.74 | 0.6 | 0.64 | 0.66 | 0.54 | 0.55 | 0.64 | 0.67 | 0.42 | 0.26 | 0.34 | 0.46 |
| 3252: 199 | 900 | 0.95 | 1 | 0.95 | 0.9 | 0.84 | 0.93 | 0.94 | 0.85 | 0.53 | 0.1 | 1 | 0.64 |
| 3252: 206 | 907 | 0.8 | 0.88 | 0.84 | 0.82 | 0.62 | 0.81 | 0.84 | 0.67 | 0.36 | 0 | 0.32 | 0.49 |
| 3252: 242 | 943 | 0.95 | NA | 0.77 | 0.74 | 0.56 | 0.68 | 0.67 | 0.74 | 0.38 | 0.34 | 1 | 0.57 |
| 3252: 297 | 998 | 1 | NA | 1 | 0.91 | 0.86 | 0.97 | 0.81 | 0.92 | 0.77 | 0.23 | 1 | 0.87 |
| 3252: 303 | 1004 | 1 | NA | 1 | 0.98 | 0.81 | 1 | 0.93 | 0.96 | 1 | 0.06 | 1 | 0.99 |
| 3252: 308 | 1009 | 0.76 | NA | 0.5 | 0.57 | 0.26 | 0.51 | 0.35 | 0.52 | 0.35 | 0.0054 | 0.95 | 0.58 |
| 3252: 330 | 1031 | 0.62 | NA | 0.61 | 0.48 | 0.39 | 0.32 | 0.38 | 0.35 | 0.28 | 0 | 0.43 | 0.51 |
| 3252: 334 | 1035 | 0.44 | NA | 0.49 | 0.31 | 0 | 0.15 | 0.21 | 0.29 | 0 | 0.054 | 0.044 | 0.33 |
| 3252: 347 | 1048 | 0.57 | NA | NA | 0.29 | NA | 0.26 | NA | 0.31 | 0 | 0.0088 | NA | 0.5 |

| | MVP | | | | | | | | | | | | | | |
|---|---|---|---|---|---|---|---|---|---|---|---|---|---|---|---|
| CpG identifier | Position in ROI | Muscle | Lung | Lung | Lung | Lung | Lung | Liver | Liver | Breast | Breast | Breast | Breast | Breast | Brain |
| 3252: 39 | 740 | 0.33 | 0.91 | NA | 1 | 0.84 | 1 | NA | 0.88 | 0.68 | 1 | 0.7 | 1 | 1 | 0.33 |
| 3252: 43 | 744 | 1 | 1 | 1 | 1 | 1 | 1 | NA | 1 | 1 | 1 | 1 | 1 | NA | 1 |
| 3252: 88 | 789 | 0.31 | 0.91 | 1 | 1 | 1 | 0.94 | NA | 1 | 1 | 0.85 | 0.86 | 0.79 | 0.75 | 1 |
| 3252: 91 | 792 | 0.31 | 1 | 1 | 1 | 1 | 0.74 | NA | 1 | 0.7 | 0.75 | 0.66 | 0.79 | 0.92 | 0.89 |
| 3252: 94 | 795 | 0.3 | 1 | 0.41 | 1 | 0.8 | 0.71 | NA | 0.85 | 0.68 | 1 | 0.71 | 0.68 | 0.65 | 0.69 |
| 3252: 152 | 853 | 0.19 | 0.86 | 0.64 | 1 | 0.72 | 1 | NA | 0.86 | 0.44 | 0.62 | 0.54 | 0.83 | 0.58 | 0.95 |
| 3252: 164 | 865 | 0.21 | 0.82 | 0.68 | 1 | 0.72 | 0.66 | NA | 0.81 | 0.41 | 0.64 | 0.51 | 0.65 | 0.56 | 0.71 |
| 3252: 175 | 876 | 0.28 | 0.85 | 1 | 1 | 0.93 | 0.63 | NA | 1 | 0.61 | 0.67 | 0.55 | 1 | 0.15 | 0.98 |
| 3252: 178 | 879 | 0.18 | 0.67 | 0.33 | 1 | 0.62 | 0.85 | NA | 0.69 | 0.33 | 0.43 | 0.49 | 0.43 | 0.49 | 0.59 |
| 3252: 199 | 900 | 0.33 | 0.91 | 1 | 1 | 0.93 | 0.86 | NA | 0.99 | 0.59 | 0.75 | 0.73 | 1 | 0.72 | 0.96 |
| 3252: 206 | 907 | 0.2 | 0.8 | 1 | 0.82 | 0.83 | 0.85 | NA | 0.87 | 0.54 | 0.24 | 0.45 | 1 | 0.57 | 0.96 |
| 3252: 242 | 943 | 0.58 | 0.77 | 0.96 | 0.79 | 0.86 | 0.97 | NA | 0.8 | 0.38 | 0.34 | 0.38 | 0.94 | 0.58 | 0.89 |
| 3252: 297 | 998 | 0.55 | 0.97 | 1 | 0.95 | 0.92 | 0.97 | NA | 1 | 0.72 | 0.82 | 0.7 | 1 | 0.52 | 1 |
| 3252: 303 | 1004 | 1 | 1 | 1 | 1 | 1 | 0.98 | NA | 1 | 0.53 | 0.85 | 0.78 | 0.84 | 0.63 | 1 |
| 3252: 308 | 1009 | 0.19 | 0.64 | 0.92 | 0.7 | 0.64 | 0.9 | NA | 0.83 | 0.31 | 0.5 | 0.27 | 0.86 | 0.36 | 0.88 |
| 3252: 330 | 1031 | 0 | 0.54 | 1 | 0.62 | 0.7 | 0.61 | NA | 0.49 | 0.45 | 0.34 | 0.51 | 0.89 | 0.25 | 0.78 |
| 3252: 334 | 1035 | 0 | 0.26 | 0.59 | 0.58 | 0.43 | 0.66 | NA | 0.11 | 0.17 | 0.15 | 0.19 | 0.74 | 0.11 | 0.5 |
| 3252: 347 | 1048 | NA | 0.47 | NA | 0 | NA | 0.52 | NA | 0.19 | 0.46 | 0.43 | NA | 0.67 | 0.093 | 0.29 |

| CpG identifier | MVP Position in ROI | Brain | Brain | Brain | Brain | Brain |
|---|---|---|---|---|---|---|
| 3252: 39 | 740 | NA | NA | NA | 1 | 0.82 |
| 3252: 43 | 744 | 0.98 | NA | NA | 1 | 1 |
| 3252: 88 | 789 | 1 | NA | NA | 0.96 | 1 |
| 3252: 91 | 792 | 0.69 | NA | 1 | 0.77 | 0.82 |
| 3252: 94 | 795 | 0.46 | NA | NA | 0.91 | 0.75 |
| 3252: 152 | 853 | 0.19 | NA | NA | 0.66 | 0.7 |
| 3252: 164 | 865 | 0.35 | NA | 0.28 | 0.8 | 0.78 |
| 3252: 175 | 876 | 1 | NA | 0.89 | 0.97 | 0.92 |
| 3252: 178 | 879 | 0.49 | NA | 0.73 | 0.51 | 0.64 |
| 3252: 199 | 900 | 1 | NA | 1 | 0.98 | 0.93 |
| 3252: 206 | 907 | 0.95 | NA | 1 | 0.85 | 0.76 |
| 3252: 242 | 943 | 0.98 | NA | NA | 0.89 | 0.67 |
| 3252: 297 | 998 | 1 | NA | 1 | 1 | 1 |
| 3252: 303 | 1004 | 1 | NA | 1 | 1 | 1 |
| 3252: 308 | 1009 | 0.86 | NA | 0.42 | 0.9 | 0.75 |
| 3252: 330 | 1031 | 1 | NA | 0.72 | 0.77 | 0.73 |
| 3252: 334 | 1035 | 0.75 | NA | 0.67 | 0.3 | 0.49 |
| 3252: 347 | 1048 | NA | NA | 0 | 0.62 | 0 |

TABLE 26
(3265):

| | MVP Position in | | | | | | | | | | | | |
|---|---|---|---|---|---|---|---|---|---|---|---|---|---|
| CpG identifier | ROI | Prostate | Prostate | Prostate | Prostate | Prostate | Prostate | Prostate | Prostate | Muscle | Muscle | Muscle | Muscle |
| 3265: 62 | 716 | 0 | 0.016 | 0.019 | 0.12 | 0.046 | 0 | 0.11 | 0 | 0.41 | 0.26 | 0.062 | 0.37 |
| 3265: 81 | 735 | 0 | 0 | 0.014 | 0 | 0 | 0 | 0 | 0 | 0.048 | 0.047 | 0 | 0.089 |
| 3265: 84 | 738 | 0.054 | 0 | 0.062 | 0.055 | 0.044 | 0.027 | 0 | 0 | 0.23 | 0.38 | 0.34 | 0.22 |
| 3265: 137 | 791 | 0.083 | 0 | 0.047 | 0.23 | 0.23 | 0.055 | 0.18 | 0.021 | 0.2 | 0.3 | 0.23 | 0.36 |
| 3265: 139 | 793 | 0.087 | 0 | 0.067 | 0.037 | 0.08 | 0.077 | 0.19 | 0.021 | 0.092 | 0.081 | 0.01 | 0.23 |
| 3265: 259 | 913 | 0 | 0.032 | 0 | 0.031 | 0.079 | 0 | 0.11 | 0.029 | 0.3 | 0.38 | 0.054 | 0.47 |
| 3265: 337 | 991 | 0.25 | 0.47 | 0.015 | 0 | 0.1 | 0.3 | 0.0081 | 0.063 | 0.37 | 0.4 | 0.39 | 0.35 |
| 3265: 350 | 1004 | 0 | 0.055 | 0.31 | 0.029 | 0.13 | 0.25 | 0.34 | 0.0035 | 0.071 | 0.27 | 0.13 | 0.2 |
| 3265: 362 | 1016 | 0 | 0 | 0 | 0.0039 | 0.024 | 0.00065 | 0.019 | 0.038 | 0.13 | 0.36 | 0.0078 | 0.27 |
| 3265: 395 | 1049 | 0.042 | 0 | 0.035 | 0.008 | 0.15 | 0.091 | 0.084 | 0.067 | 0.33 | 0.43 | 0.7 | 0.33 |
| 3265: 404 | 1058 | 0.23 | 0.11 | 0.11 | 0.06 | 0.049 | 0.14 | 0.23 | 0.08 | 0.098 | 0.35 | 0.57 | 0.51 |

| CpG | MVP | | | | | | | | | | | | | | |
|---|---|---|---|---|---|---|---|---|---|---|---|---|---|---|---|
| identifier | Position in ROI | Muscle | Lung | Lung | Lung | Lung | Lung | Liver | Liver | Breast | Breast | Breast | Breast | Breast | Breast |
| 3265: 62 | 716 | 0.11 | 0.086 | 0.18 | 0 | 0 | 0.15 | 0.11 | NA | 0.02 | 0 | 0.052 | 0 | 0.39 | 0.091 |
| 3265: 81 | 735 | 0.018 | 0 | 0 | 0 | 0.043 | 0 | 0.023 | NA | 0 | 0 | 0 | 0.2 | 0 | 0.14 |
| 3265: 84 | 738 | 0.4 | 0 | 0.033 | 0 | 0 | 0.036 | 0.098 | NA | 0.031 | 0 | 0.046 | 0.15 | 0.0074 | 0.037 |
| 3265: 137 | 791 | 0.33 | 0 | 0.29 | 0 | 0.085 | 0.14 | 0.092 | 0 | 0.22 | 0.063 | 0.076 | 0 | 0 | 0.18 |
| 3265: 139 | 793 | 0.22 | 0.13 | 0.12 | 0.14 | 0.088 | 0 | 0.12 | 0 | 0 | 0.037 | 0.097 | 0.027 | 0 | 0.22 |
| 3265: 259 | 913 | 0.43 | 0.11 | 0.033 | NA | 0 | 0 | 0.31 | 0 | 0.3 | 0.14 | 0.083 | 0 | 0.3 | 0 |
| 3265: 337 | 991 | 0.54 | 0.15 | 0.041 | 0 | 0.028 | 0.29 | 0.48 | 0.89 | 0.45 | 0.067 | 0.18 | 0.17 | 0.65 | 0.12 |
| 3265: 350 | 1004 | 0.35 | 0.11 | 0.074 | 0 | 0.058 | 0 | 0.11 | 0.22 | 0 | 0 | 0 | 0.077 | 0 | 0 |
| 3265: 362 | 1016 | 0.21 | 0.0025 | 0 | 0 | 0.096 | 0 | 0.027 | NA | 0 | 0 | 0 | 0.031 | 0 | 0 |
| 3265: 395 | 1049 | 0.4 | 0.14 | 0.085 | 0 | 0.12 | 0.018 | 0.046 | 1 | 0 | 0 | 0.11 | 0.049 | 0.21 | 0 |
| 3265: 404 | 1058 | 0.57 | 0.14 | 0.1 | 0 | 0.1 | 0.2 | 0.36 | 0.7 | 0.3 | 0.048 | 0.27 | 0.022 | 0.16 | 0.6 |

| CpG identifier | MVP Position in ROI | Brain | Brain | Brain | Brain | Brain |
|---|---|---|---|---|---|---|
| 3265: 62 | 716 | 0 | 0.095 | 0.12 | 0.25 | 0.14 |
| 3265: 81 | 735 | 0 | 0 | 0 | 0.0014 | 0.09 |
| 3265: 84 | 738 | 0.06 | 0 | 0.052 | 0.05 | 0.048 |
| 3265: 137 | 791 | 0.15 | 0 | 0.48 | 0.21 | 0.19 |
| 3265: 139 | 793 | 0.0092 | 0.077 | 0 | 0.18 | 0 |
| 3265: 259 | 913 | 0.033 | 0.13 | 0 | 0.22 | 0 |
| 3265: 337 | 991 | 0.092 | 0.5 | 0.37 | 0.26 | 0.065 |
| 3265: 350 | 1004 | 0 | 0.037 | 0.13 | 0 | 0 |
| 3265: 362 | 1016 | 0.024 | 0.024 | 0.3 | 0.0081 | 0.19 |
| 3265: 395 | 1049 | 0 | 0.02 | 0 | 0.21 | 0.11 |
| 3265: 404 | 1058 | 0.42 | 0.14 | 0.25 | 0.22 | 0.089 |

TABLE 27
(3291):

| | MVP Position in | | | | | | | | | | | | |
|---|---|---|---|---|---|---|---|---|---|---|---|---|---|
| CpG identifier | ROI | Prostate | Prostate | Prostate | Prostate | Prostate | Prostate | Prostate | Prostate | Muscle | Muscle | Muscle | Muscle |
| 3291: 42 | 247 | 0.92 | 0.75 | 1 | 0.97 | 0.89 | 0.97 | 0.95 | 0.75 | 0.85 | 0.64 | 0.8 | 0.72 |
| 3291: 64 | 269 | 0.52 | 0.45 | 0.56 | 0.55 | 0.4 | 0.63 | 0.47 | 0.34 | 0.6 | 0.42 | 0.5 | 0.44 |
| 3291: 71 | 276 | 0.8 | 0.8 | 0.74 | 0.92 | 0.83 | 0.85 | 0.36 | 0.68 | 0.84 | 0.74 | 0.82 | 0.64 |
| 3291: 81 | 286 | 0.48 | 0.43 | 0.48 | 0.41 | 0.47 | 0.52 | 0.49 | 0.34 | 0.45 | 0.27 | 0.5 | 0.48 |
| 3291: 369 | 574 | 0.89 | 0.87 | 1 | 1 | 0.91 | 1 | 1 | 0.94 | 0.91 | 1 | 0.94 | 0.91 |

| | MVP Position in | | | | | | | | | | | | | | |
|---|---|---|---|---|---|---|---|---|---|---|---|---|---|---|---|
| CpG identifier | ROI | Lung | Lung | Lung | Lung | Lung | Liver | Breast | Breast | Breast | Breast | Breast | Brain | Brain | Brain |
| 3291: 42 | 247 | 0.68 | 0.55 | 0.38 | 0.27 | 0.35 | 0.75 | 0.54 | 0.36 | 0.66 | 0.91 | 0.87 | 0.81 | 1 | 0.73 |
| 3291: 64 | 269 | 0.37 | 0.22 | 0.22 | 0.64 | 0.46 | 0.39 | 0.2 | 0.24 | 0.2 | 0.4 | 0.68 | 0.79 | 0.6 | NA |
| 3291: 71 | 276 | 0.67 | 0.55 | 0.53 | 0.57 | 0.41 | 0.88 | 0.5 | 0.5 | 0.63 | 0.62 | 0.91 | 0.86 | 0.95 | 0.84 |
| 3291: 81 | 286 | 0.41 | 0.075 | 0.0075 | 0.26 | 0.23 | 0.22 | 0.56 | 0.00053 | 0.44 | 0.23 | 0.41 | 0.65 | 0.67 | 0.61 |
| 3291: 369 | 574 | 0.92 | 0.94 | 0.76 | 0.68 | 0.76 | 0.92 | 0.86 | 1 | 0.86 | 1 | NA | 1 | 1 | NA |

| CpG identifier | MVP Position in ROI | Brain | Brain | Brain |
|---|---|---|---|---|
| 3291: 42 | 247 | 1 | 1 | 0.86 |
| 3291: 64 | 269 | 0.12 | 1 | 0.58 |
| 3291: 71 | 276 | NA | 1 | 0.87 |
| 3291: 81 | 286 | 0 | 0.79 | 0.79 |
| 3291: 369 | 574 | 1 | 1 | 0.97 |

TABLE 28
(3312):

| | MVP Position in | | | | | | | | | | | | |
|---|---|---|---|---|---|---|---|---|---|---|---|---|---|
| CpG identifier | ROI | Prostate | Prostate | Prostate | Prostate | Prostate | Prostate | Prostate | Prostate | Muscle | Muscle | Muscle | Muscle |
| 3312: 71 | 1498 | 1 | 1 | NA | 0.54 | 1 | 1 | 1 | 1 | 1 | 0.88 | 1 | 1 |
| 3312: 95 | 1522 | 1 | 1 | 1 | 0.71 | 1 | 0.74 | 1 | 1 | 1 | 1 | 0.72 | 1 |
| 3312: 103 | 1530 | 1 | 1 | 1 | 0.79 | 1 | 0.65 | 1 | 0.81 | 0.6 | 0.58 | 1 | 0.64 |
| 3312: 119 | 1546 | 1 | 1 | 1 | 1 | 1 | 1 | 1 | 1 | 0.74 | 0.71 | 0.88 | 0.82 |
| 3312: 158 | 1585 | 0.9 | 0.94 | 0.91 | 0.83 | 0.86 | 0.91 | 0.87 | 1 | 0.47 | 0.47 | 0.79 | 0.43 |
| 3312: 167 | 1594 | 1 | 1 | 1 | 1 | 1 | 1 | 1 | 1 | 0.96 | 0.92 | 0.88 | 0.9 |
| 3312: 193 | 1620 | 0.84 | 0.94 | 0.93 | 0.89 | 0.71 | 0.74 | 0.76 | 0.92 | 0.73 | 0.73 | 0.7 | 0.72 |
| 3312: 215 | 1642 | 0.88 | 0.92 | 0.88 | 0.88 | 0.94 | 0.94 | 0.88 | 1 | 0.82 | 0.8 | 1 | 0.8 |
| 3312: 223 | 1650 | 0.9 | 0.96 | 0.9 | 1 | 1 | 1 | 0.97 | 1 | 0.88 | 0.88 | 0.56 | 0.83 |
| 3312: 242 | 1669 | 0.89 | 0.93 | 0.91 | 1 | 1 | 0.96 | 0.94 | 1 | 0.9 | 0.93 | 0.9 | 0.89 |
| 3312: 259 | 1686 | 1 | 0.97 | 1 | 1 | 1 | 1 | 1 | NA | 1 | 1 | 1 | 1 |
| 3312: 273 | 1700 | 1 | 0.95 | 0.91 | 1 | 1 | 1 | 1 | 1 | 0.84 | 0.84 | 0.85 | 0.86 |
| 3312: 314 | 1741 | 0.76 | 0.7 | 0.71 | 1 | 0.73 | 1 | 0.83 | 1 | 0.73 | 0.67 | 0.29 | 0.8 |
| 3312: 404 | 1831 | 0.91 | 0.86 | 0.77 | 1 | 0.54 | 1 | 0.87 | 1 | 0.76 | 0.75 | 0.35 | 0.79 |
| 3312: 412 | 1839 | 0.95 | 1 | 0.93 | 0.98 | 1 | 0.96 | 0.97 | 1 | 1 | 1 | NA | 0.96 |

| | MVP Position in | | | | | | | | | | | | | | |
|---|---|---|---|---|---|---|---|---|---|---|---|---|---|---|---|
| CpG identifier | ROI | Muscle | Lung | Lung | Lung | Lung | Lung | Liver | Liver | Breast | Breast | Breast | Breast | Breast | Brain |
| 3312: 71 | 1498 | 1 | 1 | 1 | 1 | NA | 1 | 0.8 | 0 | 1 | 1 | 1 | NA | 1 | 1 |
| 3312: 95 | 1522 | 1 | 1 | 1 | 1 | 1 | 1 | 0.53 | 0.22 | 1 | 1 | 1 | 1 | 1 | 1 |
| 3312: 103 | 1530 | 0.58 | 1 | 1 | 1 | 1 | 0.79 | 0.51 | 0.27 | 0.87 | 1 | 0.74 | 1 | 0.71 | 0.84 |
| 3312: 119 | 1546 | 0.91 | 1 | 1 | 1 | 1 | 1 | NA | 0 | 0.82 | 1 | 0.87 | 1 | 1 | 1 |
| 3312: 158 | 1585 | 0.4 | 0.9 | 0.91 | 0.92 | 0.92 | 0.84 | 0.64 | 0 | 0.69 | 0.66 | 0.52 | 0.58 | 0.67 | 0.88 |
| 3312: 167 | 1594 | 0.95 | 1 | 1 | 1 | 1 | 1 | 0.22 | 0 | 1 | 0.96 | 1 | 1 | 1 | 1 |
| 3312: 193 | 1620 | 0.64 | 0.91 | 0.85 | 0.89 | 1 | 0.87 | 0.65 | 0.045 | 0.81 | 0.79 | 0.82 | 0.86 | 0.77 | 0.76 |
| 3312: 215 | 1642 | 0.81 | 0.88 | 0.89 | 1 | 0.91 | 0.9 | 0 | 0.3 | 0.83 | 0.84 | 0.75 | 0.73 | 0.88 | 0.82 |
| 3312: 223 | 1650 | 0.87 | 0.93 | 0.94 | 0.9 | 0.9 | 0.97 | NA | 0 | 0.78 | 0.86 | 0.82 | 0.79 | 0.82 | 0.81 |
| 3312: 242 | 1669 | 0.89 | 0.91 | 0.9 | 0.88 | 0.96 | 0.94 | NA | 0 | 0.93 | 0.87 | 0.86 | 1 | 0.91 | 0.92 |
| 3312: 259 | 1686 | 1 | 0.97 | 0.97 | 0.96 | 0.95 | 1 | 1 | 1 | 1 | 1 | 1 | 1 | 1 | 0.98 |
| 3312: 273 | 1700 | 0.85 | 0.97 | 0.89 | 0.94 | 0.91 | 1 | 0.56 | 1 | 0.91 | 0.84 | 0.93 | 0.74 | 0.84 | 0.9 |
| 3312: 314 | 1741 | 0.64 | 0.66 | 0.81 | 0.68 | 0.8 | 0.85 | 1 | 0.56 | 0.63 | 1 | 0.74 | 0.85 | 0.7 | 0.58 |
| 3312: 404 | 1831 | 0.72 | 1 | 0.79 | 1 | 0.8 | 0.75 | 1 | 0.42 | 0.81 | 0.7 | 1 | 0.63 | 0.59 | 1 |
| 3312: 412 | 1839 | 1 | 0.98 | 0.97 | 0.93 | 0.89 | 1 | NA | 0.88 | 1 | 1 | 1 | 1 | 0.97 | 1 |

| CpG identifier | MVP Position in ROI | Brain | Brain | Brain | Brain |
|---|---|---|---|---|---|
| 3312: 71 | 1498 | 1 | 1 | 1 | 1 |
| 3312: 95 | 1522 | 1 | 1 | 1 | 1 |
| 3312: 103 | 1530 | 1 | 1 | 1 | 1 |
| 3312: 119 | 1546 | 0.79 | 1 | 1 | 1 |
| 3312: 158 | 1585 | 0.88 | 1 | 0.91 | 0.93 |
| 3312: 167 | 1594 | 1 | 1 | 1 | 1 |
| 3312: 193 | 1620 | 0.66 | 0.81 | 0.83 | 0.79 |
| 3312: 215 | 1642 | 0.82 | 0.73 | 0.86 | 0.88 |
| 3312: 223 | 1650 | 0.77 | 0.95 | 0.9 | 0.92 |
| 3312: 242 | 1669 | 0.89 | 0.93 | 0.94 | 0.94 |
| 3312: 259 | 1686 | 1 | 1 | 1 | 0.97 |
| 3312: 273 | 1700 | 0.87 | 1 | 0.96 | 1 |
| 3312: 314 | 1741 | 0.85 | 0.69 | 0.68 | 0.71 |
| 3312: 404 | 1831 | 0.76 | 0.84 | 0.8 | 0.83 |
| 3312: 412 | 1839 | 0.96 | 1 | 1 | 1 |

TABLE 29
(3329):

| | MVP Position in | | | | | | | | | | | | |
|---|---|---|---|---|---|---|---|---|---|---|---|---|---|
| CpG identifier | ROI | Prostate | Prostate | Prostate | Prostate | Prostate | Prostate | Prostate | Prostate | Muscle | Muscle | Muscle | Muscle |
| 3329: 52 | 1151 | 1 | NA | 1 | NA | 1 | NA | NA | NA | 1 | NA | NA | NA |
| 3329: 135 | 1234 | 0.93 | 0.9 | 0.94 | 0.93 | 0.92 | 0.96 | 0.95 | 0.91 | 1 | NA | 0.67 | 1 |
| 3329: 154 | 1253 | 0.88 | 0.91 | 0.91 | 0.92 | 0.91 | 0.95 | 0.92 | 0.82 | 0.92 | 1 | 1 | 0.87 |
| 3329: 187 | 1286 | 0.9 | 1 | 1 | 0.92 | 0.96 | 0.96 | 0.99 | 0.92 | 0.93 | 1 | 0.8 | 0.95 |
| 3329: 241 | 1340 | 0.91 | 0.95 | 0.98 | 0.92 | 0.96 | 0.96 | 0.94 | 0.97 | 0.9 | 0.89 | 1 | 0.97 |
| 3329: 251 | 1350 | 1 | 1 | 0.96 | 0.98 | 0.98 | 1 | 1 | 0.97 | 0.99 | 1 | 0.9 | 1 |
| 3329: 303 | 1402 | 0.96 | 0.49 | 0.95 | 0.87 | 0.75 | 1 | 0.96 | 0.93 | 0.85 | NA | 0.88 | 0.67 |
| 3329: 315 | 1414 | 0.84 | 0.84 | 0.75 | 0.92 | 0.94 | 0.85 | 0.98 | 0.82 | 0.9 | 1 | 0.81 | 0.91 |
| 3329: 420 | 1519 | 0.27 | 0.4 | 0.37 | 0.48 | 0.48 | 0.36 | 0.36 | 0.18 | 0.45 | 0.57 | 0.35 | 0.53 |
| 3329: 440 | 1539 | 0.5 | 0.65 | 0.55 | 0.62 | 0.67 | 1 | 0.62 | 0.53 | 0.66 | NA | 1 | 0.57 |

| | MVP | | | | | | | | | | | | | | |
|---|---|---|---|---|---|---|---|---|---|---|---|---|---|---|---|
| CpG identifier | Position in ROI | Muscle | Lung | Lung | Lung | Lung | Lung | Liver | Liver | Breast | Breast | Breast | Breast | Breast | Breast |
| 3329: 52 | 1151 | NA | NA | NA | NA | 1 | 0.85 | 0.65 | NA | 0.83 | NA | NA | NA | 0.5 | NA |
| 3329: 135 | 1234 | 0.92 | 1 | 1 | 0.8 | 0.96 | 0.95 | 0.64 | 0 | 1 | 1 | 1 | 0.92 | 1 | 0.9 |
| 3329: 154 | 1253 | 0.84 | 0.85 | 0.84 | 0.91 | 0.94 | 0.92 | 0.55 | 0.13 | 0.94 | 0.82 | 0.9 | 0.92 | 0.91 | 0.92 |
| 3329: 187 | 1286 | 0.93 | 0.92 | 0.95 | 1 | 0.9 | 0.96 | 0.55 | 0.097 | 0.95 | 0.97 | 0.95 | 0.85 | 1 | 0.93 |
| 3329: 241 | 1340 | 0.97 | 0.98 | 1 | 1 | 1 | 0.96 | 0.57 | 0.17 | 0.95 | 1 | 0.95 | 1 | 0.94 | 0.97 |
| 3329: 251 | 1350 | 1 | 0.98 | 1 | 1 | 0.95 | 1 | 0.79 | 0.35 | 1 | 0.98 | 0.98 | 1 | 1 | 0.91 |
| 3329: 303 | 1402 | 0.87 | 0.95 | 0.92 | 0.72 | 1 | 0.95 | 0.32 | 0.079 | 0.91 | 0.98 | 0.9 | 1 | 0.97 | 0.82 |
| 3329: 315 | 1414 | 0.83 | 0.89 | 0.96 | 0.97 | 0.91 | 0.87 | 0.71 | 0.21 | 0.8 | 0.98 | 0.92 | 0.59 | 0.82 | 0.81 |
| 3329: 420 | 1519 | 0.35 | 0.33 | 0.46 | 0.39 | 0.44 | NA | 0.34 | 0.61 | 0.31 | 0.5 | 0.43 | 0.47 | 0.49 | 0.39 |
| 3329: 440 | 1539 | 0.56 | 0.62 | 0.67 | 0.63 | 0.72 | 0.56 | 0.65 | 0.87 | 0.61 | 0.59 | 1 | 0.69 | 0.59 | 0.64 |

| CpG identifier | MVP Position in ROI | Brain | Brain | Brain | Brain | Brain | Brain |
|---|---|---|---|---|---|---|---|
| 3329: 52 | 1151 | 1 | NA | NA | NA | NA | NA |
| 3329: 135 | 1234 | 1 | 0.93 | 0.91 | 1 | 1 | 1 |
| 3329: 154 | 1253 | 0.92 | 0.95 | 0.73 | 0.83 | 0.97 | 0.96 |
| 3329: 187 | 1286 | 1 | 1 | 1 | 1 | 1 | 1 |
| 3329: 241 | 1340 | 0.99 | 0.97 | 1 | 0.94 | 0.97 | 1 |
| 3329: 251 | 1350 | 1 | 1 | 0.77 | 1 | 1 | 1 |
| 3329: 303 | 1402 | 0.79 | 0.74 | 0.45 | 0.91 | 0.88 | 0.87 |
| 3329: 315 | 1414 | 1 | 0.95 | NA | 0.74 | 0.94 | 0.95 |
| 3329: 420 | 1519 | 0.48 | 0.49 | NA | 0.38 | 0.54 | 0.48 |
| 3329: 440 | 1539 | 0.75 | 0.72 | 0.22 | 0.72 | 0.76 | 0.64 |

TABLE 30
(3330):

| | MVP Position in | | | | | | | | | | | | |
|---|---|---|---|---|---|---|---|---|---|---|---|---|---|
| CpG identifier | ROI | Prostate | Prostate | Prostate | Prostate | Prostate | Prostate | Prostate | Prostate | Muscle | Muscle | Muscle | Muscle |
| 3330: 45 | 2033 | 0.9 | 0.7 | 0.85 | 0.96 | 0.94 | 0.84 | 0.9 | 0.96 | 1 | 1 | 1 | 1 |
| 3330: 127 | 2115 | 0.81 | 0.61 | 0.87 | 0.87 | 0.82 | 0.82 | 0.89 | 0.93 | 0.95 | 1 | 1 | 0.86 |
| 3330: 151 | 2139 | 0.22 | 0.2 | 0.44 | 0.45 | 0.35 | 0.41 | 0.37 | 0.41 | 0.3 | 0.37 | 0.47 | 0.37 |
| 3330: 251 | 2239 | 0.67 | 0.66 | 0.52 | 0.76 | 0.63 | 0.49 | 0.55 | 0.59 | 0.75 | 0.86 | 0.37 | 0.73 |
| 3330: 260 | 2248 | 0.68 | 0.35 | 0.62 | 0.82 | 0.8 | 0.74 | 0.74 | 0.82 | 0.84 | 0.91 | 0.8 | 0.94 |
| 3330: 265 | 2253 | 0.69 | 0.52 | 0.87 | 0.83 | 0.77 | 0.72 | 0.83 | 0.87 | 0.63 | 0.71 | 0.74 | 0.69 |
| 3330: 298 | 2286 | 0.87 | 0 | 0.61 | 0.81 | 0.73 | 0.68 | 0.7 | 0.75 | 0.71 | 0.83 | 0.97 | 0.81 |
| 3330: 311 | 2299 | 0.82 | 0.54 | 0.82 | 0.87 | 0.77 | 0.8 | 0.85 | 0.88 | 0.96 | 1 | 1 | 1 |
| 3330: 320 | 2308 | 0.76 | 0.54 | 0.81 | 0.88 | 0.86 | 0.84 | 0.76 | 0.89 | 0.95 | 0.93 | 1 | 0.91 |
| 3330: 394 | 2382 | 1 | 0 | 1 | 1 | 1 | 1 | 1 | 1 | 1 | 1 | 1 | 1 |
| 3330: 401 | 2389 | 1 | 0 | 0.57 | 1 | 1 | 0.37 | 0.58 | 1 | 1 | 1 | 1 | 0.74 |

| | MVP | | | | | | | | | | | | | | |
|---|---|---|---|---|---|---|---|---|---|---|---|---|---|---|---|
| CpG identifier | Position in ROI | Muscle | Lung | Lung | Lung | Lung | Lung | Liver | Liver | Breast | Breast | Breast | Breast | Breast | Breast |
| 3330: 45 | 2033 | 1 | 0.8 | 1 | 0 | 0.92 | 0.86 | 0.88 | 1 | 1 | 0.88 | 0.92 | 0.84 | 1 | 0.87 |
| 3330: 127 | 2115 | 0.97 | 0.81 | 0.77 | 0.74 | 0.82 | 0.92 | 0.83 | 1 | 1 | 0.92 | 0.94 | 1 | 0.96 | 0.88 |
| 3330: 151 | 2139 | 0.4 | 0.22 | 0.2 | 0 | 0.2 | 0.23 | 0.36 | 0.99 | 0.43 | 0.23 | 0.41 | 0.18 | 0.46 | 0.44 |
| 3330: 251 | 2239 | 0.74 | 0.53 | 0.66 | 0.31 | 0.52 | 0.64 | 0.51 | 0.7 | 0.57 | 0.55 | 0.76 | 0.72 | 0.68 | 0.58 |
| 3330: 260 | 2248 | 0.95 | 0.59 | 0.34 | 0 | 0.28 | 0.57 | 0.48 | 0.96 | 0.75 | 0.64 | 0.69 | 0.73 | 0.74 | 0.64 |
| 3330: 265 | 2253 | 0.71 | 0.59 | 0.52 | 0.8 | 0.61 | 0.6 | 0.48 | 0.83 | 0.83 | 0.63 | 0.82 | 0.34 | 0.87 | 0.58 |
| 3330: 298 | 2286 | 0.84 | 0.44 | 0.29 | 0.75 | 0.43 | 0.49 | 0.11 | 0.94 | 0.67 | 0.53 | 0.69 | 0.26 | 0.77 | 0.61 |
| 3330: 311 | 2299 | 1 | 0.7 | 0.35 | 0.78 | 0.63 | 0.73 | 0.66 | 0.94 | 0.97 | 0.8 | 0.86 | 0.85 | 0.94 | 0.78 |
| 3330: 320 | 2308 | 0.93 | 0.79 | 0.45 | 0.51 | 0.6 | 0.73 | 0.56 | 0.8 | 0.82 | 0.67 | 0.75 | 0.6 | 0.87 | 0.81 |
| 3330: 394 | 2382 | 1 | 0.67 | 0.3 | 0.5 | 0.81 | 0.88 | 1 | NA | 1 | 1 | 1 | 0.5 | 0.88 | 1 |
| 3330: 401 | 2389 | 1 | 1 | 0.5 | 0 | 0.5 | 1 | 0 | 0.62 | 0.44 | 1 | 1 | 0 | 1 | 1 |

| CpG identifier | MVP Position in ROI | Brain | Brain | Brain | Brain | Brain | Brain |
|---|---|---|---|---|---|---|---|
| 3330: 45 | 2033 | 1 | 1 | 1 | 0.86 | 1 | 1 |
| 3330: 127 | 2115 | 1 | 1 | 0.94 | 1 | 0.98 | 1 |
| 3330: 151 | 2139 | 0.23 | 0.41 | 1 | 0.6 | 0.49 | 0.58 |
| 3330: 251 | 2239 | 0.87 | 0.66 | 0.93 | 0.85 | 0.8 | 0.6 |
| 3330: 260 | 2248 | 1 | 0.87 | 1 | 0.58 | 0.89 | 0.86 |
| 3330: 265 | 2253 | 0.85 | 0.92 | 0.88 | 0.78 | 0.74 | 0.78 |
| 3330: 298 | 2286 | 0.93 | 0.8 | 1 | 0.71 | 0.77 | 0.79 |
| 3330: 311 | 2299 | 1 | 1 | 1 | 0.86 | 1 | 1 |
| 3330: 320 | 2308 | 1 | 1 | 0.048 | 0.81 | 1 | 0.93 |
| 3330: 394 | 2382 | 1 | 1 | 1 | 1 | 1 | 1 |
| 3330: 401 | 2389 | 1 | 1 | 1 | 1 | 1 | NA |

TABLE 31
(3347):

| | MVP | | | | | | | | | | | | |
|---|---|---|---|---|---|---|---|---|---|---|---|---|---|
| | Position | | | | | | | | | | | | |
| CpG | in | | | | | | | | | | | | |
| identifier | ROI | Prostate | Prostate | Prostate | Prostate | Prostate | Prostate | Prostate | Prostate | Muscle | Muscle | Muscle | Muscle |
| 3347: 32 | 1907 | 0.64 | 0.46 | NA | 0.042 | 0.21 | 0 | 1 | NA | 0 | 0 | 0 | NA |
| 3347: 63 | 1938 | 0.71 | 0.82 | 0.38 | 0.65 | 0.8 | 0.81 | 0.88 | 0.63 | 0.26 | 0.16 | 0 | 0.56 |
| 3347: 65 | 1940 | 0.61 | 0.7 | 0.42 | 0.4 | 0.88 | 0.66 | 0.65 | 0.52 | 0.099 | 0 | 0 | 0.14 |
| 3347: 71 | 1946 | NA | 0.12 | NA | 0.41 | 0.62 | NA | 0.63 | 0.45 | NA | NA | 0 | 0.75 |
| 3347: 85 | 1960 | 0.53 | 0.095 | 0.5 | 0.31 | 0.43 | 0.71 | 0.88 | 0.0054 | 0 | 0 | 0.011 | NA |
| 3347: 92 | 1967 | 0.37 | 0.3 | 0.13 | 0.14 | 0.38 | 0.52 | 0.75 | 0.064 | 0 | 0 | 0 | 0 |
| 3347: 100 | 1975 | 0.64 | 0.31 | 0.083 | 0.3 | 0.13 | 0.2 | 0.53 | NA | 0 | 0 | 0 | 0.21 |
| 3347: 103 | 1978 | 0.62 | 0.57 | 0.7 | 0.49 | 0.68 | 0.83 | 0.96 | 0.9 | 0 | 0 | 0.16 | 0 |
| 3347: 105 | 1980 | 0.76 | 0.21 | 0.45 | 0.2 | 0.19 | 0.84 | 1 | NA | 0 | 0 | 0 | 0.075 |
| 3347: 111 | 1986 | 0.22 | 0.37 | 0.4 | 0.099 | 0.1 | 0.33 | 0.64 | 0.038 | 0.046 | 0.72 | 0 | 0 |
| 3347: 127 | 2002 | 0.31 | 0.5 | 0.33 | 0.61 | 0.53 | 0.54 | 0.52 | 0.43 | 0.16 | 0 | 0 | 0.39 |
| 3347: 133 | 2008 | 0.5 | 0.5 | 0.47 | 0.58 | 0.63 | 0.51 | 0.34 | 0.41 | 0.39 | 0 | 0.33 | 0.3 |
| 3347: 185 | 2060 | 0.64 | 0.76 | 0.67 | 0.81 | 0.82 | 0.91 | 0.63 | 0.63 | 0.23 | 0 | 0.43 | 0.56 |
| 3347: 232 | 2107 | 0.86 | 0.89 | 0.79 | 0.93 | 0.91 | 0.92 | 1 | 0.88 | 0.82 | 1 | 0.65 | 0.73 |
| 3347: 342 | 2217 | 0.24 | 0.4 | 0.27 | 0.47 | 0.34 | 0.7 | 0.55 | 0 | 0.24 | 0 | 0.21 | 0.27 |
| 3347: 351 | 2226 | 0.77 | 0.62 | 0.67 | 0.64 | 0.66 | 0.56 | 1 | NA | 0.5 | 0.65 | 0.58 | 0.52 |

| | MVP | | | | | | | | | | | | | | |
|---|---|---|---|---|---|---|---|---|---|---|---|---|---|---|---|
| CpG | Position | | | | | | | | | | | | | | |
| identifier | in ROI | Muscle | Lung | Lung | Lung | Lung | Lung | Liver | Breast | Breast | Breast | Breast | Breast | Brain | Brain |
| 3347: 32 | 1907 | 0 | 0.041 | NA | 0 | 0.32 | NA | 0.4 | 0 | NA | 0.29 | 0 | 1 | 0 | 0 |
| 3347: 63 | 1938 | 0.55 | 0.56 | 0.64 | 0.6 | 0.76 | 0.83 | 0.82 | 0.88 | 0.95 | 0.75 | NA | 0.76 | 0.27 | 0 |
| 3347: 65 | 1940 | 0.25 | 0.075 | 0.13 | 0.94 | 0.69 | 0 | 0.054 | 0.68 | 0.66 | 0.13 | 0.034 | 0.45 | 0.29 | 0.23 |
| 3347: 71 | 1946 | 0.88 | 0.39 | 0.67 | 1 | 0.64 | 0.27 | 0.58 | 0.73 | 0.72 | 0.62 | 0 | 0.54 | 0.39 | NA |
| 3347: 85 | 1960 | 0.19 | 0.27 | 0.05 | 0 | 0.13 | 0.065 | 0 | 0 | 0.12 | 0.21 | 0 | 0.17 | 0 | 0.16 |
| 3347: 92 | 1967 | NA | 0.18 | 0.033 | 0.48 | 0.17 | 0.37 | NA | 0 | 0.66 | 0.062 | 0 | NA | 0 | 0 |
| 3347: 100 | 1975 | 0 | 0.27 | 0.75 | 1 | 0.61 | 0.41 | 0.8 | 0.25 | 0.28 | 0.077 | 0 | 0.4 | 0.091 | 0.1 |
| 3347: 103 | 1978 | 0.45 | NA | 0.66 | 0.71 | 0.56 | 0.2 | 0.88 | 0.73 | 0.47 | 0.37 | 0.27 | 0.51 | 0.19 | 0.13 |
| 3347: 105 | 1980 | 0.49 | 0.59 | 0.83 | 0.29 | 0.76 | 0.7 | 0.95 | 0.61 | 0.81 | 0.43 | 0.039 | 0.55 | 0.28 | 0.29 |
| 3347: 111 | 1986 | 0.21 | 0.053 | 0.71 | 1 | 0.57 | 0.26 | 0.63 | 0.087 | 0.68 | 0.082 | 0.11 | 0.31 | 0.071 | 0.076 |
| 3347: 127 | 2002 | 0.32 | 0.32 | 0.9 | 0.67 | 0.48 | 0.78 | 0.93 | 0.65 | 0.76 | 0.53 | 0.085 | 0.41 | 0.12 | 0 |
| 3347: 133 | 2008 | 0.25 | 0.16 | 0.77 | 0.95 | 0.7 | 0.65 | 0.8 | 0.61 | 0.79 | 0.61 | 0 | 0.43 | 0.092 | 0.95 |
| 3347: 185 | 2060 | 0.39 | 0.68 | 1 | 1 | 0.88 | 1 | 0.91 | 0.85 | 0.93 | 0.74 | 0.89 | 0.49 | 0.51 | 0.95 |
| 3347: 232 | 2107 | 0.82 | 0.79 | 0.85 | 0.98 | 0.97 | 0.98 | 0.93 | 0.89 | 0.99 | 0.87 | 1 | 0.95 | 0.81 | 0.97 |
| 3347: 342 | 2217 | 0.28 | 0.19 | 0.66 | 0.65 | 0.46 | 0.54 | 0.5 | 0.41 | 0.8 | 0.51 | 1 | 0.28 | 0.2 | 0.0077 |
| 3347: 351 | 2226 | 0.56 | 0.49 | 0.85 | 0.84 | 0.75 | 0.88 | 0.57 | 1 | 0.87 | 0.66 | NA | 0.39 | 0.51 | 0.76 |

| | MVP | | | |
|---|---|---|---|---|
| CpG | Position in | | | |
| identifier | ROI | Brain | Brain | Brain |
| 3347: 32 | 1907 | NA | 0.25 | 1 |
| 3347: 63 | 1938 | 1 | 0.35 | 0.64 |
| 3347: 65 | 1940 | 0.55 | 0.5 | 0.28 |
| 3347: 71 | 1946 | 1 | 0.39 | 0.67 |
| 3347: 85 | 1960 | 0 | 0 | 0 |
| 3347: 92 | 1967 | 0.095 | 0 | 0.067 |
| 3347: 100 | 1975 | 0.54 | 0.17 | 0.35 |
| 3347: 103 | 1978 | 0.86 | 0.39 | 0.5 |
| 3347: 105 | 1980 | 0.47 | 0.43 | 0.34 |
| 3347: 111 | 1986 | 0 | 0 | 0.11 |
| 3347: 127 | 2002 | 0.85 | 0.44 | 0.15 |
| 3347: 133 | 2008 | 0.5 | 0.4 | 0.13 |
| 3347: 185 | 2060 | 0.82 | 0.56 | 0.55 |
| 3347: 232 | 2107 | 1 | 0.63 | 0.72 |
| 3347: 342 | 2217 | 0.43 | 0.15 | 0.052 |
| 3347: 351 | 2226 | 0.41 | 0.46 | 0.27 |

TABLE 32
(3348):

| | MVP | | | | | | | | | | | | |
|---|---|---|---|---|---|---|---|---|---|---|---|---|---|
| | Position | | | | | | | | | | | | |
| CpG | in | | | | | | | | | | | | |
| identifier | ROI | Prostate | Prostate | Prostate | Prostate | Prostate | Prostate | Prostate | Prostate | Muscle | Muscle | Muscle | Muscle |
| 3348: 95 | 1651 | 1 | 1 | 0.93 | 1 | 0.92 | 0.95 | 0.99 | 1 | 0.96 | 1 | 0.54 | 0.96 |
| 3348: 112 | 1668 | 1 | 0.98 | 1 | 1 | 1 | 0.82 | 1 | 0.5 | 0.88 | 1 | 0 | 0.94 |
| 3348: 131 | 1687 | 0.98 | 1 | 1 | 1 | 1 | 0.93 | 1 | 0.61 | 0.97 | 0 | 1 | 1 |
| 3348: 154 | 1710 | 0.96 | 1 | 1 | 1 | 1 | 0.97 | 1 | 0.51 | 1 | 0.84 | 0.68 | 1 |
| 3348: 347 | 1903 | 1 | 1 | 1 | 1 | 1 | 1 | 1 | 1 | 0.87 | 1 | 1 | 0.85 |
| 3348: 352 | 1908 | 1 | 1 | 1 | 1 | 1 | 1 | 1 | 1 | 1 | 1 | 1 | 1 |
| 3348: 355 | 1911 | 0.96 | 1 | 1 | 1 | 1 | 1 | 1 | 1 | 0.84 | 0.98 | 1 | 0.81 |
| 3348: 361 | 1917 | 1 | 1 | 1 | 1 | 1 | 0.96 | 1 | 1 | 0.94 | 1 | 0.12 | 0.83 |
| 3348: 370 | 1926 | 1 | 1 | 1 | 1 | 1 | 1 | 1 | 1 | 0.99 | 1 | 1 | 0.97 |
| 3348: 397 | 1953 | 0.92 | 0.97 | 0.84 | 0.92 | 0.92 | 0.72 | 0.87 | 0.89 | 0.72 | 0.34 | 0 | 0.73 |
| 3348: 439 | 1995 | 0.8 | 1 | 0.67 | 0.93 | 0.97 | 0.91 | 0.95 | 1 | 0.33 | 0 | 1 | 0.56 |
| 3348: 445 | 2001 | 1 | 1 | 1 | 1 | 1 | 1 | 1 | 1 | 1 | 1 | 1 | 1 |

| | MVP | | | | | | | | | | | | | | |
|---|---|---|---|---|---|---|---|---|---|---|---|---|---|---|---|
| CpG | Position | | | | | | | | | | | | | | |
| identifier | in ROI | Muscle | Lung | Lung | Lung | Lung | Lung | Liver | Breast | Breast | Breast | Breast | Breast | Breast | Brain |
| 3348: 95 | 1651 | 0.93 | 0.95 | 1 | 1 | 1 | 1 | 0.56 | 0.88 | 0.86 | 0.84 | 0.87 | 1 | 1 | 1 |
| 3348: 112 | 1668 | 1 | 0.96 | 0.94 | 1 | 1 | 1 | 0.73 | 0.86 | 0.96 | 1 | 0.92 | 1 | 1 | 1 |
| 3348: 131 | 1687 | 0.94 | 1 | 1 | 0.93 | 0.97 | 1 | 0.51 | 0.89 | 0.96 | 0.93 | 0.98 | 1 | 1 | 1 |
| 3348: 154 | 1710 | 1 | 1 | 0.8 | 1 | 1 | 1 | 0.56 | 0.98 | 1 | 1 | 1 | 0.66 | 0.92 | 1 |
| 3348: 347 | 1903 | 0.83 | 1 | 1 | 1 | 1 | 1 | 0.49 | 0.94 | 1 | 0.9 | 1 | 0.88 | 0.6 | 1 |
| 3348: 352 | 1908 | 1 | 1 | 1 | 1 | 1 | 1 | 0.84 | 1 | 1 | 1 | 1 | 1 | 1 | 1 |
| 3348: 355 | 1911 | 0.88 | 1 | 1 | 1 | 1 | 1 | 0.66 | 0.97 | 0.96 | 0.95 | 1 | 0.91 | 1 | 1 |
| 3348: 361 | 1917 | 0.95 | 1 | 1 | 1 | 1 | 1 | 0.6 | 0.98 | 0.96 | 0.91 | 0.96 | 1 | 1 | 1 |
| 3348: 370 | 1926 | 1 | 1 | 1 | 1 | 1 | 1 | 0.51 | 1 | 1 | 1 | 1 | 0.73 | 1 | 1 |
| 3348: 397 | 1953 | 0.69 | 0.91 | 1 | 0.92 | 0.98 | 1 | 0.41 | 0.91 | 0.93 | 1 | 0.94 | 1 | 1 | 0.91 |
| 3348: 439 | 1995 | 0.42 | 0.92 | NA | 0.94 | 0.66 | 1 | 0.5 | 0.47 | 0.55 | 0.65 | 0.76 | 1 | 0.63 | 1 |
| 3348: 445 | 2001 | 1 | 1 | NA | 1 | 1 | 1 | 0.86 | 1 | 1 | 1 | 1 | 1 | 1 | 1 |

| CpG | MVP | | | | |
|---|---|---|---|---|---|
| identifier | Position in ROI | Brain | Brain | Brain | Brain |
| 3348: 95 | 1651 | 1 | 1 | 1 | 1 |
| 3348: 112 | 1668 | 0.89 | 1 | 1 | 1 |
| 3348: 131 | 1687 | 1 | 1 | 1 | 1 |
| 3348: 154 | 1710 | 0.8 | 0.81 | 1 | 1 |
| 3348: 347 | 1903 | 0.97 | 1 | 1 | 1 |
| 3348: 352 | 1908 | 1 | 1 | 1 | 1 |
| 3348: 355 | 1911 | 0.97 | 1 | 1 | 1 |
| 3348: 361 | 1917 | 1 | 1 | 1 | 1 |
| 3348: 370 | 1926 | 1 | 1 | 1 | 1 |
| 3348: 397 | 1953 | 1 | 1 | 0.92 | 0.9 |
| 3348: 439 | 1995 | 1 | 1 | 1 | 0.96 |
| 3348: 445 | 2001 | 1 | 1 | 1 | 1 |

TABLE 33
(3364):

| | MVP | | | | | | | | | | | | |
|---|---|---|---|---|---|---|---|---|---|---|---|---|---|
| | Position | | | | | | | | | | | | |
| CpG | in | | | | | | | | | | | | |
| identifier | ROI | Prostate | Prostate | Prostate | Prostate | Prostate | Prostate | Prostate | Prostate | Muscle | Muscle | Muscle | Muscle |
| 3364: 33 | 1921 | 0.87 | 1 | 0.73 | 0.9 | 1 | 1 | NA | 0.88 | 0.89 | NA | 1 | NA |
| 3364: 117 | 2005 | 0.62 | 0.78 | 1 | 0.78 | 0.8 | 0.93 | 1 | 0.78 | 1 | 1 | 1 | 1 |
| 3364: 142 | 2030 | 0.62 | 0.91 | 0.79 | 0.93 | 0.89 | 1 | 1 | 0.8 | 0.78 | 1 | 1 | 0.74 |
| 3364: 163 | 2051 | 0.84 | 0.95 | 1 | 1 | 1 | 1 | 1 | 1 | 1 | 1 | 1 | 1 |
| 3364: 168 | 2056 | 0.72 | 0.95 | 0.82 | 0.95 | 1 | 1 | 0.92 | 1 | 1 | 1 | 1 | 1 |
| 3364: 204 | 2092 | 0.76 | 0.9 | NA | 0.9 | NA | 0.88 | 0.95 | 0.56 | 0.57 | 0.91 | 0.98 | NA |
| 3364: 251 | 2139 | 0.54 | 0.7 | 0.61 | 0.81 | 0.62 | 0.7 | 0.75 | 0.56 | 0.45 | 0.45 | 0.6 | 0.62 |
| 3364: 423 | 2311 | 0.86 | NA | 1 | 1 | NA | 1 | 1 | 1 | 1 | 1 | 1 | NA |
| 3364: 431 | 2319 | 0.77 | NA | 0.73 | 1 | 1 | 1 | 0.91 | 1 | 0.59 | 1 | 0.93 | 1 |
| 3364: 445 | 2333 | 0.73 | NA | NA | 1 | 1 | 1 | 1 | 0.82 | 0.81 | 1 | 1 | 1 |
| 3364: 471 | 2359 | NA | NA | 0.51 | 1 | NA | 1 | NA | 0 | NA | 1 | 0.5 | 0 |
| 3364: 474 | 2362 | NA | NA | NA | 1 | NA | NA | NA | 0.85 | 0.37 | 1 | 1 | 0.72 |

| | MVP | | | | | | | | | | | | | | |
|---|---|---|---|---|---|---|---|---|---|---|---|---|---|---|---|
| CpG | Position | | | | | | | | | | | | | | |
| identifier | in ROI | Lung | Lung | Lung | Lung | Lung | Liver | Liver | Breast | Breast | Breast | Breast | Breast | Brain | Brain |
| 3364: 33 | 1921 | 1 | NA | NA | 0.87 | NA | NA | NA | NA | NA | 1 | NA | NA | 0.21 | NA |
| 3364: 117 | 2005 | 1 | 1 | 1 | 0.96 | 1 | 1 | 1 | 1 | 1 | 1 | 1 | 0.93 | 1 | NA |
| 3364: 142 | 2030 | 1 | 1 | 1 | 1 | 0.5 | 1 | 1 | 0.95 | 0.93 | 1 | 0.93 | 0.98 | 0.64 | 0.11 |
| 3364: 163 | 2051 | 1 | 1 | 1 | 1 | 1 | 1 | 1 | 1 | 1 | 1 | 1 | 1 | 0.83 | 0.062 |
| 3364: 168 | 2056 | 1 | 1 | 1 | 1 | 1 | 1 | 1 | 1 | 1 | 1 | 1 | 0.92 | 0.76 | 0.63 |
| 3364: 204 | 2092 | 0.79 | 0.84 | 0.85 | 0.83 | 1 | 1 | 1 | 1 | 0.93 | 0.85 | 0.4 | 0.96 | 0.45 | 0 |
| 3364: 251 | 2139 | 0.68 | 0.85 | 0.89 | 0.79 | 0.95 | 1 | NA | 0.66 | 0.65 | 0.64 | 0.86 | 0.63 | 0.44 | 0.46 |
| 3364: 423 | 2311 | 1 | 1 | 1 | 1 | NA | NA | NA | 1 | 1 | 1 | 1 | 1 | 0.74 | NA |
| 3364: 431 | 2319 | 0.8 | 1 | 0.64 | 0.95 | 1 | NA | NA | 1 | 0.85 | 0.89 | 0.68 | 1 | 0.45 | NA |
| 3364: 445 | 2333 | 1 | 1 | 1 | NA | 0.82 | NA | NA | 1 | 1 | 0.92 | 1 | 0.93 | 0.73 | NA |
| 3364: 471 | 2359 | 1 | 1 | 1 | 0 | NA | NA | NA | 1 | 1 | 1 | 1 | 1 | 1 | NA |
| 3364: 474 | 2362 | 1 | 1 | 1 | 1 | NA | NA | NA | NA | 1 | 1 | 1 | 1 | 1 | NA |

| | MVP | | | |
|---|---|---|---|---|
| CpG identifier | Position in ROI | Brain | Brain | Brain |
| 3364: 33 | 1921 | NA | NA | 0.59 |
| 3364: 117 | 2005 | 1 | 0.79 | 0.8 |
| 3364: 142 | 2030 | 0.93 | 0.43 | 0.48 |
| 3364: 163 | 2051 | 0.75 | 0.61 | 0.72 |
| 3364: 168 | 2056 | 0.93 | 0.55 | 0.61 |
| 3364: 204 | 2092 | 1 | 0.44 | 0.33 |
| 3364: 251 | 2139 | 0.58 | 0.47 | 0.48 |
| 3364: 423 | 2311 | NA | 0.88 | NA |
| 3364: 431 | 2319 | NA | 0.55 | 1 |
| 3364: 445 | 2333 | 0.74 | 0.73 | 0.64 |
| 3364: 471 | 2359 | 0.49 | 0.16 | NA |
| 3364: 474 | 2362 | NA | 0.65 | NA |

TABLE 34
(3374):

| | MVP | | | | | | | | | | | | |
|---|---|---|---|---|---|---|---|---|---|---|---|---|---|
| | Position | | | | | | | | | | | | |
| CpG | in | | | | | | | | | | | | |
| identifier | ROI | Prostate | Prostate | Prostate | Prostate | Prostate | Prostate | Prostate | Prostate | Muscle | Muscle | Muscle | Muscle |
| 3374: 38 | 979 | 0.82 | 0.88 | 0.46 | 0.73 | 0.56 | 0 | 0.62 | 0.73 | 0.36 | 0.54 | 0.64 | 0.22 |
| 3374: 89 | 1030 | 0.91 | 1 | 0.81 | 0.71 | 0.83 | 0.89 | 0.89 | 0.9 | 0.55 | 0.48 | 0.75 | 0.51 |
| 3374: 98 | 1039 | 1 | 1 | 1 | 0.99 | 1 | 0.98 | 1 | 1 | 0.97 | 0.98 | 0.83 | 1 |
| 3374: 117 | 1058 | 0.89 | 0.98 | 1 | 0.97 | 0.98 | 0.93 | 0.96 | 0.94 | 0.88 | 0.47 | 0.93 | 0.92 |
| 3374: 238 | 1179 | 0.98 | 1 | 1 | 1 | 1 | 1 | 1 | 1 | 1 | 1 | 1 | 0.96 |
| 3374: 255 | 1196 | 1 | 1 | 1 | 1 | 0.98 | 1 | 1 | 1 | 1 | 1 | 1 | 1 |
| 3374: 280 | 1221 | 1 | 0.98 | 1 | 1 | 0.98 | 0.98 | 1 | 1 | 0.98 | 0.98 | 1 | 0.95 |
| 3374: 309 | 1250 | 0.83 | 0.93 | 0.83 | 0.87 | 0.79 | 0.58 | 0.75 | 0.93 | 0.81 | 1 | 0.72 | 0.84 |
| 3374: 350 | 1291 | 0.95 | 1 | 0.89 | 0.92 | 0.85 | 0.92 | 0.94 | 1 | 0.93 | 0.68 | 0.96 | 0.91 |
| 3374: 449 | 1390 | 0.87 | 0.74 | 0.76 | 0.64 | 0.65 | 0.52 | 0.71 | 0.84 | 0.57 | 0.87 | 1 | 0.7 |

| CpG | MVP | | | | | | | | | | | | | |
|---|---|---|---|---|---|---|---|---|---|---|---|---|---|---|
| identifier | Position in ROI | Muscle | Lung | Lung | Lung | Lung | Lung | Liver | Liver | Breast | Breast | Breast | Breast | Breast |
| 3374: 38 | 979 | 0.49 | 0.55 | 0.85 | 1 | 0.76 | 0.87 | 0.87 | 0.44 | 0.59 | 0.55 | 0.2 | 0.18 | 0.49 |
| 3374: 89 | 1030 | 0.58 | 0.79 | 0.94 | 0.65 | 0.81 | 0.86 | 0.94 | 1 | 0.65 | 0.77 | 0.69 | 0.77 | 0.65 |
| 3374: 98 | 1039 | 1 | 1 | 1 | 0.86 | 1 | 1 | 1 | 1 | 0.97 | 1 | 0.99 | 0.68 | 0.98 |
| 3374: 117 | 1058 | 0.91 | 0.93 | 0.99 | 1 | 0.96 | 0.96 | 0.94 | 1 | 0.92 | 0.93 | 0.96 | 0.88 | 0.89 |
| 3374: 238 | 1179 | 1 | 1 | 1 | 1 | 1 | 1 | 0.99 | 1 | 1 | 1 | 1 | 1 | 1 |
| 3374: 255 | 1196 | 1 | 1 | 1 | 1 | 1 | 1 | 1 | 1 | 1 | 1 | 1 | 1 | 1 |
| 3374: 280 | 1221 | 0.99 | 1 | 0.98 | 1 | 1 | 1 | 1 | 1 | 0.96 | 1 | 1 | 1 | 0.98 |
| 3374: 309 | 1250 | 0.76 | 0.89 | 0.9 | 0.64 | 0.9 | 0.91 | 0.98 | 0.73 | 0.65 | 0.68 | 0.77 | 0.54 | 0.71 |
| 3374: 350 | 1291 | 0.92 | 0.93 | 0.97 | 0.97 | 0.95 | 0.99 | 0.93 | 0.88 | 0.91 | 0.95 | 0.88 | 0.98 | 0.84 |
| 3374: 449 | 1390 | 0.72 | 0.89 | 0.92 | 0.97 | 0.85 | 1 | 0.9 | 0.82 | 0.95 | 1 | 0.85 | 0.39 | 0.86 |

| | MVP | | | | | |
|---|---|---|---|---|---|---|
| CpG | Position in | | | | | |
| identifier | ROI | Brain | Brain | Brain | Brain | Brain |
| 3374: 38 | 979 | 0.9 | 1 | 1 | 0.92 | 0.76 |
| 3374: 89 | 1030 | 0.9 | 0.94 | 0.88 | 0.84 | 1 |
| 3374: 98 | 1039 | 1 | 0.97 | 1 | 1 | 1 |
| 3374: 117 | 1058 | 0.93 | 1 | 1 | 0.96 | 0.91 |
| 3374: 238 | 1179 | 1 | 0.96 | 1 | 1 | 1 |
| 3374: 255 | 1196 | 1 | 1 | 1 | 1 | 1 |
| 3374: 280 | 1221 | 1 | 1 | 1 | 0.97 | 1 |
| 3374: 309 | 1250 | 0.95 | 0.76 | 0.39 | 0.9 | 0.83 |
| 3374: 350 | 1291 | 1 | 0.87 | 1 | 0.99 | 0.97 |
| 3374: 449 | 1390 | 0.92 | 0.88 | 0.88 | 0.78 | 0.74 |

TABLE 35
(3377):

| | MVP | | | | | | | | | | | | |
|---|---|---|---|---|---|---|---|---|---|---|---|---|---|
| | Position | | | | | | | | | | | | |
| CpG | in | | | | | | | | | | | | |
| identifier | ROI | Prostate | Prostate | Prostate | Prostate | Prostate | Prostate | Prostate | Prostate | Muscle | Muscle | Muscle | Muscle |
| 3377: 30 | 2036 | NA | NA | NA | NA | NA | NA | NA | NA | NA | NA | NA | NA |
| 3377: 83 | 2089 | 1 | 1 | 1 | 1 | 1 | 1 | 1 | 1 | 1 | 0.93 | 1 | 1 |
| 3377: 109 | 2115 | 0.88 | 1 | 1 | 1 | 1 | 1 | 1 | 1 | 0.96 | 1 | 0.87 | 0.86 |
| 3377: 183 | 2189 | 0.79 | 0.81 | 0.72 | 0.78 | 0.75 | 0.74 | 0.79 | 0.77 | 0.74 | 1 | 0.85 | 0.68 |
| 3377: 222 | 2228 | 0.77 | 0.79 | 0.67 | 0.62 | 0.78 | 0.72 | 0.75 | 0.65 | 0.7 | 0.9 | 0.65 | 0.88 |
| 3377: 235 | 2241 | 0.96 | 1 | 1 | 1 | 1 | 1 | 1 | 0.98 | 1 | 0.68 | 0.84 | 1 |
| 3377: 261 | 2267 | 1 | 1 | 1 | 1 | 0.97 | 1 | 1 | 1 | 0.9 | 0.85 | 0.89 | 1 |
| 3377: 270 | 2276 | 0.86 | 0.94 | 1 | 1 | 0.96 | 0.91 | 1 | 1 | 0.8 | 1 | 0.77 | 1 |
| 3377: 272 | 2278 | 1 | 0.97 | 0.91 | 0.96 | 1 | 0.97 | 1 | 0.93 | 0.89 | 0.75 | 1 | 0.92 |
| 3377: 275 | 2281 | 0.82 | 0.84 | 0.42 | 0.74 | 0.35 | 0.78 | 0.85 | 0.8 | 0.7 | 1 | 0.85 | 0.77 |
| 3377: 327 | 2333 | 0.34 | 0.39 | 0.45 | 0.3 | 0.33 | 0.42 | 0.4 | 0.45 | 0.31 | 0.45 | 0.21 | 0.24 |

| CpG | MVP | | | | | | | | | | | | | |
|---|---|---|---|---|---|---|---|---|---|---|---|---|---|---|
| identifier | Position in ROI | Muscle | Lung | Lung | Lung | Lung | Lung | Liver | Liver | Breast | Breast | Breast | Breast | Breast |
| 3377: 30 | 2036 | NA | NA | NA | NA | NA | NA | NA | 0 | NA | NA | NA | NA | NA |
| 3377: 83 | 2089 | 1 | 1 | 1 | 1 | 1 | 1 | 1 | 0.82 | 1 | 1 | 1 | 0.7 | 1 |
| 3377: 109 | 2115 | 1 | 1 | 1 | 1 | 1 | 1 | 1 | 1 | 1 | 1 | 1 | 0.75 | 1 |
| 3377: 183 | 2189 | 0.68 | 0.81 | 0.93 | 0.84 | 0.79 | 0.86 | 0.95 | 0.7 | 0.65 | 0.73 | 0.68 | 1 | 0.74 |
| 3377: 222 | 2228 | 0.82 | 0.8 | 0.84 | 0.81 | 0.8 | 0.81 | 1 | 1 | 0.61 | 0.71 | 0.62 | 0.84 | 0.68 |
| 3377: 235 | 2241 | 1 | 1 | 1 | 1 | 1 | 1 | 1 | 0.95 | 0.95 | 1 | 0.96 | 0.85 | 0.96 |
| 3377: 261 | 2267 | 1 | 0.95 | 1 | 1 | 1 | 1 | 1 | 0.64 | 0.89 | 0.95 | 0.83 | 1 | 0.94 |
| 3377: 270 | 2276 | 0.89 | 0.92 | 1 | 0.96 | 1 | 0.89 | 0.96 | 1 | 0.89 | 0.77 | 0.78 | 0.96 | 0.84 |
| 3377: 272 | 2278 | 0.88 | 0.95 | 0.96 | 1 | 0.97 | 1 | 1 | 1 | 0.79 | 0.7 | 0.74 | 1 | 0.68 |
| 3377: 275 | 2281 | 0.43 | 0.52 | 0.8 | 0.47 | 0.84 | 0.89 | 0.5 | 0.89 | 0.39 | 0.55 | 0.22 | 0.47 | 0.24 |
| 3377: 327 | 2333 | 0.2 | 0.46 | 0.41 | 0.34 | 0.33 | 0.68 | 0.58 | 0.23 | 0.17 | 0.34 | 0.23 | 0.22 | 0.47 |

| | MVP | | | | | |
|---|---|---|---|---|---|---|
| CpG | Position in | | | | | |
| identifier | ROI | Brain | Brain | Brain | Brain | Brain |
| 3377: 30 | 2036 | NA | NA | NA | NA | NA |
| 3377: 83 | 2089 | 1 | 1 | 1 | 1 | 1 |
| 3377: 109 | 2115 | 1 | 1 | 0.78 | 1 | 0.93 |
| 3377: 183 | 2189 | 0.82 | 0.81 | 0.67 | 0.79 | 0.76 |
| 3377: 222 | 2228 | 0.75 | 1 | 1 | 0.7 | 0.68 |
| 3377: 235 | 2241 | 1 | 0.87 | 1 | 1 | 1 |
| 3377: 261 | 2267 | 0.93 | 1 | 0.89 | 0.92 | 0.94 |
| 3377: 270 | 2276 | 0.9 | 0.92 | 1 | 0.96 | 0.88 |
| 3377: 272 | 2278 | 0.92 | 0.77 | 1 | 0.97 | 1 |
| 3377: 275 | 2281 | 0.89 | 1 | 1 | 0.41 | 0.8 |
| 3377: 327 | 2333 | 0.6 | 0.21 | 0.42 | 0.34 | 0.59 |

TABLE 36
(3282):

| | MVP | | | | | | | | | | | | |
|---|---|---|---|---|---|---|---|---|---|---|---|---|---|
| | Position | | | | | | | | | | | | |
| CpG | in | | | | | | | | | | | | |
| identifier | ROI | Prostate | Prostate | Prostate | Prostate | Prostate | Prostate | Prostate | Prostate | Muscle | Muscle | Muscle | Muscle |
| 3382: 33 | 1224 | 0.6 | 0.63 | 0.7 | 0.66 | 0.66 | 0.5 | 0.85 | 0.51 | 0.55 | 0.84 | 0.7 | 0.71 |
| 3382: 42 | 1233 | 0.85 | 0.84 | 0.87 | 0.84 | 1 | 0.92 | 0.93 | 0.88 | 0.91 | 0.93 | 0.76 | 0.77 |
| 3382: 63 | 1254 | 0.8 | 0.89 | 0.79 | 0.88 | 0.78 | 0.85 | 0.86 | 0.76 | 0.83 | 0.58 | 0.7 | 0.71 |
| 3382: 231 | 1422 | 0.78 | 0.61 | 0.78 | 0.76 | 0.54 | 0.88 | 0.79 | 0.54 | 0.45 | 0.51 | 0.48 | 0.47 |
| 3382: 248 | 1439 | 0.67 | 0.8 | 0.71 | 0.66 | 0.68 | 0.84 | 0.73 | 0.62 | 0.51 | 0.72 | 0.61 | 0.8 |
| 3382: 257 | 1448 | 0.97 | 0.96 | 0.91 | 0.98 | 0.91 | 0.98 | 0.98 | 0.92 | 0.93 | 0.99 | 0.94 | 1 |
| 3382: 263 | 1454 | 0.84 | 0.8 | 0.86 | 0.8 | 0.79 | 0.76 | 0.83 | 0.7 | 0.66 | 0.74 | 0.67 | 0.66 |
| 3382: 284 | 1475 | 1 | 1 | 0.96 | 1 | 0.91 | 0.87 | 0.96 | 0.93 | 0.97 | 0.98 | 0.91 | 0.94 |
| 3382: 302 | 1493 | 1 | 1 | 0.94 | 1 | 0.96 | 0.99 | 1 | 1 | 0.96 | 0.93 | 1 | 0.96 |
| 3382: 308 | 1499 | 0.9 | 0.91 | 0.82 | 0.87 | 0.9 | 0.9 | 0.94 | 0.9 | 0.84 | 0.74 | 0.82 | 0.85 |
| 3382: 314 | 1505 | 0.96 | 1 | 0.99 | 1 | 0.92 | 0.97 | 1 | 1 | 1 | 0.99 | 0.96 | 0.9 |
| 3382: 326 | 1517 | 0.97 | 0.95 | 0.95 | 1 | 0.91 | 0.95 | 0.96 | 0.92 | 0.97 | 0.96 | 0.94 | 0.95 |
| 3382: 332 | 1523 | 0.96 | 1 | 1 | 0.95 | 0.97 | 1 | 1 | 0.87 | 1 | 1 | 0.98 | 1 |
| 3382: 347 | 1538 | 0.9 | 1 | 0.85 | 0.79 | 0.86 | 1 | 0.89 | 0.87 | 0.78 | 1 | 0.74 | 0.79 |

| | MVP | | | | | | | | | | | | | | |
|---|---|---|---|---|---|---|---|---|---|---|---|---|---|---|---|
| CpG | Position | | | | | | | | | | | | | | |
| identifier | in ROI | Lung | Lung | Lung | Lung | Lung | Liver | Liver | Breast | Breast | Breast | Breast | Breast | Brain | Brain |
| 3382: 33 | 1224 | 0.58 | 0.8 | 0.77 | 0.65 | 0.73 | 0.37 | 0.13 | 0.42 | 0.23 | 0.38 | 0.34 | 0.46 | 0.4 | 0.44 |
| 3382: 42 | 1233 | 0.67 | 0.93 | 0.91 | 0.78 | 0.74 | 0.73 | 0.48 | 0.84 | 0.53 | 0.77 | 0.39 | 0.72 | 1 | 0.72 |
| 3382: 63 | 1254 | 0.56 | 0.83 | 0.69 | 0.77 | 0.76 | 0.55 | 0.14 | 0.53 | 0.53 | 0.62 | 0.25 | 0.57 | 0.53 | 0.72 |
| 3382: 231 | 1422 | 0.53 | 0.63 | 0.6 | 0.66 | 0.72 | 0.87 | 0.71 | 0.28 | 0.26 | 0.46 | 0.42 | 0.39 | 0.52 | 0.42 |
| 3382: 248 | 1439 | 0.62 | 0.82 | 0.72 | 0.73 | 0.76 | 0.9 | 0.67 | 0.45 | 0.37 | 0.19 | 0.65 | 0.68 | 0.26 | 0.42 |
| 3382: 257 | 1448 | 0.83 | 0.88 | 0.72 | 1 | 0.98 | 0.91 | 0.86 | 0.8 | 0.91 | 0.62 | 0.88 | 0.52 | 0.63 | 0.82 |
| 3382: 263 | 1454 | 0.68 | 0.94 | 0.54 | 0.67 | 0.82 | 0.84 | 0.7 | 0.43 | 0.42 | 0.36 | 0.4 | 0.45 | 0.37 | 0.66 |
| 3382: 284 | 1475 | 0.93 | 0.92 | 0.96 | 0.97 | 1 | 0.97 | 1 | 0.72 | 0.73 | 0.64 | 0.98 | 0.84 | 0.81 | 0.78 |
| 3382: 302 | 1493 | 0.91 | 1 | 0.99 | 0.99 | 1 | 1 | 0.96 | 0.73 | 1 | 0.8 | 0.96 | 0.78 | 0.88 | 0.84 |
| 3382: 308 | 1499 | 0.83 | 1 | 0.91 | 0.87 | 0.96 | 0.8 | 0.79 | 0.54 | 0.52 | 0.53 | 0.57 | 0.55 | 0.43 | 0.65 |
| 3382: 314 | 1505 | 0.98 | 1 | 1 | 1 | 1 | 0.94 | 0.97 | 0.78 | 0.9 | 0.7 | 0.98 | 0.86 | 0.85 | 0.86 |
| 3382: 326 | 1517 | 0.99 | 0.93 | 0.94 | 1 | 1 | 0.97 | 0.95 | 0.83 | 0.62 | 0.59 | 0.73 | 0.69 | 0.71 | 0.84 |
| 3382: 332 | 1523 | 0.94 | 1 | 1 | 0.98 | 1 | 0.91 | 0.89 | 0.94 | 0.75 | 0.6 | 1 | 1 | 0.66 | 0.89 |
| 3382: 347 | 1538 | 0.88 | 1 | 0.85 | 0.98 | 0.86 | 0.93 | 0.98 | 0.58 | 0.71 | 0.56 | 0.62 | 0.64 | 0.6 | 0.78 |

| | MVP | | | | | |
|---|---|---|---|---|---|---|
| CpG | Position in | | | | | |
| identifier | ROI | Brain | Brain | Brain | Brain | Brain |
| 3382: 33 | 1224 | 0.67 | 1 | 0.75 | 0.43 | 0.35 |
| 3382: 42 | 1233 | 0.94 | 0.14 | 0.95 | 0.93 | 0.64 |
| 3382: 63 | 1254 | 0.79 | 0.75 | 0.91 | 0.79 | 0.69 |
| 3382: 231 | 1422 | 0.33 | 0.29 | 0.51 | 0.42 | 0.23 |
| 3382: 248 | 1439 | 0.5 | 0.12 | 0.32 | 0.51 | 0.44 |
| 3382: 257 | 1448 | 0.81 | 0.19 | 0.9 | 0.92 | 0.81 |
| 3382: 263 | 1454 | 0.65 | 0.62 | 0.61 | 0.6 | 0.48 |
| 3382: 284 | 1475 | 0.87 | 0.76 | 1 | 0.77 | 0.77 |
| 3382: 302 | 1493 | 1 | 0.74 | 0.97 | 0.94 | 0.79 |
| 3382: 308 | 1499 | 0.78 | 0.57 | 0.77 | 0.76 | 0.63 |
| 3382: 314 | 1505 | 1 | 0.64 | 0.98 | 0.9 | 0.87 |
| 3382: 326 | 1517 | 0.93 | 0.75 | 1 | 0.82 | 0.69 |
| 3382: 332 | 1523 | 0.97 | 0.55 | 1 | 0.77 | 0.79 |
| 3382: 347 | 1538 | 0.82 | 0.97 | 0.99 | 0.81 | 0.78 |

TABLE 37
(3083)

| Position of | Position of | | from | | outstanding |
|---|---|---|---|---|---|
| MVP within | MVP within | | other | | marker |
| amplificate | ROI | identifies | types | P value | position |
| 3083: 28 | 442 | liver | all | 0.0152 | ✓ |
| 3083: 31 | 445 | liver | all | 0.0476 | ✓ |
| 3083: 40 | 454 | liver | all | 0.00102 | ✓ |
| 3083: 55 | 469 | liver | all | 0.0167 | ✓ |
| 3083: 61 | 475 | liver | all | 0.0038 | ✓ |
| 3083: 95 | 509 | liver | all | 0.0287 | ✓ |
| 3083: 122 | 536 | liver | all | 0.00984 | ✓ |
| 3083: 143 | 557 | liver | all | 0.0293 | ✓ |
| 3083: 161 | 575 | liver | all | 0.0208 | ✓ |
| 3083: 202 | 616 | liver | all | 7.46E−08 | ✓ |
| 3083: 216 | 630 | liver | all | 0.0145 | ✓ |
| 3083: 235 | 649 | liver | all | 0.0206 | ✓ |
| 3083: 250 | 664 | liver | all | 0.00667 | ✓ |
| 3083: 262 | 676 | liver | all | 0.0215 | ✓ |
| 3083: 265 | 679 | liver | all | 0.0336 | ✓ |
| 3083: 269 | 683 | liver | all | 0.0219 | ✓ |
| 3083: 294 | 708 | liver | all | 0.0046 | ✓ |
| 3083: 299 | 713 | liver | all | 0.0241 | ✓ |

TABLE 38
(3084)

| Position of | Position of | | from | | outstanding |
|---|---|---|---|---|---|
| MVP within | MVP within | | other | | marker |
| amplificate | ROI | identifies | types | P value | position |
| 3084: 41 | 1017 | breast | all | 0.626 | — |
| 3084: 56 | 1032 | breast | all | 0.00904 | ✓ |
| 3084: 69 | 1045 | breast | all | 0.00536 | ✓ |
| 3084: 72 | 1048 | breast | all | 0.0607 | — |
| 3084: 77 | 1053 | breast | all | 0.198 | — |
| 3084: 101 | 1077 | breast | all | 0.0027 | ✓ |
| 3084: 201 | 1177 | breast | all | 0.0877 | — |
| 3084: 276 | 1252 | brain | all | 0.00034 | ✓ |
| 3084: 301 | 1277 | brain | all | 0.000478 | ✓ |
| 3084: 349 | 1325 | brain | all | 5.31E−06 | ✓ |
| 3084: 364 | 1340 | brain | all | 1.06E−05 | ✓ |

TABLE 39
(3091)

| Position of | Position of | | from | | outstanding |
|---|---|---|---|---|---|
| MVP within | MVP within | | other | | marker |
| amplificate | ROI | identifies | types | P value | position |
| 3091: 99 | 1766 | breast | all | 0.159 | — |
| 3091: 159 | 1826 | breast | all | 0.105 | — |
| 3091: 198 | 1865 | breast | all | 0.622 | — |
| 3091: 205 | 1872 | breast | all | 0.11 | — |
| 3091: 217 | 1884 | breast | all | 0.357 | — |
| 3091: 241 | 1908 | breast | all | 0.135 | — |
| 3091: 247 | 1914 | breast | all | 0.293 | — |
| 3091: 257 | 1924 | breast | all | 0.0351 | ✓ |
| 3091: 272 | 1939 | breast | all | 0.162 | — |
| 3091: 281 | 1948 | breast | all | 0.0678 | — |
| 3091: 286 | 1953 | breast | all | 0.592 | — |
| 3091: 303 | 1970 | breast | all | 0.00249 | ✓ |
| 3091: 320 | 1987 | breast | all | 0.00104 | ✓ |
| 3091: 334 | 2001 | breast | all | 0.548 | — |
| 3091: 337 | 2004 | breast | all | 0.00752 | ✓ |
| 3091: 370 | 2037 | breast | all | 0.152 | — |
| 3091: 379 | 2046 | breast | all | 0.0188 | ✓ |
| 3091: 391 | 2058 | breast | all | 0.0503 | — |
| 3091: 449 | 2116 | breast | all | 0.929 | — |

TABLE 40
(3093)

| Position of | Position of | | from | | outstanding |
|---|---|---|---|---|---|
| MVP within | MVP within | | other | | marker |
| amplificate | ROI | identifies | types | P value | position |
| 3093: 24 | 1122 | liver | all | 0.112 | — |
| 3093: 31 | 1129 | liver | all | 0.568 | — |
| 3093: 39 | 1137 | liver | all | 0.741 | — |
| 3093: 99 | 1197 | liver | all | 0.375 | — |
| 3093: 104 | 1202 | liver | all | 0.5 | — |
| 3093: 182 | 1280 | liver | all | 0.0428 | ✓ |
| 3093: 193 | 1291 | liver | all | 0.0354 | ✓ |
| 3093: 217 | 1315 | liver | all | NA | — |
| 3093: 232 | 1330 | liver | all | 0.163 | — |
| 3093: 240 | 1338 | liver | all | 0.139 | — |
| 3093: 247 | 1345 | liver | all | 0.0456 | ✓ |
| 3093: 256 | 1354 | liver | all | 0.491 | — |
| 3093: 258 | 1356 | liver | all | 0.0239 | ✓ |
| 3093: 269 | 1367 | liver | all | 0.893 | — |
| 3093: 277 | 1375 | liver | all | 0.0473 | ✓ |
| 3093: 319 | 1417 | liver | all | 0.0237 | ✓ |
| 3093: 347 | 1445 | liver | all | 0.0562 | — |
| 3093: 358 | 1456 | liver | all | 0.0819 | — |
| 3093: 395 | 1493 | liver | all | 0.507 | — |
| 3093: 398 | 1496 | liver | all | 0.528 | — |
| 3093: 415 | 1513 | liver | all | 0.623 | — |
| 3093: 433 | 1531 | liver | all | 0.871 | — |
| 3093: 440 | 1538 | liver | all | 0.534 | — |

TABLE 41
(3094)

| Position of | Position of | | from | | outstanding |
|---|---|---|---|---|---|
| MVP within | MVP within | | other | | marker |
| amplificate | ROI | identifies | types | P value | position |
| 3094: 79 | 549 | liver | all | 0.0144 | √ |
| 3094: 103 | 573 | liver | all | 0.124 | — |
| 3094: 118 | 588 | liver | all | 0.845 | — |
| 3094: 148 | 618 | liver | all | 0.0177 | √ |
| 3094: 151 | 621 | liver | all | 0.000113 | √ |
| 3094: 155 | 625 | liver | all | NA | — |
| 3094: 162 | 632 | liver | all | 0.0216 | √ |
| 3094: 169 | 639 | liver | all | 0.00245 | √ |
| 3094: 195 | 665 | liver | all | 0.0673 | — |
| 3094: 342 | 812 | liver | all | 0.555 | — |
| 3094: 393 | 863 | liver | all | 0.653 | — |

TABLE 42
(3103)

| Position of | Position of | | from | | outstanding |
|---|---|---|---|---|---|
| MVP within | MVP within | | other | | marker |
| amplificate | ROI | identifies | types | P value | position |
| 3103: 41 | 1752 | liver | all | NA | — |
| 3103: 47 | 1758 | liver | all | 0.643 | — |
| 3103: 76 | 1787 | liver | all | 0.324 | — |
| 3103: 89 | 1800 | liver | all | 0.564 | — |
| 3103: 106 | 1817 | liver | all | 0.263 | — |
| 3103: 152 | 1863 | liver | all | 0.186 | — |
| 3103: 163 | 1874 | liver | all | 0.0597 | — |
| 3103: 190 | 1901 | liver | all | 0.109 | — |
| 3103: 196 | 1907 | liver | all | 0.152 | — |
| 3103: 203 | 1914 | liver | all | 0.0986 | — |
| 3103: 227 | 1938 | liver | all | 0.0574 | — |
| 3103: 231 | 1942 | liver | all | 0.068 | — |
| 3103: 238 | 1949 | liver | all | 0.141 | — |
| 3103: 279 | 1990 | liver | all | 0.0399 | √ |
| 3103: 285 | 1996 | liver | all | NA | — |
| 3103: 292 | 2003 | liver | all | 0.0746 | — |
| 3103: 294 | 2005 | liver | all | 0.0671 | — |
| 3103: 306 | 2017 | liver | all | NA | — |
| 3103: 311 | 2022 | liver | all | 0.104 | — |
| 3103: 317 | 2028 | liver | all | 0.246 | — |
| 3103: 319 | 2030 | liver | all | 0.109 | — |
| 3103: 333 | 2044 | liver | all | 0.048 | √ |
| 3103: 346 | 2057 | liver | all | NA | — |
| 3103: 365 | 2076 | liver | all | NA | — |
| 3103: 378 | 2089 | liver | all | 0.0884 | — |
| 3103: 384 | 2095 | liver | all | NA | — |

TABLE 43
(3104)

| Position of | Position of | | from | | outstanding |
|---|---|---|---|---|---|
| MVP within | MVP within | | other | | marker |
| amplificate | ROI | identifies | types | P value | position |
| 3104: 75 | 1818 | liver | all | 0.0358 | √ |
| 3104: 79 | 1822 | liver | all | 0.0199 | √ |
| 3104: 132 | 1875 | liver | all | 0.163 | — |
| 3104: 137 | 1880 | liver | all | 0.0506 | — |
| 3104: 245 | 1988 | liver | all | 0.0402 | √ |
| 3104: 249 | 1992 | liver | all | 0.00809 | √ |
| 3104: 254 | 1997 | liver | all | 0.209 | — |
| 3104: 302 | 2045 | liver | all | 0.316 | — |
| 3104: 306 | 2049 | liver | all | 0.826 | — |
| 3104: 333 | 2076 | liver | all | 0.0609 | — |
| 3104: 349 | 2092 | liver | all | 0.308 | — |
| 3104: 361 | 2104 | liver | all | 0.474 | — |
| 3104: 386 | 2129 | liver | all | 0.411 | — |
| 3104: 425 | 2168 | liver | all | 0.957 | — |
| 3104: 475 | 2218 | liver | all | NA | — |

TABLE 44
(3105)

| Position of | Position of | | from | | outstanding |
|---|---|---|---|---|---|
| MVP within | MVP within | | other | | marker |
| amplificate | ROI | identifies | types | P value | position |
| 3105: 45 | 300 | breast | all | 4.86e−05 | √ |
| 3105: 64 | 319 | breast | all | 0.026 | √ |
| 3105: 73 | 328 | breast | all | 3.78E−05 | √ |
| 3105: 85 | 340 | breast | all | 6.74E−05 | √ |
| 3105: 97 | 352 | breast | all | 0.152 | — |
| 3105: 132 | 387 | breast | all | 0.000617 | √ |
| 3105: 136 | 391 | breast | all | 0.00215 | √ |
| 3105: 151 | 406 | breast | all | 0.000385 | √ |
| 3105: 163 | 418 | breast | all | 0.000556 | √ |
| 3105: 172 | 427 | breast | all | 0.00529 | √ |
| 3105: 193 | 448 | breast | all | 0.000129 | √ |
| 3105: 202 | 457 | breast | all | 0.00136 | √ |
| 3105: 256 | 511 | breast | all | 0.00171 | √ |
| 3105: 280 | 535 | breast | all | 0.00685 | √ |
| 3105: 301 | 556 | breast | all | 0.21 | — |
| 3105: 337 | 592 | breast | all | 0.0455 | √ |
| 3105: 364 | 619 | breast | all | 0.00288 | √ |
| 3105: 367 | 622 | breast | all | 0.174 | — |
| 3105: 375 | 630 | breast | all | 0.0666 | — |
| and | | | | | |
| 3105: 45 | 300 | muscle | pros- | 0.243 | — |
| | | | tate, | | |
| | | | liver, | | |
| | | | brain, | | |
| | | | lung | | |
| 3105: 64 | 319 | muscle | all | 0.00724 | √ |
| 3105: 73 | 328 | muscle | all | 0.961 | — |
| 3105: 85 | 340 | muscle | all | 0.493 | — |
| 3105: 97 | 352 | muscle | all | 0.159 | — |
| 3105: 132 | 387 | muscle | all | 0.206 | — |
| 3105: 136 | 391 | muscle | all | 0.0999 | — |
| 3105: 151 | 406 | muscle | all | 0.516 | — |
| 3105: 163 | 418 | muscle | all | 0.0952 | — |
| 3105: 172 | 427 | muscle | all | 0.689 | — |
| 3105: 193 | 448 | muscle | all | 0.285 | — |
| 3105: 202 | 457 | muscle | all | 0.752 | — |
| 3105: 256 | 511 | muscle | all | 0.0069 | √ |
| 3105: 280 | 535 | muscle | all | 0.00173 | √ |
| 3105: 301 | 556 | muscle | all | 0.00199 | √ |
| 3105: 337 | 592 | muscle | all | 0.000502 | √ |
| 3105: 364 | 619 | muscle | all | 0.331 | — |
| 3105: 367 | 622 | muscle | all | 0.0113 | √ |
| 3105: 375 | 630 | muscle | all | 0.00565 | √ |

TABLE 45
(3107)

| Position of | | | | | outstanding |
|---|---|---|---|---|---|
| MVP within | Position of | | from other | | marker |
| amplificate | MVP within ROI | identifies | types | P value | position |
| 3107: 58 | 336 | brain | breast, lung | 0.161 | — |
| 3107: 60 | 338 | brain | breast, lung | 0.572 | — |
| 3107: 80 | 358 | brain | breast, lung | 0.352 | — |
| 3107: 97 | 375 | brain | breast, lung | 0.352 | — |
| 3107: 100 | 378 | brain | breast, lung | 0.527 | — |
| 3107: 120 | 398 | brain | breast, lung | 0.028 | √ |
| 3107: 137 | 415 | brain | breast, lung | 0.667 | — |
| 3107: 139 | 417 | brain | breast, lung | 0.668 | — |
| 3107: 148 | 426 | brain | breast, lung | 0.853 | — |
| 3107: 164 | 442 | brain | breast, lung | 0.354 | — |
| 3107: 187 | 465 | brain | breast, lung | 0.371 | — |
| 3107: 190 | 468 | brain | breast, lung | 0.513 | — |
| 3107: 209 | 487 | brain | breast, lung | 0.0142 | √ |
| 3107: 224 | 502 | brain | breast, lung | 0.0193 | √ |
| 3107: 233 | 511 | brain | breast, lung | 0.00466 | √ |
| 3107: 243 | 521 | brain | breast, lung | 0.0127 | √ |
| 3107: 257 | 535 | brain | breast, lung | 0.0127 | √ |
| 3107: 265 | 543 | brain | breast, lung | 0.00799 | √ |
| 3107: 400 | 678 | brain | breast, lung | 0.0773 | — |
| and | | | | | |
| 3107: 58 | 336 | breast, lung | all | 0.124 | — |
| 3107: 60 | 338 | breast, lung | all | 0.807 | — |
| 3107: 80 | 358 | breast, lung | all | 0.333 | — |
| 3107: 97 | 375 | breast, lung | all | 0.685 | — |
| 3107: 100 | 378 | breast, lung | all | 0.211 | — |
| 3107: 120 | 398 | breast, lung | all | 0.0493 | √ |
| 3107: 137 | 415 | breast, lung | all | 0.273 | — |
| 3107: 139 | 417 | breast, lung | all | 0.125 | — |
| 3107: 148 | 426 | breast, lung | all | 0.161 | — |
| 3107: 164 | 442 | breast, lung | all | 0.0666 | — |
| 3107: 187 | 465 | breast, lung | all | 0.266 | — |
| 3107: 190 | 468 | breast, lung | all | 0.266 | — |
| 3107: 209 | 487 | breast, lung | all | 0.0139 | √ |
| 3107: 224 | 502 | breast, lung | all | 0.00911 | √ |
| 3107: 233 | 511 | breast, lung | all | 0.0185 | √ |
| 3107: 243 | 521 | breast, lung | all | 0.000884 | √ |
| 3107: 257 | 535 | breast, lung | all | 0.0045 | √ |
| 3107: 265 | 543 | breast, lung | all | 0.000936 | √ |
| 3107: 400 | 678 | breast, lung | all | 0.0902 | — |

TABLE 46
(3110)

| Position of | | | | | outstanding |
|---|---|---|---|---|---|
| MVP within | Position of | | from other | | marker |
| amplificate | MVP within ROI | identifies | types | P value | position |
| 3110: 32 | 442 | breast, brain, | liver, lung, | 0.2150 | — |
| | | muscle | prostate | | |
| 3110: 84 | 445 | breast, brain, | liver, lung, | 0.00146 | √ |
| | | muscle | prostate | | |
| 3110: 286 | 454 | breast, brain, | liver, lung, | 0.000644 | √ |
| | | muscle | prostate | | |
| 3110: 310 | 469 | breast, brain, | liver, lung, | 0.000156 | √ |
| | | muscle | prostate | | |
| 3110: 366 | 475 | breast, brain, | liver, lung, | 0.0045 | √ |
| | | muscle | prostate | | |
| 3110: 370 | 509 | breast, brain, | liver, lung, | 0.0246 | √ |
| | | muscle | prostate | | |
| 3110: 415 | 536 | breast, brain, | liver, lung, | 0.108 | — |
| | | muscle | prostate | | |

TABLE 47
(3113)

| Position of | | | | | outstanding |
|---|---|---|---|---|---|
| MVP within | Position of | | from other | | marker |
| amplificate | MVP within ROI | identifies | types | P value | position |
| 3113: 42 | 61 | breast, liver, | brain, lung | 0.0432 | √ |
| | | muscle | | | |
| 3113: 47 | 66 | breast, liver, | brain, lung | 0.321 | — |
| | | muscle | | | |
| 3113: 72 | 91 | breast, liver, | brain, lung | 0.013 | √ |
| | | muscle | | | |
| 3113: 78 | 97 | breast, liver, | brain, lung | 0.0000741 | √ |
| | | muscle | | | |
| 3113: 86 | 105 | breast, liver, | brain, lung | 0.0000488 | √ |
| | | muscle | | | |
| 3113: 116 | 135 | breast, liver, | brain, lung | 0.0000893 | √ |
| | | muscle | | | |
| 3113: 156 | 175 | breast, liver, | brain, lung | 0.000525 | √ |
| | | muscle | | | |
| 3113: 160 | 179 | breast, liver, | brain, lung | 0.000508 | √ |
| | | muscle | | | |
| 3113: 164 | 183 | breast, liver, | brain, lung | 0.000217 | √ |
| | | muscle | | | |
| 3113: 182 | 201 | breast, liver, | brain, lung | 0.000637 | √ |
| | | muscle | | | |
| 3113: 189 | 208 | breast, liver, | brain, lung | 0.000212 | √ |
| | | muscle | | | |
| 3113: 197 | 216 | breast, liver, | brain, lung | 0.0027 | √ |
| | | muscle | | | |
| 3113: 298 | 317 | breast, liver, | brain, lung | 0.8 | — |
| | | muscle | | | |
| 3113: 303 | 322 | breast, liver, | brain, lung | 0.00676 | √ |
| | | muscle | | | |
| 3113: 378 | 397 | breast, liver, | brain, lung | 0.00615 | √ |
| | | muscle | | | |
| 3113: 400 | 419 | breast, liver, | brain, lung | 0.0046 | √ |
| | | muscle | | | |
| 3113: 406 | 425 | breast, liver, | brain, lung | 0.0585 | — |
| | | muscle | | | |

TABLE 48
(3127)

| Position of | Position of | | from | | outstanding |
|---|---|---|---|---|---|
| MVP within | MVP within | | other | | marker |
| amplificate | ROI | identifies | types | P value | position |
| 3127: 25 | 1756 | breast | all | 0.00132 | √ |
| 3127: 28 | 1759 | breast | all | 0.0106 | √ |
| 3127: 63 | 1794 | breast | all | 0.00176 | √ |
| 3127: 73 | 1804 | breast | all | 0.00104 | √ |
| 3127: 124 | 1855 | breast | all | 0.0011 | √ |
| 3127: 127 | 1858 | breast | all | 0.0022 | √ |
| 3127: 175 | 1906 | breast | all | 0.0279 | √ |

TABLE 49
(3129)

| Position of | Position of | | from | | outstanding |
|---|---|---|---|---|---|
| MVP within | MVP within | | other | | marker |
| amplificate | ROI | identifies | types | P value | position |
| 3129: 99 | 1999 | liver | all | 0.887 | — |
| 3129: 111 | 2011 | liver | all | 0.76 | — |
| 3129: 125 | 2025 | liver | all | 0.672 | — |
| 3129: 137 | 2037 | liver | all | 0.435 | — |
| 3129: 139 | 2039 | liver | all | 0.275 | — |
| 3129: 144 | 2044 | liver | all | 0.31 | — |
| 3129: 148 | 2048 | liver | all | 0.888 | — |
| 3129: 157 | 2057 | liver | all | 0.212 | — |
| 3129: 162 | 2062 | liver | all | 0.698 | — |
| 3129: 178 | 2078 | liver | all | 0.0875 | — |
| 3129: 184 | 2084 | liver | all | 0.0933 | — |
| 3129: 216 | 2116 | liver | all | 0.606 | — |
| 3129: 261 | 2161 | liver | all | 0.0444 | √ |
| 3129: 341 | 2241 | liver | all | 0.0134 | √ |
| 3129: 353 | 2253 | liver | all | 0.105 | — |
| 3129: 357 | 2257 | liver | all | 0.000186 | √ |
| 3129: 368 | 2268 | liver | all | 0.0288 | √ |
| 3129: 371 | 2271 | liver | all | 0.0346 | √ |
| 3129: 377 | 2277 | liver | all | 0.00985 | √ |
| 3129: 384 | 2284 | liver | all | 0.0281 | √ |
| 3129: 402 | 2302 | liver | all | 0.019 | √ |
| 3129: 438 | 2338 | liver | all | 0.286 | — |
| 3129: 453 | 2353 | liver | all | 0.242 | — |
| 3129: 475 | 2375 | liver | all | 0.539 | — |

TABLE 50
(3145)

| | Position | | | | out- |
|---|---|---|---|---|---|
| | of | | | | stand- |
| Position of | MVP | | from | | ing |
| MVP within | within | | other | | marker |
| amplificate | ROI | identifies | types | P value | position |
| 3145: 46 | 664 | liver, muscle | breast, brain | 0.0589 | — |
| 3145: 94 | 712 | liver, muscle | breast, brain | 0.0143 | √ |
| 3145: 102 | 720 | liver, muscle | breast, brain | 0.000709 | √ |
| 3145: 110 | 728 | liver, muscle | breast, brain | 0.000756 | √ |
| 3145: 140 | 758 | liver, muscle | breast, brain | 0.0143 | √ |
| 3145: 158 | 776 | liver, muscle | breast, brain | 0.00656 | √ |
| 3145: 268 | 886 | liver, muscle | breast, brain | 0.0233 | √ |
| 3145: 354 | 972 | liver, muscle | breast, brain | 0.00123 | √ |
| 3145: 388 | 1006 | liver, muscle | breast, brain | 0.00139 | √ |
| 3145: 445 | 1063 | liver, muscle | breast, brain | 0.385 | — |

TABLE 51
(ROI 3152)

| Position of | Position of | | | | outstanding |
|---|---|---|---|---|---|
| MVP within | MVP within | | | | marker |
| amplificate | ROI | identifies | from other types | P value | position |
| 3152: 26 | 1818 | brain, breast, | lung, prostate | 0.808 | — |
| | | muscle | | | |
| 3152: 56 | 1851 | brain, breast, | lung, prostate | 0.0464 | √ |
| | | muscle | | | |
| 3152: 138 | 1933 | brain, breast, | lung, prostate | 0.0516 | — |
| | | muscle | | | |
| 3152: 234 | 2029 | brain, breast, | lung, prostate | 0.000278 | √ |
| | | muscle | | | |
| 3152: 283 | 2078 | brain, breast, | lung, prostate | 0.000919 | √ |
| | | muscle | | | |
| 3152: 361 | 2156 | brain, breast, | lung, prostate | 0.00859 | √ |
| | | muscle | | | |

TABLE 52
(3170)

| Position of | Position of | | from | | outstanding |
|---|---|---|---|---|---|
| MVP within | MVP within | | other | | marker |
| amplificate | ROI | identifies | types | P value | position |
| 3170: 170 | 1858 | lung | all | 0.673 | — |
| 3170: 175 | 1863 | lung | all | 0.755 | — |
| 3170: 353 | 2041 | lung | all | 0.0714 | — |
| 3170: 385 | 2073 | lung | all | 0.0118 | √ |
| 3170: 396 | 2084 | lung | all | 0.00962 | √ |
| 3170: 409 | 2097 | lung | all | 0.0159 | √ |
| 3170: 412 | 2100 | lung | all | 0.0308 | √ |

TABLE 53
(3192)

| Position of | Position of | | | | |
|---|---|---|---|---|---|
| MVP within | MVP within | | | | outstanding |
| amplificate | ROI | identifies | from other types | P value | marker position |
| 3192: 29 | 375 | lung | breast, prostate, | 0.0256 | √ |
| | | | muscle, liver | | |
| 3192: 108 | 454 | lung | breast, prostate, | 0.000715 | √ |
| | | | muscle, liver | | |
| 3192: 128 | 474 | lung | breast, prostate, | 0.00125 | √ |
| | | | muscle, liver | | |
| 3192: 160 | 506 | lung | breast, prostate, | 0.000213 | √ |
| | | | muscle, liver | | |
| 3192: 166 | 512 | lung | breast, prostate, | 0.000715 | √ |
| | | | muscle, liver | | |
| 3192: 172 | 518 | lung | breast, prostate, | 0.000899 | √ |
| | | | muscle, liver | | |
| 3192: 191 | 537 | lung | breast, prostate, | 0.000213 | √ |
| | | | muscle, liver | | |
| 3192: 265 | 611 | lung | breast, prostate, | 0.00221 | √ |
| | | | muscle, liver | | |
| 3192: 268 | 614 | lung | breast, prostate, | 0.00985 | √ |
| | | | muscle, liver | | |
| 3192: 362 | 708 | lung | breast, prostate, | 0.000213 | √ |
| | | | muscle, liver | | |
| 3192: 368 | 714 | lung | breast, prostate, | 0.000882 | √ |
| | | | muscle, liver | | |
| 3192: 427 | 773 | lung | breast, prostate, | 0.178 | — |
| | | | muscle, liver | | |

TABLE 54
(3200)

| Position of | Position of | | from | | outstanding |
|---|---|---|---|---|---|
| MVP within | MVP within | | other | | marker |
| amplificate | ROI | identifies | types | P value | position |
| 3200: 36 | 1897 | liver | all | 0.0534 | — |
| 3200: 49 | 1910 | liver | all | 0.193 | — |
| 3200: 66 | 1927 | liver | all | 0.0276 | √ |
| 3200: 78 | 1939 | liver | all | 0.0043 | √ |
| 3200: 83 | 1944 | liver | all | 0.0086 | √ |
| 3200: 99 | 1960 | liver | all | 0.46 | — |
| 3200: 127 | 1988 | liver | all | 0.0086 | √ |
| 3200: 155 | 2016 | liver | all | 0.294 | — |
| 3200: 160 | 2021 | liver | all | 0.0086 | √ |
| 3200: 169 | 2030 | liver | all | 0.0086 | √ |
| 3200: 178 | 2039 | liver | all | 0.0043 | √ |
| 3200: 192 | 2053 | liver | all | 0.184 | — |
| 3200: 199 | 2060 | liver | all | 0.0086 | √ |
| 3200: 225 | 2086 | liver | all | 0.0086 | √ |
| 3200: 305 | 2166 | liver | all | 0.0219 | √ |
| 3200: 312 | 2173 | liver | all | 0.0043 | √ |
| 3200: 361 | 2222 | liver | all | 0.0644 | — |

TABLE 55
(3208)

| Position of | Position of | | from | | outstanding |
|---|---|---|---|---|---|
| MVP within | MVP within | | other | | marker |
| amplificate | ROI | identifies | types | P value | position |
| 3208: 33 | 729 | liver | all | 0.0376 | √ |
| 3208: 45 | 741 | liver | all | 0.0219 | √ |
| 3208: 69 | 765 | liver | all | 0.048 | √ |
| 3208: 111 | 807 | liver | all | 0.093 | — |
| 3208: 119 | 815 | liver | all | 0.0219 | √ |
| 3208: 127 | 823 | liver | all | 0.00403 | √ |
| 3208: 148 | 844 | liver | all | 0.039 | √ |
| 3208: 164 | 860 | liver | all | 0.0293 | √ |
| 3208: 303 | 999 | liver | all | 0.0321 | √ |
| 3208: 338 | 1034 | liver | all | 0.355 | — |
| 3208: 349 | 1045 | liver | all | 0.11 | — |
| 3208: 371 | 1067 | liver | all | 0.358 | — |
| 3208: 392 | 1088 | liver | all | 0.404 | — |
| 3208: 403 | 1099 | liver | all | 0.695 | — |
| 3208: 436 | 1132 | liver | all | 0.358 | — |
| 3208: 455 | 1151 | liver | all | NA | — |
| 3208: 461 | 1157 | liver | all | NA | — |

TABLE 56
(3239)

| Position of | Position of | | | | outstanding |
|---|---|---|---|---|---|
| MVP within | MVP within | | from other | | marker |
| amplificate | ROI | identifies | types | P value | position |
| 3239: 38 | 623 | breast, prostate | brain, lung, | 0.00402 | √ |
| | | | liver | | |
| 3239: 44 | 629 | breast, prostate | brain, lung, | 0.0622 | — |
| | | | liver | | |
| 3239: 49 | 634 | breast, prostate | brain, lung, | 0.00448 | √ |
| | | | liver | | |
| 3239: 71 | 656 | breast, prostate | brain, lung, | 0.000516 | √ |
| | | | liver | | |
| 3239: 75 | 660 | breast, prostate | brain, lung, | 0.41 | — |
| | | | liver | | |
| 3239: 88 | 673 | breast, prostate | brain, lung, | 0.354 | — |
| | | | liver | | |
| 3239: 141 | 726 | breast, prostate | brain, lung, | 0.212 | — |
| | | | liver | | |
| 3239: 163 | 748 | breast, prostate | brain, lung, | 0.00371 | √ |
| | | | liver | | |
| 3239: 169 | 754 | breast, prostate | brain, lung, | 0.00107 | √ |
| | | | liver | | |
| 3239: 178 | 763 | breast, prostate | brain, lung, | 0.00141 | √ |
| | | | liver | | |
| 3239: 197 | 782 | breast, prostate | brain, lung, | 0.000187 | √ |
| | | | liver | | |
| 3239: 212 | 797 | breast, prostate | brain, lung, | 0.00002020 | √ |
| | | | liver | | |
| 3239: 218 | 803 | breast, prostate | brain, lung, | 0.0152 | √ |
| | | | liver | | |
| 3239: 233 | 818 | breast, prostate | brain, lung, | 0.000225 | √ |
| | | | liver | | |
| 3239: 236 | 821 | breast, prostate | brain, lung, | 0.000271 | √ |
| | | | liver | | |
| 3239: 242 | 827 | breast, prostate | brain, lung, | 8.75E−05 | √ |
| | | | liver | | |
| 3239: 250 | 835 | breast, prostate | brain, lung, | 0.00547 | √ |
| | | | liver | | |
| 3239: 256 | 841 | breast, prostate | brain, lung, | 0.00632 | √ |
| | | | liver | | |
| 3239: 262 | 847 | breast, prostate | brain, lung, | 0.00615 | √ |
| | | | liver | | |
| 3239: 285 | 870 | breast, prostate | brain, lung, | 0.0299 | √ |
| | | | liver | | |
| 3239: 300 | 885 | breast, prostate | brain, lung, | 0.934 | — |
| | | | liver | | |
| 3239: 319 | 904 | breast, prostate | brain, lung, | 0.0123 | √ |
| | | | liver | | |
| 3239: 328 | 913 | breast, prostate | brain, lung, | 0.00291 | √ |
| | | | liver | | |
| 3239: 337 | 922 | breast, prostate | brain, lung, | 0.484 | — |
| | | | liver | | |
| 3239: 340 | 925 | breast, prostate | brain, lung, | 0.056 | — |
| | | | liver | | |
| 3239: 343 | 928 | breast, prostate | brain, lung, | 0.275 | — |
| | | | liver | | |
| 3239: 348 | 933 | breast, prostate | brain, lung, | 0.68 | — |
| | | | liver | | |
| 3239: 354 | 939 | breast, prostate | brain, lung, | 0.00231 | √ |
| | | | liver | | |
| 3239: 360 | 945 | breast, prostate | brain, lung, | 0.261 | — |
| | | | liver | | |
| 3239: 366 | 951 | breast, prostate | brain, lung, | 0.479 | — |
| | | | liver | | |
| 3239: 377 | 962 | breast, prostate | brain, lung, | 0.369 | — |
| | | | liver | | |
| 3239: 421 | 1006 | breast, prostate | brain, lung, | 0.332 | — |
| | | | liver | | |

TABLE 57
(3243)

| Position of | Position of | | from | | outstanding |
|---|---|---|---|---|---|
| MVP within | MVP within | | other | | marker |
| amplificate | ROI | identifies | types | P value | position |
| 3243: 57 | 1576 | Breast | all | 0.196 | — |
| 3243: 63 | 1582 | Breast | all | NA | — |
| 3243: 132 | 1651 | Breast | all | 0.105 | — |
| 3243: 138 | 1657 | Breast | all | 0.0133 | √ |
| 3243: 140 | 1659 | Breast | all | 0.0144 | √ |
| 3243: 155 | 1674 | Breast | all | 0.000866 | √ |
| 3243: 182 | 1701 | Breast | all | 0.00148 | √ |
| 3243: 229 | 1748 | Breast | all | 0.00163 | √ |
| 3243: 252 | 1771 | Breast | all | 0.0695 | — |
| 3243: 263 | 1782 | Breast | all | 0.0194 | √ |
| 3243: 311 | 1830 | Breast | all | 0.0102 | √ |
| 3243: 392 | 1911 | Breast | all | NA | — |

TABLE 58
(3244)

| Position of | Position of | | from | | outstanding |
|---|---|---|---|---|---|
| MVP within | MVP within | iden- | other | | marker |
| amplificate | ROI | tifies | types | P value | position |
| 3244: 40 | 141 | Muscle | all | 0.0149 | √ |
| 3244: 79 | 180 | Muscle | all | 0.714 | — |
| 3244: 173 | 274 | Muscle | all | 0.000189 | √ |
| 3244: 208 | 309 | Muscle | all | 0.00001990 | √ |
| 3244: 217 | 318 | Muscle | all | 0.00000993 | √ |
| 3244: 223 | 324 | Muscle | all | 0.00001990 | √ |
| 3244: 228 | 329 | Muscle | all | 0.0048 | √ |
| 3244: 240 | 341 | Muscle | all | 0.00252 | √ |

TABLE 59
(3252)

| Position of | Position of | | | | outstanding |
|---|---|---|---|---|---|
| MVP within | MVP within | | | | marker |
| amplificate | ROI | identifies | from other types | P value | position |
| 3252: 39 | 740 | breast, muscle | all | 0.251 | — |
| 3252: 43 | 744 | breast, muscle | all | 0.508 | — |
| 3252: 88 | 789 | breast, muscle | all | 0.000727 | √ |
| 3252: 91 | 792 | breast, muscle | all | 0.000777 | √ |
| 3252: 94 | 795 | breast, muscle | all | 0.192 | — |
| 3252: 152 | 853 | breast, muscle | all | 0.00432 | √ |
| 3252: 164 | 865 | breast, muscle | all | 0.00191 | √ |
| 3252: 175 | 876 | breast, muscle | all | 0.00113 | √ |
| 3252: 178 | 879 | breast, muscle | all | 0.0000139 | √ |
| 3252: 199 | 900 | breast, muscle | all | 0.00449 | √ |
| 3252: 206 | 907 | breast, muscle | all | 0.000445 | √ |
| 3252: 242 | 943 | breast, muscle | all | 0.0079 | √ |
| 3252: 297 | 998 | breast, muscle | all | 0.00325 | √ |
| 3252: 303 | 1004 | breast, muscle | all | 0.0107 | √ |
| 3252: 308 | 1009 | breast, muscle | all | 0.04 | √ |
| 3252: 330 | 1031 | breast, muscle | all | 0.0118 | √ |
| 3252: 334 | 1035 | breast, muscle | all | 0.0135 | √ |
| 3252: 347 | 1048 | breast, muscle | all | 0.865 | — |

TABLE 60
(3265)

| Position of | Position of | | from | | outstanding |
|---|---|---|---|---|---|
| MVP within | MVP within | | other | | marker |
| amplificate | ROI | identifies | types | P value | position |
| 3265: 62 | 716 | muscle | all | 0.0285 | √ |
| 3265: 81 | 735 | muscle | all | 0.0393 | √ |
| 3265: 84 | 738 | muscle | all | 0.000496 | √ |
| 3265: 137 | 791 | muscle | all | 0.00386 | √ |
| 3265: 139 | 793 | muscle | all | 0.137 | — |
| 3265: 259 | 913 | muscle | all | 0.00383 | √ |
| 3265: 337 | 991 | muscle | all | 0.0499 | √ |
| 3265: 350 | 1004 | muscle | all | 0.0195 | √ |
| 3265: 362 | 1016 | muscle | all | 0.00732 | √ |
| 3265: 395 | 1049 | muscle | all | 0.00131 | √ |
| 3265: 404 | 1058 | muscle | all | 0.0547 | — |

TABLE 61
(3291)

| Position of | Position of | | from | | outstanding |
|---|---|---|---|---|---|
| MVP within | MVP within | | other | | marker |
| amplificate | ROI | identifies | types | P value | position |
| 42 | 247 | brain | all | 0.0461 | √ |
| 64 | 269 | brain | all | 0.121 | — |
| 71 | 276 | brain | all | 0.00305 | √ |
| 81 | 286 | brain | all | 0.0113 | √ |
| 369 | 574 | brain | all | 0.0304 | √ |

TABLE 62
(3312)

| Position of | Position of | | from | | outstanding |
|---|---|---|---|---|---|
| MVP within | MVP within | | other | | marker |
| amplificate | ROI | identifies | types | P value | position |
| 3312: 71 | 1498 | liver | all | 0.000433 | √ |
| 3312: 95 | 1522 | liver | all | 0.000429 | √ |
| 3312: 103 | 1530 | liver | all | 0.0131 | √ |
| 3312: 119 | 1546 | liver | all | NA | — |
| 3312: 158 | 1585 | liver | all | 0.0738 | — |
| 3312: 167 | 1594 | liver | all | 0.00331 | √ |
| 3312: 193 | 1620 | liver | all | 0.0092 | √ |
| 3312: 215 | 1642 | liver | all | 0.0222 | √ |
| 3312: 223 | 1650 | liver | all | NA | — |
| 3312: 242 | 1669 | liver | all | NA | — |
| 3312: 259 | 1686 | liver | all | 0.456 | — |
| 3312: 273 | 1700 | liver | all | 0.735 | — |
| 3312: 314 | 1741 | liver | all | 0.967 | — |
| 3312: 404 | 1831 | liver | all | 0.867 | — |
| 3312: 412 | 1839 | liver | all | NA | — |

TABLE 63
(3329)

| Position of | Position of | | from | | outstanding |
|---|---|---|---|---|---|
| MVP within | MVP within | | other | | marker |
| amplificate | ROI | identifies | types | P value | position |
| 3329: 52 | 1151 | liver | all | NA | — |
| 3329: 135 | 1234 | liver | all | 0.0182 | √ |
| 3329: 154 | 1253 | liver | all | 0.0216 | √ |
| 3329: 187 | 1286 | liver | all | 0.0191 | √ |
| 3329: 241 | 1340 | liver | all | 0.0206 | √ |
| 3329: 251 | 1350 | liver | all | 0.0144 | √ |
| 3329: 303 | 1402 | liver | all | 0.0219 | √ |
| 3329: 315 | 1414 | liver | all | 0.027 | √ |
| 3329: 420 | 1519 | liver | all | 0.777 | — |
| 3329: 440 | 1539 | liver | all | 0.278 | — |

TABLE 64
(3330)

| Position of | Position of | | from | | outstanding |
|---|---|---|---|---|---|
| MVP within | MVP within | | other | | marker |
| amplificate | ROI | identifies | types | P value | position |
| 3330: 45 | 2033 | lung | muscle | 0.0254 | √ |
| 3330: 127 | 2115 | lung | muscle | 0.0212 | √ |
| 3330: 151 | 2139 | lung | muscle | 0.00794 | √ |
| 3330: 251 | 2239 | lung | muscle | 0.0952 | — |
| 3330: 260 | 2248 | lung | muscle | 0.00794 | √ |
| 3330: 265 | 2253 | lung | muscle | 0.151 | — |
| 3330: 298 | 2286 | lung | muscle | 0.0159 | √ |
| 3330: 311 | 2299 | lung | muscle | 0.0097 | √ |
| 3330: 320 | 2308 | lung | muscle | 0.00794 | √ |
| 3330: 394 | 2382 | lung | muscle | 0.00749 | √ |
| 3330: 401 | 2389 | lung | muscle | 0.156 | — |

TABLE 65
(3347)

| | Position of | | | | |
|---|---|---|---|---|---|
| Position of | MVP | | from | | outstanding |
| MVP within | within | | other | | marker |
| amplificate | ROI | identifies | types | P value | position |
| 3347: 32 | 1907 | muscle, brain | all | 0.0917 | — |
| 3347: 63 | 1938 | muscle, brain | all | 0.00198 | √ |
| 3347: 65 | 1940 | muscle, brain | all | 0.063 | — |
| 3347: 71 | 1946 | muscle, brain | all | 0.525 | — |
| 3347: 85 | 1960 | muscle, brain | all | 0.018 | √ |
| 3347: 92 | 1967 | muscle, brain | all | 0.00117 | √ |
| 3347: 100 | 1975 | muscle, brain | all | 0.0173 | √ |
| 3347: 103 | 1978 | muscle, brain | all | 0.00232 | √ |
| 3347: 105 | 1980 | muscle, brain | all | 0.00776 | √ |
| 3347: 111 | 1986 | muscle, brain | all | 0.00825 | √ |
| 3347: 127 | 2002 | muscle, brain | all | 0.00412 | √ |
| 3347: 133 | 2008 | muscle, brain | all | 0.0132 | √ |
| 3347: 185 | 2060 | muscle, brain | all | 0.00307 | √ |
| 3347: 232 | 2107 | muscle, brain | all | 0.0769 | — |
| 3347: 342 | 2217 | muscle, brain | all | 0.00181 | √ |
| 3347: 351 | 2226 | muscle, brain | all | 0.0062 | √ |

TABLE 66
(3348)

| Position of | Position of | | from | |
|---|---|---|---|---|
| MVP within | MVP within | | other | |
| amplificate | ROI | identifies | types | P value |
| 3348: 95 | 1651 | liver | all | NA |
| 3348: 112 | 1668 | liver | all | NA |
| 3348: 131 | 1687 | liver | all | NA |
| 3348: 154 | 1710 | liver | all | NA |
| 3348: 347 | 1903 | liver | all | NA |
| 3348: 352 | 1908 | liver | all | NA |
| 3348: 355 | 1911 | liver | all | NA |
| 3348: 361 | 1917 | liver | all | NA |
| 3348: 370 | 1926 | liver | all | NA |
| 3348: 397 | 1953 | liver | all | NA |
| 3348: 439 | 1995 | liver | all | NA |
| 3348: 445 | 2001 | liver | all | NA |

TABLE 67
(3364)

| | Position of | | | | |
|---|---|---|---|---|---|
| Position of | MVP | | from | | outstanding |
| MVP within | within | | other | | marker |
| amplificate | ROI | identifies | types | P value | position |
| 3364: 33 | 1921 | brain | all | 0.0289 | √ |
| 3364: 117 | 2005 | brain | all | 0.566 | — |
| 3364: 142 | 2030 | brain | all | 0.00399 | √ |
| 3364: 163 | 2051 | brain | all | 0.000004760 | √ |
| 3364: 168 | 2056 | brain | all | 0.000311 | √ |
| 3364: 204 | 2092 | brain | all | 0.043 | √ |
| 3364: 251 | 2139 | brain | all | 0.0023 | √ |
| 3364: 423 | 2311 | brain | all | 0.000826 | √ |
| 3364: 431 | 2319 | brain | all | 0.169 | — |
| 3364: 445 | 2333 | brain | all | 0.00148 | √ |
| 3364: 471 | 2359 | brain | all | 0.365 | — |
| 3364: 474 | 2362 | brain | all | 0.404 | — |

TABLE 68
(3374)

| | Position of | | | | out- |
|---|---|---|---|---|---|
| Position of | MVP | | from | | standing |
| MVP within | within | | other | | marker |
| amplificate | ROI | identifies | types | P value | position |
| 3374: 38 | 979 | breast, muscle | all | 0.00165 | √ |
| 3374: 89 | 1030 | breast, muscle | all | 0.000046800 | √ |
| 3374: 98 | 1039 | breast, muscle | all | 0.0101 | √ |
| 3374: 117 | 1058 | breast, muscle | all | 0.00102 | √ |
| 3374: 238 | 1179 | breast, muscle | all | 0.766 | — |
| 3374: 255 | 1196 | breast, muscle | all | 0.525 | — |
| 3374: 280 | 1221 | breast, muscle | all | 0.0562 | — |
| 3374: 309 | 1250 | breast, muscle | all | 0.0906 | — |
| 3374: 350 | 1291 | breast, muscle | all | 0.0554 | — |
| 3374: 449 | 1390 | breast, muscle | all | 0.947 | — |

TABLE 69
(3377)

| Position of | Position of | | from | | outstanding |
|---|---|---|---|---|---|
| MVP within | MVP within | | other | | marker |
| amplificate | ROI | identifies | types | P value | position |
| 3377: 30 | 2036 | breast | all | NA | — |
| 3377: 83 | 2089 | breast | all | 0.393 | — |
| 3377: 109 | 2115 | breast | all | 1 | — |
| 3377: 183 | 2189 | breast | all | 0.156 | — |
| 3377: 222 | 2228 | breast | all | 0.0842 | — |
| 3377: 235 | 2241 | breast | all | 0.0263 | √ |
| 3377: 261 | 2267 | breast | all | 0.139 | — |
| 3377: 270 | 2276 | breast | all | 0.0148 | √ |
| 3377: 272 | 2278 | breast | all | 0.0225 | √ |
| 3377: 275 | 2281 | breast | all | 0.00537 | √ |
| 3377: 327 | 2333 | breast | all | 0.208 | — |

TABLE 70
(3382)

| | Position | | | | |
|---|---|---|---|---|---|
| | of | | | | |
| Position of | MVP | | from | | outstanding |
| MVP within | within | | other | | marker |
| amplificate | ROI | identifies | types | P value | position |
| 3382: 33 | 1224 | brain, breast | all | 0.0284 | √ |
| 3382: 42 | 1233 | brain, breast | all | 0.311 | — |
| 3382: 63 | 1254 | brain, breast | all | 0.0775 | — |
| 3382: 231 | 1422 | brain, breast | all | 0.000001370 | √ |
| 3382: 248 | 1439 | brain, breast | all | 0.000003850 | √ |
| 3382: 257 | 1448 | brain, breast | all | 0.000331 | √ |
| 3382: 263 | 1454 | brain, breast | all | 6.38E−07 | √ |
| 3382: 284 | 1475 | brain, breast | all | 0.00073 | √ |
| 3382: 302 | 1493 | brain, breast | all | 0.00394 | √ |
| 3382: 308 | 1499 | brain, breast | all | 0.000000099 | √ |
| 3382: 314 | 1505 | brain, breast | all | 0.000719 | √ |
| 3382: 326 | 1517 | brain, breast | all | 0.00016 | √ |
| 3382: 332 | 1523 | brain, breast | all | 0.0108 | √ |
| 3382: 347 | 1538 | brain, breast | all | 0.00285 | √ |

The following examples provide a description of how the above disclosed markers are used for identification, classification or cataloguing of a tissue, and/or for distinguishing between or among tissues of different tissue types.

### EXAMPLE 2

### The Marker ROI 3083 and the Attendant Epigenetic Map is Used to Identify Liver Tissue as the Source of Origin of a Sample Containing Genomic DNA. A HeavyMethyl™ Assay is Used for Differentiation of Liver Tissue Amongst Other Tissues

The experiments of the following example occur in the setting of a diagnostic laboratory where two tubes, each containing isolated genomic DNA from one of two different tissue samples, are accidentally randomized. It is known, however, that one sample is obtained from a liver biopsy (intended for use in a molecular cancer test), whereas the other sample is derived from muscle cells of a dead body (intended for use with a SNP-based test for forensic studies). A lack of sufficient tissue material to repeat the extraction (DNA isolation) leads to a decision to quickly test each DNA for its source of origin using one of the inventive liver markers out of a group of several, as disclosed herein above according to the present invention.

According to the present invention, the marker used is the ROI 3083 (nt 571 to nt 3071 in properdin (BF); gene accession gi: 25070930). As disclosed herein, specific regions of said gene are unmethylated in liver but methylated in other tissues (see Tables 3 and 37, herein above). It is also disclosed that this can be utilized in a test by performing a sensitive detection assay (e.g., HeavyMethyl™ assay) on said ROI according to the present invention. To perform such an assay, the primers, probes and blockers are first designed using the sequence information given in SEQ ID NOS:1 and 2. The following primers, probes and blockers are designed using ROI SEQ ID NO:1 as template:

forward primer: (SEQ ID NO:206; 5′-GGG GTT TTA GGT TTT AGT GTT TAT TT-3′);

reverse primer: (SEQ ID NO:207; 5′-CTC CAA AAA CCA CCT TCC TAA CAC-3′);

blocker oligonucleotide: (specific to block amplification of CG containing template) (SEQ ID NO:218; 5′-CCT AAC ACg TTCg CCg CTA AAA ACC ACg CAA AAT AAA CC-3′);

blocker oligonucleotide control: (specific to block amplification of TG containing template) (SEQ ID NO:210; 5′-CCT AAC ACa TTC aCC aCT AAA AAC CAC aCA AAA TAA ACC-3′);

fluorescein anchor probe: (SEQ ID NO:216; 5′-AAT TtG GGT ATT TTT ATT GGT ATA AGG AAG GTG GGT AG-fluo);

detection probe: (SEQ ID NO:217; red640-GTA TtG TTT TGA AGA TAG tGT TAT TTA TTA TTG TAG TtG G-phosphate;

fluorescein anchor probe—control; (SEQ ID NO:208; 5′-AAT TCG GGT ATT TTT ATT GGT ATA AGG AAG GTG GGT AG-fluo); and

detection probe—control: (SEQ ID NO:209; red640-GTA TCG TTT TGA AGA TAG CGT TAT TTA TTA TTG TAG TCG G-phosphate).

The test (for determining the DNA source) is performed as follows:

Genomic DNA from one of these samples is treated with a solution of bisulfite as described in Olek et al. Nucleic Acids Res. 24:5064-6, 1996. As a result of this treatment, cytosine bases that are unmethylated are converted to thymine. The amount of DNA after bisulfite treatment is measured by UV absorption at 260 nm. About 100 pg of the pretreated DNA is used as template.

The HeavyMethyl™ assay is performed in a total volume of 20 μl using a LightCycler™ device (Roche Diagnostics). The real-time PCR reaction mix contains: 10 μl of template DNA (500 pg in total); 2 μl of FastStart LightCycler™ reaction mix for hybridization probes (Roche Diagnostics, Penzberg); 0.30 μM forward primer (SEQ ID NO:206; 5′-GGG GTT TTA GGT TTT AGT GTT TAT TT-3′); 0.30 μM reverse primer (SEQ ID NO:207; 5′-CTC CAA AAA CCA CCT TCC TAA CAC-3′); 0.15 μM fluorescein anchor probe (SEQ ID NO:216; 5′-AAT TtG GGT ATT TTT ATT GGT ATA AGG AAG GTG GGT AG-fluo; TIB-MolBiol, Berlin); 0.15 μM detection probe (SEQ ID NO:217; red640-GTA ttG ttT TGA AGA tAG tGT tAt tTA ttA tTG tAG ttG G-phosphate; TIB-MolBiol, Berlin); 1 μM blocker oligonucleotide (SEQ ID NO:218; 5′-CCT AAC Acg TTC gCC gCT AAA AAC CAC gCA AAA TAA ACC-3′); and 3 mM MgCl₂.

As a control, a parallel experiment is performed in a second PCR tube to detect the presence of methylated cytosines in said region. In this case, an amplificate and therefore a fluorescent signal, would indicate that the DNA is derived from a tissue other than liver, as for example brain or breast tissue. The real-time PCR reaction mix contains: 10 μl of template DNA (500 pg in total); 2 μl of FastStart LightCycler™ reaction mix for hybridization probes (Roche Diagnostics, Penzberg); 0.30 μM forward primer (SEQ ID NO:206; 5′-GGG GTT TTA GGT TTT AGT GTT TAT TT-3′); 0.30 μM reverse primer (SEQ ID NO:207; 5′-CTC CAA AAA CCA CCT TCC TAA CAC-3′); 0.15 μM fluorescein anchor probe (SEQ ID NO:208; 5′-AAT TCG GGT ATT TTT ATT GGT ATA AGG AAG GTG GGT AG-fluo; TIB-MolBiol, Berlin); 0.15 μM detection probe (SEQ ID NO:209; red640-GTA tCG ttT TGA AGA tAG CGT tAt tTA ttA tTG tAG tCG G-phosphate; TIB-MolBiol, Berlin); 1 μM blocker oligonucleotide (SEQ ID NO:210; 5′-CCT AAC ACA TTC ACC ACT AAA AAC CAC ACA AAA TAA ACC-3′); and 3 mM MgCl₂.

Thermocycling conditions are the same in both cases, and begin with a 95° C. incubation for 10 minutes, then 55 cycles of the following steps: 95° C. for 10 seconds, 56° C. for 30 seconds, and 72° C. for 10 seconds. Fluorescence is detected after the annealing phase at 56° C. in each cycle, however, only for the non-methylation sensitive assay (at the top) an intense signal can be achieved. From comparing this result with the data disclosed herein (see FIG. 1, and see Tables 3 and 37, herein above), it is concluded that the DNA analyzed is derived from liver.

### EXAMPLE 3

### The Marker ROI 3105 and the Attendant Epigenetic Map is Used in a Sensitive Detection Assay for Unambiguous Identification of Breast Tissue as the Source of Origin of Genomic DNA. A HeavyMethyl™ Assay is Used for Differentiation of Breast Tissue Amongst Other Tissues

The experiments of this example are in the context of a diagnostic laboratory, where two tubes arrive at the same day from the same practitioner, who has sent in biopsy samples from two of his female patients both named Smith. No other description is deciphered, but it is known that one sample is taken from a breast biopsy (to monitor the clearance of tumor cells after surgical removal and radiation therapy), whereas the other sample comes from a lung biopsy. The genomic DNA is already isolated when the ambiguity is noticed, so that a visual differentiation is no longer possible.

According to the present invention, only a quick test employing one of the breast markers disclosed herein is required to determine which DNA belonges to which patient Smith. The marker ROI 3105 (nt 512 to nt 3012 of DAXX gene, accession GI:3319283) is chosen, as it clearly differentiates between breast, which is highly unmethylated, and lung (or liver or brain) tissue, which is methylated to a higher degree (see Tables 10 and 44, herein above). The sequence information disclosed herein (3105 in SEQ ID NOS:15 and 16 and SEQ ID NOS:83 und 84), combined with the position of the MVPs, allows for the design of an appropriate assay (e.g., a HeavyMethyl™ assay, as described below).

Genomic DNA from the two samples is treated with a solution of bisulfite as it is described in Olek et al. Nucleic Acids Res. 1996 Dec. 15;24(24):5064-6. As a result of this treatment, cytosine bases that are unmethylated are converted to thymine. The amount of DNA after bisulfite treatment is measured by UV absorption at 260 nm, and 100 pg of the pretreated DNA is used as template.

The HeavyMethyl™ assay specific for unmethylated MVPs is performed in a total volume of 20 μl using a LightCycler™ device (Roche Diagnostics). The real-time PCR reaction mix contains: 10 μl of template DNA (100 pg in total); 2 μl of FastStart LightCycler™ reaction mix for hybridization probes (Roche Diagnostics, Penzberg); 0.30 μM forward primer (SEQ ID NO:211; 5′-GTA TTT TGA GTT ATG AGT TGG AGT TGT TGT-3′); 0.30 μM reverse primer (SEQ ID NO:212; 5′-AAC TAT ATA AAC TAA AAA ACT ACT CTT CAC TAACC-3′); 0.15 μM fluorescein anchor probe (SEQ ID NO:219; 5′-TTT GGT TTG TTG ATG AGT TGT TTA ATG TGT T-fluo; TIB-MolBiol, Berlin); 0.15 μM detection probe (SEQ ID NO:220; red640-TTA ATT TTT GGG TAG TGG GTG TTA TGG TA-phosphate; TIB-MolBiol, Berlin); 1 μM blocker oligonucleotide (SEQ ID NO:221; 5′-CTC TTC ACT AAC CgA CCg TAT CAT AAA ACA ACg CAT CCc-3′); and 3 mM MgCl₂.

An intense fluorescent signal is detected, indicating that an amplificate is obtained, which demonstrates that the methylation specific blocker employed in this assay is not binding to the template, indicating that the template contains TGs instead of CGs. From knowing that the MVPs covered by the blocker's sequence are unmethylated, it is concluded, by comparing the result with FIG. 8 or Table 10, that the sample DNA is derived from breast tissue.

As a control, a parallel experiment is performed in a second PCR tube to detect the presence of methylated cytosines in said region. The HeavyMethyl™ assay specific for upmethylated MVP is performed in a total volume of 20 μl using a LightCycler™ device (Roche Diagnostics). The real-time PCR reaction mix contains; 10 μl of template DNA (100 pg in total); 2 μl of FastStart LightCycler™ reaction mix for hybridization probes (Roche Diagnostics, Penzberg); 0.30 μM forward primer (SEQ ID NO:211; 5′-GTA TTT TGA GTT ATG AGT TGG AGT TGT TGT-3′); 0.30 μM reverse primer (SEQ ID NO:212; 5′-AAC TAT ATA AAC TAA AAA ACT ACT CTT CAC TAA CC-3′); 0.15 μM fluorescein anchor probe (SEQ ID NO:213; 5′-TTT GGT TTG TTG ATG AGT CGT TTA ATG CGT T-fluo; TIB-MolBiol, Berlin); 0.15 μM detection probe (SEQ ID NO:214; red640-TTA ATT TTT GGG TAG CGG GTG TTA CGG TA-phosphate; TIB-MolBiol, Berlin); 1 μM blocker oligonucleotide (SEQ ID NO:215; 5′-CTC TTC ACT AAC CAA CCA TAT CAT AAA ACA ACA CAT CCc-3′); and 3 mM MgCl₂.

Thermocycling conditions begin with a 95° C. incubation for 10 minutes, then 55 cycles of the following steps: 95° C. for 10 seconds, 56° C. for 30 seconds, and 72° C. for 10 seconds. Fluorescence is detected after the annealing phase at 56° C. in each cycle.

In this case an amplificate and hence a fluorescent signal, would indicate that the DNA is derived from a tissue other than breast, as for example brain, liver or lung tissue. No signal can be detected here, however.

The sample analyzed can be identified as DNA from breast tissue and therefore further analyses on both samples as demanded by the practitioner are enabled.

It is preferred, that the assays are performed as duplex PCR assays which enable the quantitative determination of the amount of a specific ROI sequence, methylated prior to bisulfite treatment, by methylation-specific amplification of the ROI fragment. The additional determination of the total amount of template DNA can be achieved by employing a suitable control fragment as template in a simultaneously performed control PCR in the same real-time PCR tube.

### EXAMPLE 4

### The Location/source of Free-floating DNA is Detected by a Sensitive Analysis Method

The experiments of the following example involve a blood sample that is taken from a patient who becomes aware of the fact that he has been exposed to high levels of radiation during his years of service in the army. Now the patient wishes to know whether he has developed a neoplastic disease like a tumour. His physician has not yet found any typical symptoms other than the patient complaining about unspecific pain at different organs, including headache.

A 20 ml blood sample is collected in heparin. Plasma and lymphocytes are separated by Ficoll gradient. Control lymphocyte and plasma DNA are purified on Qiagen columns (Qiamp Blood Kit, Qiagen, Basel, Switzerland) according to the “blood and body fluid protocol”. Plasma is passed on the same column. After purification of about 10 ml of plasma, 350 ng of DNA are obtained. The DNA is subjected to a sodium bisulfite treatment as described in Olek A, et al., Nucleic Acids Res. 24:5064-6, 1996. Aliquots of this bisulfite-treated DNA are used for a set of methylation assays.

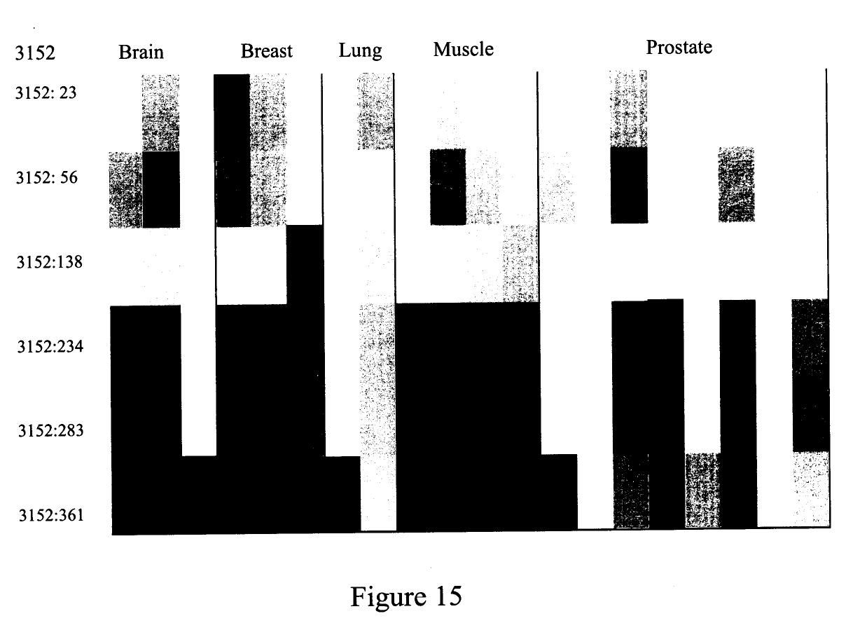

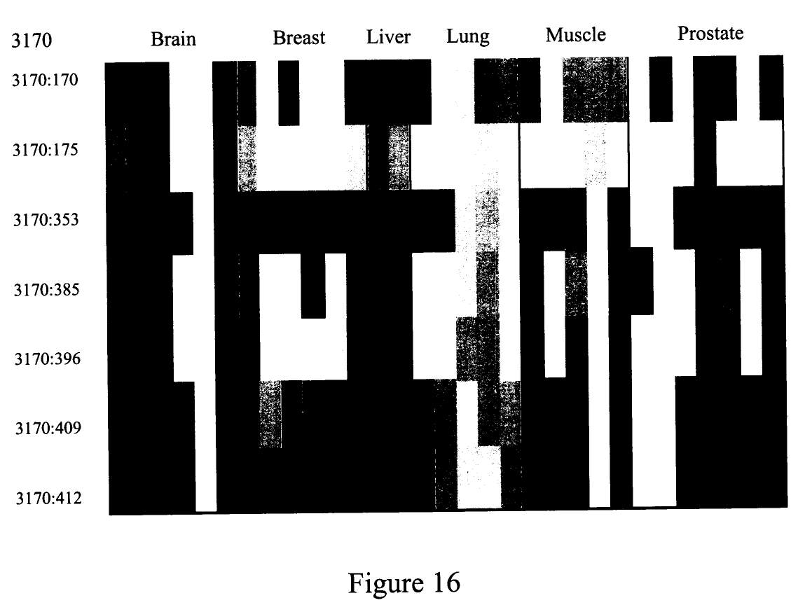

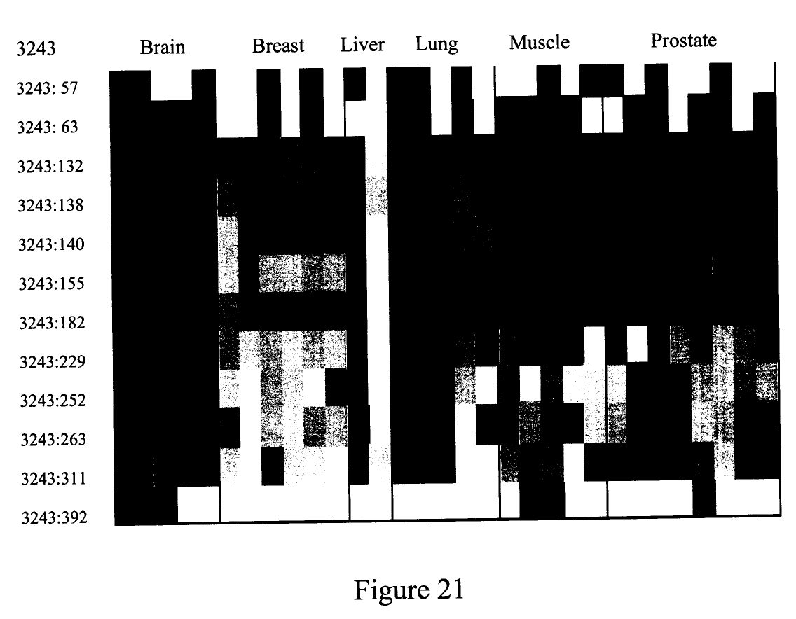

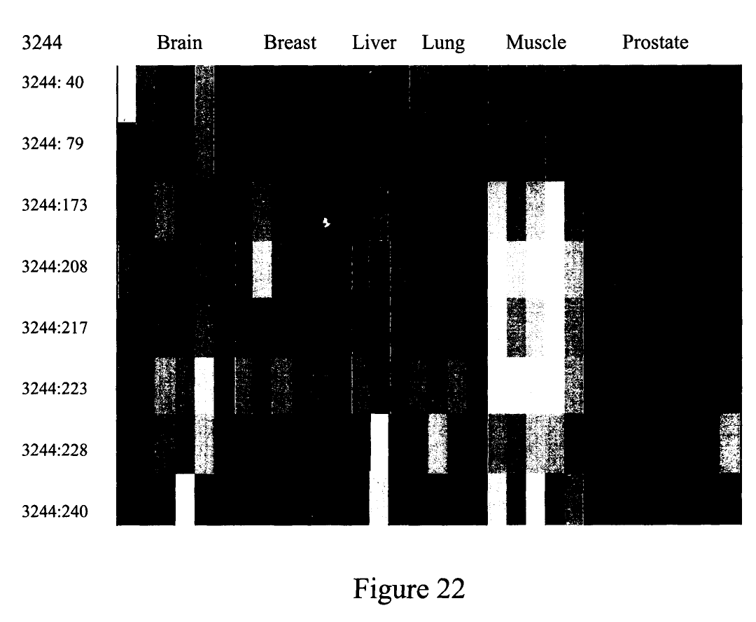

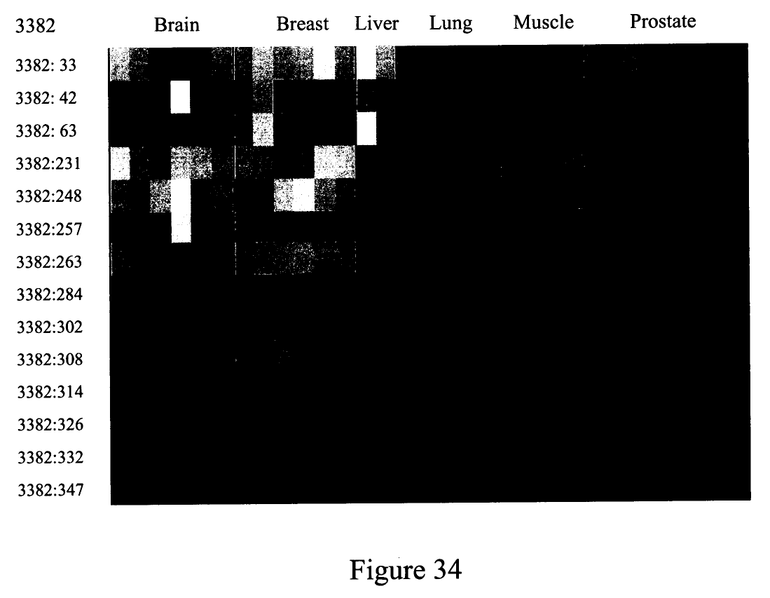

The regions analyzed are picked from the FIGS. 1-34. ROIs 3083 (BF, FIG. 1), 3152 (HLA-DMA, FIG. 15), 3170 (HLA-DRB3, FIG. 16), 3243 (TNF, FIG. 21), 3244 (TNXB, FIG. 22), and 3382 (DDX16, FIG. 34) are selected. Those sections of those ROIs that comprise a number of at least three MVPs are analyzed with an assay suitable to detect the levels of methylation at the MVPs disclosed (e.g., the MSP assay, or the HeavyMethyl™ assay). The individual's test result is compared with the dataset disclosed in FIGS. 1, 15, 16, 21, 22 and 34 and Tables 3, 17, 18, 23, 24 and 36. From these, it is concluded that a significant portion of the DNA in the patient's blood is derived from his lung. In this case, a single assay on ROI 3170 as template would also be sufficient, however, because it is not known that the free floating DNA was derived from lung, it is necessary to screen with a couple of markers at a time to get an accurate reliable result as fast as possible. Said result is sent back to the physician who then refers the patient to a hospital specializing in inflammatory or cell proliferative diseases of the lung.

### EXAMPLE 5

### A Routine Testing Assay is Introduced into a Tissue Analysis Laboratory

The experiments of the following example are performed in the context of a tissue analysis laboratory that works on a high-throughput basis, to introduce a step of quality assurance into the process. The quality assurance step comprises a routine testing of every tissue sample arriving at the laboratory, and prior to the sample entering the different analytical ‘tracks’ required for its further analyses. With the qualtity assurance step, the lab confirms the nature of the sample by an easy test on a molecular level.

According to the present invention, genomic DNA from each sample is extracted and treated with bisulfite as described herein above. The bisulfite-treated DNA is then prepared for sequence analysis runs.

ROIs 3083 (FIG. 1), 3152 (FIG. 15), 3170 (FIG. 16), 3243 (FIG. 21), 3244 (FIG. 22), and 3382 (FIG. 34) are selected. Each ROI is sequenced at those sections (regions) containing the MVPs disclosed. The primer pairs SEQ ID NOS:137, 138, 165, 166, 167, 168, 177, 178, 179, 180 and 203, 204, given in table 1, are used as sequencing primers.

Each section is sequenced once from both ends. Therefore, 12 sequencing runs are analyzed. Each test result is compared with the dataset disclosed in FIGS. 1, 15, 16, 21, 22 and 34 and Tables 3, 17, 18, 23, 24 and 36.

Further analysis of the sample in various analytical tracts will only be started if these quality assurance results confirm the sample information given upon arrival of the sample at the laboratory.

### EXAMPLE 6

### Forensic Case

The experiments of this example are performed in the context of a forensic case, where one of the relevant pieces of evidence was a piece of tissue that was found attached to a knife, suspected to be the weapon that killed a victim. For this case, it is of high importance to identify the kind of tissue that is attached to the knife, as there are several suspects, all of whom wounded the victim with their respective knives. The deadly wound was rendered by the knife that attacked the victim's liver. As the material has not been frozen, but is found 2 hot summer days after the murder at the crime scene in New York, the DNA is the material of choice to be used for this kind of analysis.

According to the present invention, and without great difficulties, intact genomic DNA is isolated from the weapons and a couple of sensitive detection assays (e.g., employing the liver markers ROI 3312 (gene SKIV2L) and ROI 3348 (gene DDX16), and the muscle markers 3265 and 3347 (both within genomic clone DASS-97D12)) are used to reveal whether the respective tissues in question are indeed derived from liver and not from muscle. Two MSP/MethyLight™ assays are designed to detect the methylation levels in said tissue, and are designed to only amplify a product that is detected by a Taqman™ probe.

According to the present invention, the tissue sample of the murder weapon may be contaminated with muscle tissue, but when compared to a pure muscle sample that is used as a control, the difference in signal intensities facilitates identification of the murder weapon, and makes it a clear case.

### EXAMPLE 7

### Computer and On-line Applications of the Present Invention; Online Epigenomic Map Subscription Service

In particular embodiments, the present invention relates to information systems theories and expert systems theories. The present invention provides a method and apparatus for providing information on samples comprising genomic DNA (e.g., DNA, cells, tissues, bodily fluids, etc.) to a user or subscriber. The method and apparatus allows for identifying, or for distinguishing between or among such samples, based on a database containing tissue-specific quantitative methylation data.

The quantitative methylation data is initially afforded by using DNA sequence trace analysis software, such as the preferred ESME embodiment described herein. ESME is a software program that considers or accounts for the unequal distribution of bases in bisulfite converted DNA and normalizes the sequence traces (electropherograms) to allow for quantitation of methylation signals within the sequence traces. Additionally, it calculates a bisulfite conversion rate, by comparing signal intensities of thymines at specific positions, based on the information about the corresponding untreated DNA sequence.

In preferred embodiments, the invention provides a computer implemented method for providing information on tissue specimens to a user or subscriber comprising: obtaining DNA, cell or tissue samples corresponding to a plurality of tissue types from a subset of a population of subjects with shared characteristics, said samples having genomic DNA; assaying the genomic DNA of each of the tissue samples; determining for each tissue type, based on said assaying, a distribution of values for each of location, type and level of methylated CpG positions within one or more genomic DNA regions; calculating average indices for each of the distribution of values; calculating dispersion indices for each of the average indices; storing the average indices and dispersion indices in a database; and providing to the user or subscriber, in exchange for a fee, access to said average indices and dispersion indices in said database, wherein the number of tissue samples includes a sufficient number of samples such that the dispersion and average indices correspond to a statistically significant representation of those indices for the population as a whole.

Preferably, the tissue samples comprise normal tissue, or abnormal tissue. Preferably, where the tissue samples comprise normal and abnormal tissue of the same tissue type, data from normal tissue is used to determine a distribution of values and corresponding indices for normal tissue, and data from abnormal tissue is used to determine a distribution of values and corresponding indices for abnormal tissue. Preferably, the tissue types comprise a type selected from the group consisting of breast, liver, prostate, muscle, brain, lung and combinations thereof.

Consumers do not have an intelligent, fast and reliable method for accessing quantified methylation-based information services. The present invention addresses this need by creating a software program able to link the consumer/user to one or more functional epigenomic databases, such as an ‘MVP database’. An MVP database refers to a database containing the methylation levels and an epigenomic database comprising locations of differentially methylated CpG positions, in relation to the detailed description of samples including, for example, all, or a portion of all available phenotypical characteristics, and clinical parameters. The database is searchable, for example, for CpG positions that are differentially methylated between or among two or more phenotypically distinct types of tissues/samples. A consumer can access the Internet using a computer or electronic hand-held device. The software program of the present invention is usable in a stand-alone computer system.

The apparatus of the present invention is a computer, or computer network comprising a server, at least one user subsystem connected to the server via a network connecting means (e.g., user modem). Although referred to as a modem, the user modem can be any other communication means that enables network communication, for example, ethernet links. The modem can be connected to the server by a variety of connecting means, including public telephone land lines, dedicated data lines, cellular links, microwave links, or satellite communication.

The server is essentially a high-capacity, high-speed computer that includes a processing unit connected to one or more relatable data bases, comprising an “MVP database” that contains methylation levels, and an epigenomic database comprising locations of differentially methylated CpG positions (MVP positions), in relation to the detailed description of samples including, for example, all, or a portion of all available phenotypical characteristics, and clinical parameters. The database is searchable, for example, for CpG positions that are differentially methylated between or among two or more phenotypically distinct types of tissues/samples. Additional databases are optionally added to the server. For example, a searchable database comprising a listing of which MVP positions have utility for distinguishing between which sample types may be included.

Also connected to the processing unit is sufficient memory and appropriate communication hardware. The communication hardware may be modems, ethernet connections, or any other suitable communication hardware. Although the server can be a single computer having a single processing unit, it is also possible that the server could be spread over several networked computers, each having its processor and having one or more databases resident thereon.

In addition to the elements described above, the server further comprises an operating system and communication software allowing the server to communicate with other computers. Various operating systems and communication software may be employed. For example, the operating system may be Microsoft Windows NT™, and the communication software Microsoft IIS™ (Internet Information Server) server with associated programs.

The databases on the server contain the information necessary to make the apparatus and process work. The databases are relatable and are assembled and accessed using any commercially available database software, such as Microsoft Access™, Oracle™, Microsoft SQL™ Version 6.5, etc.

A user subsystem generally includes a processor attached to storage unit, a communication controller, and a display controller. The display controller runs a display unit through which the user interacts with the subsystem. In essence, the user subsystem is a computer able to run software providing a means for communicating with the server. This software, for example, is an Internet web browser such as Microsoft Internet Explorer, Netscape Navigator, Mozilla, or other suitable Internet web browsers. The user subsystem can be a computer or hand-held electron device, such as a telephone or other device allowing for Internet access.

Particular embodiments comprise a basic computer model with a central processing unit (“CPU”), Hard Storage (“Hard Disk”), Soft Storage (“RAM”), and an Input and Output interface (“Input/Output”). A consumer/user, at a user interface, is either interested in specific information, access to services, or is concerned about identification or differentiation of one or more samples. Once they log on to a host site, a main window screen is displayed giving the options to login as a registered user, use a ‘smart’ search, or directly access the online epigenomic map subscription service interface. In preferred embodiments, the system is implemented as a full, interactive service.

1. A method for generating a genome-wide methylation map, comprising:
a) obtaining, for each of at least two biological sample types, a plurality or group of biological samples having genomic DNA;
b) pretreating the genomic DNA of the samples by contacting the samples, or isolated DNA from the samples, with an agent, or series of agents that modifies unmethylated cytosine but leaves methylated cytosine essentially unmodified;
c) amplifying segments of the pretreated DNA, said amplified segments representing the entire genome, or a portion thereof, and comprising in each case at least one dinucleotide sequence position corresponding to a CpG dinucleotide position in the corresponding untreated genomic DNA, and wherein said amplification is by means of primer molecules that do not comprise a dinucleotide sequence position corresponding to a CpG dinucleotide position in the corresponding untreated genomic DNA;
d) sequencing the amplified pretreated nucleic acids;
e) analyzing the sequences to quantify a level of methylation at specific CpG positions;
f) comparing said quantified levels of methylation at specific CpG positions between the different sample groups corresponding to the at least two biological sample types; and
g) identifying methylation variable positions, wherein a methylation variable position is a genomic CpG position, for which there is a detectable difference in the quantified level of methylation between different biological sample types, and whereby an epigenomic map over the entire genome, or a portion thereof is, at least in part, afforded.

2. The method of claim 1, wherein the biological sample type is of a tissue, organ or cell.

3. The method of claim 1, wherein in c), the dinucleotide sequence position corresponding to a CpG dinucleotide position in the corresponding untreated genomic DNA is a CpG or a TpG dinucleotide sequence position.

4. The method of claim 1, wherein sequencing in d) comprises generating a sequence trace, or electropherogram for use in quantifying the level of methylation.

5. The method of claim 1, wherein analyzing the sequences in e), comprises creating a profile of the quantified level of methylation over the entire genome, or a portion thereof.

6. The method of any one of the above claims, wherein quantifying the level of methylation in e) involves the use of a software program suitable therefore.

7. The method of claim 6, wherein the suitable software program is ESME, which considers or accounts for an unequal distribution of bases in bisulfite converted DNA and normalizes sequence traces (electropherograms) to allow for quantitation of methylation signals within the sequence traces.

8. The method of claim 1, wherein the agent, or series of agents of b) comprises a bisulfite reagent.

9. The method of claim 1, wherein the agent, or series of agents of b) comprises an enzyme.

10. The method of claim 1, wherein pretreating in b) comprises modification of cytosine to uracil.

11. The method of claim 1, wherein amplifying segments in c), comprises amplification of at least one segment located in, or comprising a regulatory region of a gene.

12. The method of claim 1, wherein amplifying in c) comprises use of a polymerase chain reaction (PCR).

13. A nucleic acid or an oligomer comprising at least one contiguous base sequence having a length of at least 16 nucleotides that is complementary to, or hybridizes under moderately stringent or stringent conditions to a pretreated genomic DNA sequence selected from the group consisting of SEQ ID NOS:1-136, and sequences complementary thereto, wherein said contiguous sequence comprises at least one methylation variable position, or at least one CpG, tpG, or Cpa dinucleotide sequence, and wherein pretreatment comprises treating the genomic DNA with an agent, or series of agents, that modifies unmethylated, but leaves methylated, cytosine essentially unmodified.

14. A set of oligomers, said set comprising a first oligomer and a second oligomer, wherein the first oligomer, and the second oligomer each comprises at least one contiguous base sequence of at least 16 nucleotides in length that is complementary to, or hybridizes under moderately stringent or stringent conditions to a pretreated genomic DNA sequence selected from, in the case of the first oligomer, a first sequence group consisting of SEQ ID NOS:1-136, and selected from, in the case of the second oligomer, a second sequence group consisting of sequences complementary to the sequences of the first sequence group, and wherein pretreatment comprises treating the genomic DNA with an agent, or series of agents, that modifies unmethylated, but leaves methylated, cytosine essentially unmodified.

15. The set of oligomers of claim 14, wherein the set is suitable for use in generating nucleic acid amplificates.

16. A nucleic acid or oligomer, comprising a sequence selected from the group consisting of SEQ ID NOS:137 through 204 and SEQ ID NOS:206 through 221.

17. A nucleic acid or an oligomer comprising at least one contiguous base sequence having a length of at least 16 nucleotides that is complementary to, or hybridizes under moderately stringent or stringent conditions to a pretreated genomic DNA sequence selected from a group consisting of SEQ ID NOS:1, 2, 69, 70; SEQ ID NOS:3, 4, 71, 72; SEQ ID NOS:5, 6, 73, 74; SEQ ID NOS:7, 8, 75, 76; SEQ ID NOS:9, 10, 77, 78; SEQ ID NOS:11, 12, 79, 80; SEQ ID NOS:13, 14, 81, 82; SEQ ID NOS:15, 16, 83, 84; SEQ ID NOS:17, 18, 85, 86; SEQ ID NOS:19, 20, 87, 88; SEQ ID NOS:21, 22, 89, 90; SEQ ID NOS:23, 24, 91, 92; SEQ ID NOS:25, 26, 93, 94; SEQ ID NOS:27, 28, 95, 96; SEQ ID NOS:29, 30, 97, 98; SEQ ID NOS:31, 32, 99, 100; SEQ ID NOS:33, 34, 101, 102; SEQ ID NOS:35, 36, 103, 104; SEQ ID NOS:37, 38, 105, 106; SEQ ID NOS:39, 40, 107, 108; SEQ ID NOS:41, 42, 109, 110; SEQ ID NOS:43, 44, 111, 112; SEQ ID NOS:45, 46, 113, 114; SEQ ID NOS:47, 48, 115, 116; SEQ ID NOS:49, 50, 117, 118; SEQ ID NOS:51, 52, 119, 120; SEQ ID NOS:53, 54, 121, 122; SEQ ID NOS:55, 56, 123, 124; SEQ ID NOS:57, 58, 125, 126; SEQ ID NOS:59, 60, 127, 128; SEQ ID NOS: 61, 62, 129, 130; SEQ ID NOS:63, 64, 131, 132; SEQ ID NOS:65, 66, 133, 134 and SEQ ID NOS:67, 68, 135, 136, and sequences complementary thereto, wherein said contiguous sequence comprises at least one methylation variable position, or at least one CpG, tpG, or Cpa dinucleotide sequence, and wherein pretreatment comprises treating the genomic DNA with an agent, or series of agents, that modifies unmethylated, but leaves methylated, cytosine essentially unmodified.

18. A set of oligomers, said set comprising a first oligomer and a second oligomer, wherein the first oligomer, and the second oligomer each comprises at least one contiguous base sequence of at least 16 nucleotides in length that is complementary to, or hybridizes under moderately stringent or stringent conditions to a pretreated genomic DNA sequence selected from, in the case of the first oligomer, a sequence subgroup selected from a first group of 4-sequence subgroups consisting of SEQ ID NOS:1, 2, 69, 70; SEQ ID NOS:3, 4, 71, 72; SEQ ID NOS:5, 6, 73, 74; SEQ ID NOS:7, 8, 75, 76; SEQ ID NOS:9, 10, 77, 78; SEQ ID NOS:11, 12, 79, 80; SEQ ID NOS:13, 14, 81, 82; SEQ ID NOS:15, 16, 83, 84; SEQ ID NOS:17, 18, 85, 86; SEQ ID NOS:19, 20, 87, 88; SEQ ID NOS:21, 22, 89, 90; SEQ ID NOS:23, 24, 91, 92; SEQ ID NOS:25, 26, 93, 94; SEQ ID NOS:27, 28, 95, 96; SEQ ID NOS:29, 30, 97, 98; SEQ ID NOS:31, 32, 99, 100; SEQ ID NOS:33, 34, 101, 102; SEQ ID NOS:35, 36, 103, 104; SEQ ID NOS:37, 38, 105, 106; SEQ ID NOS:39, 40, 107, 108; SEQ ID NOS:41, 42, 109, 110; SEQ ID NOS:43, 44, 111, 112; SEQ ID NOS:45, 46, 113, 114; SEQ ID NOS:47, 48, 115, 116; SEQ ID NOS:49, 50, 117, 118; SEQ ID NOS:51, 52, 119, 120; SEQ ID NOS:53, 54, 121, 122; SEQ ID NOS:55, 56, 123, 124; SEQ ID NOS:57, 58, 125, 126; SEQ ID NOS:59, 60, 127, 128; SEQ ID NOS:61, 62, 129, 130; SEQ ID NOS:63, 64, 131, 132; SEQ ID NOS:65, 66, 133, 134 and SEQ ID NOS:67, 68, 135, 136, and selected from, in the case of the second oligomer, a corresponding complementary sequence subgroup selected from a second group of 4-sequence subgroups consisting of sequences complementary to the respective subgroup sequences of the first sequence group, and wherein pretreatment comprises treating the genomic DNA with an agent, or series of agents, that modifies unmethylated, but leaves methylated, cytosine essentially unmodified.

19. The set of oligomers of claim 18, wherein the set is suitable for use in generating nucleic acid amplificates.

20. A method for at least one of identifying liver cells, organ or tissue, distinguishing liver cells, organ or tissue from one or more other cell or tissue types, or identifying liver cells, organ or tissue as the source of a DNA sample, comprising:
a) obtaining at least one cell, tissue, bodily fluid or other sample, wherein the sample comprises genomic DNA;
b) determining, for the at least one sample and using a suitable assay, a methylation state or a level of methylation for at least one methylation variable position within a genomic DNA sequence selected from the group consisting of SEQ ID NO:205, a fragment thereof at least 16 contiguous nucleotides in length, and sequences that are complementary to, or hybridize under moderately stringent or stringent conditions to SEQ ID NO:205 or to a fragment thereof at least 16 contiguous nucleotides in length; and
c) comparing said at least one methylation state or level of methylation with a suitable standard or control, or comparing said at least one methylation state or level of methylation between or among corresponding methylation variable positions of the samples, whereby at least one of identifying liver cells, organ or tissue, distinguishing liver cells, organ or tissue from one or more other cell, organ or tissue types, or identifying liver cells, organ or tissue as the source of a DNA sample is, at least in part afforded.

21. The method of claim 20, wherein determining in b), comprises at least one of: use of one or more nucleic acid or oligomers comprising, in each case, at least one contiguous base sequence having a length of at least 16 nucleotides that is complementary to, or hybridizes under moderately stringent or stringent conditions to a pretreated genomic DNA sequence selected from a group consisting of SEQ ID NOS:1, 2, 69, 70; SEQ ID NOS:7, 8, 75, 76; SEQ ID NOS:9, 10, 77, 78; SEQ ID NOS:11, 12, 79, 80; SEQ ID NOS:13, 14, 81, 82; SEQ ID NOS:25, 26, 93, 94; SEQ ID NOS:27, 28, 95, 96; SEQ ID NOS:35, 36, 103, 104; SEQ ID NOS:37, 38, 105, 106; SEQ ID NOS:51, 52, 119, 120; SEQ ID NOS:53, 54, 121, 122; SEQ ID NOS:59, 60, 127, 128; and sequences complementary thereto; or use of a methylation-sensitive restriction enzyme on a genomic DNA sequence selected from the group consisting of SEQ ID NO:205 or a fragment thereof at least 16 contiguous nucleotides in length, and sequences that are complementary to, or hybridize under moderately stringent or stringent conditions to SEQ ID NO: 205 or a fragment thereof at least 16 contiguous nucleotides in length.

22. A method for at least one of identifying brain cells, organ or tissue, distinguishing brain cells, organ or tissue from one or more other cell or tissue types, or identifying brain cells, organ or tissue as the source of a DNA sample, comprising:
a) obtaining at least one cell, tissue, bodily fluid or other sample, wherein the sample comprises genomic DNA;
b) determining, for the at least one sample and using a suitable assay, a methylation state or a level of methylation for at least one methylation variable position within a genomic DNA sequence selected from the group consisting of SEQ ID NO:205, a fragment thereof at least 16 contiguous nucleotides in length, and sequences that are complementary to, or hybridize under moderately stringent or stringent conditions to SEQ ID NO:205 or to a fragment thereof at least 16 contiguous nucleotides in length; and
c) comparing said at least one methylation state or level of methylation with a suitable standard or control, or comparing said at least one methylation state or level of methylation between or among corresponding methylation variable positions of the samples, whereby at least one of identifying brain cells, organ or tissue, distinguishing brain cells, organ or tissue from one or more other cell, organ or tissue types, or identifying brain cells, organ or tissue as the source of a DNA sample is, at least in part afforded.

23. The method of claim 22, wherein determining in b), comprises at least one of: use of one or more nucleic acid or oligomers comprising, in each case, at least one contiguous base sequence having a length of at least 16 nucleotides that is complementary to, or hybridizes under moderately stringent or stringent conditions to a pretreated genomic DNA sequence selected from a group consisting of SEQ ID NOS:3, 4, 71, 72; SEQ ID NOS:17, 18, 85, 86; SEQ ID NOS:19, 20, 87, 88; SEQ ID NOS:29, 30, 97, 98; SEQ ID NOS: 49, 50, 117, 118; SEQ ID NOS:57, 58, 125, 126; SEQ ID NOS:61, 62, 129, 130; SEQ ID NOS:67, 68, 135, 136; and sequences complementary thereto; or use of a methylation-sensitive restriction enzyme on a genomic DNA sequence selected from the group consisting of SEQ ID NO:205 or a fragment thereof at least 16 contiguous nucleotides in length, and sequences that are complementary to, or hybridize under moderately stringent or stringent conditions to SEQ ID NO:205 or a fragment thereof at least 16 contiguous nucleotides in length.

24. A method for at least one of identifying breast cells, organ or tissue, distinguishing breast cells, organ or tissue from one or more other cell or tissue types, or identifying breast cells, organ or tissue as the source of a DNA sample, comprising:
a) obtaining at least one cell, tissue, bodily fluid or other sample, wherein the sample comprises genomic DNA;
b) determining, for the at least one sample and using a suitable assay, a methylation state or a level of methylation for at least one methylation variable position within a genomic DNA sequence selected from the group consisting of SEQ ID NO:205, a fragment thereof at least 16 contiguous nucleotides in length, and sequences that are complementary to, or hybridize under moderately stringent or stringent conditions to SEQ ID NO:205 or to a fragment thereof at least 16 contiguous nucleotides in length; and
c) comparing said at least one methylation state or level of methylation with a suitable standard or control, or comparing said at least one methylation state or level of methylation between or among corresponding methylation variable positions of the samples, whereby at least one of identifying breast cells, organ or tissue, distinguishing breast cells, organ or tissue from one or more other cell, organ or tissue types, or identifying breast cells, organ or tissue as the source of a DNA sample is, at least in part afforded.

25. The method of claim 24, wherein determining in b), comprises at least one of: use of one or more nucleic acid or oligomers comprising, in each case, at least one contiguous base sequence having a length of at least 16 nucleotides that is complementary to, or hybridizes under moderately stringent or stringent conditions to a pretreated genomic DNA sequence selected from a group consisting of SEQ ID NOS:3, 4, 71, 72; SEQ ID NOS:5, 6, 73, 74; SEQ ID NOS;15, 16, 83, 84; SEQ ID NOS:19, 20, 87, 88; SEQ ID NOS:21, 22, 89, 90; SEQ ID NOS:23, 24, 91, 92; SEQ ID NOS:29, 30, 97, 98; SEQ ID NOS:39, 40, 107, 108; SEQ ID NOS;41, 42, 109, 110; SEQ ID NOS;45, 46, 113, 114; SEQ ID NOS;63, 64, 131, 132; SEQ ID NOS:65, 66, 133, 134; SEQ ID NOS:67, 68, 135, 136; and sequences complementary thereto; or use of a methylation-sensitive restriction enzyme on a genomic DNA sequence selected from the group consisting of SEQ ID NO:205 or a fragment thereof at least 16 contiguous nucleotides in length, and sequences that are complementary to, or hybridize under moderately stringent or stringent conditions to SEQ ID NO:205 or a fragment thereof at least 16 contiguous nucleotides in length.

26. A method for at least one of identifying muscle cells, organ or tissue, distinguishing muscle cells, organ or tissue from one or more other cell or tissue types, or identifying muscle cells, organ or tissue as the source of a DNA sample, comprising:
a) obtaining at least one cell, tissue, bodily fluid or other sample, wherein the sample comprises genomic DNA;
b) determining, for the at least one sample and using a suitable assay, a methylation state or a level of methylation for at least one methylation variable position within a genomic DNA sequence selected from the group consisting of SEQ ID NO:205, a fragment thereof at least 16 contiguous nucleotides in length, and sequences that are complementary to, or hybridize under moderately stringent or stringent conditions to SEQ ID NO:205 or to a fragment thereof at least 16 contiguous nucleotides in length; and
c) comparing said at least one methylation state or level of methylation with a suitable standard or control, or comparing said at least one methylation state or level of methylation between or among corresponding methylation variable positions of the samples, whereby at least one of identifying muscle cells, organ or tissue, distinguishing muscle cells, organ or tissue from one or more other cell, organ or tissue types, or identifying muscle cells, organ or tissue as the source of a DNA sample is, at least in part afforded.

27. The method of claim 26, wherein determining in b), comprises at least one of: use of one or more nucleic acid or oligomers comprising, in each case, at least one contiguous base sequence having a length of at least 16 nucleotides that is complementary to, or hybridizes under moderately stringent or stringent conditions to a pretreated genomic DNA sequence selected from a group consisting of SEQ ID NOS:15, 16, 83, 84; SEQ ID NOS:19, 20, 87, 88; SEQ ID NOS:21, 22, 89, 90; SEQ ID NOS:27, 28, 95, 96; SEQ ID NOS:29, 30, 97, 98; SEQ ID NOS:43, 44, 111, 112; SEQ ID NOS:45, 46, 113, 114; SEQ ID NOS:47, 48, 115, 116; SEQ ID NOS:55, 56, 123, 124; SEQ ID NOS:57, 58, 125, 126; SEQ ID NOS:63, 64, 131, 132; and sequences complementary thereto; or use of a methylation-sensitive restriction enzyme on a genomic DNA sequence selected from the group consisting of SEQ ID NO:205 or a fragment thereof at least 16 contiguous nucleotides in length, and sequences that are complementary to, or hybridize under moderately stringent or stringent conditions to SEQ ID NO:205 or a fragment thereof at least 16 contiguous nucleotides in length.

28. A method for at least one of identifying lung cells, organ or tissue, distinguishing lung cells, organ or tissue from one or more other cell, organ or tissue types, or identifying lung cells, organ or tissue as the source of a DNA sample, comprising:
a) obtaining at least one cell, tissue, bodily fluid or other sample, wherein the sample comprises genomic DNA;
b) determining, for the at least one sample and using a suitable assay, a methylation state or a level of methylation for at least one methylation variable position within a genomic DNA sequence selected from the group consisting of SEQ ID NO:205, a fragment thereof at least 16 contiguous nucleotides in length, and sequences that are complementary to, or hybridize under moderately stringent or stringent conditions to SEQ ID NO:205 or to a fragment thereof at least 16 contiguous nucleotides in length; and
c) comparing said at least one methylation state or level of methylation with a suitable standard or control, or comparing said at least one methylation state or level of methylation between or among corresponding methylation variable positions of the samples, whereby at least one of identifying lung cells, organ or tissue, distinguishing lung cells, organ or tissue from one or more other cell, organ or tissue types, or identifying lung cells, organ or tissue as the source of a DNA sample is, at least in part afforded.

29. The method of claim 28, wherein determining in b), comprises at least one of: use of one or more nucleic acid or oligomers comprising, in each case, at least one contiguous base sequence having a length of at least 16 nucleotides that is complementary to, or hybridizes under moderately stringent or stringent conditions to a pretreated genomic DNA sequence selected from a group consisting of SEQ ID NOS:21, 22, 89, 99; SEQ ID NOS:29, 30, 97, 98; SEQ ID NOS:31, 32, 99, 100; SEQ ID NOS:33, 34, 101, 102; SEQ ID NOS:55, 56, 123, 124, and sequences complementary thereto; or use of a methylation-sensitive restriction enzyme on a genomic DNA sequence selected from the group consisting of SEQ ID NO:205 or a fragment thereof at least 16 contiguous nucleotides in length, and sequences that are complementary to, or hybridize under moderately stringent or stringent conditions to SEQ ID NO:205 or a fragment thereof at least 16 contiguous nucleotides in length.

30. Use of a nucleic acid or oligomer, in a method for the identification or distinguishing of liver cells, organ or tissue or a nucleic acid derived there from, or for the identification of liver cells, organ or tissue as the source of said nucleic acid, wherein said nucleic acid or oligomer comprises at least one contiguous base sequence having a length of at least 16 nucleotides that is complementary to, or hybridizes under moderately stringent or stringent conditions to a pretreated genomic DNA sequence selected from the group consisting of SEQ ID NOS:1, 2, 69, 70; SEQ ID NOS:7, 8, 75, 76; SEQ ID NOS:9, 10, 77, 78; SEQ ID NOS:11, 12, 79, 80; SEQ ID NOS:13, 14, 81, 82; SEQ ID NOS:25, 26, 93, 94; SEQ ID NOS:27, 28, 95, 96; SEQ ID NOS:35, 36, 103, 104; SEQ ID NOS:37, 38, 105, 106; SEQ ID NOS:51, 52, 119, 120; SEQ ID NOS:53, 54, 121, 122; SEQ ID NOS:59, 60, 127, 128; and sequences complementary thereto, said method comprising determining the level of methylation of at least one methylation variable positions (MVPs) within one or more sequences of the sequence group.

31. The use of a nucleic acid or oligomer, in a method for the identification or distinguishing of liver cells, organ or tissue, or a nucleic acid derived there from, or for the identification of liver cells, organ or tissue as the source of said nucleic acid, wherein said nucleic acid or oligomer comprises at least one contiguous base sequence at least 16 nucleotides in length selected from the group consisting of SEQ ID NOS:137, 138; 143, 144: 145, 146; 147, 148; 149, 150; 161, 162; 163, 164; 171, 172; 173, 174; 187, 188; 189, 190; 19, and SEQ ID NO:196.

32. Use of a nucleic acid or oligomer, in a method for the identification or distinguishing of brain cells, organ or tissue or a nucleic acid derived there from, or for the identification of brain cells, organ or tissue as the source of said nucleic acid, wherein said nucleic acid or oligomer comprises at least one contiguous base sequence having a length of at least 16 nucleotides that is complementary to, or hybridizes under moderately stringent or stringent conditions to a pretreated genomic DNA sequence selected from the group consisting of SEQ ID NOS:3, 4, 71, 72; SEQ ID NOS:17, 18, 85, 86; SEQ ID NOS:19, 20, 87, 88; SEQ ID NOS:29, 30, 97, 98; SEQ ID NOS:49, 50, 117, 118; SEQ ID NOS:57, 58, 125, 126; SEQ ID NOS:61, 62, 129, 130; SEQ ID NOS:67, 68, 135, 136; and sequences complementary thereto, said method comprising determining the level of methylation of at least one methylation variable positions (MVPs) within one or more sequences of the sequence group.

33. The use of a nucleic acid or oligomer, in a method for the identification or distinguishing of brain cells, organ or tissue, or a nucleic acid derived there from, or for the identification of brain cells, organ or tissue as the source of said nucleic acid, wherein said nucleic acid or oligomer comprises at least one contiguous base sequence at least 16 nucleotides in length selected from the group consisting of SEQ ID NOS:139, 140; 153, 154; 155, 156; 157, 158; 165, 166; 185, 186; 193, 194; 197, 198; 203 and SEQ ID NO:204.

34. Use of a nucleic acid or oligomer, in a method for the identification or distinguishing of breast cells, organ or tissue or a nucleic acid derived there from, or for the identification of breast cells, organ or tissue as the source of said nucleic acid, wherein said nucleic acid or oligomer comprises at least one contiguous base sequence having a length of at least 16 nucleotides that is complementary to, or hybridizes under moderately stringent or stringent conditions to a pretreated genomic DNA sequence selected from the group consisting of SEQ ID NOS:3, 4, 71, 72; SEQ ID NOS:5, 6, 73, 74; SEQ ID NOS:15, 16, 83, 84; SEQ ID NOS:19, 20, 87, 88; SEQ ID NOS:21, 22, 89, 90; SEQ ID NOS:23, 24, 91, 92; SEQ ID NOS:29, 30, 97, 98; SEQ ID NOS:39, 40, 107, 108; SEQ ID NOS:41, 42, 109, 110; SEQ ID NOS:45, 46, 113, 114; SEQ ID NOS:63, 64, 131, 132; SEQ ID NOS:65, 66, 133, 134; SEQ ID NOS:67, 68, 135, 136; and sequences complementary thereto, said method comprising determining the level of methylation of at least one methylation variable positions (MVPs) within one or more sequences of the sequence group.

35. The use of a nucleic acid or oligomer, in a method for the identification or distinguishing of breast cells, organ or tissue, or a nucleic acid derived there from, or for the identification of breast cells, organ or tissue as the source of said nucleic acid, wherein said nucleic acid or oligomer comprises at least one contiguous base sequence at least 16 nucleotides in length selected from the group consisting of SEQ ID NOS:139, 140; 141, 142; 151, 152; 155, 156, 157, 158; 159, 160; 165, 166, 175, 176; 177, 178; 181, 182; 199, 200; 201, 202; 203 and SEQ ID NO:204.

36. Use of a nucleic acid or oligomer, in a method for the identification or distinguishing of muscle cells, organ or tissue or a nucleic acid derived there from, or for the identification of muscle cells, organ or tissue as the source of said nucleic acid, wherein said nucleic acid or oligomer comprises at least one contiguous base sequence having a length of at least 16 nucleotides that is complementary to, or hybridizes under moderately stringent or stringent conditions to a pretreated genomic DNA sequence selected from the group consisting of SEQ ID NOS:15, 16, 83, 84; SEQ ID NOS:19, 20, 87, 88; SEQ ID NOS:21, 22, 89, 90; SEQ ID NOS:27, 28, 95, 96; SEQ ID NOS:29, 30, 97, 98; SEQ ID NOS:43, 44, 111, 112; SEQ ID NOS:45, 46, 113, 114; SEQ ID NOS:47, 48, 115, 116; SEQ ID NOS:55, 56, 123, 124; SEQ ID NOS:57, 58, 125, 126; SEQ ID NOS:63, 64, 131, 132; and sequences complementary thereto, said method comprising determining the level of methylation of at least one methylation variable positions (MVPs) within one or more sequences of the sequence group.

37. The use of a nucleic acid or oligomer, in a method for the identification or distinguishing of muscle cells, organ or tissue, or a nucleic acid derived there from, or for the identification of muscle cells, organ or tissue as the source of said nucleic acid, wherein said nucleic acid or oligomer comprises at least one contiguous base sequence at least 16 nucleotides in length selected from the group consisting of SEQ ID NOS:152, 152; 155, 156; 157, 158; 163, 164; 165, 166; 179, 180; 181, 182; 183, 184; 191, 192; 193, 194; 199 and SEQ ID NO:200.

38. Use of a nucleic acid or oligomer, in a method for the identification or distinguishing of lung cells, organ or tissue or a nucleic acid derived there from, or for the identification of lung cells, organ or tissue as the source of said nucleic acid, wherein said nucleic acid or oligomer comprises at least one contiguous base sequence having a length of at least 16 nucleotides that is complementary to, or hybridizes under moderately stringent or stringent conditions to a pretreated genomic DNA sequence selected from the group consisting of SEQ ID NOS:19, 20, 87, 88; SEQ ID NOS:21, 22, 89, 99; SEQ ID NOS:29, 30, 97, 98; SEQ ID NOS:31, 32, 99, 100; SEQ ID NOS:33, 34, 101, 102; SEQ ID NOS:55, 56, 123, 124; and sequences complementary thereto, said method comprising determining the level of methylation of at least one methylation variable positions (MVPs) within one or more sequences of the sequence group.

39. The use of a nucleic acid or oligomer, in a method for the identification or distinguishing of lung cells, organ or tissue, or a nucleic acid derived there from, or for the identification of lung cells, organ or tissue as the source of said nucleic acid, wherein said nucleic acid or oligomer comprises at least one contiguous base sequence at least 16 nucleotides in length selected from the group consisting of SEQ ID NOS:155, 156; 157, 158; 165, 166; 167, 168; 169, 170; 191 and SEQ ID NO:192.

40. Use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:19, 20, 87, 88 and sequences complementary thereto, or use in said method of a nucleic acid or oligomer comprising at least one contiguous base sequence having a length of at least 16 nucleotides selected from a second group of SEQ ID NOS:155 and 156, said method comprising determining the methylation state or level of methylation of at least one methylation variable positions (MVPs) within one or more sequences of the first sequence group; wherein the first group of tissues or cells comprises breast, brain and muscle cells or tissues, and the second group of tissues or cells comprises liver; lung and prostate cells or tissues.

41. Use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:21, 22, 89, 90 and sequences complementary thereto, or use in said method of a nucleic acid or oligomer comprising at least one contiguous base sequence having a length of at least 16 nucleotides selected from a second group of SEQ ID NOS:157 and 158, said method comprising determining the methylation state or level of methylation of at least one methylation variable position (MVPs) within one or more sequences of the first sequence group; wherein the first group of tissues or cells comprises breast, liver and muscle cells or tissues, and the second group of tissues or cells comprises lung and brain cells or tissues.

42. Use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:27, 28, 95, 96 and sequences complementary thereto, or use in said method of a nucleic acid or oligomer comprising at least one contiguous base sequence having a length of at least 16 nucleotides selected from a second group of SEQ ID NOS:163 and 164, said method comprising determining the methylation state or level of methylation of at least one methylation variable position (MVPs) within one or more sequences of the first sequence group; wherein the first group of tissues or cells comprises liver and muscle cells or tissues, and the second group of tissues or cells comprises breast and brain cells or tissues.

43. Use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:29, 30, 97, 98 and sequences complementary thereto, or use in said method of a nucleic acid or oligomer comprising at least one contiguous base sequence having a length of at least 16 nucleotides selected from a second group of SEQ ID NOS:165 and 166, said method comprising determining the methylation state or level of methylation of at least one methylation variable position (MVPs) within one or more sequences of the first sequence group; wherein the first group of tissues or cells comprises breast, brain and muscle cells or tissues, and the second group of tissues or cells comprises lung and prostate cells or tissues.

44. Use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:39, 40, 107, 108 and sequences complementary thereto, or use in said method of a nucleic acid or oligomer comprising at least one contiguous base sequence having a length of at least 16 nucleotides selected from a second group of SEQ ID NOS:175 and 176, said method comprising determining the methylation state or level of methylation of at least one methylation variable position (MVPs) within one or more sequences of the first sequence group; wherein the first group of tissues or cells comprises breast, and prostate cells or tissues, and the second group of tissues or cells comprises brain, lung and liver cells or tissues.

45. Use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:45, 46, 113, 114; 63, 64, 131, 132 and sequences complementary thereto, or use in said method of a nucleic acid or oligomer comprising at least one contiguous base sequence having a length of at least 16 nucleotides selected from a second group of SEQ ID NOS:181, 182, 199 and 200, said method comprising determining the methylation state or level of methylation of at least one methylation variable position (MVPs) within one or more sequences of the first sequence group; wherein the first group of tissues or cells comprises breast and muscle cells or tissues, and the second group of tissues or cells comprises lung, brain, liver and prostate cells or tissues.

46. Use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:67, 68, 135, 136 and sequences complementary thereto, or use in said method of a nucleic acid or oligomer comprising at least one contiguous base sequence having a length of at least 16 nucleotides selected from a second group of SEQ ID NOS:203 and 204, said method comprising determining the methylation state or level of methylation of at least one methylation variable position (MVPs) within one or more sequences of the first sequence group; wherein the first group of tissues or cells comprises breast and brain cells or tissues, and the second group of tissues or cells comprises lung, muscle, liver and prostate cells or tissues.

47. Use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:57, 58, 125, 126 and sequences complementary thereto, or use in said method of a nucleic acid or oligomer comprising at least one contiguous base sequence having a length of at least 16 nucleotides selected from a second group of SEQ ID NOS:193 and 194, said method comprising determining the methylation state or level of methylation of at least one methylation variable position (MVPs) within one or more sequences of the first sequence group; wherein the first group of tissues or cells comprises brain and muscle cells or tissues, and the second group of tissues or cells comprises lung, breast, liver and prostate cells or tissues.

48. Use of a nucleic acid or oligomer, in a method for distinguishing as the source of a nucleic acid sample, a first group of tissue or cells from a second group of tissues or cells, wherein said nucleic acid or oligomer comprises at least one contiguous base sequence having a length of at least 16 nucleotides that is complementary to, or hybridizes under moderately stringent or stringent conditions to a pretreated genomic DNA sequence selected from a first group consisting of SEQ ID NOS:17, 18, 85, 86 and sequences complementary thereto, or use in said method of a nucleic acid or oligomer comprising at least one contiguous base sequence having a length of at least 16 nucleotides selected from a second group of SEQ ID NOS:153 and 154, said method comprising determining the methylation state or level of methylation of at least one methylation variable position (MVPs) within one or more sequences of the first sequence group; wherein the first group of tissues or cells comprises breast and lung cells or tissues, and the second group of tissues or cells comprises brain, muscle, liver and prostate cells or tissues.

49. A method for diagnosing a condition or disease characterized by specific methylation levels or methylation states of one or more methylation variable genomic DNA positions in a disease-associated cell or tissue or in a sample derived from a bodily fluid, comprising:
a) obtaining a test cell, tissue sample or bodily fluid sample comprising genomic DNA having one or more methylation variable positions in one or more regions thereof;
b) determining the methylation state or quantified methylation level at the one or more methylation variable positions; and
c) comparing said methylation state or level to that of a genome wide methylation map according to claim 1, said map comprising methylation level values for at least one of corresponding normal, or diseased cells or tissue, whereby a diagnosis of a condition or disease is, at least in part afforded.

50. A method for detecting the absence or presence of a medical condition in an organ, cell type or tissue, comprising:
a) retrieving a bodily fluid sample;
b) determining at least one of the amount or presence, of free-floating DNA that exhibits a tissue-, organ- or cell type-specific DNA methylation pattern by use of a nucleic acid or oligomer comprising at least one contiguous base sequence having a length of at least 16 nucleotides that is complementary to, or hybridizes under moderately stringent or stringent conditions to a pretreated genomic DNA sequence selected from the group consisting of SEQ ID NOS:1 through SEQ ID NO:204 and SEQ ID NOS:206 through SEQ ID NO:221, and sequences complementary thereto;
c) determining whether there is an abnormal level of free floating DNA that originates from said tissue, cell type or organ, thereby concluding, whether a medical condition associated with said tissue, cell type or organ is absent or present.

51. A method for diagnosing a condition or disease of an individual characterized by the presence of organ- or tissue-specific free-floating DNA in said individual's bodily fluid, comprising:
a) retrieving a bodily fluid sample;
b) determining at least one of the amount or presence, of free floating DNA that exhibits a tissue-, organ- or cell type-characteristic DNA methylation pattern with the use of at least one nucleic acid or oligomer comprising at least one contiguous base sequence having a length of at least 16 nucleotides that is complementary to, or hybridizes under moderately stringent or stringent conditions to a pretreated genomic DNA sequence selected from the group consisting of SEQ ID NOS:1 through SEQ ID NO:204 and SEQ ID NOS:206 through SEQ ID NO:221, and sequences complementary thereto; and
c) further determining, whether there is an abnormal level of free-floating DNA that originates from said tissue, cell type or organ, and, at least in part thereby, concluding whether a medical condition associated with said tissue, cell type or organ is absent or present.

52. A method for diagnosing a condition or disease of an individual characterized by the presence of organ- or tissue-specific free-floating DNA in said individual's bodily fluid, comprising:
a) retrieving a bodily fluid sample;
b) determining the methylation states or methylation levels of MVPs within at least one nucleic acid or oligomer comprising at least one contiguous base sequence having a length of at least 16 nucleotides that is complementary to, or hybridizes under moderately stringent or stringent conditions to a pretreated genomic DNA sequence selected from the group consisting of SEQ ID NOS:1 through SEQ ID NO:204 and SEQ ID NOS:206 through SEQ ID NO:221 and sequences complementary thereto;
c) comparing said methylation states or levels to that of a genome-wide methylation map according to claim 1, said map comprising methylation level values of the corresponding nucleic acids for a plurality of normal organs, cells or tissues; and
d) determining whether the methylation states or levels of b) match with known values and whether a specific organ or tissue is dominant, whereby a diagnosis of a condition or disease is, at least in part, afforded.

53. A method according to claims 49-52 wherein said free-floating DNA is derived from a tissue or organ selected from the group consisting of lung, liver, muscle, breast, brain or prostate.

54. A method for at least one of choosing or monitoring a course of treatment, comprising, obtaining a diagnosis according to claims 49 to 52, whereby at least one of choosing or monitoring a course of treatment is, at least in part, afforded.

55. Use of a method according to any one of claims 49-53 for diagnosing a disease of an individual, diagnosing a condition of an individual, prognosing a disease of an individual, monitoring disease progression, monitoring treatment response, monitoring the occurrence of treatment side affects, or for classification, differentiation, grading, staging, or diagnosing of a cell proliferative disease or for a combination thereof.

56. A method for at least one of, identifying one organ, cell or tissue type, or distinguishing one organ, cell or tissue type from another as the source of a nucleic acid sample, comprising:
a) obtaining a nucleic acid sample having genomic DNA;
b) pretreating the genomic DNA, or a fragment thereof, with one or more agents to convert 5-position unmethylated cytosine bases to uracil or to another base that is detectably dissimilar to cytosine in terms of hybridization properties;
c) contacting the pretreated genomic DNA, or the pretreated fragment thereof, with an amplification enzyme and at least one primer set, each said set comprising first and second primer each having a contiguous sequence at least 16 nucleotides in length that is complementary to, or hybridizes under moderately stringent or stringent conditions to a sequence selected from, in the case of the first primer, a first group consisting of SEQ ID NOS:1-136, and selected from, in the case of the second primer, a second group consisting of sequences complementary to the sequences of the first group, wherein the pretreated DNA, or the fragment thereof is either amplified to produce one or more amplificates, or is not amplified; and
d) determining, based on the presence or absence of, or on a property of said amplificate, the methylation state or level of methylation of at least one MVP within the pretreated version of SEQ ID NO:205 or within a contiguous region thereof, or an average, or a value reflecting an average methylation state of a plurality of MVPs within the pretreated version of SEQ ID NO:205 or within a contiguous region thereof, whereby at least one of identifying one organ, cell or tissue type, or distinguishing one organ, cell or tissue type from another as the source of the nucleic acid sample is, at least in part afforded.

57. The method of claim 56, wherein in step b) treating the genomic DNA, or the fragment thereof, comprises use of a solution selected from the group consisting of bisulfite, hydrogen sulfite, disulfite, and combinations thereof.

58. The method according to any one of claims 56 or 57, wherein at least one of contacting in c), or determining in d) comprises use of a method selected from the group consisting of MSP, MethyLight™, HeavyMethyl™, MS-SNuPE™, and combination thereof.

59. The method according to any one of claims 56 or 57, wherein at least one of said primers comprises a sequence selected from the group consisting of SEQ ID NO:137 through SEQ ID NO:204.

60. The method according to any of claims 56 through 58, wherein the contiguous sequence of one or more of said primers comprises at least one 5′-CG-3′, 5′-tG-3′ or 5′-Ca-3′ dinucleotide.

61. The method according to any one of claims 55 through 59, further comprising in step c) the use of at least one oligomer comprising a contiguous sequence at least 16 nucleotides in length having one or more 5′-CG-3′, 5′-tG-3′ or 5′-Ca-3′ dinucleotides that were CG dinucleotides prior to pretreating in b) of claim 54, and wherein the contiguous sequence of said oligomer is complementary or identical to a sequence selected from the group consisting of SEQ ID NOS:1-136, and complements thereof, and wherein said oligomer suppresses amplification of the nucleic acid to which it is hybridized.

62. The method according to any one of claims 56 through 61, wherein determining the methylation state, or level of methylation or the average methylation state or average level of methylation comprises use of at least one reporter or probe oligomer that hybridizes to one or more 5′-CG-3′, 5′-TG-3′ or 5′-CA-3′ dinucleotides, at positions which were 5′-CG-3′ dinucleotides prior to pretreating, whereby amplification of one or more target sequences is, at least in part, afforded.

63. The use of a method according to any one of claims 56 through 62 for the analysis, characterization, classification, differentiation, grading, staging, diagnosis, or prognosis of cell proliferative disorders, or the predisposition to cell proliferative disorders, or combination thereof.

64. The use of a method according to any one of claims 56 through 62 for the the analysis, characterisation, classification, differentiation, grading, staging, or diagnosis or a combination thereof of prostate cancer, breast cancer, lung cancer, liver cancer or brain cancer, or the predisposition to said types of cancer.

65. A kit useful for identifying one tissue, organ or cell type as the source of a nucleic acid, or for distinguishing one tissue, organ or cell type from another among a group of tissue organ or cell types, as the source of a nucleic acid comprising:
a) a bisulfite reagent or a methylation-sensitive deamination enzyme; and
b) at least one oligomer comprising, in each case a contiguous sequence of at least 9 nucleotides in length that is complementary to, or hybridizes under moderately stringent or stringent conditions to a sequence selected from the group consisting of SEQ ID NOS:1-136, and complements thereof.

66. The kit of claim 65, wherein the tissue type group comprises at least two tissue types selected from the group consisting of prostate, breast, lung, liver, muscle and brain.

67. A kit useful for detecting, diagnosing, prognosing or differentiating cell proliferative disorders of the prostate, breast, lung, liver, muscle or brain, or for distinguishing between cell proliferative disorders of the prostate, breast, lung, liver, muscle or brain, comprising:
a) a bisulfite reagent or a methylation sensitive deamination enzyme; and
b) at least one nucleic acid molecule or peptide nucleic acid molecule comprising, in each case a contiguous sequence at least 9 nucleotides in length that is complementary to, or hybridizes under moderately stringent or stringent conditions to a sequence selected from the group consisting of SEQ ID NOS:1-136, and complements thereof.

68. The kit of any one of claims 65 through 67, further comprising standard reagents for performing a methylation assay selected from the group consisting of MS-SNuPE™, MSP, MethylLight™, HeavyMethyl™, COBRA™, nucleic acid sequencing, and combinations thereof.

69. A method of providing diagnostic information relating to cancer, comprising:
a) determining the relative amount of free-floating DNA derived from a specific organ or tissue within the total amount of free-floating DNA in a bodily fluid sample of a patient suspected of suffering from a cell proliferative disorder, wherein said determining comprises determination of the level of methylation of at least three MVPs or CpGs selected from the group identified in Tables 37-70 in said bodily fluid sample, and wherein a methylation pattern is provided;
b) comparing said methylation pattern with methylation patterns found in a plurality of samples that have been identified to be characteristic for specific organs or tissues out of a group of other organs or tissues;
c) determining, in relation to samples from healthy donors, whether the methylation pattern determined in a) indicates an increased relative amount of free-floating DNA derived from a specific organ or tissue within the total amount of free-floating DNA in said bodily fluid, whereby a conclusion as to whether said patient has an increased risk of developing cancer is, at least in part, afforded.

70. The method of claim 67, wherein the methylation pattern in a) comprises the levels of methylation of at least 5 CpG positions.

71. The method of claim 67, wherein at least three MVPs or CpG positions of which the level of methylation is determined, are located within a 500 bp genomic region.
